SEQUENCE LISTING (86 sequences)

SEQ ID NO: 1 - DNA, 10 bp, synthetic construct
  source 1..10: mol_type = other DNA
dgagtrrghv

SEQ ID NO: 2-12 - skipped (11 entries)

SEQ ID NO: 13 - DNA, 10 bp, synthetic construct
  source 1..10: mol_type = other DNA
agaggtagag

SEQ ID NO: 14 - DNA, 10 bp, synthetic construct
  source 1..10: mol_type = other DNA
tgaggttgag

SEQ ID NO: 15 - DNA, 10 bp, synthetic construct
  source 1..10: mol_type = other DNA
ggaggtggag

SEQ ID NO: 16-20 - skipped (5 entries)

SEQ ID NO: 21 - DNA, 556 bp, synthetic construct
  source 1..556: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttggagtcaatccacaccatggaggaggttcatgattgtgtagagtaagacataattttgttgaggtttaactctgaatacttaatgtggtactgaatacttggtgtggtactgagaggcagcctaactgaccacacagcattcacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 22 - DNA, 556 bp, synthetic construct
  source 1..556: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttggagtcaatccaagtaatgcaggaggttcatgattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 23 - DNA, 556 bp, synthetic construct
  source 1..556: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttggagtcaatccaagtaacgcaggacaccatggattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 24 - DNA, 556 bp, synthetic construct
  source 1..556: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttggagtcaatccaagtaacgcaggaggttcacaccatggtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 25 - DNA, 556 bp, synthetic construct
  source 1..556: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttggagtcaatccacaccatggaggaggttcatgaaagggatgagtaagcagtcattttgttgaggtttaactctgaatacttaatgtggtactgaatacttggtgtggtactgagaggcagcctaactgaccacacagcattcacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 26 - DNA, 1008 bp, synthetic construct
  source 1..1008: mol_type = other DNA
atgtttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgccaagtcgtgtgcctattgctgtaagacatactgagatgtgtaagacctgatctttgatcttgaatttgacatcaggttgggtaaaaagaatgtctgaaggtgtttgtctggttatcagagagtaatttaaataaagggaaggttggaccttggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttggagtcaatccaagtaatgcaggaggttcatgattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 27 - DNA, 1108 bp, synthetic construct
  source 1..1108: mol_type = other DNA
atgtttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgccaagtcgtgtgcctattgctgtaagacatactgagatgtgtaagacctgatctttgatcttgaatttgacatcaggttgggtaaaaagaatgtctgaaggtgtttgtctggttatcagagagtaatttaaataaagggaaggttggaccttggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttggagtcaatccaagtaatgcaggaggttcatgattgtgtagagtaagacataattttgttgaggtttaactctgaatacttaatgtggtactgaatacttaatgtggtactgagaggcagcctaactgaccacacagcattcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 28 - DNA, 1005 bp, synthetic construct
  source 1..1005: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgccaagtcgtgtgcctattgctgtaagacatactgagatgtgtaagacctgatctttgatcttgaatttgacatcaggttgggtaaaaagaatgtctgaaggtgtttgtctggttatcagagagtaatttaaataaagggaaggttggaccttggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttggagtcaatccaagtaatgcaggaggttcatgattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 29 - DNA, 1005 bp, synthetic construct
  source 1..1005: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgccaagtcgtgtgcctattgctgtaagacatactgagatgtgtaagacctgatctttgatcttgaatttgacatcaggttgggtaaaaagaatgtctgaaggtgtttgtctggttatcagagagtaatttaaataaagggaaggttggaccttggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttggagtcaatccacaccatggaggaggttcatgattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 30 - DNA, 905 bp, synthetic construct
  source 1..905: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgccaagtcgtgtgcctattgctgtaagacatactgagatgtgtaagacctgatctttgatcttgaatttgacatcaggttgggtaaaaagaatgtctgaaggtgtttgtctggttatcagagagtaatttaaataaagggaaggttggaccttggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttggagtcaatccacaccatggaggaggttcatgattgtgtagagtaagacataattttgttgaggtttaactctgaatacttaatgtggtactggccaggctgggttctggttgtttatgatctttattttttggtgatctaggaaccaacaacaagaaattgttgtttcccgtggg

SEQ ID NO: 31 - DNA, 928 bp, synthetic construct
  source 1..928: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgccaagtcgtgtgcctattgctgtaagacatactgagatgtgtaagacctgatctttgatcttgaatttgacatcaggttgggtaaaaagaatgtctgaaggtgtttgtctggttatcagagagtaatttaaataaagggaaggttggaccttggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttggagtcaatccacaccatggaggaggttcatgattgtgtagagtaagacataattttgttgaggtttaactctgaatacttaatgtggtacttagagacggggtttcaccatggtggccaggctgggttctggttgtttatgatctttattttttggtgatctaggaaccaacaacaagaaattgttgtttcccgtggg

SEQ ID NO: 32 - DNA, 506 bp, synthetic construct
  source 1..506: mol_type = other DNA
tttctgtacaacttaaccttgcagagagccactggcatcagctttgccattcttggaaacttttctggtaagttctctcgttaccatcttttgaaattttaagtgaattaatacatagaattgtgtgaaatttcaaatattggattaggaaatacaaagttactgaaagtgaggtactaatgtttataaaataaaaactttttcttgccatttgcagatttaacatttttggagtcaatccacaccatggaggaggttcatgattgtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 33 - DNA, 977 bp, synthetic construct
  source 1..977: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgccaagtcgtgtgcctattgctgtaagacatactgagatgtgtaagacctgatctttgatcttgaatttgacatcaggttgggtaaaaagaatgtctgaaggtgtttgtctggttatcagagagtaatttaaataaagggaaggttggaccttggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacacacaccatggaggtgtagagtaagacataattttgttgaggtttaactctgaatacttaatgtggtactgaatacttaatgtggtactgagaggcagcctaactgaccacacagcattcacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 34 - DNA, 960 bp, synthetic construct
  source 1..960: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgccaagtcgtgtgcctattgctgtaagacatactgagatgtgtaagacctgatctttgatcttgaatttgacatcaggttgggtaaaaagaatgtctgaaggtgtttgtctggttatcagagagtaatttaaataaagggaaggttggaccttggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttataaaatgcttaaattatggataacattttcaatgaaattaactatattttcaaatgttctgccttttttctctgtttcttttgcccttcctgatcgaagtcatgggagagtaagtcaatactttttatgagaatatattgtcctacttgtatgtctaaagaccatgttctcgttctgcagttcaacgttgttgattttatttcttacatacgcctggctaatttttgtatttttagtagagacggggtttcaccatggtggccaggctgggttctggttgtttatgatctttattttttggtgatctaggaaccaacaacaagaaattgttgtttcccgtggg

SEQ ID NO: 35 - DNA, 540 bp, synthetic construct
  source 1..540: mol_type = other DNA
ggctagcgcttttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttggagtcaatccacaccatggaggaggttcatgattgtgtagagtaagacataattttgttgaggtttaactctgaatacttaatgtggtactgaatacttggtgtggtactgagaggcagcctaactgaccacacagcattagtagagacggggtttcaccatggtggccaggctgggttctggttgtttatgatctttattttttggtgatctaggaaccaacaacaagaaattgttgtttcccgtggg

SEQ ID NO: 36 - DNA, 1199 bp, synthetic construct
  source 1..1199: mol_type = other DNA
atggtcttcttaaacacttccagtaaccatgaaaactcagacttggaaatgccttctttaacttccacccccctcattggtcacaaattgactgttacaacaccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataattttactgaccatttatatataatcatgtctattcccacattgaatatatctgtcctgggcatattacaactacctacaggtagaagatgggaagataaactagattccttttgtaacttactccaaaatttcctatatatgcacaagtagttttcataaaacttttccattcggtttgtttgttccctccataaggccttcttatgaaagccagattttcaacctcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttatttcgaagtacaaaaagtaaattttaaaagtttaacatgttttgtagtatgggctctgttttcatgaatagggaaacctgatttgtaaatgacatcaggtcttcttgatgctcatagtcttatacagatcataggcactacatgtgtcttcagagccattaaggaaagatgttaaatcatttaatgcttttagtcttcgaggtataatattttgaaatatttgaaaatatgtgatatgcatattctctttttcttaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaaccccaggg

SEQ ID NO: 37 - DNA, 523 bp, synthetic construct
  source 1..523: mol_type = other DNA
atggtcccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattgtttattgtattcagtggaaaaatgttaaatgtaacaggtaaagccatgtagggacatttattcttttgtattgagccactgcttgttttctttttaaagttttagaaccccag

SEQ ID NO: 38 - DNA, 623 bp, synthetic construct
  source 1..623: mol_type = other DNA
atggtcccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaaccccag

SEQ ID NO: 39 - DNA, 686 bp, synthetic construct
  source 1..686: mol_type = other DNA
atggtcccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaaccccagggctcgagcgagggcaggggaagtcttctaacatgcggggacgtggaggaaaatcccggccca

SEQ ID NO: 40 - DNA, 619 bp, synthetic construct
  source 1..619: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaaccccaggg

SEQ ID NO: 41 - DNA, 617 bp, synthetic construct
  source 1..617: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccacaccatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaaccccag

SEQ ID NO: 42 - DNA, 623 bp, synthetic construct
  source 1..623: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttggcggctcgagcggcgtg

SEQ ID NO: 43 - DNA, 625 bp, synthetic construct
  source 1..625: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttaggggctcgagcggcgtg

SEQ ID NO: 44 - DNA, 628 bp, synthetic construct
  source 1..628: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaaggggctcgagcggcgtg

SEQ ID NO: 45 - DNA, 631 bp, synthetic construct
  source 1..631: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaacccggggctcgagcggcgtg

SEQ ID NO: 46 - DNA, 620 bp, synthetic construct
  source 1..620: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaaccccaggtg

SEQ ID NO: 47 - DNA, 623 bp, synthetic construct
  source 1..623: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttgtgggctcgagcggcgtg

SEQ ID NO: 48 - DNA, 611 bp, synthetic construct
  source 1..611: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagtg

SEQ ID NO: 49 - DNA, 614 bp, synthetic construct
  source 1..614: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaagtg

SEQ ID NO: 50 - DNA, 617 bp, synthetic construct
  source 1..617: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattcagagaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaacccgtg

SEQ ID NO: 51 - DNA, 618 bp, synthetic construct
  source 1..618: mol_type = other DNA
ccatttcatagagaccagactgtgaaaactcaaaaggaaaatactgtgtaagtctttggttcaggaataaaatgatttatcttatttacttatatttaattaatggaatatagttcccagatgtctgatccacttgtatgtgcataatcctttcctctctaattgtaagcttcagaataagaacgagtatggtttcttacctagtttgtatttttatttaattttatttgtttattatttatttatttttagtttgtattttttaatcttattttttctttttgaatttatgtgtagatcggacctcttctcacatccccaggaatattatatgattagaagccaagggatgagtaagcagtcatttttattcaataaagccttaatcaattccatgaaatggtagagaaagtgtgaattatcagaaaccttgaaaagaaatgcactgttattaacgaataacgtgattaatcagcacaccccaattccatatccggaacagagtttattgtattcagtggaaaaatgttaaatgtaacaggtaaagccatgtagggacatttattcttttgtattgagccactgcttgttttctttttaaagttttagaaccccagg

SEQ ID NO: 52 - DNA, 437 bp, synthetic construct
  source 1..437: mol_type = other DNA
atggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtttgtgacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 53 - DNA, 437 bp, synthetic construct
  source 1..437: mol_type = other DNA
acggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtttgcgacttaattattttgggggctgctatgttaaagtgtgtcagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 54 - DNA, 439 bp, synthetic construct
  source 1..439: mol_type = other DNA
atggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtttgtgacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttcccccctatgttcttagtctcaggatgcagggg

SEQ ID NO: 55 - DNA, 437 bp, synthetic construct
  source 1..437: mol_type = other DNA
atggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtttgtgacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcatgctttccccccttgtcttagtctcaggatgcagggg

SEQ ID NO: 56 - DNA, 437 bp, synthetic construct
  source 1..437: mol_type = other DNA
atggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtttgtgacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatgagcttttccccccttgtcttagtctcaggatgcagggg

SEQ ID NO: 57 - DNA, 439 bp, synthetic construct
  source 1..439: mol_type = other DNA
atggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtttgtgacttaattattttgggggctgctatgttaaagtgtgttagtttaagaatactagttctgtctgtagctctaatcctctgagagcttgggtttctaaattattctgtgttgctcttgtatgggttttgtttctgttgtttttggtttgttttgttgtactatcagcttttccccccttgtcttagtctcaggatgcagggg

SEQ ID NO: 58 - DNA, 438 bp, synthetic construct
  source 1..438: mol_type = other DNA
atggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtttgtgacttaattattttgggggctgctatgttaaagtgtgttagtttaagaatactagttctgtctgtagctctaatcctctgagagcttgggtttctaaattattctgtgatggctcttgtaggttttgtttctgttgtttttggtttgttttgttgtactatcagcttttccccccttgtcttagtctcaggatgcagggg

SEQ ID NO: 59 - DNA, 437 bp, synthetic construct
  source 1..437: mol_type = other DNA
atggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtaagtgacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 60 - DNA, 437 bp, synthetic construct
  source 1..437: mol_type = other DNA
atggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtatgtgacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 61 - DNA, 437 bp, synthetic construct
  source 1..437: mol_type = other DNA
atggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactcagagtaagtgacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 62 - DNA, 431 bp, synthetic construct
  source 1..431: mol_type = other DNA
ggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtatgtgacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 63 - DNA, 437 bp, synthetic construct
  source 1..437: mol_type = other DNA
atggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtatgtcacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 64 - DNA, 437 bp, synthetic construct
  source 1..437: mol_type = other DNA
atggtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtatgacacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 65 - DNA, 437 bp, synthetic construct
  source 1..437: mol_type = other DNA
agtgtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcactatgagtatgacacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 66 - DNA, 437 bp, synthetic construct
  source 1..437: mol_type = other DNA
agtgtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaattcaccatgagtatgacacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 67 - DNA, 437 bp, synthetic construct
  source 1..437: mol_type = other DNA
agtgtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagatgaaatgactatgagtatgacacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 68 - DNA, 437 bp, synthetic construct
  source 1..437: mol_type = other DNA
agtgtcggtttaccattgaggtaagaaaatgttagtcatcaacttaagtatagaaatttataggagcgatcctaatacttctggctggaattagtattaaagggtaatgtatcaatatgctttattatttgaaattaacagttgctgaaatgtcaaaccatgcactaatgggaattttctgtttttccctttaacagtattggatatgacagggaagacaccatggctatgagtatgacacttaattattttgggggctgctatgttaaagtgtgttagtttaagaatactagttctgtctgtagctctaatcctctgagagcttgggtttctaaattattctgtgttgctcttgtaggttttgtttctgttgtttttggtttgttttgttgtactatcagcttttccccccttgtcttagtctcaggatgcagggg

SEQ ID NO: 69 - DNA, 554 bp, synthetic construct
  source 1..554: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttggagtcaatccaagtaacgcaggaggttcatgattgtgtagagtaagacataattttgttgaggtttaactctgaatacttaatgtggtactgaatacttggtgtggtactgagaggcagcctaactgaccacacagcattcacgcctggctaatttttgtatttttagtagagacggggtttcaccatggtggccaggctgggttctggttgtttatgatctttattttttggtgatctagtggccaacaacaagaaattgttgtttcccgtg

SEQ ID NO: 70 - DNA, 554 bp, synthetic construct
  source 1..554: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttggagtcaatccaagtaacgcaggaggttcttgattgtgtagagtaagacataattttgttgaggtttaactctgaatacttaatgtggtactgaatacttggtgtggtactgagaggcagcctaactgaccacacagcattcacgcctggctaatttttgtatttttagtagagacggggtttcaccatggtggccaggctgggttctggttgtttatgatctttattttttggtgatctagtggccaacaacaagaaattgttgtttcccgtg

SEQ ID NO: 71 - DNA, 554 bp, synthetic construct
  source 1..554: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttggagtcaatccaagtaacgcaggaggttcttgattgtcaagagtaagacataattttgttgaggtttaactctgaatacttaatgtggtactgaatacttggtgtggtactgagaggcagcctaactgaccacacagcattcacgcctggctaatttttgtatttttagtagagacggggtttcaccatggtggccaggctgggttctggttgtttatgatctttattttttggtgatctagtggccaacaacaagaaattgttgtttcccgtg

SEQ ID NO: 72 - DNA, 554 bp, synthetic construct
  source 1..554: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttggagtcaatccaagtaacgcaggaggttcttggccgccaagagtaagacataattttgttgaggtttaactctgaatacttaatgtggtactgaatacttggtgtggtactgagaggcagcctaactgaccacacagcattcacgcctggctaatttttgtatttttagtagagacggggtttcaccatggtggccaggctgggttctggttgtttatgatctttattttttggtgatctagtggccaacaacaagaaattgttgtttcccgtg

SEQ ID NO: 73 - DNA, 525 bp, synthetic construct
  source 1..525: mol_type = other DNA
tttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacaggaattgtgtgaaatttcaaatattggattaggaaatacaaagttactgaaagtgaggtactaatgtttataaaataaaaactttttcttgccatttgcagatttaacatttttggagtcaatccaagtaacgcaggaggttcttggccgccaagagtaagacataattttgttgaggtttaactctgaatacttaatgtggtactgaatacttggtgtggtactgagaggcagcctaactgaccacacagcattcacgcctggctaatttttgtatttttagtagagacggggtttcaccatggtggccaggctgggttctggttgtttatgatctttattttttggtgatctagtgg

SEQ ID NO: 74 - DNA, 1650 bp, synthetic construct
  source 1..1650: mol_type = other DNA
gaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 75 - DNA, 12 bp, synthetic construct
  source 1..12: mol_type = other DNA
tagagtaagaca

SEQ ID NO: 76 - DNA, 12 bp, synthetic construct
  source 1..12: mol_type = other DNA
atgagtatgaca

SEQ ID NO: 77 - DNA, 12 bp, synthetic construct
  source 1..12: mol_type = other DNA
atgagtaagcag

SEQ ID NO: 78 - DNA, 10 bp, synthetic construct
  source 1..10: mol_type = other DNA
atgagtatgt

SEQ ID NO: 79 - DNA, 10 bp, synthetic construct
  source 1..10: mol_type = other DNA
atgagtttgt

SEQ ID NO: 80 - DNA, 10 bp, synthetic construct
  source 1..10: mol_type = other DNA
atgagtaagt

SEQ ID NO: 81 - DNA, 10 bp, synthetic construct
  source 1..10: mol_type = other DNA
atgagttagt

SEQ ID NO: 82 - DNA, 10 bp, synthetic construct
  source 1..10: mol_type = other DNA
dgagtddghv

SEQ ID NO: 83 - skipped

SEQ ID NO: 84 - DNA, 10 bp, synthetic construct
  source 1..10: mol_type = other DNA
dgagtttghv

SEQ ID NO: 85 - protein, 549 aa, synthetic construct
EDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 86 - DNA, 10 bp, synthetic construct
  source 1..10: mol_type = other DNA
  misc_feature 1..5: note = RNA
  misc_feature 6: note = DNA
  misc_feature 7..10: note = RNA
tgaggttgag

(19) United States
(12) Patent Application Publication
(10) Pub. No.: US 2026/0327655 A1
(43) Pub. Date: Oct. 22, 2026

(54) SMALL-MOLECULE REGULATED ALTERNATIVE SPLICING

(71) Applicant: Rgenta Therapeutics, Inc., Woburn, MA (US)

(72) Inventors: Samuel Hasson, Woburn, MA (US); Hualin Xi, Woburn, MA (US); Travis T. Wager, Brookline, MA (US); Jae Lee, Woburn, MA (US); Kai Li, Woburn, MA (US); Zhiping Weng, Woburn, MA (US); Ian Mclachlan, Woburn, MA (US)

(21) Appl. No.: 19/475,291

(22) PCT Filed: Apr. 13, 2024
(86) PCT No.: PCT/US2024/024522

§ 371 Date: Oct. 14, 2025

Related U.S. Application Data
(60) Provisional application No. 63/496,199, filed on Apr. 14, 2023.

(51) Int. Cl. C12N 15/864; A61K 31/711; A61K 48/00

(52) U.S. Cl. CPC C12N 15/8645; A61K 31/711; A61K 48/0058; A61K 48/0066; C12N 2310/141; C12N 2320/33; C12N 2750/14132; C12N 2750/14143; C12N 2750/14171; C12N 2800/10; C12N 2830/34; C12N 2830/50; C12N 2840/44

(57) ABSTRACT

The present disclosure relates to nucleic acids for use in regulating transcription of a linked transgene by an exon-inclusion mechanism.

### CROSS-REFERENCE TO RELATED APPLICATIONS

This application claims the benefit of and priority to U.S. Provisional Patent Application No. 63/496,199, filed Apr. 14, 2023, the disclosure of which is hereby incorporated by reference in its entirety for all purposes.

### FIELD OF THE DISCLOSURE

The present disclosure provides a nucleic acid molecule for use in regulating transcription of a linked transgene by an exon-inclusion mechanism.

### BACKGROUND

Regulation of gene expression includes a wide range of mechanisms that are used by cells to increase or decrease the production of specific gene products. Sophisticated programs of gene expression are widely observed in biology, for example to trigger developmental pathways, respond to environmental stimuli, or adapt to new food sources. In the pathology of diseases or disorders, the regulation of various genetic pathways affects the initiation and development of a number of the diseases or disorders. Several steps of gene expression can be modulated—from transcriptional initiation, to RNA processing, and to the post-translational modification of a protein. In a gene regulatory network, often, one gene regulator controls a second gene regulator, the second gene regulator controls a third, and the pattern may continue to form a subsequent cascade of gene regulators controlling other gene regulators.

Regulation of transcription controls when transcription occurs and how much RNA is created. Transcription of a gene by RNA polymerase and subsequent splicing of pre-mRNA can be regulated by several endogenous mechanisms. Translation of a protein can also be regulated. For example, one mechanism includes the regulation of the translation of mRNA at the level of initiation, which occurs by the recruitment of a small ribosomal subunit that can be modulated by mRNA secondary structure, antisense RNA binding, protein, or ligand (e.g., a small molecule) binding. In some embodiments, a class of transcripts (e.g., riboswitches) act as ribozymes (RNAs having self-catalytic activity) and self-regulate their expression, acting as either a negative or a positive feedback loop.

Regulators of transcription, splicing, and translation can be used in synthetic biology to affect an increase or decrease of the levels of a protein of interest. Without being bound by theory, increases in the level of polypeptides may provide therapeutic effects by providing a polypeptide whose expression is reduced or missing in a subject's tissue or decreases in the level of a polypeptide may provide therapeutic benefits by providing for the reduction of a polypeptide whose expression is increased or aberrant in a subject's tissue. Without being bound by theory, controlling the timing or location of expression of genes, e.g., by application or withdrawal of a splice modulator, may improve the effectiveness and/or safety of such a therapeutic protein by ensuring expression is conditionally (e.g., temporally or tissue-specifically) regulated. Non-limiting examples of genes that can be expressed include a protein, an RNA, microRNA (miRNA) or short hairpin RNA (shRNA). Accordingly, the present disclosure provides, in part, nucleic acid molecules that are useful to turn on expression of a transgene using a splice modulator (e.g., small molecule). The disclosure also provides vectors and pharmaceutical compositions including such nucleic acid molecules and splice modulators (e.g., small molecules), and contemplates their use in methods of regulate gene expression.

### SUMMARY

Provided herein, in certain embodiments, is a nucleic acid molecule comprising a minigene positioned immediately 5′ to a transgene, the minigene comprising: (a) a first exon immediately 5′ to a first intron; (b) a start codon; and (c) a splice modulator binding site, the splice modulator binding site comprises the nucleic acid sequence of DGAGTDDGHV (SEQ ID NO: 82) or DGAGTDDNHV (SEQ ID NO: 83), wherein D is A, G, or T; wherein R is A, or G; wherein N is A, C, G, or T; wherein H is A, C, or T; and wherein V is A, G, or C.

In some embodiments, the splice modulator binding site comprises the nucleic acid sequence of DGAGTRRGHV (SEQ ID NO: 1) or DGAGTRRNHV (SEQ ID NO: 2), wherein D is A, G, or T; wherein R is A or G; wherein N is A, C, G, or T; wherein H is A, C, or T; and wherein V is A, G, or C.

In some embodiments, the splice modulator binding site comprises the nucleic acid sequence of DGAGTTTGHV (SEQ ID NO: 84), wherein D is A, G, or T; wherein H is A, C, or T; and wherein V is A, G, or C.

In some embodiments, the nucleic acid comprises the splice modulator binding site comprising any one of SEQ ID NOs: 75-81.

In some embodiments, the nucleic acid comprises in 5′ to 3′ order the first exon comprising the start codon, the first intron, and the transgene.

In some embodiments, the nucleic acid further comprises a stop codon.

In some embodiments, the nucleic acid further comprises a second exon.

In some embodiments, the nucleic acid comprises in 5′ to 3′ order the first exon comprising the start codon, the second exon, and the transgene.

In some embodiments, the nucleic acid comprises in 5′ to 3′ order the first exon comprising the start codon, the first intron, the second exon, and the transgene.

In some embodiments, the nucleic acid comprises in 5′ to 3′ order the first exon, the second exon comprising the start codon, and the transgene.

In some embodiments, the nucleic acid comprises in 5′ to 3′ order the first exon, the second exon comprising the start codon, the first intron, and the transgene.

In some embodiments, the nucleic acid comprises in 5′ to 3′ order the first exon, the first intron, the second exon comprising the start codon, a second intron, and the transgene

In some embodiments, the nucleic acid comprises at least about 80% sequence identity to any one of SEQ ID NOs: 21-73.

In some embodiments, the nucleic acid comprises at least about 90% sequence identity to any one of SEQ ID NOs: 21-73.

In some embodiments, the nucleic acid comprises any one of SEQ ID NOs: 21-73.

In some embodiments, the nucleic acid further comprises a third exon. In some embodiments, the nucleic acid further comprises a third intron.

In some embodiments, the nucleic acid comprises in 5′ to 3′ order the first exon, the second exon comprising the stop codon, the third exon comprising the start codon, and the transgene.

In some embodiments, the nucleic acid comprises in 5′ to 3′ order the first exon, the first intron, the second exon comprising the stop codon, the second intron, the third exon comprising the start codon, the third intron, and the transgene.

In some embodiments, the nucleic acid comprises in 5′ to 3′ order the first exon, the first intron, the second exon comprising the stop codon, the third exon comprising the start codon, the second intron, and the transgene.

In some embodiments, the nucleic acid comprises in 5′ to 3′ order the first exon, the second exon comprising the start codon, the third exon comprising the stop codon, and the transgene.

In some embodiments, the nucleic acid comprises in 5′ to 3′ order the first exon, the first intron, the second exon comprising the start codon, the second intron, the third exon comprising the stop codon, the third intron, and the transgene.

In some embodiments, the nucleic acid comprises in 5′ to 3′ order the first exon, the first intron, the second exon comprising the start codon, the third exon comprising the stop codon, the second intron, and the transgene.

Provided herein, in certain embodiments, is a nucleic acid molecule comprising a minigene positioned immediately 5′ to a transgene, the minigene comprising: (a) a first exon 5′ to a first intron; (b) a start codon comprising a first portion and a second portion, wherein the first portion and second portion of the start codon are not in the same exon, and (c) a splice modulator binding site, In some embodiments, the splice modulator binding site comprises the nucleic acid sequence of DGAGTDDGHV (SEQ ID NO: 82) or DGAGTDDNHV (SEQ ID NO: 83), wherein D is A, G, or T; wherein R is A, or G; wherein N is A, C, G, or T; wherein H is A, C, or T; and wherein V is A, G, or C.

In some embodiments, the splice modulator binding site comprises the nucleic acid sequence of DGAGTRRGHV (SEQ ID NO: 1) or DGAGTRRNHV (SEQ ID NO: 2), wherein D is A, G, or T; wherein R is A or G; wherein Nis A, C, G, or T; wherein H is A, C, or T; and wherein V is A, G, or C.

In some embodiments, the splice modulator binding site comprises the nucleic acid sequence of DGAGTTTGHV (SEQ ID NO: 84), wherein D is A, G, or T; wherein H is A, C, or T; and wherein V is A, G, or C.

In some embodiments, the first portion of the start codon comprises one or two nucleotides of the start codon, and the second portion of the start codon has one or two nucleotides of the start codon.

In some embodiments, the first portion of the start codon is located in the first exon, and wherein the second portion of the start codon is located in the transgene.

In some embodiments, the nucleic acid comprises in 5′ to 3′ order the first exon comprising the first portion of the start codon, the first intron, and the transgene comprising the second portion of the start codon.

In some embodiments, the nucleic acid further comprises a second exon. In some embodiments, the nucleic acid further comprises a second intron.

In some embodiments, the nucleic acid comprises in 5′ to 3′ order the first exon comprising the first portion of the start codon, the second exon comprising the second portion of the start codon, and the transgene.

In some embodiments, the nucleic acid comprises in 5′ to 3′ order the first exon comprising the first portion of the start codon, the first intron, the second exon comprising the second portion of the start codon, the second intron, and the transgene.

In some embodiments, the nucleic acid comprises in 5′ to 3′ order the first exon, the second exon comprising the first portion of the start codon, and the transgene comprising the second portion of the start codon.

In some embodiments, the nucleic acid comprises in 5′ to 3′ order the first exon, the second exon comprising the first portion of the start codon, the first intron, and the transgene comprising the second portion of the start codon.

In some embodiments, the nucleic acid comprises in 5′ to 3′ order the first exon comprising the first portion of the start codon, the second exon, and the transgene comprising the second portion of the start codon.

In some embodiments, the nucleic acid comprises in 5′ to 3′ order the first exon comprising the first portion of the start codon, the second exon, the first intron, and the transgene comprising the second portion of the start codon.

In some embodiments, the nucleic acid comprises in 5′ to 3′ order the first exon comprising the first portion of the start codon, the second exon comprising the second portion of the start codon, and the transgene.

In some embodiments, the nucleic acid comprises in 5′ to 3′ order the first exon comprising the first portion of the start codon, the first intron, the second exon comprising the second portion of the start codon, and the transgene.

In some embodiments, the nucleic acid further comprises a stop codon.

In some embodiments, the nucleic acid comprises in 5′ to 3′ order the first exon comprising the first portion of the start codon, the second exon comprising the stop codon, and the transgene comprising the second portion of the start codon.

In some embodiments, the nucleic acid comprises in 5′ to 3′ order the first exon comprising the first portion of the start codon, the first intron, the second exon comprising the stop codon, and the transgene comprising the second portion of the start codon.

In some embodiments, the second exon comprises the splice modulator binding site.

In some embodiments, the nucleic acid further comprises a third exon.

In some embodiments, the nucleic acid further comprises a third intron.

In some embodiments, the nucleic acid comprises in 5′ to 3′ order the first exon comprising the first portion of the start codon, the second exon comprising the stop codon, the third exon comprising the second portion of the start codon, and the transgene.

In some embodiments, the nucleic acid comprises in 5′ to 3′ order the first exon comprising the first portion of the start codon, the first intron, the second exon comprising the stop codon, the third exon comprising the second portion of the start codon, the second intron, and the transgene.

In some embodiments, the nucleic acid comprises in 5′ to 3′ order the first exon, the second exon comprising first portion of the start codon, the third exon comprising the stop codon, and the transgene comprising the second portion of the start codon.

In some embodiments, the nucleic acid comprises in 5′ to 3′ order the first exon, the first intron, the second exon comprising first portion of the start codon, the third exon comprising the stop codon, the second intron, and the transgene comprising the second portion of the start codon.

In some embodiments, the nucleic acid comprises in 5′ to 3′ order the first exon, the second exon comprising the stop codon, the third exon comprising first portion of the start codon, and the transgene comprising the second portion of the start codon.

In some embodiments, the nucleic acid comprises in 5′ to 3′ order the first exon, the first intron, the second exon comprising the stop codon, the third exon comprising first portion of the start codon, the second intron, and the transgene comprising the second portion of the start codon.

In some embodiments, the nucleic acid comprises in 5′ to 3′ order the first exon, the second exon, the third exon comprising the first portion of the start codon, and the transgene comprising the second portion of the start codon.

In some embodiments, the nucleic acid comprises in 5′ to 3′ order the first exon, the first intron, the second exon, the third exon comprising the first portion of the start codon, the second intron, and the transgene comprising the second portion of the start codon.

In some embodiments, the nucleic acid comprises in 5′ to 3′ order the first exon, the second exon comprising the stop codon, the third exon comprising the first portion of the start codon, and the transgene comprising the second portion of the start codon.

In some embodiments, the nucleic acid comprises in 5′ to 3′ order the first exon, the first intron, the second exon comprising the stop codon, the second intron, the third exon comprising the first portion of the start codon, the third intron, and the transgene comprising the second portion of the start codon.

In some embodiments, the nucleic acid comprises in 5′ to 3′ order the first exon, the second exon comprising the first portion of the start codon, the third exon comprising the stop codon, and the transgene comprising the second portion of the start codon.

In some embodiments, the nucleic acid comprises in 5′ to 3′ order the first exon, the first intron, the second exon comprising the first portion of the start codon, the second intron, the third exon comprising the stop codon, the third intron, and the transgene comprising the second portion of the start codon.

In some embodiments, the first intron comprises the splice modulator binding site. In some embodiments, the splice modulator binding site is located at the junction between the second exon and the second intron. In some embodiments, the splice modulator binding site is located within the second intron.

Provided herein, in certain embodiments, is a nucleic acid molecule comprising in 5′ to 3′: (a) a first exon comprising a first portion of a start codon at the 3′ end of the first exon; (b) a second exon comprising a second portion of a start codon at the 5′ end of the second exon; and (c) a third exon. In some embodiments, the third exon is a transgene.

Provided herein, in certain embodiments, is a nucleic acid molecule comprising in 5′ to 3′ order a first exon, a second exon comprising a stop codon, a third exon comprising a first portion of a start codon, and a transgene comprising a second portion of the start codon.

Provided herein, in certain embodiments, is a nucleic acid molecule comprising in 5′ to 3′ order a first exon, a second exon comprising a first portion of a start codon, a third exon comprising a stop codon, and a transgene comprising a second portion of the start codon.

In some embodiments, the nucleic acid molecule further comprises a first intron, a second intron, and a third intron interposed between the first exon, the second exon, the third exon, and the transgene, respectively.

In some embodiments, the splice modulator binding site does not comprise the nucleic acid sequence AAGAGT (SEQ ID NO: 3), ATGAGT (SEQ ID NO: 4), TAGAGT (SEQ ID NO: 5), TTGAGT (SEQ ID NO: 6), GAGAGT (SEQ ID NO: 7), GTGAGT (SEQ ID NO: 8), AAGAGT (SEQ ID NO: 9), ATGAGT (SEQ ID NO: 10), ACGAGT (SEQ ID NO: 11), AGGAGT (SEQ ID NO: 12), AGAGGTAGAG (SEQ ID NO: 13), TGAGGTTGAG (SEQ ID NO: 14), GGAGGTGGAG (SEQ ID NO: 15), TAG (SEQ ID NO: 16), CAG (SEQ ID NO: 17), TAG (SEQ ID NO: 18), or NAGAGTNNNN (SEQ ID NO: 19), wherein N is A, C, G, or T.

In some embodiments, the splice modulator binding site comprises the splice modulator binding site comprising any one of SEQ ID NOs: 75-81.

Provided herein, in certain embodiments, is a composition comprising the nucleic acid molecule of any of the embodiments described herein and a splice modulator, wherein the splice modulator comprises a compound of Formula (I):

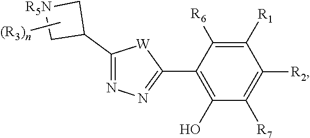

or a pharmaceutically acceptable salt, solvate, or prodrug thereof, wherein: W is —S— or —HC═CH—; R₁ is H, halogen, hydroxyl, cyano, C₁-C₆ alkyl, C₂-C₆ alkenyl, C₂-C₆ alkynyl, C₁-C₆ haloalkyl, C₁-C₆ alkoxyl, C₁-C₆ haloalkoxyl, —(CH₂)₀₋₂—C₃-C₈cycloalkyl, NH₂, NH(C₁-C₆ alkyl), N(C₁-C₆ alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4-to 7-membered heterocyclyl comprising 1, 2, or 3 heteroatoms independently selected from N, O, and S; R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄; each R₃ is independently halogen, C₁-C₆ alkyl, C₂-C₆ alkenyl, C₂-C₆ alkynyl, C₁-C₆ alkoxyl, C₃-C₈ cycloalkyl, NH₂, NH(C₁-C₆ alkyl), or N(C₁-C₆ alkyl)₂, wherein the alkyl, alkenyl, alkynyl, alkoxyl, and cycloalkyl are optionally substituted with one or more hydroxyl or NH₂; each R₄ is independently halogen, hydroxyl, cyano, nitro, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₈ cycloalkyl, NH₂, NH(C₁-C₆ alkyl), N(C₁-C₆ alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆ alkyl), or N(C₁-C₆ alkyl)₂; R₅ is H, C₁-C₆ alkyl, C₂-C₆ alkenyl, C₂-C₆ alkynyl, C₁-C₆ alkoxyl, C₃-C₈ cycloalkyl, —CH₂C₃-C₈ cycloalkyl, heterocyclyl, —CH₂ heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆ alkyl, C₁-C₆ haloalkyl, C₁-C₆ heteroalkyl, C₁-C₆ alkoxyl, C₃-C₈ cycloalkyl, spiro C₃-C₈ cycloalkyl, spiro 4-7 membered heterocyclyl, 5-6 membered heteroaryl, oxo, cyano, or hydroxyl; R₆ is H, halogen, C₁-C₆ alkyl or C₁-C₆ haloalkyl; R₇ is H, halogen, C₁-C₆ alkyl or C₁-C₆ haloalkyl; and n is 0, 1, 2, 3, 4, or 5.

Provided herein, in certain embodiments, is a composition comprising: (i) a nucleic acid molecule comprising a minigene positioned immediately 5′ to a transgene, the minigene comprising: a) an exon positioned 5′ to an intron; and b) a start codon having a first portion and a second portion, wherein the first portion and second portion of the start codon are not in the same exon; c) a splice modulator binding site; and (ii) a splice modulator that binds the splice modulator binding site and comprises a structure according to Formula (I):

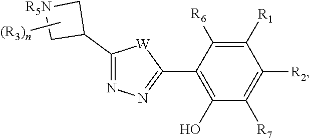

or a pharmaceutically acceptable salt, solvate, or prodrug thereof, wherein: W is —S— or —HC═CH—; R₁ is H, halogen, hydroxyl, cyano, C₁-C₆ alkyl, C₂-C₆ alkenyl, C₂-C₆ alkynyl, C₁-C₆ haloalkyl, C₁-C₆ alkoxyl, C₁-C₆ haloalkoxyl, —(CH₂)₀₋₂-C₃-C₈ cycloalkyl, NH₂, NH(C₁-C₆ alkyl), N(C₁-C₆ alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈ cycloalkyl, aryl, or 4-to 7-membered heterocyclyl comprising 1, 2, or 3 heteroatoms independently selected from N, O, and S; R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄; each R₃ is independently halogen, C₁-C₆ alkyl, C₂-C₆ alkenyl, C₂-C₆ alkynyl, C₁-C₆ alkoxyl, C₃-C₈ cycloalkyl, NH₂. NH(C₁-C₆ alkyl), or N(C₁-C₆ alkyl)₂, wherein the alkyl, alkenyl, alkynyl, alkoxyl, and cycloalkyl are optionally substituted with one or more hydroxyl or NH₂; each R₄ is independently halogen, hydroxyl, cyano, nitro, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₈ cycloalkyl, NH₂, NH(C₁-C₆ alkyl), N(C₁-C₆ alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆ alkyl), or N(C₁-C₆ alkyl)₂; R₅ is H, C₁-C₆ alkyl, C₂-C₆ alkenyl, C₂-C₆ alkynyl, C₁-C₆ alkoxyl, C₃-C₈ cycloalkyl, —CH₂C₃-C₈ cycloalkyl, heterocyclyl, —CH₂ heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆ alkyl, C₁-C₆ haloalkyl, C₁-C₆ heteroalkyl, C₁-C₆ alkoxyl, C₃-C₈ cycloalkyl, spiro C₃-C₈ cycloalkyl, spiro 4-7 membered heterocyclyl, 5-6 membered heteroaryl, oxo, cyano, or hydroxyl; R₆ is H, halogen, C₁-C₆ alkyl or C₁-C₆ haloalkyl: R₇ is H, halogen, C₁-C₆ alkyl or C₁-C₆ haloalkyl; and n is 0, 1, 2, 3, 4, or 5.

Provided herein, in certain embodiments, is composition comprising: (i) a nucleic acid molecule comprising a minigene positioned immediately 5′ to a transgene, the minigene comprising: (a) a first exon immediately 5′ to a first intron; (b) a start codon; and (c) a splice modulator binding site; (ii) a splice modulator that binds the splice modulator binding site and comprises a structure according to Formula (I):

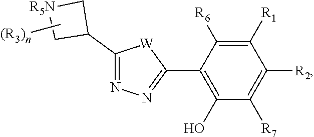

or a pharmaceutically acceptable salt, solvate, or prodrug thereof, wherein: W is —S— or —HC═CH—; R₁ is H, halogen, hydroxyl, cyano, C₁-C₆ alkyl, C₂-C₆ alkenyl, C₂-C₆ alkynyl, C₁-C₆ haloalkyl, C₁-C₆ alkoxyl, C₁-C₆ haloalkoxyl, —(CH₂)₀₋₂—C₃-C₈ cycloalkyl, NH₂, NH(C₁-C₆ alkyl), N(C₁-C₆ alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈ cycloalkyl, aryl, or 4-to 7-membered heterocyclyl comprising 1, 2, or 3 heteroatoms independently selected from N. O, and S; R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄; each R₃ is independently halogen, C₁-C₆ alkyl, C₂-C₆ alkenyl, C₂-C₆ alkynyl, C₁-C₆ alkoxyl, C₃-C₈ cycloalkyl, NH₂, NH(C₁-C₆ alkyl), or N(C₁-C₆ alkyl)₂, wherein the alkyl, alkenyl, alkynyl, alkoxyl, and cycloalkyl are optionally substituted with one or more hydroxyl or NH₂; each R₄ is independently halogen, hydroxyl, cyano, nitro, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₈ cycloalkyl, NH₂, NH(C₁-C₆ alkyl), N(C₁-C₆ alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆ alkyl), or N(C₁-C₆ alkyl)₂; R₅ is H, C₁-C₆ alkyl, C₂-C₆ alkenyl, C₂-C₆ alkynyl, C₁-C₆ alkoxyl, C₃-C₈ cycloalkyl, —CH₂C₃-C₈ cycloalkyl, heterocyclyl, —CH₂ heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆ alkyl, C₁-C₆ haloalkyl, C₁-C₆ heteroalkyl, C₁-C₆ alkoxyl, C₃-C₈ cycloalkyl, spiro C₃-C₈ cycloalkyl, spiro 4-7 membered heterocyclyl, 5-6 membered heteroaryl, oxo, cyano, or hydroxyl; R₆ is H, halogen, C₁-C₆ alkyl or C₁-C₆haloalkyl; R₇ is H, halogen, C₁-Cs alkyl or C₁-C₆haloalkyl; and n is 0, 1, 2, 3, 4, or 5.

Provided herein, in certain embodiments, is composition comprising:(i) a nucleic acid molecule comprising a minigene positioned immediately 5′ to a transgene, the minigene comprising: (a) a first exon immediately 5′ to a first intron; (b) a start codon; and (c) a splice modulator binding site; (ii) a splice modulator that binds the splice modulator binding site and comprises a structure according to Formula (II):

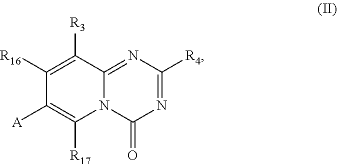

or a pharmaceutically acceptable salt, solvate, or prodrug thereof, wherein: is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆; R₁ is heterocycloalkyl comprising I nitrogen ring atom, optionally substituted with 1, 2, 3, or 4 R₆; R₂ is hydrogen, C₁₋₇alkyl, or C₃₋₈cycloalkyl; R₃ is H, halo, C₁₋₇alkyl, OR₅, N(R₅)₂, C₃₋₈cycloalkyl, or heterocycloalkyl; R₄ is aryl or bicyclic 9-membered heteroaryl comprising 2, 3, or 4 heteroatoms independently selected from N, O, and S, wherein R₄ is optionally substituted with 1, 2, or 3 R₇; each R₅ is independently C₁₋₇alkyl, C₃₋₈cycloalkyl, or heterocycloalkyl; each R₆ is independently selected from the group consisting of halogen, hydroxy, cyano, —COOH, —C(O)—C₁-C₆alkyl, —C(O)O—C₁-C₆alkyl, C₁-C₇alkyl, C₁-C₈heteroalkyl, C₁₋₇alkoxy-heterocycloalkyl, C₂-C₆alkenyl, C₂-C₆alkynyl, C₁-C₆alkoxy, —(CH₂)₀₋₂—C₃-C₈cycloalkyl, 4-7-membered monocyclic heterocycloalkyl, NH₂, NH(C₁-C₆alkyl), N(C₁-C₆alkyl)₂, —NHC(O)—C₁-C₆alkyl, —N(C₁-C₆alkyl)-C(O)—C₁-C₆alkyl, —C(O)—NH₂, —C(O)—NH(C₁-C₆alkyl), and —C(O)—N(C₁-C₆alkyl)₂, wherein the alkyl, alkenyl, alkynyl, and alkoxy are optionally substituted with one or more halogen, hydroxyl or NH₂, and wherein the cycloalkyl and heterocycloalkyl are optionally substituted with one or more halogen, hydroxyl, C₁-C₆alkyl, C₁-C₆heteroalkyl, C₁-C₆alkoxy, or NH₂; or two R⁶ on the same carbon can be taken together as keto (=O); or two R₆ together form C₁₋₇alkylene; each R₇ is independently halo, cyano, C₁₋₇alkyl, C₁₋₇haloalkyl, C₁₋₇alkoxy, C₁₋₇haloalkoxy, or C₃₋₈cycloalkyl, wherein the C₁₋₇alkyl is optionally substituted with OH; R₁₆ is H, halo, C₁₋₇alkyl, OR₅, N(R₅)₂, C₃₋₈cycloalkyl, or heterocycloalkyl; and R₁₇ is H, halo, C₁₋₇alkyl, OR₅, N(R₅)₂, C₃₋₈cycloalkyl, or heterocycloalkyl.

Provided herein, in certain embodiments, is composition comprising: (i) a nucleic acid molecule comprising a minigene positioned immediately 5′ to a transgene, the minigene comprising: (a) a first exon immediately 5′ to a first intron; (b) a start codon; and (c) a splice modulator binding site; (ii) a splice modulator that binds the splice modulator binding site and comprises a structure according to Formula (III):

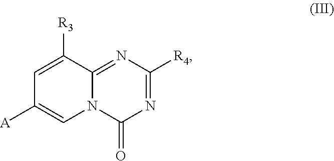

or a pharmaceutically acceptable salt, solvate, or prodrug thereof, wherein: A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆; R₁ is heterocycloalkyl comprising 1 nitrogen ring atom, optionally substituted with 1, 2, 3, or 4 R₆; R₂ is hydrogen, C₁₋₇alkyl, or C₃₋₈cycloalkyl; R₃ is H, halo, C₁₋₇alkyl, OR₅, N(R₅)₂, C₃₋₈cycloalkyl, or heterocycloalkyl; R₄ is aryl or bicyclic 9-membered heteroaryl comprising 2, 3, or 4 heteroatoms independently selected from N, O, and S, wherein R₄ is optionally substituted with 1, 2, or 3 R₇; each R₅ is independently C₁₋₇alkyl, C₃₋₈cycloalkyl, or heterocycloalkyl; each R₆ is independently C₁₋₇alkyl, amino, amino-C₁₋₇alkyl, C₃₋₈cycloalkyl, heterocycloalkyl, or C₁₋₇alkoxy-heterocycloalkyl, or two R₆ together form C₁₋₇alkylene; and each R₇ is independently halo, cyano, C₁₋₇alkyl, C₁₋₇haloalkyl, C₁₋₇alkoxy, C₁₋₇haloalkoxy, or C₃₋₈cycloalkyl, wherein the C₁₋₇alkyl is optionally substituted with OH.

In some embodiments, the splice modulator that binds the splice modulator binding site is selected from the group consisting of compounds 1A-192 A and 100B-135B.

In some embodiments, the splice modulator that binds the splice modulator binding site is selected from the group comprising 3A, 6A, 8A, 10A, 15A, 24A, 86A, 100B, 111B, 117B, 121B, 135B, and 192A.

In some embodiments, the splice modulator that binds the splice modulator binding site is selected from the group comprising 116B, 100B, 1A, 22A, 24A, 2A, and 34A.

In some embodiments, the splice modulator binding site comprises the nucleic acid sequence of DGAGTDDGHV (SEQ ID NO: 82) or DGAGTDDNHV (SEQ ID NO: 83), wherein D is A, G, or T; wherein R is A, or G; wherein N is A, C, G, or T; wherein H is A, C, or T; and wherein V is A, G, or C.

In some embodiments, the splice modulator binds to the splice modulator binding site comprising the nucleic acid sequence of DGAGTRRGHV (SEQ ID NO: 1) or DGAGTRRNHV (SEQ ID NO: 2), wherein D is A, G, or T; and wherein R is A, or G; and wherein N is A, C, G, or T; wherein H is A, C, or T; wherein V is A, G, or C.

In some embodiments, the splice modulator binding site comprises the nucleic acid sequence of DGAGTTTGHV (SEQ ID NO: 84), wherein D is A, G, or T; wherein H is A, C, or T; and wherein V is A, G, or C.

In some embodiments, the splice modulator binding site does not comprise the nucleic acid sequence AAGAGT (SEQ ID NO: 3), ATGAGT (SEQ ID NO: 4), TAGAGT (SEQ ID NO: 5), TTGAGT (SEQ ID NO: 6), GAGAGT (SEQ ID NO: 7), GTGAGT (SEQ ID NO: 8), AAGAGT (SEQ ID NO: 9), ATGAGT (SEQ ID NO: 10), ACGAGT (SEQ ID NO: 11), AGGAGT (SEQ ID NO: 12), AGAGGTAGAG (SEQ ID NO: 13), TGAGGTTGAG (SEQ ID NO: 14), GGAGGTGGAG (SEQ ID NO: 15), TAG (SEQ ID NO: 16), CAG (SEQ ID NO: 17), TAG (SEQ ID NO: 18), or NAGAGTNNNN (SEQ ID NO: 19), wherein N is A, C, G, or T.

In some embodiments, the splice modulator binds to the splice modulator binding site comprising a nucleic acid sequence of any one of SEQ ID NOs: 75-81.

In some embodiments, the transgene encodes a protein of interest. In some embodiments, the transgene encodes a miRNA of interest. In some embodiments, the transgene encodes a shRNA of interest. In some embodiments, the transgene encodes a functional or regulatory RNA of interest.

In some embodiments, the nucleic acid molecule further comprises a promoter. In some embodiments, the promoter is a GFAP promoter, Nestin promoter, S100B promoter, Nefh promoter, dystrophin promoter, H1 promoter, 7SK promoter, apolipoprotein E-human-alpha 1-antitrypsin promoter, CK8 promoter, mU1a, EF-1α promoter, TBG promoter, PKG promoter, CAG, the SV40 early promoter, murine mammary tumor virus LTR promoter, Ad MLP: HSV promoter, a CMV promoter such as CMV-IE, RSV promoter, U6 promoter or variants thereof, hSyn promoter, hexaribonucleotide binding protein-3 (NeuN) promoter. CaMKII promoter, Tα-1 promoter, neuron-specific enolase (NSE) promoter, PDGFβ promoter, VGLUT promoter, SST promoter. NPY promoter, VIP promoter. PV promoter, GAD65 or GAD67 promoter, promoter of DRD1 and DRD2, MAP1B, Clql2 promoter, POMC promoter, PROX1 promoter, or any suitable promoter.

In some embodiments, the molecule comprises a polyA, optionally wherein the polyA is an SV40 polyA, a HGH polyA, a BGH polyA, a beta-globin polyA, an alpha-globin polyA, an ovalbumin polyA, a kappa-light chain polyA, a synthetic polyA, or any suitable polyA.

Provided herein, in certain embodiments, is a vector comprising the nucleic acid molecule or the composition of any of the embodiments described herein, optionally wherein the vector is a plasmid, a DNA vector, an RNA vector, a virion, or a viral vector.

In some embodiments, the vector is a viral vector. In some embodiments, the viral vector is an adeno-associated virus (AAV), lentivirus, adenovirus, simian virus 40, vaccinia virus, measles virus, herpes virus, or poxvirus. In some embodiments, the viral vector is an AAV. In some embodiments, the AAV comprises capsid proteins from an AAV serotype selected from the group consisting of AAV1, AAV2, AAV3, AAV4, AAV5, AAV6, AAV7, AAV8, AAV9, AAVrh10, and AAVrh74. In some embodiments, the AAV is a pseudotyped AAV.

Provided herein, in certain embodiments, is a pharmaceutical composition comprising the nucleic acid molecule of any of the embodiments described herein or the composition of any of the embodiments described herein or the vector of any of the embodiments described herein and a pharmaceutically acceptable carrier, diluent, or excipient.

Provided herein, in certain embodiments, is a method of modulating the expression of a protein, RNA, or other biomolecule in a subject in need thereof, the method comprising administering to the subject a therapeutically effective amount of the nucleic acid molecule of any of the embodiments described herein or the composition of any of the embodiments described herein, or the vector of any of the embodiments described herein or the pharmaceutical composition of any of the embodiments described herein.

In some embodiments, the method further comprises administering to the subject a therapeutically effective amount of a splice modulator.

In some embodiments, the protein is expressed in the presence of the splice modulator.

In some embodiments, the method includes administering to the subject a therapeutically effective amount of the splice modulator causes an inclusion of one of the two or more exons, one or more exons, the first exon, or the second exon. In some embodiments, one of the two or more exons or one of the one or more exons is the second exon.

In some embodiments, the splice modulator binds to an RNA binding protein and/or a segment of the splice modulator binding site of the nucleic acid of any of the embodiments described herein.

### BRIEF DESCRIPTION OF THE DRAWINGS

FIG. 1A is a schematic depiction of a splicing event that may occur in an exemplary nucleic acid molecule of the disclosure. The nucleic acid molecule may include a start codon (e.g., ATG or AUG) having at least one nucleotide located in an exon, which is flanked by 5′ and 3′ splice sites located in flanking introns, respectively. Such a start codon may be excised during splicing, such that a downstream transgene is not translated (“off”) due to the absence of a start codon. FIG. 1B depicts the same exemplary nucleic acid molecule, except that a splice modulator (e.g., a small molecule) binds to the exon which includes a splice modulator binding site. The binding of a splice modulator to the splice modulator binding site initiates an alternative splicing event of the exemplary nucleic acid molecule, such that the start codon is incorporated at the 5′-end of the transgene and the transgene is translated (“on”).

FIG. 2 is a schematic depiction of an exemplary nucleic acid molecule (e.g., a DNA molecule) of the disclosure including a minigene linked to a transgene, in which the minigene includes a first exon, a first intron, a second exon, a start codon, and a second intron, and a polyadenylation signal (polyA). In the “original” nucleic acid molecule, three codons of a start codon are located in the second exon. In an exemplary nucleic acid molecule of the disclosure (e.g., “Start”), at least one nucleotide of the start codon is located in the exon and at least one nucleotide of the start codon is located in the transgene. In some embodiments, one nucleotide of the start codon is located in the second exon and two nucleotides of the start codon are located in the transgene. Alternatively, for example, in some embodiments, two nucleotides of the start codon are located in the second exon and one nucleotide of the start codon is located in the transgene.

FIG. 3 is a schematic depiction of an adeno-associated virus (AAV) encoding a nucleic acid molecule (e.g., a DNA molecule) including a minigene linked to a transgene, in which the minigene includes a first exon, a first intron, a second exon including i) at least one nucleotide of a start codon and ii) a splice modulator binding site, a second intron, and a polyA, flanked by inverted terminal repeats (ITR). As described in FIG. 1B, in the presence of a splice modulator (e.g., a small molecule) binding to the splice modulator binding site located in the second exon, a splicing event takes place such that the start codon (e.g., a start codon having at least one nucleotide of the start codon located in the second exon and at least one nucleotide of the start codon located in the transgene) is incorporated at the 5′-end of the transgene and the transgene is translated.

FIGS. 4A-4E are a set of schematic depictions of numerous exemplary mechanisms by which a nucleic acid molecule of the disclosure may perform exon-inclusion by way of a splicing event. All panels provide a nucleic acid molecule, which includes a bipartite start codon (e.g., ATG or AUG) having at least one nucleotide located in an exon (e.g., the second exon in the schematic), which is flanked by 5′ and 3′ splice sites located in the flanking introns, respectively. In all panels, the third depicted exon may be a transgene, which is not translated in the absence of the reconstituted bipartite start codon. FIG. 4A depicts a nucleic acid molecule in which the second exon is excised during splicing (left), unless a splice modulator (e.g., a ‘small molecule’) binds to and stabilizes the binding of the spliceosome to the exon-intron junction of the second exon and the second intron, such that an alternative splicing event is initiated and the start codon is reconstituted at the 5′-end of the third depicted exon (e.g., a transgene) and the third exon is translated (right). FIG. 4B depicts a nucleic acid molecule in which the second exon is excised by the spliceosome due to the presence of a splice repressor, which represses splicing at the exon-intron junction of the second exon and second intron (left), unless a splice modulator (e.g., a ‘small molecule’) binds to a segment of the nucleic acid molecule and blocks or de-stabilizes the binding of the repressor, such that an alternative splicing event is initiated and the start codon is reconstituted at the 5′-end of the third depicted exon (e.g., a transgene) and the third exon is translated (right). FIG. 4C depicts a nucleic acid molecule in which the second exon is excised by the spliceosome due to the absence of a splice enhancer, which would support splicing at the exon-intron junction of the second exon and second intron (left), unless a splice modulator (e.g., a ‘small molecule’) binds to a segment of the nucleic acid molecule and stabilizes binding of an enhancer, such that an alternative splicing event is initiated and the start codon is reconstituted at the 5′-end of the third depicted exon (e.g., a transgene) and the third exon is translated (right). FIG. 4D depicts a nucleic acid molecule in which the second exon is excised during splicing (left), unless a splice modulator (e.g., a ‘small molecule’) binds and stabilizes a splice modulator binding site (e.g., a riboswitch e.g., an aptamer). Such an aptamer stabilizes the binding of the spliceosome to the exon-intron junction of the second exon and the second intron, such that an alternative splicing event is initiated and the start codon is reconstituted at the 5′-end of the third depicted exon (e.g., a transgene) and the third exon is translated (right). FIG. 4E depicts a nucleic acid molecule in which the second exon is excised during splicing due to the presence of a splice repressor, which represses splicing at the exon-intron junction of the second exon and second intron (left), unless a splice modulator (e.g., a ‘small molecule’) binds and stabilizes a splice modulator binding site (e.g., a riboswitch e.g., an aptamer). Such an aptamer blocks or de-stabilizes the binding of the spliceosome to the splicing repressor, such that an alternative splicing event is initiated and the start codon is reconstituted at the 5′-end of the third depicted exon (e.g., a transgene) and the third exon is translated (right).

FIG. 5 is a schematic depiction of an exemplary nucleic acid molecule (e.g., a DNA molecule) of the disclosure including a minigene linked to a transgene, in which the minigene includes a first exon, a first intron, a second exon, a start codon, a second intron, and a polyadenylation signal (polyA). In the “original” nucleic acid molecule, three codons of a start codon are located in the second exon. In an exemplary nucleic acid molecule of the disclosure, at least one nucleotide of the start codon is located in the first exon and at least one nucleotide of the start codon is located in the second exon.

FIGS. 6A-6K are a set of schematic depictions of numerous exemplary mechanisms by which a nucleic acid molecule of the disclosure (e.g., including one or more exons and/or one or more introns) may perform exon-inclusion by way of a splicing event. FIG. 6A is an exemplary nucleic acid molecule of the disclosure wherein at least one nucleotide of a start codon is located in a first exon and at least one nucleotide of the start codon is located in a transgene. FIG. 6B is an exemplary nucleic acid molecule of the disclosure wherein at least one nucleotide of a start codon is located in a first exon and at least one nucleotide of the start codon is located in a second exon. FIG. 6C is an exemplary nucleic acid molecule of the disclosure wherein at least one nucleotide of a start codon is located in a first exon, at least one nucleotide of the start codon is located in a second exon, and a stop codon is located in the second exon which is upstream of the at least one nucleotide of the start codon located in the second exon. FIG. 6D is an exemplary nucleic acid molecule of the disclosure wherein at least one nucleotide of a start codon is located in a second exon, a stop codon is located in the second exon downstream of the at least one nucleotide of the start codon in the second exon, and at least one nucleotide of the start codon is located in a transgene. FIG. 6E is an exemplary nucleic acid molecule of the disclosure wherein a stop codon is located in a second exon which is upstream of at least one nucleotide of a start codon located in the second exon, and wherein at least one nucleotide of the start codon is located in a transgene. FIG. 6F is an exemplary nucleic acid molecule of the disclosure wherein at least one nucleotide of a start codon is located in a second exon, and at least one nucleotide of the start codon is located in a transgene. FIG. 6G is an exemplary nucleic acid molecule of the disclosure wherein at least one nucleotide of a start codon is located in at the 3′ end of a second exon and at least one nucleotide of the start codon is located in a transgene. FIG. 6H is an exemplary nucleic acid molecule of the disclosure wherein at least one nucleotide of a start codon is located in a first exon, and at least one nucleotide of the start codon is located in a transgene. FIG. 6I is an exemplary nucleic acid molecule of the disclosure wherein at least one nucleotide of a start codon is located in a first exon, at least one nucleotide of the start codon is located in a transgene, and a stop codon is located in the transgene upstream of the at least one nucleotide of the start codon that is located in the transgene. FIG. 6J is an exemplary nucleic acid molecule of the disclosure wherein a stop codon is located in a second exon, at least one nucleotide of a start codon is located in a third exon, and at least one nucleotide of the start codon is located in a transgene. FIG. 6K is an exemplary nucleic acid molecule of the disclosure wherein at least one nucleotide of a start codon is located in a second exon, which is upstream of a stop codon in a third exon, and at least one nucleotide of the start codon is located in a transgene.

FIGS. 7A-7H are a set of schematic depictions of numerous exemplary mechanisms by which a nucleic acid molecule of the disclosure may perform exon-inclusion by way of a splicing event. FIG. 7A is an exemplary nucleic acid molecule of the disclosure wherein a start codon is located in a first exon, which is upstream of a single intron and a transgene. FIG. 7B is an exemplary nucleic acid molecule of the disclosure wherein a start codon is located in a first exon, which is upstream of an intron and a stop codon located in a transgene. FIG. 7C is an exemplary nucleic acid molecule of the disclosure wherein a start codon is located at the 3′ end of a second exon, which is upstream of an intron and a transgene. FIG. 7D is an exemplary nucleic acid molecule of the disclosure wherein a stop codon is located in a second exon and a start codon is located in a third exon, which is upstream of a transgene. FIG. 7E is an exemplary nucleic acid molecule of the disclosure wherein a start codon is located in a second exon and a stop codon is located in a third exon, which is upstream of a transgene. FIG. 7F is an exemplary nucleic acid molecule of the disclosure wherein a stop codon is located in a second exon 5′ to a start codon in the same exon, which is upstream of a transgene. FIG. 7G is an exemplary nucleic acid molecule of the disclosure wherein a start codon is located in a second exon 5′ to a stop codon in the same exon, which is upstream of a transgene. FIG. 7H is an exemplary nucleic acid molecule of the disclosure wherein a start codon is located in a second exon 5′, which is upstream of a transgene.

FIG. 8A is a scheme of a splicing assay used to optimize switch sequences.

FIG. 8B is a graph of luciferase signal from HEK-293T cells transiently transfected with expression plasmids containing either RS1-10 controlling a firefly luciferase gene or a variant switch identified from the switch sequence screen containing a 28 nucleotide intronic deletion. Transfected cells were treated for 24 hours with 85 nM 24 A. Bars show mean±standard deviation.

FIG. 9A is a graph of the fold-induction of luciferase signal relative to vehicle of RS1-1 and variants that were screened for induction of luminescent signal with either DMSO vehicle or the specified dose of 1 A. Bars represent mean of >3 wells normalized to vehicle controls.

FIG. 9B is a graph of the fold-induction of luciferase signal relative to vehicle of RS1-1 variants that were screened for induction of luminescent signal with either DMSO vehicle or the specified dose of 1 A. Bars represent mean of >3 wells normalized to vehicle controls.

FIG. 9C is a bar graph the fold-induction of luciferase signal relative to vehicle of RS1-10 with 100 nM or 1,000 nM of the indicated compound or DMSO.

FIG. 9D is a graph of the fold-induction of luciferase signal relative to vehicle of RS1-10 in response to the specified concentrations of 22 A.

FIG. 9E is a graph of the fold-induction of luciferase signal relative to vehicle of RS1-10 in response to the specified concentrations of 24 A.

FIG. 9F is a graph of the fold-induction of luciferase signal relative to vehicle of RS1-10 in response to the specified concentrations of 34 A.

FIG. 9G is a graph of the percent of luciferase signal in a Fln-In 293 cell line containing a single-copy insertion of the RS1-10 sequence fused to a Luciferase gene in response to treatment with the specified concentrations of 24 A compared to the luciferase signal of a Fln-In 293 cell line that constitutively expresses Luciferase.

FIG. 10A is a graph of the fold-induction of luciferase signal relative to vehicle of the fold-induction of luciferase signal relative to vehicle of RS1-10 in response to the specified concentrations of 24 A in HEK-293T cells.

FIG. 10B is a graph of the fold-induction of luciferase signal relative to vehicle of the fold-induction of luciferase signal relative to vehicle of RS1-10 in response to the specified concentrations of 24 A in NIH-3T3 cells.

FIG. 10C is a graph of the fold-induction of luciferase signal relative to vehicle of the fold-induction of luciferase signal relative to vehicle of RS1-10 in response to 1 A in SH-SY5Y cells with either a CBA promoter or human Synapsin promoter.

FIG. 10D is a graph of the fold-induction of luciferase signal relative to vehicle of the fold-induction of luciferase signal relative to vehicle of RS1-10 in response to 1 A in HepG2 cells with a CBA promoter.

FIG. 11A is a scheme of a vector containing RS1-10 injected into C57BL/6 mice and the resulting AAV-PHP.eB capsid.

FIG. 11B are images of C57BL/6 mice 6 hours post dose of vehicle or 24 A.

FIG. 11C shows images of C57BL/6 mice 7 days post dose of vehicle or 24 A.

FIG. 12 is a graph showing the quantification of the radiance in the head region of mice 6 hours post-dose of the respective treatment.

FIG. 13A is a graph of the fold-induction of luciferase signal relative to vehicle of RS2-1 and variants that were screened for induction of luminescent signal with either DMSO vehicle or 100B. Bars represent mean+/−standard deviation of 3 wells normalized to vehicle controls.

FIG. 13B is a graph of the fold-induction of luciferase signal relative to vehicle of RS2-1 and additional variants that were screened for induction of luminescent signal with either DMSO vehicle or 100B. Bars represent mean+/−standard deviation of 3 wells normalized to vehicle controls.

FIG. 13C is a graph of the fold-induction of luciferase signal relative to vehicle of RS2-3 with 100 nM or 1,000 nM of the indicated compound or DMSO.

FIG. 14A is a graph of the fold-induction of luciferase signal relative to vehicle of RS3-1 and variants that were screened for induction of luminescent signal with either DMSO vehicle or 116B. Bars represent mean+/−standard deviation of 3 wells normalized to vehicle controls.

FIG. 14B is a graph of the fold-induction of luciferase signal relative to vehicle of RS3-1 variants that were screened for induction of luminescent signal with either DMSO vehicle or 116B. Bars represent mean+/−standard deviation of 3 wells normalized to vehicle controls.

FIG. 14C is a graph of the fold-induction of luciferase signal relative to vehicle of RS3-1 variants that were screened for induction of luminescent signal with either DMSO vehicle or the specified concentrations of 116B. Bars represent mean+/−standard deviation of 3 wells normalized to vehicle controls.

FIG. 14D is a graph of the fold-induction of luciferase signal relative to vehicle of RS3-13 with 100 nM or 1,000 nM of the indicated compound or DMSO.

FIG. 15A is a graph of the fold-induction of luciferase signal relative to vehicle of RS4-1 and variants that were screened for induction of luminescent signal with either DMSO vehicle or 24 A. Bars represent mean+/−standard deviation of 3 wells normalized to vehicle controls.

FIG. 15B is a graph of the fold-induction of luciferase signal relative to vehicle of RS4-1 and variants that were screened for induction of luminescent signal with either DMSO vehicle or 116B. Bars represent mean+/−standard deviation of 3 wells normalized to vehicle controls

### DETAILED DESCRIPTION

### Nucleic Acid Molecules of the Disclosure

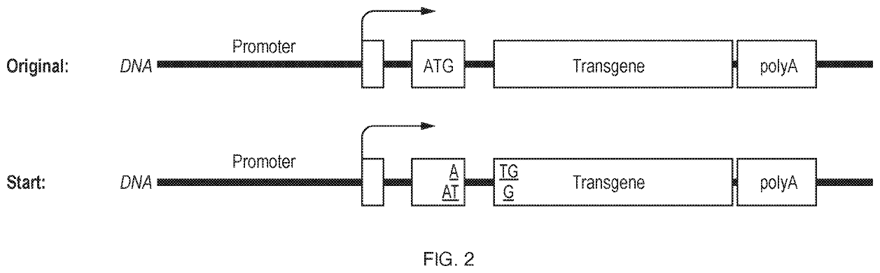

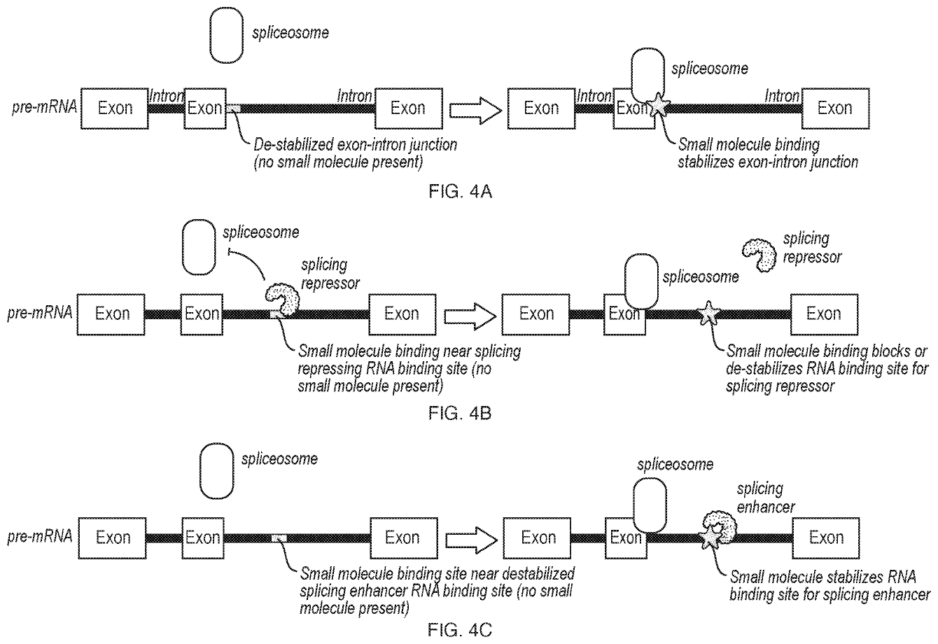

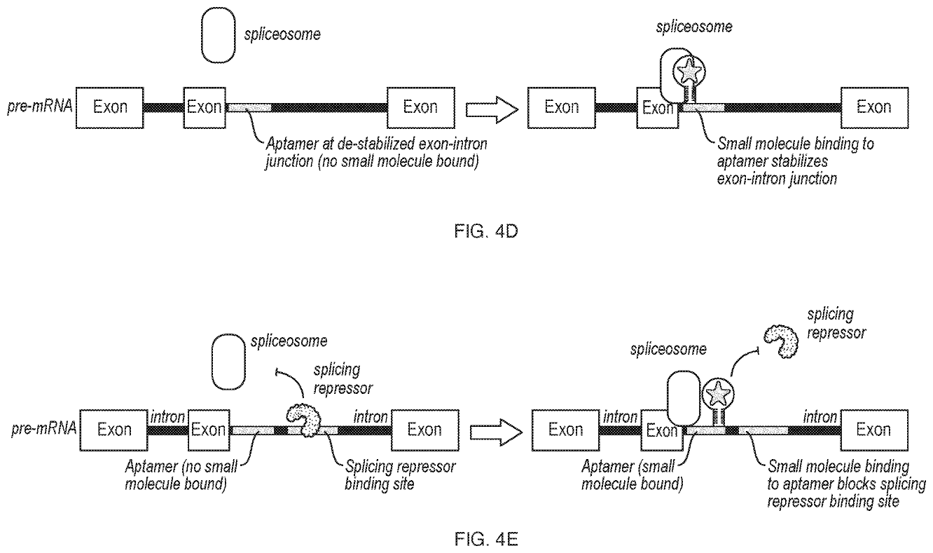

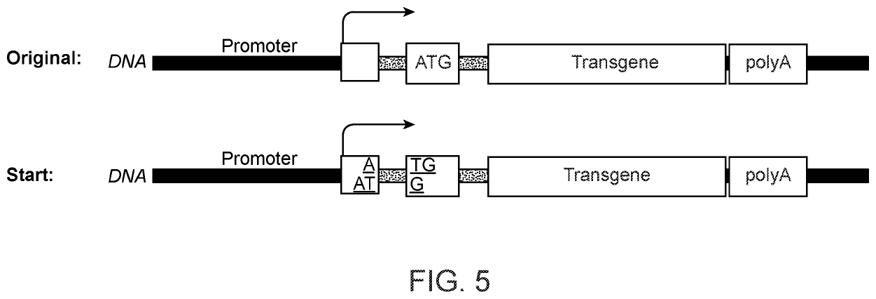

The disclosure is based, at least in part, on the surprising discovery that a splicing event regulated by an exogenous splice modulator can affect the inclusion of a bipartite start codon (e.g., a start codon including at least one nucleotide in an exon and at least one nucleotide in a transgene) and provides a highly efficient means for regulating expression of a transgene. The disclosure is also based, at least in part, in the surprising discovery of a nucleic acid molecule design which improves upon the idea of using splicing to regulate transgene expression. For example, by splitting a start codon across exons (see e.g., molecule design depicted in FIG. 2, “Start”), one can avoid engagement of translation machinery on the transcript unless the ‘on’ state is generated by reconstitution of the start codon when the splice modulator is present. This design is expected to reduce background expression and prevent truncated protein production. In the embodiments of the disclosure, the nucleic acid molecules herein generally function by way of an exon-inclusion mechanism. In some embodiments, the splice modulator binds to and agonize the function of a RNA binding protein (RBP) that endogenously promotes a splicing event. In some embodiments, a splice modulator of the disclosure promotes the binding of the RBP to the nucleic acid molecule, thereby enabling splicing to occur at the junction between the second exon and the second intron, such that an exon is included. In some embodiments, the splice modulator binds to a riboswitch, such that splicing may occur at the junction between the second exon and the second intron, such that an exon is included. In both examples, and in all embodiments of the disclosure, the nucleic acid molecules herein function by way of an exon-inclusion mechanism. Five exemplary mechanisms of action by which a nucleic acid molecule of the disclosure performs exon-inclusion by way of a splicing event are described in FIGS. 4A-4E. In some embodiments, the compositions and methods described herein are used to modulate the expression of a protein, functional RNA, regulatory RNA, microRNA (miRNA), short-hairpin RNA (shRNA) (e.g., a protein, miRNA, or shRNA encoded by a transgene) in a subject in need thereof.

In all embodiments, a nucleic acid molecule of the disclosure functions by way of an exon-inclusion mechanism. In some embodiments, the splice modulator binds to and agonizes the function of an RBP that endogenously promotes a splicing event. In such an embodiment, a splice modulator of the disclosure promotes the binding of the RBP to the spliceosome, thereby enabling splicing to occur at the junction between the second exon and the second intron, such that an exon is included. In another embodiment of the disclosure, the splice modulator binds to a riboswitch, such that splicing occurs at the junction between the second exon and the second intron, such that an exon is included. In both examples, and in all embodiments of the disclosure, the nucleic acid molecules herein function by way of an exon-inclusion mechanism.

In some embodiments, the second exon of a nucleic acid molecule is excised during splicing (FIG. 4A, left), unless a splice modulator (e.g., a ‘small molecule’) binds to and stabilizes the binding of the spliceosome to the exon-intron junction of the second exon and the second intron, such that an alternative splicing event is initiated and the start codon is reconstituted at the 5′-end of the third depicted exon (e.g., a transgene) and the third exon is translated (FIG. 4A, right). In some embodiments, the second exon of a nucleic acid molecule is excised by the spliceosome due to the presence of a splicing repressor, which represses splicing at the exon-intron junction of the second exon and second intron (FIG. 4B, left), unless a splice modulator (e.g., a ‘small molecule’) binds to a segment of the nucleic acid molecule and blocks or de-stabilizes the binding of the repressor, such that an alternative splicing event is initiated and the start codon is reconstituted at the 5′-end of the third depicted exon (e.g., a transgene) and the third exon is translated (FIG. 4B, right). In some embodiments, the second exon of a nucleic acid molecule is excised by the spliceosome due to the absence of a splice enhancer, which would support splicing at the exon-intron junction of the second exon and second intron (FIG. 4C, left), unless a splice modulator (e.g., a ‘small molecule’) binds to a segment of the nucleic acid molecule and stabilizes binding of an enhancer, such that an alternative splicing event is initiated and the start codon is reconstituted at the 5′-end of the third depicted exon (e.g., a transgene) and the third exon is translated (FIG. 4C, right). In some embodiments, the second exon of a nucleic acid molecule is excised during splicing (FIG. 4D, left), unless a splice modulator (e.g., a ‘small molecule’) binds and stabilizes a splice modulator binding site (e.g., a riboswitch e.g., an aptamer). Such an aptamer stabilizes the binding of the spliceosome to the exon-intron junction of the second exon and the second intron, such that an alternative splicing event is initiated and the start codon is reconstituted at the 5′-end of the third depicted exon (e.g., a transgene) and the third exon is translated (FIG. 4D, right) (FIG. 4D). In some embodiments, the second exon of a nucleic acid molecule is excised during splicing due to the presence of a splice repressor, which represses splicing at the exon-intron junction of the second exon and second intron (FIG. 4E, left), unless a splice modulator (e.g., a ‘small molecule’) binds and stabilizes a splice modulator binding site (e.g., a riboswitch e.g., an aptamer). Such an aptamer blocks or de-stabilizes the binding of the spliceosome to the splicing repressor, such that an alternative splicing event is initiated and the start codon is reconstituted at the 5′-end of the third depicted exon (e.g., a transgene) and the third exon is translated (FIG. 4E, right).

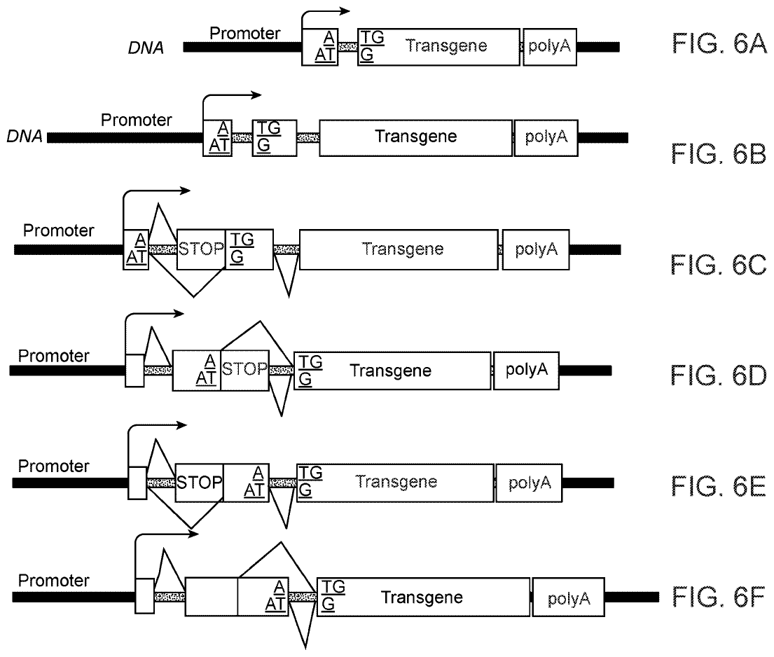

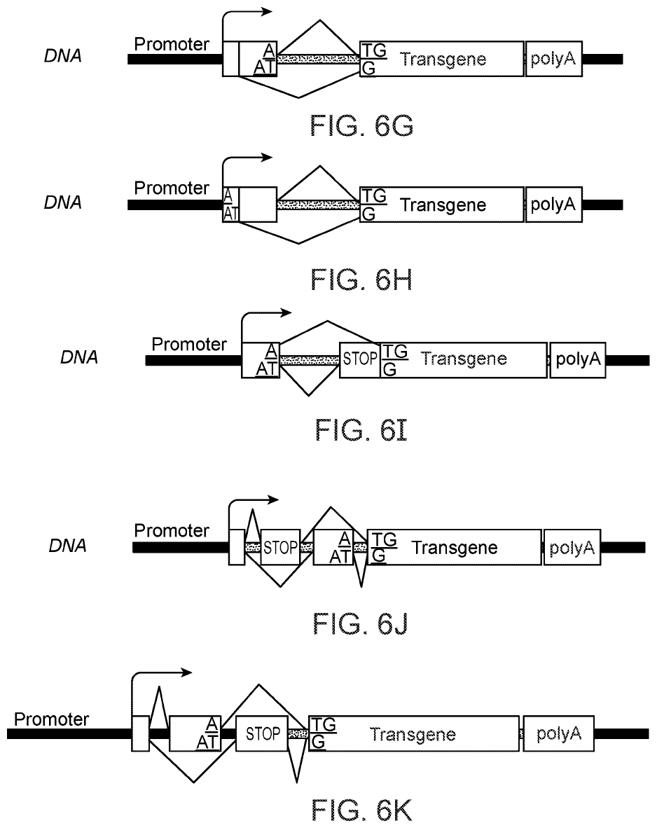

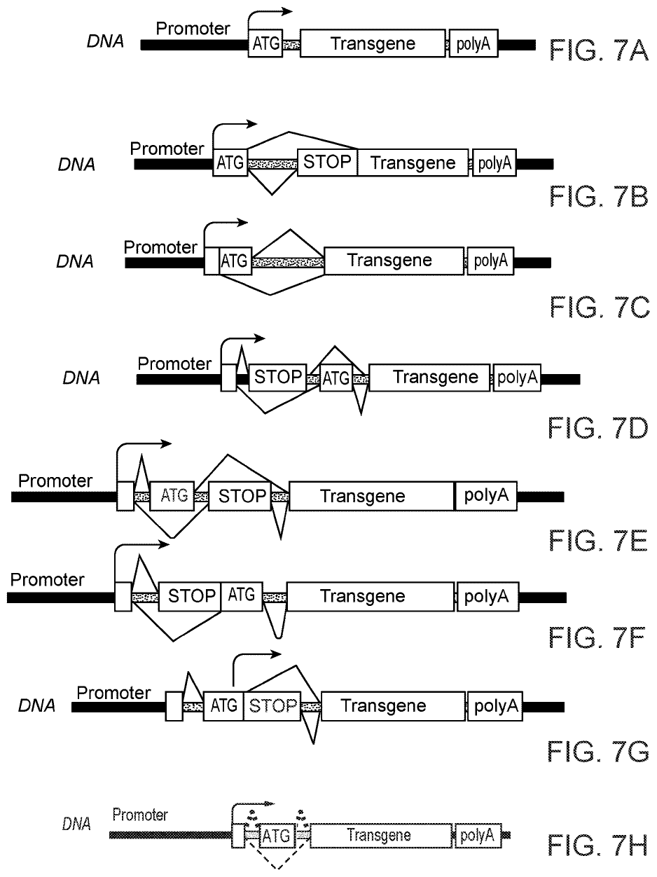

Described herein, in certain embodiments, is a nucleic acid molecule of the disclosure provides a nucleic acid molecule comprising a minigene positioned immediately 5′ to a transgene, the minigene comprising: (a) a first exon 5′ to a first intron: (b) a start codon comprising a first portion and a second portion, wherein the first portion and second portion of the start codon are not in the same exon, and (c) a splice modulator binding site, the splice modulator binding site comprises the nucleic acid sequence of DGAGTDDGHV (SEQ ID NO: 82) or DGAGTDDNHV (SEQ ID NO: 83), wherein D is A, G, or T; wherein R is A, or G; wherein N is A, C, G, or T; wherein H is A, C, or T; and wherein V is A, G, or C. In some embodiments, the splice modulator binding site comprises the nucleic acid sequence of DGAGTTTGHV (SEQ ID NO: 84), wherein D is A, G, or T; wherein H is A, C, or T; and wherein V is A, G, or C. Further described herein, in certain embodiments, is a nucleic acid molecule of the disclosure provides a nucleic acid molecule comprising a minigene positioned immediately 5′ to a transgene, the minigene comprising: (a) a first exon 5′ to a first intron; (b) a start codon comprising a first portion and a second portion, wherein the first portion and second portion of the start codon are not in the same exon, and (c) a splice modulator binding site, the splice modulator binding site comprises the nucleic acid sequence of DGAGTRRGHV (SEQ ID NO: 1) or DGAGTRRNHV (SEQ ID NO: 2), wherein D is adenine (A), guanine (G), or thymidine (T); wherein N is A, C, G, or T; and wherein R is A, or G; and wherein H is A, C, or T; and where in V is A, G, or C. See FIGS. 6A-6K.

In some embodiments, the first portion of the start codon comprises one or two nucleotides of the start codon. In some embodiments, the second portion of the start codon has one or two nucleotides of the start codon. In some embodiments, the first portion of the start codon is located in the first exon, and the second portion of the start codon is located in the transgene.

In some embodiments, the nucleic acid comprises in 5′ to 3′ order the first exon comprising the first portion of the start codon, the first intron, and the transgene comprising the second portion of the start codon. See FIG. 6A.

In some embodiments, the nucleic acid molecule further comprises a second exon. In some embodiments, the nucleic acid molecule further comprises a second intron.

In some embodiments, the nucleic acid comprises in 5′ to 3′ order the first exon comprising the first portion of the start codon, the second exon comprising the second portion of the start codon, and the transgene. See FIG. 6B.

In some embodiments, the nucleic acid comprises in 5′ to 3′ order the first exon comprising the first portion of the start codon, the first intron, the second exon comprising the second portion of the start codon, the second intron, and the transgene. See FIG. 6B.

In some embodiments, the nucleic acid comprises in 5′ to 3′ order the first exon, the second exon comprising the first portion of the start codon, and the transgene comprising the second portion of the start codon. See FIG. 6G.

In some embodiments, the nucleic acid comprises in 5′ to 3′ order the first exon, the second exon comprising the first portion of the start codon, the first intron, and the transgene comprising the second portion of the start codon. See FIG. 6G.

In some embodiments, the nucleic acid comprises in 5′ to 3′ order the first exon comprising the first portion of the start codon, the second exon, and the transgene comprising the second portion of the start codon. See FIG. 6H.

In some embodiments, the nucleic acid comprises in 5′ to 3′ order the first exon comprising the first portion of the start codon, the second exon, the first intron, and the transgene comprising the second portion of the start codon. See FIG. 6H.

In some embodiments, the nucleic acid comprises in 5′ to 3′ order the first exon comprising the first portion of the start codon, the second exon comprising the second portion of the start codon, and the transgene. See FIG. 6B.

In some embodiments, the nucleic acid comprises in 5′ to 3′ order the first exon comprising the first portion of the start codon, the first intron, the second exon comprising the second portion of the start codon, and the transgene. See FIG. 6B.

In some embodiments, the nucleic acid molecule further comprises a stop codon.

In some embodiments, the nucleic acid comprises in 5′ to 3′ order the first exon comprising the first portion of the start codon, the second exon comprising the stop codon, and the transgene comprising the second portion of the start codon. See FIG. 6I.

In some embodiments, the nucleic acid comprises in 5′ to 3′ order the first exon comprising the first portion of the start codon, the first intron, the second exon comprising the stop codon, and the transgene comprising the second portion of the start codon. See FIG. 6I.

In some embodiments, the second exon comprises the splice modulator binding site.

In some embodiments, the nucleic acid molecule further comprises a third exon. In some embodiments, the nucleic acid molecule further comprises a third intron.

In some embodiments, the nucleic acid comprises in 5′ to 3′ order the first exon comprising the first portion of the start codon, the second exon comprising the stop codon, the third exon comprising the second portion of the start codon, and the transgene. See FIG. 6C.

In some embodiments, the nucleic acid comprises in 5′ to 3′ order the first exon comprising the first portion of the start codon, the first intron, the second exon comprising the stop codon, the third exon comprising the second portion of the start codon, the second intron, and the transgene. See FIG. 6C.

In some embodiments, the nucleic acid comprises in 5′ to 3′ order the first exon, the second exon comprising first portion of the start codon, the third exon comprising the stop codon, and the transgene comprising the second portion of the start codon. See FIG. 6D.

In some embodiments, the nucleic acid comprises in 5′ to 3′ order the first exon, the first intron, the second exon comprising first portion of the start codon, the third exon comprising the stop codon, the second intron, and the transgene comprising the second portion of the start codon. See FIG. 6D.

In some embodiments, the nucleic acid comprises in 5′ to 3′ order the first exon, the second exon comprising the stop codon, the third exon comprising first portion of the start codon, and the transgene comprising the second portion of the start codon. See FIG. 6E.

In some embodiments, the nucleic acid comprises in 5′ to 3′ order the first exon, the first intron, the second exon comprising the stop codon, the third exon comprising first portion of the start codon, the second intron, and the transgene comprising the second portion of the start codon. See FIG. 6D.

In some embodiments, the nucleic acid comprises in 5′ to 3′ order the first exon, the second exon, the third exon comprising the first portion of the start codon, and the transgene comprising the second portion of the start codon. See FIG. 6F.

In some embodiments, the nucleic acid comprises in 5′ to 3′ order the first exon, the first intron, the second exon, the third exon comprising the first portion of the start codon, the second intron, and the transgene comprising the second portion of the start codon. See FIG. 6F.

In some embodiments, the nucleic acid comprises in 5′ to 3′ order the first exon, the second exon comprising the stop codon, the third exon comprising the first portion of the start codon, and the transgene comprising the second portion of the start codon. See FIG. 6J.

In some embodiments, the nucleic acid comprises in 5′ to 3′ order the first exon, the first intron, the second exon comprising the stop codon, the second intron, the third exon comprising the first portion of the start codon, the third intron, and the transgene comprising the second portion of the start codon. See FIG. 6J.

In some embodiments, the nucleic acid comprises in 5′ to 3′ order the first exon, the second exon comprising the first portion of the start codon, the third exon comprising the stop codon, and the transgene comprising the second portion of the start codon. See FIG. 6K.

In some embodiments, the nucleic acid comprises in 5′ to 3′ order the first exon, the first intron, the second exon comprising the first portion of the start codon, the second intron, the third exon comprising the stop codon, the third intron, and the transgene comprising the second portion of the start codon. See FIG. 6K.

In some embodiments, the first intron comprises the splice modulator binding site.

In some embodiments, the splice modulator binding site is located at the junction between the second exon and the second intron.

In some embodiments, the splice modulator binding site is located within the second intron.

Described herein, in certain embodiments, is a nucleic acid molecule comprising a minigene positioned immediately 5′ to a transgene, the minigene comprising: (a) a first exon immediately 5′ to a first intron; (b) a start codon; and (c) a splice modulator binding site, the splice modulator binding site comprises the nucleic acid sequence of DGAGTDDGHV (SEQ ID NO: 82) or DGAGTDDNHV (SEQ ID NO: 83), wherein D is A, G, or T; wherein R is A, or G; wherein N is A, C, G, or T; wherein H is A, C, or T; and wherein V is A, G, or C. In some embodiments, the splice modulator binding site comprises the nucleic acid sequence of DGAGTTTGHV, wherein D is A, G, or T; wherein H is A, C, or T; and wherein V is A, G, or C. Further described herein, in certain embodiments, is a nucleic acid molecule comprising a minigene positioned immediately 5′ to a transgene, the minigene comprising: (a) a first exon immediately 5′ to a first intron; (b) a start codon; and (c) a splice modulator binding site, the splice modulator binding site comprises the nucleic acid sequence of DGAGTRRGHV (SEQ ID NO: I) or DGAGTRRNHV (SEQ ID NO: 2), wherein D is A, G, or T; and wherein R is A, or G; wherein N is A, C, G, or T; wherein H is A, C, or T; and wherein V is A, G, or C. See FIGS. 7A-7G. In some embodiments, the splice modulator binding site comprises the nucleic acid sequence of DGAGTTTGHV (SEQ ID NO: 84), wherein D is A, G, or T; wherein H is A, C, or T; and wherein V is A, G, or C. In some embodiments, the splice modulator binding site comprises TAGAGTAAGACA (SEQ ID NO: 75). In some embodiments, the splice modulator binding site comprises ATGAGTATGACA (SEQ ID NO: 76). In some embodiments, the splice modulator binding site comprises ATGAGTAAGCAG (SEQ ID NO: 77). In some embodiments, the splice modulator binding site comprises ATGAGTATGT (SEQ ID NO: 78). In some embodiments, the splice modulator binding site comprises ATGAGTTTGT (SEQ ID NO: 79). In some embodiments, the splice modulator binding site comprises ATGAGTAAGT (SEQ ID NO: 80). In some embodiments, the splice modulator binding site comprises ATGAGTTAGT (SEQ ID NO: 81).

In some embodiments, the nucleic acid comprises in 5′ to 3′ order the first exon comprising the start codon, the first intron, and the transgene. See FIG. 7A.

In some embodiments, the nucleic acid molecule further comprises a stop codon.

In some embodiments, the nucleic acid molecule further comprises a second exon.

In some embodiments, the nucleic acid comprises in 5′ to 3′ order the first exon comprising the start codon, the second exon comprising the stop codon, and the transgene. See FIG. 7B.

In some embodiments, the nucleic acid comprises in 5′ to 3′ order the first exon comprising the start codon, the first intron, the second exon comprising the stop codon, and the transgene. See FIG. 7B.

In some embodiments, the nucleic acid comprises in 5′ to 3′ order the first exon, the second exon comprising the start codon, and the transgene. See FIG. 7C.

In some embodiments, the nucleic acid comprises in 5′ to 3′ order the first exon, the second exon comprising the start codon, the first intron, and the transgene. See FIG. 7C.

In some embodiments, the nucleic acid molecule further comprises a third exon.

In some embodiments, the nucleic acid molecule further comprises a third intron.

In some embodiments, the nucleic acid comprises in 5′ to 3′ order the first exon, the second exon comprising the stop codon, the third exon comprising the start codon, and the transgene. See FIG. 7D.

In some embodiments, the nucleic acid comprises in 5′ to 3′ order the first exon, the first intron, the second exon comprising the stop codon, the second intron, the third exon comprising the start codon, the third intron, and the transgene. See FIG. 7D.

In some embodiments, the nucleic acid comprises in 5′ to 3′ order the first exon, the first intron, the second exon comprising the stop codon, the third exon comprising the start codon, the second intron, and the transgene. See FIG. 7F.

In some embodiments, the nucleic acid comprises in 5′ to 3′ order the first exon, the second exon comprising the start codon, the third exon comprising the stop codon, and the transgene. See FIGS. 7E and 7G.

In some embodiments, the nucleic acid comprises in 5′ to 3′ order the first exon, the first intron, the second exon comprising the start codon, the second intron, the third exon comprising the stop codon, the third intron, and the transgene. See FIG. 7E.

In some embodiments, the nucleic acid comprises in 5′ to 3′ order the first exon, the first intron, the second exon comprising the start codon, the third exon comprising the stop codon, the second intron, and the transgene. See FIG. 7G.

In some embodiments, the nucleic acid comprises in 5′ to 3′ order the first exon, the first intron, the second exon comprising the start codon, the second intron, and the transgene. See FIG. 7H.

In some embodiments, the nucleic acid comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 21-73. In some embodiments, the nucleic acid comprises a sequence having at least about 70% identity to any one of SEQ ID NOs: 21-73. In some embodiments, the nucleic acid comprises a sequence having at least about 75% identity to any one of SEQ ID NOs: 21-73. In some embodiments, the nucleic acid comprises a sequence having at least about 80% identity to any one of SEQ ID NOs: 21-73. In some embodiments, the nucleic acid comprises a sequence having at least about 85% identity to any one of SEQ ID NOs: 21-73. In some embodiments, the nucleic acid comprises a sequence having at least about 90% identity to any one of SEQ ID NOs: 21-73. In some embodiments, the nucleic acid comprises a sequence having at least about 95% identity to any one of SEQ ID NOs: 21-73. In some embodiments, the nucleic acid comprises a sequence having at least about 96% identity to any one of SEQ ID NOs: 21-73. In some embodiments, the nucleic acid comprises a sequence having at least about 97% identity to any one of SEQ ID NOs: 21-73. In some embodiments, the nucleic acid comprises a sequence having at least about 98% identity to any one of SEQ ID NOs: 21-73. In some embodiments, the nucleic acid comprises a sequence having at least about 99% identity to any one of SEQ ID NOs: 21-73. In some embodiments, the nucleic acid comprises a sequence having 100% identity to any one of SEQ ID NOs: 21-73. In some embodiments, SEQ ID NOs: 21-73 contain a first exon, a first intron, a second exon containing a start codon, and a second intron. In some embodiments, the design of SEQ ID NOs: 21-73 is reflected in FIG. 7H.

Described herein, in certain embodiments, is nucleic acid molecule comprising in 5′ to 3′: (a) a first exon comprising a first portion of a start codon at the 3′ end of the first exon; (b) a second exon comprising a second portion of a start codon at the 5′ end of the second exon; and (c) a third exon. See FIG. 6B.

In some embodiments, the third exon is a transgene.

Described herein, in certain embodiments, is a nucleic acid molecule comprising in 5′ to 3′ order a first exon, a second exon comprising a stop codon, a third exon comprising a first portion of a start codon, and a transgene comprising a second portion of the start codon. See FIG. 6J.

Described herein, in certain embodiments, is a nucleic acid molecule comprising in 5′ to 3′ order a first exon, a second exon comprising a first portion of a start codon, a third exon comprising a stop codon, and a transgene comprising a second portion of the start codon. See FIG. 6K.

In some embodiments, the nucleic acid molecule further comprises a first intron, a second intron, and a third intron interposed between the first exon, the second exon, the third exon, and the transgene, respectively.

In some embodiments, the splice modulator binding site does not include the nucleic acid sequence AAGAGT (SEQ ID NO: 3), ATGAGT (SEQ ID NO: 4), TAGAGT (SEQ ID NO: 5), TTGAGT (SEQ ID NO: 6), GAGAGT (SEQ ID NO: 7), GTGAGT (SEQ ID NO: 8), AAGAGT (SEQ ID NO: 9). ATGAGT (SEQ ID NO: 10), ACGAGT (SEQ ID NO: 11). AGGAGT (SEQ ID NO: 12), AGAGGTAGAG (SEQ ID NO: 13). TGAGGTTGAG (SEQ ID NO: 14). GGAGGTGGAG (SEQ ID NO: 15), TAG (SEQ ID NO: 16), CAG (SEQ ID NO: 17), TAG (SEQ ID NO: 18), or NAGAGTNNNN (SEQ ID NO: 19), wherein N is A, C, G, or T.

Described herein, in certain embodiments, are nucleic acid molecules comprising a minigene linked to a transgene, in which the minigene is designed to regulate transcription of the transgene by an exon-inclusion mechanism. In some embodiments, the minigene comprises: (a) two or more (e.g., about three, about four, or about five) introns; and (b) two or more (e.g., about three, about four, or about five) exons. In some embodiments, a minigene comprises: (a) two introns; and (b) two exons. In some embodiments, one or two nucleotides at the 5′-end of a start codon is located in an exon positioned 3′ to the first intron in the minigene. In some embodiments, one or two nucleotides at the 3′-end of the start codon is located in the transgene. In some embodiments, the nucleic acid molecule of the disclosure does not comprise a bipartite stop codon.

In some embodiments, the minigene comprises (a) a first intron and a second intron; and (b) a first exon and a second exon.

Described herein, in certain embodiments, are nucleic acid molecules comprising a minigene designed to regulate transcription of a transgene by an exon-exclusion mechanism; the minigene comprises a first exon, a first intron, one or more (e.g., about two, about three, about four, or about five) exons positioned 3′ to the first intron, a start codon, and a second intron. In some embodiments, one or two nucleotides at the 5′-end of the start codon is located in an exon of the one or more exons (e.g., the exons positioned 3′ to the first intron). In some embodiments, one or two nucleotides at the 3′-end of the start codon is located in the transgene. In some embodiments, the nucleic acid molecule of the disclosure functions by an exon-inclusion mechanism. In some embodiments, the nucleic acid molecule of the disclosure does not comprise a bipartite stop codon.

In some embodiments, the one or more exons positioned 3′ to the first exon comprise a second exon.

In some embodiments, the one or two nucleotides at the 5′-end of the start codon is located in the second exon.

Described herein, in certain embodiments, are nucleic acid molecules comprising a minigene designed to regulate transcription of a transgene by an exon-inclusion mechanism; the minigene comprises a first exon, a first intron, a second exon, a start codon, and a second intron, and is linked to a transgene in which one or two nucleotides at the 5′-end of the start codon is located in the second exon and one or two nucleotides at the 3′-end of the start codon is located in the transgene.

In some embodiments, two nucleotides at the 5′-end of the start codon are located in the second exon and one nucleotide at the 3′-end of the start codon is located in the transgene. In some embodiments, one nucleotide at the 5′-end of the start codon is located in the second exon and two nucleotides at the 3′-end of the start codon are located in the transgene. In some embodiments, the nucleic acid molecule of the disclosure functions by an exon-inclusion mechanism. In some embodiments, the nucleic acid molecule of the disclosure does not comprise a bipartite stop codon.

In some embodiments, the start codon is noncontiguous (e.g., split). Such a division may occur across the exons, introns, and/or transgene. For example, in some embodiments, at least one (e.g., one or two) nucleotide of the start codon is located in the second exon. In some embodiments, at least one (e.g., one or two) nucleotide of the start codon is located in a transgene. In some embodiments, at least one (e.g., one or two) nucleotide of the start codon is located in the second exon and at least one (e.g., one or two) nucleotide of the start codon is located in a transgene.

In some embodiments, the nucleic acid molecule comprises at least one splicing element (e.g., a 5′ splice site and/or a 3′ splice site). In some embodiments, the splicing element is adjacent to a start codon. In some embodiments, the splicing element is on one or both sides of a start codon.

None of the nucleic acid molecules of the disclosure include a bipartite stop codon.

### Start Codons

The nucleic acid molecules described herein encode a translation initiation sequence, e.g., a start codon (START). In some embodiments, the translation initiation sequence includes a Kozak or Shine-Dalgamo sequence. In some embodiments, the translation initiation sequence includes a Kozak sequence. Further examples of translation initiation sequences are described in paragraphs [0163]-[0165] of International Patent Publication No. WO 2019/118919.

In some embodiments, the nucleic acid molecule comprises a start codon which is adjacent to or located in an exon and/or an expression sequence (e.g., a transgene).

In some embodiments, the start codon is noncontiguous (e.g., split). Such a division may occur across the exons, introns, and/or transgene. For example, in some embodiments, at least one (e.g., one or two) nucleotide of the start codon is located in the second exon. In some embodiments, at least one (e.g., one or two) nucleotide of the start codon is located in a transgene. In some embodiments, at least one (e.g., one or two) nucleotide of the start codon is located in the second exon and at least one (e.g., one or two) nucleotide of the start codon is located in a transgene. In some embodiments, at least one nucleotide of start codon is located in the second exon and at least one nucleotide of start codon is located in a transgene. In some embodiments, two nucleotides of a start codon are located in the second exon and one nucleotide of a start codon is located in a transgene. In some embodiments, one nucleotide of a start codon is located in the second exon and two nucleotides of a start codon are located in a transgene.

In some embodiments, the start codon is a non-coding start codon.

Any suitable start codon may be used.

In some embodiments, the start codon is a three-nucleotide codon.

### Splice Modulator Binding Sites

The nucleic acid molecules described herein may include a splice modulator binding site. For example, in some embodiments, such a splice modulator binding site comprises the nucleic acid sequence of DGAGUNNGBD (SEQ ID NO: 1) or DGAGUNNNBD (SEQ ID NO: 2), wherein D is adenine (A), guanine (G), or uracil (U); wherein N is A, cytosine (C), G, or U; and wherein B is A, C, or U. For example, in some embodiments, a splice modulator binding site comprises the nucleic acid sequence of DGAGUNNGBD (SEQ ID NO: 1). In some embodiments, a splice modulator binding site comprises the nucleic acid sequence of DGAGUNNNBD (SEQ ID NO: 2).

In some embodiments, the splice modulator binding site does not include the nucleic acid sequence AAGAGU (SEQ ID NO: 3), AUGAGU (SEQ ID NO: 4), UAGAGU (SEQ ID NO: 5), UUGAGU (SEQ ID NO: 6). GAGAGU (SEQ ID NO: 7). GUGAGU (SEQ ID NO: 8), AAGAGU (SEQ ID NO: 9), AUGAGU (SEQ ID NO: 10). ACGAGU (SEQ ID NO: 11), AGGAGU (SEQ ID NO: 12), AGAGGTAGAG (SEQ ID NO: 13), UGAGGTUGAG (SEQ ID NO: 14), GGAGGTGGAG (SEQ ID NO: 15), TAG (SEQ ID NO: 16), CAG (SEQ ID NO: 17), UAG (SEQ ID NO: 18), or NAGAGTNNNN (SEQ ID NO: 19), wherein N is A, C, G, or T. For example, in some embodiments, the splice modulator binding site does not include the nucleic acid sequence AAGAGU (SEQ ID NO: 3). In some embodiments, the splice modulator binding site does not include the nucleic acid sequence AUGAGU (SEQ ID NO: 4). In some embodiments, the splice modulator binding site does not include the nucleic acid sequence UAGAGU (SEQ ID NO: 5). In some embodiments, the splice modulator binding site does not include the nucleic acid sequence UUGAGU (SEQ ID NO: 6). In some embodiments, the splice modulator binding site does not include the nucleic acid sequence GAGAGU (SEQ ID NO: 7). In some embodiments, the splice modulator binding site does not include the nucleic acid sequence GUGAGU (SEQ ID NO: 8). In some embodiments, the splice modulator binding site does not include the nucleic acid sequence AAGAGU (SEQ ID NO: 9). In some embodiments, the splice modulator binding site does not include the nucleic acid sequence AUGAGU (SEQ ID NO: 10). In some embodiments, the splice modulator binding site does not include the nucleic acid sequence ACGAGU (SEQ ID NO: 11). In some embodiments, the splice modulator binding site does not include the nucleic acid sequence AGGAGU (SEQ ID NO: 12). In some embodiments, the splice modulator binding site does not include the nucleic acid sequence AGAGGTAGAG (SEQ ID NO: 13). In some embodiments, the splice modulator binding site does not include the nucleic acid sequence UGAGGTUGAG (SEQ ID NO: 14). In some embodiments, the splice modulator binding site does not include the nucleic acid sequence GGAGGTGGAG (SEQ ID NO: 15). In some embodiments, the splice modulator binding site does not include the nucleic acid sequence TAG (SEQ ID NO: 16). In some embodiments, the splice modulator binding site does not include the nucleic acid sequence CAG (SEQ ID NO: 17). In some embodiments, the splice modulator binding site does not include the nucleic acid sequence UAG (SEQ ID NO: 18). In some embodiments, the splice modulator binding site does not include the nucleic acid sequence NAGAGTNNNN (SEQ ID NO: 19).

In some embodiments, a splice modulator binding site is located in the second exon of the nucleic acid molecule. Furthermore, a splice modulator may bind a splice modulator binding site, such that binding may effectuate a splicing event and/or the transcription of a linked transgene.

The splice modulator binding site may include a set of four or more (e.g., five, six, seven, or eight) nucleotides.

In some embodiments, splice modulator binding site includes a sequence that is recognized by an RNA binding protein (RBP). In some embodiments, the RBP is tissue-specific. In some embodiments, an RBP is exclusively expressed in the brain. In accordance with this principle, in some embodiments, a nucleic acid molecule of the disclosure operates by an exon-inclusion principle by using a splice modulator that targets an RBP with tissue-specific expression.

In some embodiments, the RBP is a splicing enhancer (e.g.; a serine and arginine-rich (SR) protein) (for a review, see e.g., Jeong S. Mol Cells. 2017 January; 40(1):1-9). In some embodiments, the RBP is a splicing repressor (e.g.: a heterogeneous nuclear ribonucleoprotein (hnRNP)), for a review, see e.g Han et al. Biochem J. 2010 Sep. 15; 430(3):379-9, 2.

In some embodiments, the splice modulator binding site is located at the junction between the second exon and the second intron.

In some embodiments, a splice modulator binding site is designed by a screen of greater than 100 (e.g., greater than 1,000, greater than 10,000, greater than 100,000, or greater than 1,000,000) candidate splice modulator binding sites for their ability to functionally respond to a specific splice modulator. In some embodiments, the screen assesses both basal and splice modulator-stimulated inclusion of a bipartite start codon as well as the resulting increases or decreases in respective transgene expression. In some embodiments, the screen, for example, utilizes a DNA construct including a minigene derived from a naturally occurring mammalian gene or a synthetic intron-containing construct with canonical 5′ and 3′ splice site sequences. In some embodiments, each minigene is linked to a reporter gene such as, for example, firefly luciferase, and assembled through DNA synthesis and molecular cloning techniques known in the art. Libraries of minigene constructs with splicing modulator binding site point mutations, insertions, and/or deletions can be generated and introduced into mammalian cells using electroporation, chemical transfection, viral-mediated integration, or viral mediated episomal introduction. In some embodiments, assessment of the candidate splice modulator binding site and position in the minigene is accomplished by including a reporter gene, such as a luminescent enzyme (e.g., firefly luciferase, nanoluc luciferase. renilla luciferase, and gaussia luciferase), fluorescent protein (e.g., green fluorescent protein, blue fluorescent protein, and red fluorescent protein), or colorimetric enzyme (e.g., beta lactamase or secreted embryonic alkaline phosphatase) in the DNA construct that generates a quantifiable signal proportional to the splicing of an exon (e.g., the second exon), readable by, for example, flow cytometry, microscopy, or multi-modal microplate readers using photo-multiplier tubes. Alternatively, for example, the candidate splice modulator-dependent biological activity is assessed by sequencing the mRNA transcripts produced by the DNA construct with RNASeq or quantitative reverse transcription PCR. In some embodiments, following such an exemplary screen, candidate splice modulators are applied to cells containing the DNA construct for up to 6, 12, 24, or 72 hours and then assessed for start-codon inclusion activity. In some embodiments, successive rounds of candidate splice modulator binding site design, synthesis, and assay assessment is performed to optimize splice modulator binding sites for the transgene regulating minigene.

In some embodiments, a splice modulator binding site is designed by a screen of various substances and/or chemical derivations of a substance for their ability to regulate the expression of a reporter gene linked to a minigene construct in mammalian cells. In some embodiments, the minigene template is derived from a naturally occurring mammalian gene or a synthetic intron-containing construct with canonical 5′ and 3′ splice site sequences. In some embodiments, the minigene template is linked to a reporter gene such as, for example, firefly luciferase to enable detection of gene expression changes resulting from chemical substances that act as splice modulators to mediate alternative splicing of the minigene.

In some embodiments, the splice modulator binding site comprises an intronic or exonic splice silencer or enhancer element. For example, in some embodiments, the splice modulator binding site comprises an intronic splice silencer. In some embodiments, the splice modulator binding site comprises an exonic splice silencer. In some embodiments, the splice modulator binding site comprises an enhancer element.

In some embodiments, the splice modulator binding site is an aptamer. In some embodiments, the aptamer is part of a riboswitch.

### Riboswitches

A riboswitch is a regulatory segment of a nucleic acid molecule, that affects the expression of a genetic element. Thus, a nucleic acid molecule that contains a riboswitch is directly involved in regulating its activity in response to the concentrations of its effector molecule by forming alternative structures in response to this effector binding.

More particularly, riboswitches consist of (i) a splice modulator binding site domain (e.g. an aptamer) that binds defined ligands with high affinity and (ii) an expression platform, which translates splice modulator binding (e.g., binding of a splice modulator to an aptamer) into an effect on gene expression. The regulation occurs at either the level of transcription (e.g., by formation of terminator or antiterminator structures) or translation (e.g., by presentation or sequestering of the ribosomal binding site). Due to a modular composition, these elements can be manipulated by combining different splice modulator binding sites (e.g., aptamers) and expression platforms.

Riboswitches are known to respond to RNA derivatives, including coenzymes (13 riboswitch classes, nucleotide derivatives (7 riboswitch classes), signaling molecules (5 riboswitch classes) as well as ions (5 riboswitch classes), amino acids (3 riboswitch classes), and other metabolites (5 riboswitch classes). To date, 38 different classes of riboswitches have been discovered with their cognate ligands (for a review, see e.g., McCown, Phillip J., et al. Rna 23.7. 2017. 995-1011). In particular, more than 100,000 representative riboswitches can be classified into 38 validated riboswitch classes. Each riboswitch class is named according to its ligand. As described in Table 1, below, these classes include Thiamin pyrophosphate (TPP), adenosylcobalamin (AdoCbl) or coenzyme B₁₂, S-adenosylmethionine (SAM), cyclic-di-GMP (C-di-GMP), glycine, favin mononucleotide (FMN), divalent manganese (Mn²⁺), lysine, cyclic-di-AMP (C-di-AMP), fluoride, prequeuosinei (PreQi), Guanine, 5-aminoimidazole-4-carboxamide ribonucleoside-5′-triphosphate (ZTP), glucosamine-6-phosphate (GlcN6P), tetrahydrofolate (THF), glutamine, molybdenum cofactor (Moco), divalent magnesium (Mg²⁻), S-adenosylhomocysteine (SAH), guanidine, azaaromatic, tungsten cofactor (Wco), aquacobalamin (AqCbl), divalent nickel and divalent cobalt (NiCo), cyclic AMP-GMP (c-AMP-GMP), 2′-deoxyguanosine (2′-dG), and FMN riboswitch variant (FMVN-Var). In some embodiments, multiple structural classes for the same ligand have been identified (e.g., as is the case for the S-adenosylmethionine (SAM) class). A riboswitch of any of the described classes or any other now known or later discovered class is included in a nucleic acid molecule described herein. Such a riboswitch may serve as a splice modulator binding site described herein.

TABLE 1
Riboswitch Classes and Number of Representatives

| | Approximate Number |
|---|---|
| Riboswitch Class | of Representatives |
| Thiamin pyrophosphate (TPP) | >15,000 |
| Adenosylcobalamin (AdoCbl) or | ~15,000 |
| coenzyme B₁₂ | |
| S-adenosylmethionine (SAM) | >15,000 |
| Cyclic-di-GMP (C-di-GMP) | >5,000 |
| Glycine | >5,000 |
| Flavin mononucleotide (FMN) | >5,000 |
| Divalent manganese (Mn²⁺) | ~5,000 |
| Lysine | <5,000 |
| Cyclic-di-AMP (C-di-AMP) | <5,000 |
| Fluoride | <5,000 |
| Prequeuosine₁ (PreQ₁) | <5,000 |
| Guanine | <5,000 |
| 5-aminoimidazole-4-carboxamide | <5,000 |
| Ribonucleoside-5′-triphosphate (ZTP) | |
| Glucosamine-6-phosphate (GlcN6P) | <5,000 |
| Tetrahydrofolate (THF) | <5,000 |
| Glutamine | <5,000 |
| Molybdenum cofactor (Moco) | <5,000 |
| Divalent magnesium (Mg²⁺) | <5,000 |
| S-adenosylhomocysteine (SAH) | <5,000 |
| Guanidine | <5,000 |
| Azaaromatic | <5,000 |
| Tungsten cofactor (Wco) | <5,000 |
| Aquacobalamin (AqCbl) | <5,000 |
| Divalent nickel and divalent cobalt (NiCo) | <5,000 |
| Cyclic AMP-GMP (c-AMP-GMP) | <5,000 |
| 2′-deoxyguanosine (2′-dG) | <5,000 |
| FMN riboswitch variant (FMN-Var) | <5,000 |

In some embodiments, a riboswitch is a riboswitch in the class of TPP riboswitches. In some embodiments, a riboswitch is a riboswitch in the class of AdoCbl or coenzyme B₁₂ riboswitches. In some embodiments, a riboswitch is a riboswitch in the class of SAM riboswitches. In some embodiments, a riboswitch is a riboswitch in the class of C-di-GMP riboswitches. In some embodiments, a riboswitch is a riboswitch in the class of glycine riboswitches. In some embodiments, a riboswitch is a riboswitch in the class of FMN riboswitches. In some embodiments, a riboswitch is a riboswitch in the class of Mn²⁺ riboswitches. In some embodiments, a riboswitch is a riboswitch in the class of lysine riboswitches. In some embodiments, a riboswitch is a riboswitch in the class of C-di-AMP riboswitches. In some embodiments, a riboswitch is a riboswitch in the class of fluoride riboswitches. In some embodiments, a riboswitch is a riboswitch in the class of PreQi riboswitches. In some embodiments, a riboswitch is a riboswitch in the class of guanine riboswitches. In some embodiments, a riboswitch is a riboswitch in the class of ZTP riboswitches. In some embodiments, a riboswitch is a riboswitch in the class of GlcN6P riboswitches. In some embodiments, a riboswitch is a riboswitch in the class of THF riboswitches. In some embodiments, a riboswitch is a riboswitch in the class of glutamine riboswitches. In some embodiments, a riboswitch is a riboswitch in the class of Moco riboswitches. In some embodiments, a riboswitch is a riboswitch in the class of Mg²⁺ riboswitches. In some embodiments, a riboswitch is a riboswitch in the class of SAH riboswitches. In some embodiments, a riboswitch is a riboswitch in the class of guanidine riboswitches. In some embodiments, a riboswitch is a riboswitch in the class of azaaromatic riboswitches. In some embodiments, a riboswitch is a riboswitch in the class of Wco riboswitches. In some embodiments, a riboswitch is a riboswitch in the class of AqCbl riboswitches. In some embodiments, a riboswitch is a riboswitch in the class of NiCo riboswitches. In some embodiments, a riboswitch is a riboswitch in the class of c-AMP-GMP riboswitches. In some embodiments, a riboswitch is a riboswitch in the class of 2′-dG riboswitches. In some embodiments, a riboswitch is a riboswitch in the class of FMN-Var riboswitches.

In some embodiments, the riboswitch responds to RNA-based coenzymes thiamin pyrophosphate (TPP), B12, SAM, and FMN.

In some embodiments, the riboswitch responds to c-di-GMP (c-di-AMP and c-AMP-GMP). 5-aminoimidazole-4-carboxamide riboside 5′-monophosphate (ZTP), glucosamine-6-phosphate (GlcN6P), an azaaromatic ligand, guanidine, lysine, glutamine, a divalent cation (e.g., Mg²⁺, Ni²⁺, and Co²⁺), a monoanion, and/or fluoride. In some embodiments, the riboswitch responds to c-di-GMP (c-di-AMP and c-AMP-GMP). In some embodiments, the riboswitch responds to ZMP or its triphosphorylated form ZTP. In some embodiments, the riboswitch responds to GlcN6P. In some embodiments, the riboswitch responds to an azaaromatic ligand. In some embodiments, the riboswitch responds to guanidine. In some embodiments, the riboswitch responds to lysine. In some embodiments, the riboswitch responds to glutamine. In some embodiments, the riboswitch responds to a divalent cation (e.g., Mg²⁺, Ni²⁺, and Co²⁺). In some embodiments, the riboswitch responds to a monoanion. In some embodiments, the riboswitch responds to fluoride.

A riboswitch is located in an exon of a nucleic acid molecule of the disclosure.

As described herein, in some embodiments, a riboswitch is a riboswitch of any suitable riboswitch class now known (e.g., including, but not limited, by a riboswitch class described herein) or later discovered. In some embodiments, a splice modulator binding site described herein is an aptamer that is part of a riboswitch. Any suitable aptamer is used.

### Transgenes

In some embodiments, a nucleic acid molecule of the disclosure includes a minigene construct linked to a transgene. In some embodiments, a transgene encodes a protein, RNA (e.g., a functional or regulatory RNA of interest), miRNA, or shRNA of interest.

In some embodiments, the protein, RNA, miRNA, or shRNA of interest is expressed in the presence of the splice modulator (see Splice Modulator section). In some embodiments, the protein, RNA, miRNA, or shRNA of interest is solely expressed in the presence of the splice modulator.

In some embodiments, a transgene encodes a protein. In some embodiments, the protein is solely expressed in the presence of the splice modulator.

In some embodiments, the start codon is in the second exon of the minigene.

In some embodiments, two nucleotides at the 5′-end of the bipartite start codon are located in the second exon and one nucleotide at the 3′-end of the start codon is located in the transgene.

In some embodiments, one nucleotide at the 5′-end of the bipartite start codon is located in the second exon and two nucleotides at the 3′-end of the start codon are located in the transgene.

### Regulatory Sequences

The nucleic acid molecules disclosed herein may be desired to be expressed at sufficiently high levels to elicit a therapeutic benefit. Accordingly, polynucleotide expression may be mediated by a promoter sequence capable of driving robust expression of the disclosed nucleic acid molecules. According to the methods and compositions disclosed herein, the promoter, in some embodiments, is a heterologous promoter. Useful heterologous control sequences generally include those derived from sequences encoding mammalian or viral genes. For purposes of the present disclosure, both heterologous promoters and other control elements, such as tissue-specific and inducible promoters, enhancers, splicing enhancers, splicing silencers, and the like will be of particular use.

In some embodiments, a promoter is derived in its entirety from a native gene or is composed of different elements derived from different naturally-occurring promoters. In some embodiments, a promoter includes a synthetic polynucleotide sequence. Different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g., tetracycline-responsive promoters) are well known in the art.

Exemplary promoters that are useful for the expression of the disclosed nucleic acid molecule agents in mammalian cells include ubiquitous promoters such as, e.g., a GFAP, Nestin, S100B, Nefh, dystrophinH1 promoter, 7SK promoter, apolipoprotein E-human-alpha 1-antitrypsin promoter, CK8 promoter, murine U1 promoter (mU1a), elongation factor 1α (EF-1α) promoter, thyroxine binding globulin (TBG) promoter, phophoglycerate kinase (PKG) promoter. CAG (composite of the (CMV) cytomegalovirus enhancer the chicken beta actin promoter (CBA) and the rabbit beta globin intron), the SV40 early promoter, murine mammary tumor virus LTR promoter; adenovirus major late promoter (Ad MLP); a herpes simplex virus (HSV) promoter, a CMV promoter such as the CMV immediate early promoter region (CMV-IE), rous sarcoma virus (RSV) promoter, and U6 promoter or variants thereof. For the purpose of driving cell-type specific expression of nucleic acid molecules disclosed herein, cell-type specific promoters may be used. In some embodiments, neuron-specific expression of a nucleic acid molecule is conferred using neuronal-specific promoters, such as, e.g., a human synapsin 1 (hSyn) promoter, hexaribonucleotide binding protein-3 (NeuN) promoter, Ca2+/calmodulin-dependent protein kinase II (CaMKII) promoter, tubulin alpha I (Tα-1) promoter, neuron-specific enolase (NSE) promoter, platelet-derived growth factor beta chain (PDGFβ) promoter, vesicular glutamate transporter (VGLUT) promoter, somatostatin (SST) promoter, neuropeptide Y (NPY) promoter, vasoactive intestinal peptide (VIP) promoter, parvalbumin (PV) promoter, glutamate decarboxylase (GAD65 or GAD67) promoter, promoter of Dopamine-1 receptor (DRD1) and Dopamine-2 receptor (DRD2), microtubule-associated protein 1B (MAP1B), complement component 1 q subcomponent-like 2 (Clql2) promoter, pro-opiomelanocortin (POMC) promoter, and prospero homeobox protein 1 (PROX1) promoter. In some embodiments, the promoter is in its entirety or derived from the functional regulatory elements of any of the exemplified promoters.

Synthetic promoters, hybrid promoters, and the like may also be used in conjunction with the methods and compositions disclosed herein. In addition, sequences derived from non-viral genes, such as the murine metallothionein gene, will also find use herein. Such promoter sequences are commercially available from, e.g., Stratagene (San Diego, CA).

In some embodiments, the promoter is a chimeric promoter or a hybrid promoter. For example, in some embodiments, the promoter is a chimeric promoter. In some embodiments, the promoter is a hybrid promoter.

In some embodiments, the promoter does or does not contain intragenic introns. For example, in some embodiments, the promoter does contain intragenic introns. In some embodiments, the promoter does not contain intragenic introns.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Polynucleotides for use in the compositions and methods described herein, in some embodiments, include a mammalian enhancer sequence. In some embodiments, enhancers are from the genes that encode mammalian globin, elastase, albumin, a-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982). In some embodiments, an enhancer is spliced into a vector containing a polynucleotide encoding a nucleic acid molecule of the disclosure, for example, at a position 5′ or 3′ to this gene. In some embodiments, the enhancer is positioned at the 5′ side of the promoter, which in turn is located 5′ relative to the polynucleotide encoding a nucleic acid molecule of the disclosure.

Additional regulatory elements of the disclosure include polyadenylation sequences (polyA sequences). Exemplary polyAs of the disclosure include, but are not limited to an SV40 polyA, a human growth hormone (HGH) polyA, a bovine growth hormone (BGH) polyA, a beta-globin polyA, an alpha-globin polyA, an ovalbumin polyA, a kappa-light chain polyA, or a synthetic polyA. For example, in some embodiments, the polyA is an SV40 polyA. In some embodiments, the polyA is a human growth hormone (HGH) polyA. In some embodiments, the polyA is a bovine growth hormone (BGH) polyA. In some embodiments, the polyA is a beta-globin polyA. In some embodiments, the polyA is an alpha-globin polyA. In some embodiments, the polyA is an ovalbumin polyA. In some embodiments, the polyA is a kappa-light chain polyA. In some embodiments, the polyA is a synthetic polyA.

In some embodiments, a regulatory element includes an intronic or exonic splice silencer or enhancer element. For example, in some embodiments, a regulatory element includes an intronic splice silencer. In some embodiments, regulatory element includes an exonic splice silencer. In some embodiments, regulatory element includes an enhancer element.

In some embodiments, the nucleic acid molecule further comprises a regulatory site capable of being recognized by a spliceosome complex.

### Splice Modulators

The present disclosure also provides a composition comprising the nucleic acid molecule of any one of the foregoing aspects and a splice modulator, wherein the splice modulator comprises a structure of Formula (I) or a pharmaceutically acceptable salt, solvate, or prodrug thereof.

Described herein, in certain embodiments, is a composition comprising: (i) a nucleic acid molecule comprising a minigene positioned immediately 5′ to a transgene, the minigene comprising: a) an exon positioned 5′ to an intron; and b) a start codon having a first portion and a second portion, wherein the first portion and second portion of the start codon are not in the same exon; c) a splice modulator binding site; and (ii) a splice modulator that binds the splice modulator binding site and comprises a structure according to Formula (I) or a pharmaceutically acceptable salt, solvate, or prodrug thereof. See FIGS. 6A-6K.

Described herein, in certain embodiments, is a composition comprising: (i) a nucleic acid molecule comprising a minigene positioned immediately 5′ to a transgene, the minigene comprising: (a) a first exon immediately 5′ to a first intron: (b) a start codon; and (c) a splice modulator binding site; (ii) a splice modulator that binds the splice modulator binding site and comprises a structure according to Formula (I) or a pharmaceutically acceptable salt, solvate, or prodrug thereof. See FIGS. 7A-7G.

In some embodiments, the splice modulator binding site comprises the nucleic acid sequence of DGAGTDDGHV (SEQ ID NO: 82) or DGAGTDDNHV (SEQ ID NO: 83), wherein D is A, G, or T; wherein R is A, or G; wherein N is A, C, G, or T; wherein H is A, C, or T; and wherein V is A, G, or C. In some embodiments, the splice modulator binding site comprises the nucleic acid sequence of DGAGTTTGHV (SEQ ID NO: 84), wherein D is A, G, or T; wherein H is A, C, or T; and wherein V is A, G, or C. In some embodiments, the splice modulator binds to the splice modulator binding site comprising the nucleic acid sequence of DGAGTRRGHV (SEQ ID NO: 1) or DGAGTRRNHV (SEQ ID NO: 2), wherein D is A, G, or T; and wherein R is A, or G; and wherein N is A, C, G, or T; wherein H is A, C, or T; wherein V is A, G, or C.

In some embodiments, the splice modulator binding site does not include the nucleic acid sequence AAGAGT (SEQ ID NO: 3), ATGAGT (SEQ ID NO: 4), TAGAGT (SEQ ID NO: 5), TTGAGT (SEQ ID NO: 6), GAGAGT (SEQ ID NO: 7), GTGAGT (SEQ ID NO: 8), AAGAGT (SEQ ID NO: 9). ATGAGT (SEQ ID NO: 10), ACGAGT (SEQ ID NO: 11). AGGAGT (SEQ ID NO: 12). AGAGGTAGAG (SEQ ID NO: 13), TGAGGTTGAG (SEQ ID NO: 14), GGAGGTGGAG (SEQ ID NO: 15), TAG (SEQ ID NO: 16), CAG (SEQ ID NO: 17), TAG (SEQ ID NO: 18), or NAGAGTNNNN (SEQ ID NO: 19), wherein N is A, C, G, or T.

Splice modulators of the present disclosure include a structure of Formula (I):

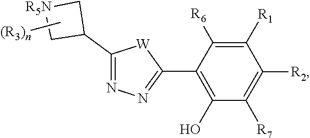

or a pharmaceutically acceptable salt, solvate, or prodrug thereof, wherein:
W is —S— or —HC═CH—;
R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, —(CH₂)₀₋₂—C₃-C₈cycloalkyl, NH₂, NH(C₁-C₆alkyl), N(C₁-C₆alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4-to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
R₅ is H, C₁-C₆alkyl, C₂-C₆alkenyl, C₂-C₆alkynyl, C₁-C₆alkoxyl, C₃-C₈cycloalkyl, —CH₂C₃-C₈cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆alkyl, C₁-C₆haloalkyl, C₁-C₆heteroalkyl, C₁-C₆alkoxyl, C₃-C₈cycloalkyl, spiro C₃-C₈cycloalkyl, spiro 4-7 membered heterocyclyl, 5-6 membered heteroaryl, oxo, cyano, or hydroxyl;
R₇ is H, halogen, C₁-C₆alkyl or C₁-C₆haloalkyl; and
n is 0, 1, 2, 3, 4, or 5.

In some aspects, the present disclosure provides, inter alia, a structure of Formula (Ic):

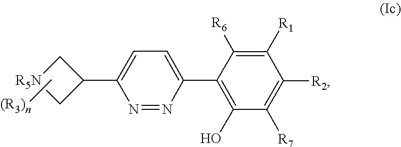

or a pharmaceutically acceptable salt, solvate, or prodrug thereof, wherein:
R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, —(CH₂)₀₋₂—C₃-C₈cycloalkyl, NH₂, NH(C₁-C₆alkyl), N(C₁-C₆alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4-to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
R₅ is H, C₁-C₆alkyl, C₂-C₆alkenyl, C₂-C₆alkynyl, C₁-C₆alkoxyl, C₃-C₈cycloalkyl, —CH₂C₃-C₈cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆alkyl, C₁-C₆haloalkyl, C₁-C₆heteroalkyl, C₁-C₆alkoxyl, C₃-C₈cycloalkyl, spiro C₃-C₈cycloalkyl, spiro 4-7 membered heterocyclyl, 5-6 membered heteroaryl, oxo, cyano, or hydroxyl;
R₆ is H, halogen, C₁-C₆alkyl or C₁-C₆haloalkyl;
R₇ is H, halogen, C₁-C₆alkyl or C₁-C₆haloalkyl; and
n is 0, 1, 2, 3, 4, or 5.

In some aspects, the present disclosure provides, inter alia, a structure of Formula (Id):

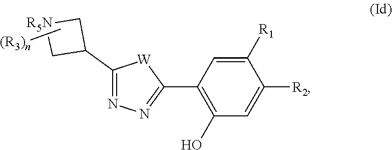

or a pharmaceutically acceptable salt, solvate, or prodrug thereof, wherein:
W is —S— or —HC═CH—;
R₁ is H, halogen, hydroxyl, cyano, C₁-C₆alkyl, C₂-C₆alkenyl, C₂-C₆alkynyl, C₁-C₆haloalkyl, C₁-C₆alkoxyl, CL-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂;
R₅ is H, C₁-C₆alkyl, C₂-C₆alkenyl, C₂-C₆alkynyl, C₁-C₆alkoxyl, C₃-C₈cycloalkyl, —CH₂C₃-C₈cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆alkyl, C₁-C₆haloalkyl, C₁-C₆heteroalkyl, C₁-C₆alkoxyl, C₃-C₈cycloalkyl, spiro C₃-C₈cycloalkyl, spiro 4-7 membered heterocyclyl, 5-6 membered heteroaryl, oxo, cyano, or hydroxyl; and
n is 0, 1, 2, 3,4, or 5.

In some embodiments, W is —S—.

In some embodiments, W is —HC═CH—.

In some embodiments, R₁ is H, halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S.

In some embodiments, R₁ is H.

In some embodiments, R₁ is halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S heterocyclyl, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S.

In some embodiments, R₁ is halogen, hydroxyl, cyano, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S heterocyclyl.

In some embodiments, R₁ is halogen, hydroxyl, or cyano.

In some embodiments, R₁ is halogen. In some embodiments, R₁ is F, Cl, Br, or I. In some embodiments, R₁ is F, Cl, or Br. In some embodiments, R₁ is F or Cl. In some embodiments, R₁ is F. In some embodiments, R₁ is Cl. In some embodiments, R₁ is Br. In some embodiments, R₁ is I.

In some embodiments, R₁ is hydroxyl.

In some embodiments, R₁ is cyano.

In some embodiments, R₁ is C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S.

In some embodiments, R₁ is C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S.

In some embodiments, R₁ is C₁-C₆alkyl, C₂-C₆alkenyl, or C₂-C₆alkynyl.

In some embodiments, R₁ is C₁-C₆alkyl.

In some embodiments, R₁ is methyl. In some embodiments, R₁ is ethyl. In some embodiments, R₁ is propyl. In some embodiments, R₁ is butyl. In some embodiments, R₁ is iso-propyl. In some embodiments, R₁ is iso-butyl. In some embodiments, R₁ is sec-butyl. In some embodiments, R₁ is tert-butyl.

In some embodiments, R₁ is C₁-C₆alkyl optionally substituted with one or more C₃-C₈cycloalkyl, aryl, or 4- to 7-membered heterocyclyl comprising 1, 2, or 3 heteroatoms independently selected from N, O, and S. In some embodiments, R₁ is C₁-C₆alkyl substituted with one or more C₃-C₈cycloalkyl, aryl, or 4- to 7-membered heterocyclyl comprising 1, 2, or 3 heteroatoms independently selected from N, O, and S. In some embodiments, R₁ is C₁-C₆alkyl substituted with one C₃-C₈cycloalkyl, aryl, or 4- to 7-membered heterocyclyl comprising 1, 2, or 3 heteroatoms independently selected from N, O, and S, In some embodiments, R₁ is C₁-C₆alkyl substituted with two C₃-C₈cycloalkyl, aryl, or 4- to 7-membered heterocyclyl comprising 1, 2, or 3 heteroatoms independently selected from N, O, and S. In some embodiments, R₁ is C₁-C₆alkyl substituted with three C₃-C₈cycloalkyl, aryl, or 4- to 7-membered heterocyclyl comprising 1, 2, or 3 heteroatoms independently selected from N, O, and S,

In some embodiments, R₁ is C₂-C₆alkenyl. In some embodiments, R₁ is C₂alkenyl. In some embodiments, R₁ is C₃alkenyl. In some embodiments, R₁ is C₄alkenyl. In some embodiments, R₁ is C₅alkenyl. In some embodiments, R₁ is C₆alkenyl.

In some embodiments, R₁ is C₂-C₆alkenyl optionally substituted with one or more C₃-C₈cycloalkyl, aryl, or 4- to 7-membered heterocyclyl comprising 1, 2, or 3 heteroatoms independently selected from N, O, and S. In some embodiments, R₁ is C₂-C₆alkenyl substituted with one or more C₃-C₈cycloalkyl, aryl, or 4- to 7-membered heterocyclyl comprising 1, 2, or 3 heteroatoms independently selected from N, O, and S, In some embodiments, R₁ is C₂-C₆alkenyl substituted with one C₃-C₈cycloalkyl, aryl, or 4- to 7-membered heterocyclyl comprising 1, 2, or 3 heteroatoms independently selected from N, O, and S. In some embodiments, R₁ is C₂-C₆alkenyl substituted with two C₃-C₈cycloalkyl, aryl, or 4- to 7-membered heterocyclyl comprising 1, 2, or 3 heteroatoms independently selected from N, O, and S, In some embodiments, R₁ is C₂-C₆alkenyl substituted with three C₃-C₈cycloalkyl, aryl, or 4- to 7-membered heterocyclyl comprising 1, 2, or 3 heteroatoms independently selected from N, O, and S.

In some embodiments, R₁ is C₂-C₆alkynyl. In some embodiments, R₁ is C₂alkynyl. In some embodiments, R₁ is C₃alkynyl. In some embodiments, R₁ is C₄alkynyl. In some embodiments, R₁ is C₅alkynyl. In some embodiments, R₁ is C₆alkynyl.

In some embodiments, R₁ is C₂-C₆alknyl optionally substituted with one or more C₃-C₈cycloalkyl, aryl, or 4- to 7-membered heterocyclyl comprising 1, 2, or 3 heteroatoms independently selected from N, O, and S. In some embodiments, R₁ is C₂-C₆alkynyl substituted with one or more C₃-C₈cycloalkyl, aryl, or 4- to 7-membered heterocyclyl comprising 1, 2, or 3 heteroatoms independently selected from N, O, and S, In some embodiments, R₁ is C₂-C₆alkynyl substituted with one C₃-C₈cycloalkyl, aryl, or 4- to 7-membered heterocyclyl comprising 1, 2, or 3 heteroatoms independently selected from N, O, and S. In some embodiments, R₁ is C₂-C₆alkynyl substituted with two C₃-C₈cycloalkyl, aryl, or 4- to 7-membered heterocyclyl comprising 1, 2, or 3 heteroatoms independently selected from N, O, and S, In some embodiments, R₁ is C₂-C₆alkynyl substituted with three C₃-C₈cycloalkyl, aryl, or 4- to 7-membered heterocyclyl comprising 1, 2, or 3 heteroatoms independently selected from N, O, and S.

In some embodiments, R₁ is C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S, wherein the alkoxyl, cycloalkyl, and heterocyclyl are optionally substituted with one or more C₃-C₈cycloalkyl, aryl, or 4- to 7-membered heterocyclyl comprising 1, 2, or 3 heteroatoms independently selected from N, O, and S.

In some embodiments, R₁ is C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or 4- to 7-membered heterocyclyl comprising 1, 2, or 3 heteroatoms independently selected from N, O, and S.

In some embodiments, R₁ is C₁-C₆haloalkyl, C₁-C₆alkoxyl, or C₁-C₆haloalkoxyl, wherein the alkoxyl is optionally substituted with one or more C₃-C₈cycloalkyl, aryl, or 4- to 7-membered heterocyclyl comprising 1, 2, or 3 heteroatoms independently selected from N, O, and S.

In some embodiments, R₁ is C₁-C₆haloalkyl, C₁-C₆alkoxyl, or C₁-C₆haloalkoxyl.

In some embodiments, R₁ is C₁-C₆haloalkyl. In some embodiments, R₁ is halomethyl. In some embodiments, R₁ is haloethyl. In some embodiments, R₁ is halopropyl. In some embodiments. R₁ is halobutyl. In some embodiments, R₁ is halopentyl. In some embodiments, R₁ is halohexyl.

In some embodiments, R₁ is C₁-C₆alkoxyl. In some embodiments, R₁ is methoxyl. In some embodiments, R₁ is ethoxyl. In some embodiments, R₁ is propoxyl. In some embodiments. Ri is butyoxyl. In some embodiments, R₁ is pentyoxyl. In some embodiments, R₁ is hexoxyl.

In some embodiments, R₁ is C₁-C₆haloalkoxyl. In some embodiments, R₁ is halomethoxyl. In some embodiments, R₁ is haloethoxyl. In some embodiments, R₁ is halopropoxyl. In some embodiments, R₁ is halobutoxyl. In some embodiments, R₁ is halopentoxyl. In some embodiments, R₁ is halohexoxyl.

In some embodiments, R₁ is C₃-C₈cycloalkyl or 4- to 7-membered heterocyclyl comprising 1, 2, or 3 heteroatoms independently selected from N, O, and S, wherein the cycloalkyl and heterocyclyl are optionally substituted with one or more C₃-C₈cycloalkyl, aryl, or 4- to 7-membered heterocyclyl comprising 1, 2, or 3 heteroatoms independently selected from N, O, and S.

In some embodiments, R₁ is C₃-C₈cycloalkyl or 4- to 7-membered heterocyclyl comprising 1, 2, or 3 heteroatoms independently selected from N, O, and S.

In some embodiments, R₁ is C₃-C₈cycloalkyl. In some embodiments, R₁ is cyclopropyl. In some embodiments, R₁ is cyclobutyl. In some embodiments, R₁ is cyclopentyl. In some embodiments, R₁ is cyclohexyl.

In some embodiments, R₁ is bridged C₃-C₈cycloalkyl. In some embodiments, R₁ is fused C₃-C₈cycloalkyl. In some embodiments, R₁ is spiro C₃-C₈cycloalkyl.

In some embodiments, R₁ is C₃-C₈cycloalkyl optionally substituted with one or more C₃-C₈cycloalkyl, aryl, or 4- to 7-membered heterocyclyl comprising 1, 2, or 3 heteroatoms independently selected from N, O, and S. In some embodiments, R₁ is C₃-C₈cycloalkyl optionally substituted with one or more C₃-C₈cycloalkyl or aryl. In some embodiments, R₁ is C₃-C₈cycloalkyl substituted with one or more C₃-C₈cycloalkyl or aryl. In some embodiments, R₁ is C₃-C₈cycloalkyl substituted with one or more C₃-C₈cycloalkyl. In some embodiments, R₁ is C₃-C₈cycloalkyl substituted with one or more aryl.

In some embodiments, R₁ is 4- to 7-membered heterocyclyl comprising 1, 2, or 3 heteroatoms independently selected from N, O, and S.

In some embodiments, R₁ is 4-membered heterocyclyl comprising 1, 2, or 3 heteroatoms independently selected from N, O, and S.

In some embodiments, R₁ is 5-membered heterocyclyl comprising 1, 2, or 3 heteroatoms independently selected from N, O, and S.

In some embodiments, R₁ is 6-membered heterocyclyl comprising 1, 2, or 3 heteroatoms independently selected from N, O, and S.

In some embodiments, R₁ is 7-membered heterocyclyl comprising 1, 2, or 3 heteroatoms independently selected from N, O, and S.

In some embodiments, R₁ is 4- to 7-membered heterocyclyl comprising 1, 2, or 3 heteroatoms independently selected from N, O, and S optionally substituted with one or more C₃-C₈cycloalkyl, aryl, or 4- to 7-membered heterocyclyl comprising 1, 2, or 3 heteroatoms independently selected from N, O, and S. In some embodiments, R₁ is 4- to 7-membered heterocyclyl comprising 1, 2, or 3 heteroatoms independently selected from N, O, and S optionally substituted with one or more C₃-C₈cycloalkyl or aryl. In some embodiments, R₁ is 4- to 7-membered heterocyclyl comprising 1, 2, or 3 heteroatoms independently selected from N, O, and S substituted with one or more C₃-C₈cycloalkyl or aryl. In some embodiments, R₁ is 4- to 7-membered heterocyclyl comprising 1, 2, or 3 heteroatoms independently selected from N, O, and S substituted with one or more C₃-C₈cycloalkyl. In some embodiments, R₁ is 4- to 7-membered heterocyclyl comprising 1, 2, or 3 heteroatoms independently selected from N, O, and S substituted with one or more aryl.

In some embodiments, R₁ is NH₂, NH(C₁-C₆alkyl), or N(C₁-C₆alkyl)₂.

In some embodiments, R₁ is NH₂.

In some embodiments, R₁ is NH(C₁-C₆alkyl) or N(C₁-C₆alkyl)₂.

In some embodiments, R₁ is NH(C₁-C₆alkyl). In some embodiments, R₁ is NH(methyl). In some embodiments, R₁ is NH(ethyl). In some embodiments, R₁ is NH(propyl). In some embodiments, R₁ is NH(butyl). In some embodiments, R₁ is NH(pentyl). In some embodiments, R₁ is NH(hexyl).

In some embodiments, R₁ is N(C₁-C₆alkyl)₂. In some embodiments, R₁ is N(methyl)₂. In some embodiments, R₁ is N(ethyl)₂. In some embodiments, R₁ is N(propyl)₂. In some embodiments. R₁ is N(butyl)₂. In some embodiments, R₁ is N(pentyl)₂. In some embodiments, R₁ is N(hexyl)₂.

In some embodiments, R₁ is H or F.

In some embodiments, R₂ is aryl.

In some embodiments, R₂ is aryl optionally substituted with one or more R₄. In some embodiments, R₂ is aryl substituted with one or more R₄. In some embodiments, R₂ is aryl substituted with one R₄. In some embodiments, R₂ is aryl substituted with two R₄. In some embodiments, R₂ is aryl substituted with three R₄.

In some embodiments, R₂ is 5- to 7-membered cycloalkyl.

In some embodiments, R₂ is 5- to 7-membered saturated cycloalkyl. In some embodiments, R₂ is 5- to 7-membered partially saturated cycloalkyl.

In some embodiments, R₂ is 5- to 7-membered cycloalkyl optionally substituted with one or more R₄. In some embodiments, R₂ is 5- to 7-membered cycloalkyl substituted with one or more R₄. In some embodiments, R₂ is 5- to 7-membered cycloalkyl substituted with one R₄. In some embodiments, R₂ is 5- to 7-membered cycloalkyl substituted with two R₄. In some embodiments, R₂ is 5- to 7-membered cycloalkyl substituted with three R₄.

In some embodiments, R₂ is a 5-, 6-, or 9-membered heterocyclyl comprising 1, 2, or 3 heteroatoms independently selected from N, O, and S,

In some embodiments, R₂ is a 5-, 6-, or 9-membered heterocyclyl comprising 1, 2, or 3 heteroatoms independently selected from N and O.

In some embodiments, R₂ is a 5-, 6-, or 9-membered heterocyclyl comprising 1 or 2 heteroatoms independently selected from N, O, and S,

In some embodiments, R₂ is a 5-, 6-, or 9-membered heterocyclyl comprising 1 or 2 heteroatoms independently selected from N and O.

In some embodiments, R₂ is a 5-, 6-, or 9-membered heterocyclyl comprising 1, 2, or 3 heteroatoms independently selected from N, O, and S, wherein R₂ is optionally substituted with one or more R₄.

In some embodiments, R₂ is a 5-, 6-, or 9-membered heteroaryl comprising 2, 3, or 4 heteroatoms independently selected from N, O, and S.

In some embodiments, R₂ is a 5-, 6-, or 9-membered heteroaryl comprising 2, 3, or 4 heteroatoms independently selected from N and O.

In some embodiments, R₂ is a 5-, 6-, or 9-membered heteroaryl comprising 2, 3, or 4 heteroatoms independently selected from N, O, and S, wherein the heteroaryl is optionally substituted with one or more R₄.

In some embodiments, R₂ is a 5-, 6-, or 9-membered heteroaryl comprising 2, 3, or 4 heteroatoms independently selected from N and O, wherein the heteroaryl is optionally substituted with one or more R₄.

In some embodiments, R₂ is a 5-, 6-, or 9-membered heteroaryl comprising 2, 3, or 4 heteroatoms independently selected from N, O, and S, wherein the heteroaryl is substituted with one or more R₄.

In some embodiments, R₂ is a 5-, 6-, or 9-membered heteroaryl comprising 2, 3, or 4 heteroatoms independently selected from N and O, wherein the heteroaryl is substituted with one or more R₄.

In some embodiments, R₂ is a 5-, 6-, or 9-membered heteroaryl comprising 2, 3, or 4 heteroatoms independently selected from N, O, and S, wherein the heteroaryl is substituted with one R₄.

In some embodiments, R₂ is a 5-, 6-, or 9-membered heteroaryl comprising 2, 3, or 4 heteroatoms independently selected from N and O, wherein the heteroaryl is substituted with one R₄.

In some embodiments, R₂ is a 5-, 6-, or 9-membered heteroaryl comprising 2, 3, or 4 heteroatoms independently selected from N, O, and S, wherein the heteroaryl is substituted with two R₄.

In some embodiments, R₂ is a 5-, 6-, or 9-membered heteroaryl comprising 2, 3, or 4 heteroatoms independently selected from N and O, wherein the heteroaryl is substituted with two R₄.

In some embodiments, R₂ is a 5-, 6-, or 9-membered heteroaryl comprising 2, 3, or 4 heteroatoms independently selected from N, O, and S, wherein the heteroaryl is substituted with three R₄.

In some embodiments, R₂ is a 5-, 6-, or 9-membered heteroaryl comprising 2, 3, or 4 heteroatoms independently selected from N and O, wherein the heteroaryl is substituted with three R₄.

In some embodiments, R₂ is a 9-membered heteroaryl comprising 2, 3, or 4 heteroatoms independently selected from N, O, and S, wherein the heteroaryl is optionally substituted with one or more R₄.

In some embodiments, R₂ is a 9-membered heteroaryl comprising 2, 3, or 4 heteroatoms independently selected from N and O, wherein the heteroaryl is optionally substituted with one or more R₄.

In some embodiments, R₂ is a 9-membered heteroaryl comprising 2, 3, or 4 heteroatoms independently selected from N, O, and S, wherein the heteroaryl is substituted with one or more R₄.

In some embodiments, R₂ is a 9-membered heteroaryl comprising 2, 3, or 4 heteroatoms independently selected from N and O, wherein the heteroaryl is substituted with one or more R₄.

In some embodiments, R₂ is a 9-membered heteroaryl comprising 2, 3, or 4 heteroatoms independently selected from N, O, and S, wherein the heteroaryl is substituted with one R₄.

In some embodiments, R₂ is a 9-membered heteroaryl comprising 2, 3, or 4 heteroatoms independently selected from N and O, wherein the heteroaryl is substituted with one R₄.

In some embodiments, R₂ is a 9-membered heteroaryl comprising 2, 3, or 4 heteroatoms independently selected from N, O, and S, wherein the heteroaryl is substituted with two R₄.

In some embodiments, R₂ is a 9-membered heteroaryl comprising 2, 3, or 4 heteroatoms independently selected from N and O, wherein the heteroaryl is substituted with two R₄.

In some embodiments, R₂ is a 9-membered heteroaryl comprising 2, 3, or 4 heteroatoms independently selected from N, O, and S, wherein the heteroaryl is substituted with three R₄.

In some embodiments, R₂ is a 9-membered heteroaryl comprising 2, 3, or 4 heteroatoms independently selected from N and O, wherein the heteroaryl is substituted with three R₄.

In some embodiments, R₂ is a 9-membered heteroaryl comprising 2 heteroatoms independently selected from N, O, and S, wherein the heteroaryl is optionally substituted with one or more R₄.

In some embodiments, R₂ is a 9-membered heteroaryl comprising 2 heteroatoms independently selected from N and O, wherein the heteroaryl is optionally substituted with one or more R₄.

In some embodiments, R₂ is a 9-membered heteroaryl comprising 2 heteroatoms independently selected from N, O, and S, wherein the heteroaryl is substituted with one or more R₄.

In some embodiments, R₂ is a 9-membered heteroaryl comprising 2 heteroatoms independently selected from N and O, wherein the heteroaryl is substituted with one or more R₄.

In some embodiments, R₂ is a 9-membered heteroaryl comprising 2 heteroatoms independently selected from N, O, and S, wherein the heteroaryl is substituted with one R₄.

In some embodiments, R₂ is a 9-membered heteroaryl comprising 2 heteroatoms independently selected from N and O, wherein the heteroaryl is substituted with one R₄.

In some embodiments, R₂ is a 9-membered heteroaryl comprising 2 heteroatoms independently selected from N, O, and S, wherein the heteroaryl is substituted with two R₄.

In some embodiments, R₂ is a 9-membered heteroaryl comprising 2 heteroatoms independently selected from N and O, wherein the heteroaryl is substituted with two R₄.

In some embodiments, R₂ is a 9-membered heteroaryl comprising 2 heteroatoms independently selected from N, O, and S, wherein the heteroaryl is substituted with three R₄.

In some embodiments, R₂ is a 9-membered heteroaryl comprising 2 heteroatoms independently selected from N and O, wherein the heteroaryl is substituted with three R₄.

In some embodiments, R₂ is a 9-membered heteroaryl comprising 3 heteroatoms independently selected from N, O, and S, wherein the heteroaryl is optionally substituted with one or more R₄.

In some embodiments, R₂ is a 9-membered heteroaryl comprising 3 heteroatoms independently selected from N and O, wherein the heteroaryl is optionally substituted with one or more R₄.

In some embodiments, R₂ is a 9-membered heteroaryl comprising 3 heteroatoms independently selected from N, O, and S, wherein the heteroaryl is substituted with one or more R₄.

In some embodiments, R₂ is a 9-membered heteroaryl comprising 3 heteroatoms independently selected from N and O, wherein the heteroaryl is substituted with one or more R₄.

In some embodiments, R₂ is a 9-membered heteroaryl comprising 3 heteroatoms independently selected from N, O, and S, wherein the heteroaryl is substituted with one R₄.

In some embodiments, R₂ is a 9-membered heteroaryl comprising 3 heteroatoms independently selected from N and O, wherein the heteroaryl is substituted with one R₄.

In some embodiments, R₂ is a 9-membered heteroaryl comprising 3 heteroatoms independently selected from N, O, and S, wherein the heteroaryl is substituted with two R₄.

In some embodiments, R₂ is a 9-membered heteroaryl comprising 3 heteroatoms independently selected from N and O, wherein the heteroaryl is substituted with two R₄.

In some embodiments, R₂ is a 9-membered heteroaryl comprising 3 heteroatoms independently selected from N, O, and S, wherein the heteroaryl is substituted with three R₄.

In some embodiments, R₂ is a 9-membered heteroaryl comprising 3 heteroatoms independently selected from N and O, wherein the heteroaryl is substituted with three R₄.

In some embodiments, R₂ is a 9-membered heteroaryl comprising 4 heteroatoms independently selected from N, O, and S, wherein the heteroaryl is optionally substituted with one or more R₄.

In some embodiments, R₂ is a 9-membered heteroaryl comprising 4 heteroatoms independently selected from N and O, wherein the heteroaryl is optionally substituted with one or more R₄.

In some embodiments, R₂ is a 9-membered heteroaryl comprising 4 heteroatoms independently selected from N, O, and S, wherein the heteroaryl is substituted with one or more R₄.

In some embodiments, R₂ is a 9-membered heteroaryl comprising 4 heteroatoms independently selected from N and O, wherein the heteroaryl is substituted with one or more R₄.

In some embodiments, R₂ is a 9-membered heteroaryl comprising 4 heteroatoms independently selected from N, O, and S, wherein the heteroaryl is substituted with one R₄.

In some embodiments, R₂ is a 9-membered heteroaryl comprising 4 heteroatoms independently selected from N and O, wherein the heteroaryl is substituted with one R₄.

In some embodiments, R₂ is a 9-membered heteroaryl comprising 4 heteroatoms independently selected from N, O, and S, wherein the heteroaryl is substituted with two R₄.

In some embodiments, R₂ is a 9-membered heteroaryl comprising 4 heteroatoms independently selected from N and O, wherein the heteroaryl is substituted with two R₄.

In some embodiments, R₂ is a 9-membered heteroaryl comprising 4 heteroatoms independently selected from N, O, and S, wherein the heteroaryl is substituted with three R₄.

In some embodiments, R₂ is a 9-membered heteroaryl comprising 4 heteroatoms independently selected from N and O, wherein the heteroaryl is substituted with three R₄.

In some embodiments, R₂ is a bicyclic 9-membered heteroaryl.

In some embodiments, R₂ is

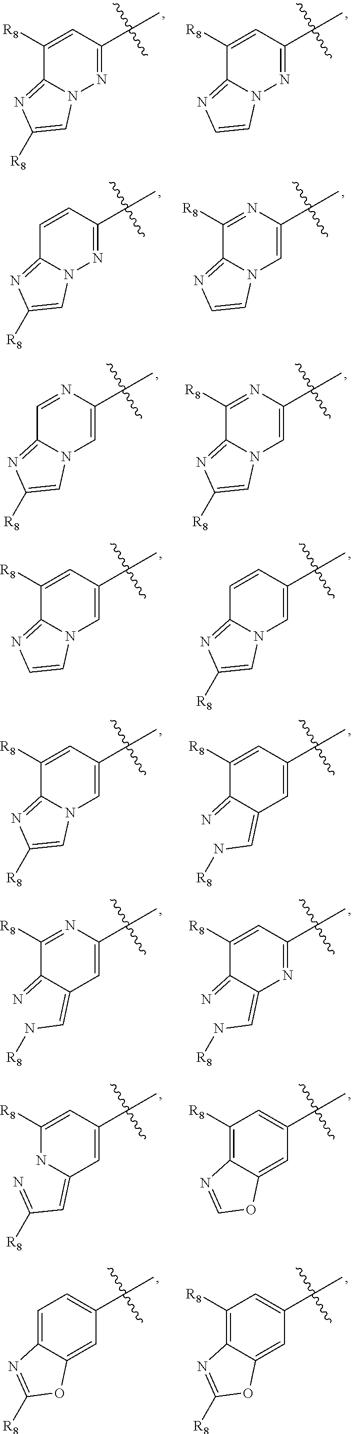

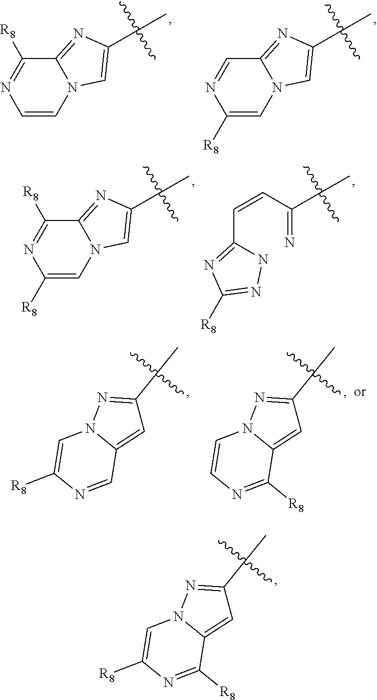

wherein each R₈ is independently R₄.

In some embodiments, R₂ is

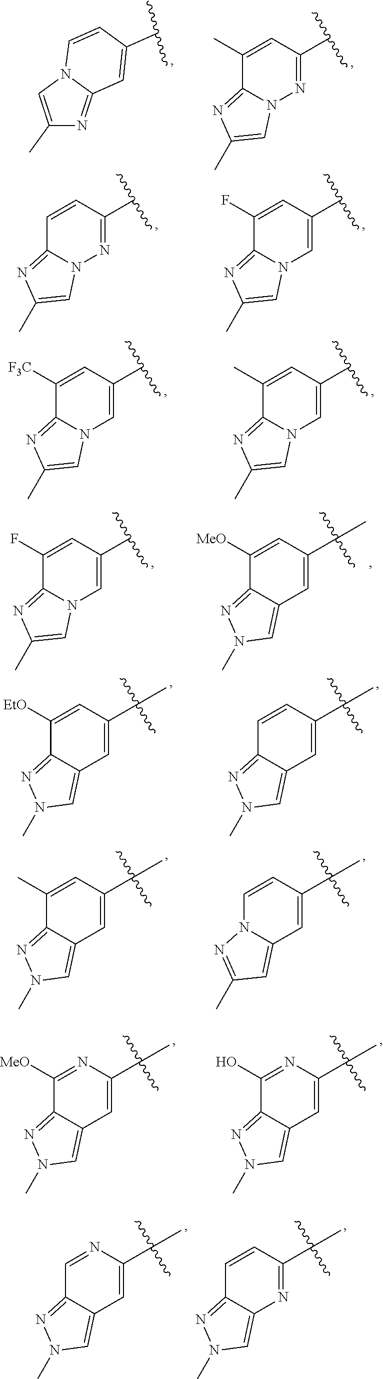

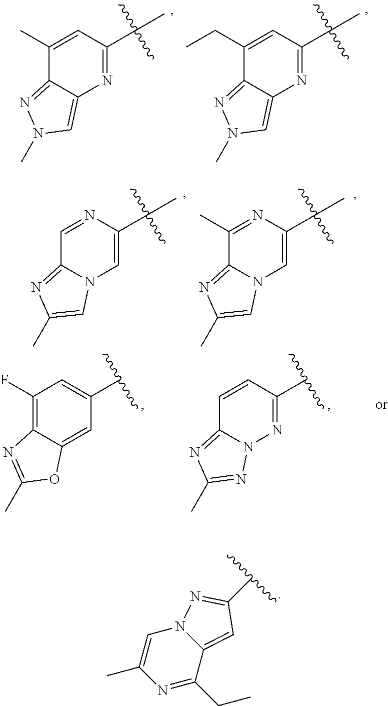

In some embodiments, each R₃ is independently halogen, C₁-C₆alkyl, C₂-C₆alkenyl, C₂-C₆alkynyl, C₁-C₆alkoxyl, C₃-C₈cycloalkyl, NH₂, NH(C₁-C₆alkyl), or N(C₁-C₆alkyl)₂.

In some embodiments, at least one R₃ is halogen. In some embodiments, at least one R₃ is F, Cl, Br, or I. In some embodiments, at least one R₃ is F, Cl, or Br. In some embodiments, at least one R₃ is F or Cl. In some embodiments, at least one R₃ is F. In some embodiments, at least one R₃ is Cl. In some embodiments, at least one R₃ is Br. In some embodiments, at least one R₃ is I.

In some embodiments, each R₃ is independently C₁-C₆alkyl, C₂-C₆alkenyl, C₂-C₆alkynyl, C₁-C₆alkoxyl, or C₃-C₈cycloalkyl.

In some embodiments, each R₃ is independently C₁-C₆alkyl, C₂-C₆alkenyl, C₂-C₆alkynyl, C₁-C₆alkoxyl, or C₃-C₈cycloalkyl, wherein the alkyl, alkenyl, alkynyl, alkoxyl, and cycloalkyl are optionally substituted with one or more hydroxyl or NH₂.

In some embodiments, each R₃ is independently C₁-C₆alkyl, C₂-C₆alkenyl, or C₂-C₆alkynyl.

In some embodiments, each R₃ is independently C₁-C₆alkyl, C₂-C₆alkenyl, or C₂-C₆alkynyl, wherein the alkyl, alkenyl, or alkynyl are optionally substituted with one or more hydroxyl or NH₂.

In some embodiments, at least one R₃ is C₁-C₆alkyl. In some embodiments, at least one R₃ is methyl. In some embodiments, at least one R₃ is ethyl. In some embodiments, at least one R₃ is propyl. In some embodiments, at least one R₃ is butyl. In some embodiments, R₃ is iso-propyl. In some embodiments, at least one R₃ is iso-butyl. In some embodiments, at least one R₃ is sec-butyl. In some embodiments, at least one R₃ is tert-butyl.

In some embodiments, each R₃ is C₁-C₆alkyl optionally substituted with one or more hydroxyl or NH₂.

In some embodiments, R₃ is C₂-C₆alkenyl. In some embodiments, R₃ is C₂alkenyl. In some embodiments, R₃ is C₃alkenyl. In some embodiments, R₃ is C₄alkenyl. In some embodiments, R₃ is C₅alkenyl. In some embodiments, R₃ is C₆alkenyl.

In some embodiments, R₃ is C₂-C₆alkenyl optionally substituted with one or more hydroxyl or NH₂.

In some embodiments, R₃ is C₂-C₆alkynyl. In some embodiments, R₃ is C₂alkynyl. In some embodiments, R₃ is C₃alkynyl. In some embodiments, R₃ is C₄alkynyl. In some embodiments. R₃ is C₅alkynyl. In some embodiments, R₃ is C₆alkynyl.

In some embodiments, R₃ is C₂-C₆alkynyl optionally substituted with one or more hydroxyl or NH₂.

In some embodiments, each R₃ is independently C₁-C₆alkoxyl or C₃-C₈cycloalkyl.

In some embodiments, each R₃ is independently C₁-C₆alkoxyl or C₃-C₈cycloalkyl, wherein the alkoxyl and cycloalkyl are optionally substituted with one or more hydroxyl or NH₂.

In some embodiments, each R₃ is independently C₁-C₆alkoxyl optionally substituted with one or more hydroxyl or NH₂.

In some embodiments, at least one R₃ is C₁-C₆alkoxyl. In some embodiments, at least one R₃ is methoxyl. In some embodiments, at least one R₃ is ethoxyl. In some embodiments, at least one R₃ is propoxyl. In some embodiments, at least one R₃ is butoxyl. In some embodiments, at least one R₃ is pentoxyl. In some embodiments, at least one R₃ is hexoxyl.

In some embodiments, each R₃ is independently C₃-C₈cycloalkyl optionally substituted with one or more hydroxyl or NH₂.

In some embodiments, at least one R₃ is independently C₃-C₈cycloalkyl. In some embodiments, at least one R₃ is independently cyclopropyl. In some embodiments, at least one R₃ is independently cyclobutyl. In some embodiments, at least one R₃ is independently cyclopentyl. In some embodiments, at least one R₃ is independently cyclohexyl. In some embodiments, at least one R₃ is independently cycloheptyl. In some embodiments, at least one R₃ is independently cyclooctyl.

In some embodiments, each R₃ is independently NH₂, NH(C₁-C₆alkyl), or N(C₁-C₆alkyl)₂.

In some embodiments, at least one R₃ is NH₂.

In some embodiments, each R₃ is NH(C₁-C₆alkyl), or N(C₁-C₆alkyl)₂.

In some embodiments, at least one R₃ is NH(C₁-C₆alkyl).

In some embodiments, at least one R₃ is NH(methyl). In some embodiments, at least one R₃ is NH(ethyl). In some embodiments, at least one R₃ is NH(propyl). In some embodiments, at least one it is NH(butyl). In some embodiments, at least one R₃ is NH(pentyl). In some embodiments, at least one R₃ is NH(hexyl).

In some embodiments, at least one it is N(C₁-C₆alkyl)₂. In some embodiments, at least one R₃ is N(methyl)₂. In some embodiments, at least one R₃ is N(ethyl)₂. In some embodiments, at least one R₃ is N(propyl)₂. In some embodiments, at least one R₃ is N(butyl)₂. In some embodiments, at least one R₃ is N(pentyl)₂. In some embodiments, at least one R₃ is N(hexyl)₂.

In some embodiments, R₃ is F or methyl.

In some embodiments, each R₄ is independently halogen, hydroxyl, cyano, nitro, C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂.

In some embodiments, each R₄ is independently halogen, hydroxyl, cyano, or nitro.

In some embodiments, at least one R₄ is halogen. In some embodiments, at least one R₄ is F, Cl, Br, or I. In some embodiments, at least one R₄ is F, Cl, or Br. In some embodiments, at least one R₄ is F or Cl. In some embodiments, at least one R₄ is F. In some embodiments, at least one R₄ is Cl. In some embodiments, at least one R₄ is Br. In some embodiments, at least one R₄ is I.

In some embodiments, each R₄ is independently hydroxyl, cyano, or nitro.

In some embodiments, at least one R₄ is independently hydroxyl.

In some embodiments, at least one R₄ is independently cyano.

In some embodiments, at least one R₄ is independently nitro.

In some embodiments, each R₄ is independently C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂.

In some embodiments, each R₄ is independently C₁-C₆alkyl, C₂-C₆alkenyl, C₂-C₆alkynyl, C₁-C₆haloalkyl, C₁-C₆alkoxyl, C₁-C₆haloalkoxyl, C₃-C₈cycloalkyl, NH₂, NH(C₁-C₆alkyl), N(C₁-C₆alkyl)₂, or C(O)NH₂.

In some embodiments, each R₄ is independently C₁-C₆alkyl, C₂-C₆alkenyl, or C₂-C₆alkynyl, wherein the alkyl, alkenyl, or alkynyl are optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂.

In some embodiments, each R₄ is independently C₁-C₆alkyl, C₂-C₆alkenyl, or C₂-C₆alkynyl.

In some embodiments, R₄ is C₁-C₆alkyl. In some embodiments, R₄ is methyl. In some embodiments, R₄ is ethyl. In some embodiments, R₄ is propyl. In some embodiments, R₄ is butyl. In some embodiments, R₄ is iso-propyl. In some embodiments, R₄ is iso-butyl. In some embodiments, R₄ is sec-butyl. In some embodiments, R₄ is tert-butyl.

In some embodiments, R₄ is C₁-C₆alkyl optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂.

In some embodiments, R₄ is C₂-C₆alkenyl. In some embodiments, R₄ is C₂alkenyl. In some embodiments, R₄ is C₃alkenyl. In some embodiments, R₄ is C₄alkenyl. In some embodiments, R₄ is C₆alkenyl. In some embodiments, R₄ is C₆alkenyl.

In some embodiments, R₄ is C₂-C₆alkenyl optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂.

In some embodiments, R₄ is C₂-C₆alkynyl. In some embodiments, R₄ is C₂alkynyl. In some embodiments, R₄ is C₃alkynyl. In some embodiments, R₄ is C₄alkynyl. In some embodiments, R₄ is C₅alkynyl. In some embodiments, R₄ is C₆alkynyl.

In some embodiments, R₄ is C₂-C₆alkynyl optionally substituted with one or more hydroxyl, 4- to 7-membered heterocyclyl comprising 1, 2, or 3 heteroatoms independently selected from N, O, and S, NH₂, NH(C₁-C₆alkyl), or N(C₁-C₆alkyl)₂.

In some embodiments, each R₄ is independently C₁-C₆alkoxyl or C₃-C₈cycloalkyl.

In some embodiments, each R₄ is independently C₁-C₆alkoxyl or C₃-C₈cycloalkyl, wherein the alkoxyl and cycloalkyl are optionally substituted with one or more hydroxyl, 4- to 7-membered heterocyclyl, NH₂, NH(C₁-C₆alkyl), or N(C₁-C₆alkyl)₂.

In some embodiments, each R₄ is independently C₁-C₆alkoxyl. In some embodiments, each R₄ is independently methoxyl. In some embodiments, each R₄ is independently ethoxyl. In some embodiments, each R₄ is independently propoxyl. In some embodiments, each R₄ is independently butoxyl. In some embodiments, each R₄ is independently pentoxyl. In some embodiments, each R₄ is independently hexoxyl.

In some embodiments, each R₄ is independently C₁-C₆alkoxyl optionally substituted with one or more hydroxyl, 4- to 7-membered heterocyclyl, NH₂, NH(C₁-C₆alkyl), or N(C₁-C₆alkyl)₂.

In some embodiments, each R₄ is independently C₃-C₈cycloalkyl. In some embodiments, each R₄ is independently cyclopropyl. In some embodiments, each R₄ is independently cyclobutyl. In some embodiments, each R₄ is independently cyclopentyl. In some embodiments, each R₄ is independently cyclohexyl. In some embodiments, each R₄ is independently cycloheptyl. In some embodiments, each R₄ is independently cyclooctyl.

In some embodiments, each R₄ is independently C₃-C₈cycloalkyl optionally substituted with one or more hydroxyl, 4- to 7-membered heterocyclyl, NH₂, NH(C₁-C₆alkyl), or N(C₁-C₆alkyl)₂.

In some embodiments, each R₄ is independently C₁-C₆haloalkyl or C₁-C₆haloalkoxyl.

In some embodiments, each R₄ is independently C₁-C₆haloalkyl. In some embodiments, each R₄ is independently halomethyl. In some embodiments, each R₄ is independently haloethyl. In some embodiments, each R₄ is independently halopropyl. In some embodiments, each R₄ is independently halobutyl. In some embodiments, each R₄ is independently halopentyl. In some embodiments, each R₄ is independently halohexyl.

In some embodiments, each R₄ is independently CF₃, CHF₂, or CH₂F.

In some embodiments, each R₄ is independently C₁-C₆haloalkoxyl. In some embodiments, each R₄ is independently halomethoxyl. In some embodiments, each R₄ is independently haloethoxyl. In some embodiments, each R₄ is independently halopropoxyl. In some embodiments, each R₄ is independently halobutoxyl. In some embodiments, each R₄ is independently halopentoxyl. In some embodiments, each R₄ is independently halohexoxyl.

In some embodiments, each R₄ is independently NH₂, NH(C₁-C₆alkyl), N(C₁-C₆alkyl)₂, or C(O)NH₂.

In some embodiments, each R₄ is independently NH₂, NH(C₁-C₆alkyl), or N(C₁-C₆alkyl)₂.

In some embodiments, each R₄ is independently NH₂.

In some embodiments, each R₄ is independently C(O)NH₂.

In some embodiments, each R₄ is independently NH(C₁-C₆alkyl) or N(C₁-C₆alkyl)₂.

In some embodiments, each R₄ is independently NH(C₁-C₆alkyl). In some embodiments, each R₄ is independently H(methyl). In some embodiments, each R₄ is independently NH(ethyl). In some embodiments, each R₄ is independently NH(propyl). In some embodiments, each R₄ is independently NH(butyl). In some embodiments, each R₄ is independently NH(pentyl). In some embodiments, each R₄ is independently NH(hexyl).

In some embodiments, each R₄ is independently N(C₁-C₆alkyl)₂. In some embodiments, each R₄ is independently N(methyl)₂. In some embodiments, each R₄ is independently N(ethyl)₂. In some embodiments, each R₄ is independently N(propyl)₂. In some embodiments, each R₄ is independently N(butyl)₂. In some embodiments, each R₄ is independently N(pentyl)₂. In some embodiments, each R₄ is independently N(hexyl)₂.

In some embodiments, each R₄ is independently methyl, ethyl, F, or CF₃.

In some embodiments, each R₄ is independently methyl or ethyl.

In some embodiments, each R₄ is independently F or CF₃.

In some embodiments, R₅ is H.

In some embodiments, R₅ is C₁-C₆alkyl, C₁-C₆haloalkyl, C₂-C₆alkenyl, C₂-C₆alkynyl, C₁-C₆alkoxyl, C₃-C₈cycloalkyl, or heterocyclyl, wherein heterocyclyl is a 4- to 7-membered ring and comprises 1, 2, or 3 heteroatoms independently selected from N, O, and S, wherein the cycloalkyl and heterocyclyl are optionally substituted with one or more halogen, C₁-C₆alkyl or hydroxyl.

In some embodiments, R₅ is C₁-C₆alkyl, C₂-C₆alkenyl, or C₂-C₆alkynyl.

In some embodiments, R₅ is C₁-C₆alkyl. In some embodiments, R₅ is methyl. In some embodiments, R₅ is ethyl. In some embodiments, R₅ is propyl. In some embodiments, R₅ is butyl. In some embodiments, R₅ is iso-propyl. In some embodiments, R₅ is iso-butyl. In some embodiments, R₅ is sec-butyl. In some embodiments, R₅ is tert-butyl.

In some embodiments, R₅ is C₂-C₆alkenyl. In some embodiments, R₅ is C₂-C₆alkenyl. In some embodiments, R₅ is C₂alkenyl. In some embodiments, R₅ is C₃alkenyl. In some embodiments, R₅ is C₄alkenyl. In some embodiments, R₅ is C₅alkenyl. In some embodiments, R₅ is C₆alkenyl.

In some embodiments, R₅ is C₂-C₆alkynyl. In some embodiments, R₅ is C₂-C₆alkynyl. In some embodiments, R₅ is C₂alkynyl. In some embodiments, R₅ is C₃alkynyl. In some embodiments, R₅ is C₄alkynyl. In some embodiments, R₅ is C₅alkynyl. In some embodiments, R₅ is C₆alkynyl.

In some embodiments, R₅ is C₁-C₆alkoxyl or C₃-C₈cycloalkyl.

In some embodiments, R₅ is independently C₁-C₆alkoxyl. In some embodiments, R₅ is independently methoxyl. In some embodiments, R₅ is independently ethoxyl. In some embodiments, R₅ is independently propoxyl. In some embodiments, R₅ is independently butoxyl. In some embodiments, R₅ is independently pentoxyl. In some embodiments, R₅ is independently hexoxyl.

In some embodiments, R₅ is C₃-C₈cycloalkyl. In some embodiments, R₅ is cyclopropyl. In some embodiments, R₅ is cyclobutyl. In some embodiments, R₅ is cyclopentyl. In some embodiments, R₅ is cyclohexyl. In some embodiments, R₅ is heptyl. In some embodiments, R₅ is cyclooctyl.

In some embodiments, R₅ is heterocyclyl, wherein heterocyclyl is a 4- to 7-membered ring and comprises 1, 2, or 3 heteroatoms independently selected from N, O, and S, wherein the cycloalkyl and heterocyclyl are optionally substituted with one or more halogen, C₁-C₆alkyl or hydroxyl. In some embodiments, R₅ is heterocyclyl, wherein heterocyclyl is a 4- to 6-membered ring and comprises 1 or 2 heteroatoms independently selected from N and O, wherein the cycloalkyl and heterocyclyl are optionally substituted with one or more halogen, C₁-C₆alkyl or hydroxyl. In some embodiments, R₅ is tetrahydropyranyl or tetrahydrofuranyl.

In some embodiments, R₅ is H. C₁₋₆alkyl, or heterocyclyl, wherein heterocyclyl is a 4- to 7-membered ring and comprises 1 or 2 heteroatoms independently selected from N and 0.

In some embodiments, R₅ is H. In some embodiments, R₆ is H. In some embodiments both R₅ and R₆ are H.

In some embodiments of the compounds of Formula I, n is 0, 1, 2, 3, 4, or 5. In some embodiments of the compounds of Formula I, n is 0, 1, 2, 3, or 4. In some embodiments of the compounds of Formula I, n is 0, 1, 2, or 3. In some embodiments of the compounds of Formula I, n is 0, 1, or 2. In some embodiments of the compounds of Formula I, n is 0 or 1. In some embodiments of the compounds of Formula I, n is 0. In some embodiments of the compounds of Formula I, n is 1. In some embodiments of the compounds of Formula I, n is 2. In some embodiments of the compounds of Formula I, n is 3. In some embodiments of the compounds of Formula I, n is 4. In some embodiments of the compounds of Formula I, n is 5.

Splice modulators of the present disclosure include a structure of Formula (II):

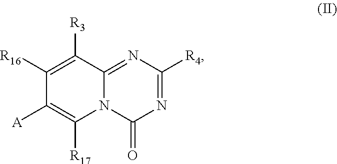

or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, ORs, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently selected from the group consisting of halogen, hydroxy, cyano, —COOH, —C(O)—C₁-C₆alkyl, —C(O)O—C₁-C₆alkyl, C₁-C₇alkyl, C₁-C₈heteroalkyl, C₁₋₇alkoxy-heterocycloalkyl, C₂-C₆alkenyl, C₂-C₆alkynyl, C₁-C₆alkoxy. —(CH₂)₀₋₂—C₃-C₈cycloalkyl, 4-7-membered monocyclic heterocycloalkyl, NH₂, NH(C₁-C₆alkyl), N(C₁-C₆alkyl)₂, —NHC(O)—C₁-C₆alkyl, —N(C₁-C₆alkyl)-C(O)—C₁-C₆alkyl, —C(O)—NH₂, —C(O)—NH(C₁-C₆alkyl), and —C(O)—N(C₁-C₆alkyl)₂, wherein the alkyl. alkenyl, alkynyl, and alkoxy are optionally substituted with one or more halogen, hydroxyl or NH₂, and wherein the cycloalkyl and heterocycloalkyl are optionally substituted with one or more halogen, hydroxyl, C₁-C₆alkyl, C₁-C₆heteroalkyl, C₁-C₆alkoxy, or NH₂; or
two R⁶ on the same carbon can be taken together as keto (═O); or
two R₆ together form C₁₋₇alkylene;
each R₇ is independently halo, cyano, C₁₋₇alkyl, C₁₋₇haloalkyl, C₁₋₇alkoxy, C₁₋₇haloalkoxy, or C₃₋₈cycloalkyl, wherein the C₁₋₇alkyl is optionally substituted with OH;
R₁₆ is H, halo, C₁₋₇alkyl, OR₅, N(R₅)₂, C₃₋₈cycloalkyl, or heterocycloalkyl; and
R₁₇ is H, halo, C₁₋₇alkyl, OR₅, N(R₅)₂, C₃₋₈cycloalkyl, or heterocycloalkyl.

Splice modulators of the present disclosure include a structure of Formula (III):

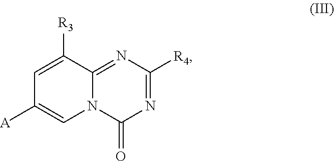

or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇: each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, or C₁₋₇alkoxy-heterocycloalkyl, or two R₆ together form C₁₋₇alkylene; and
each R₇ is independently halo, cyano, C₁₋₇alkyl, C₁₋₇haloalkyl, C₁₋₇alkoxy, C₁₋₇haloalkoxy, or C₃₋₈cycloalkyl, wherein the C₁₋₇alkyl is optionally substituted with OH.

In some embodiments, the compound is selected from the prodrugs of compounds described in Table 3 and pharmaceutically acceptable salts thereof.

TABLE 3

| Compound | |
|---|---|
| Number | Compound |
| 1A | 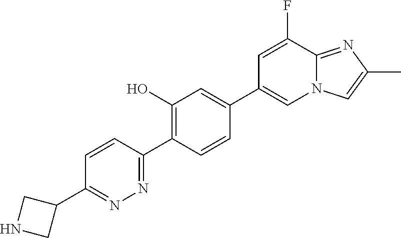 [SMILES: Cc1cn2cc(-c3ccc(-c4ccc(C5CNC5)nn4)c(O)c3)cc(F)c2n1] |
| 2A | 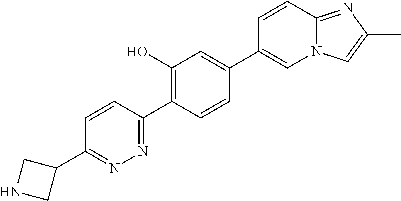 [SMILES: Cc1cn2cc(-c3ccc(-c4ccc(C5CNC5)nn4)c(O)c3)ccc2n1] |
| 3A | 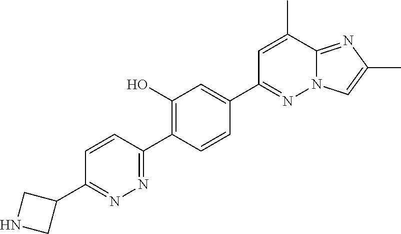 [SMILES: Cc1cn2nc(-c3ccc(-c4ccc(C5CNC5)nn4)c(O)c3)cc(C)c2n1] |
| 4A | 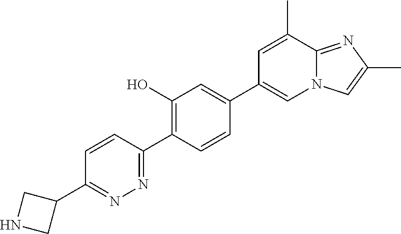 [SMILES: Cc1cn2cc(-c3ccc(-c4ccc(C5CNC5)nn4)c(O)c3)cc(C)c2n1] |
| 5A | 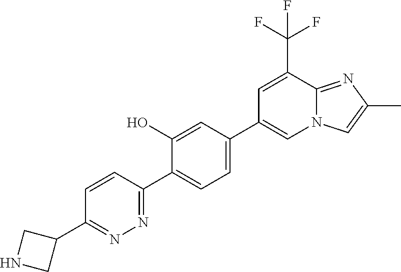 [SMILES: Cc1cn2cc(-c3ccc(-c4ccc(C5CNC5)nn4)c(O)c3)cc(C(F)(F)F)c2n1] |
| 6A | 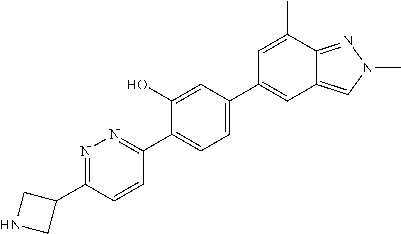 [SMILES: Cc1cc(-c2ccc(-c3ccc(C4CNC4)nn3)c(O)c2)cc2cn(C)nc12] |
| 7A | 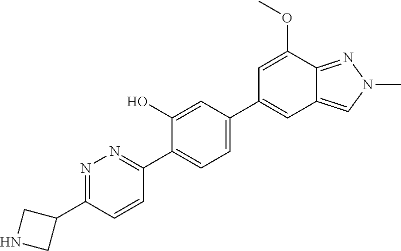 [SMILES: COc1cc(-c2ccc(-c3ccc(C4CNC4)nn3)c(O)c2)cc2cn(C)nc12] |
| 8A | 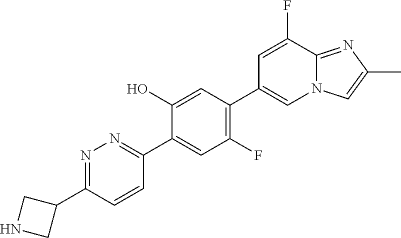 [SMILES: Cc1cn2cc(-c3cc(O)c(-c4ccc(C5CNC5)nn4)cc3F)cc(F)c2n1] |
| 9A | 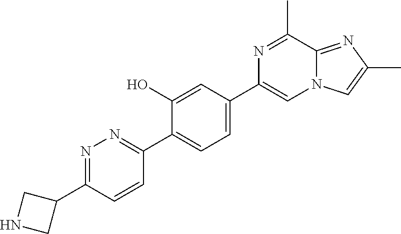 [SMILES: Cc1cn2cc(-c3ccc(-c4ccc(C5CNC5)nn4)c(O)c3)nc(C)c2n1] |
| 10A | 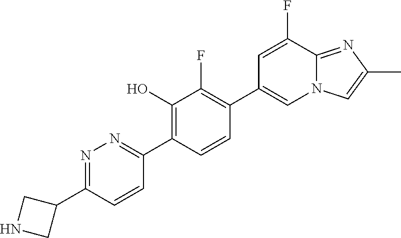 [SMILES: Cc1cn2cc(-c3ccc(-c4ccc(C5CNC5)nn4)c(O)c3F)cc(F)c2n1] |
| 11A | 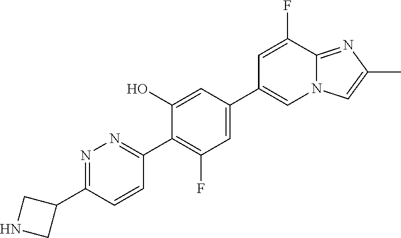 [SMILES: Cc1cn2cc(-c3cc(O)c(-c4ccc(C5CNC5)nn4)c(F)c3)cc(F)c2n1] |
| 12A | 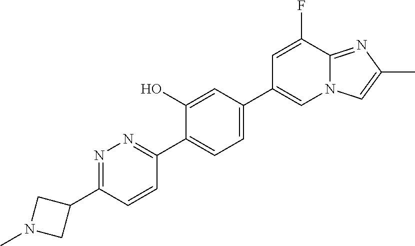 |
| 13A | 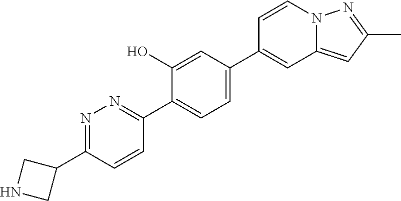 [SMILES: Cc1cc2cc(-c3ccc(-c4ccc(C5CNC5)nn4)c(O)c3)ccn2n1] |
| 14A | 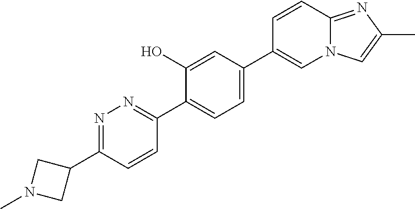 |
| 15A | 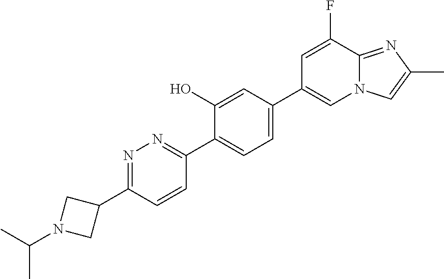 [SMILES: Cc1cn2cc(-c3ccc(-c4ccc(C5CN(C(C)C)C5)nn4)c(O)c3)cc(F)c2n1] |
| 16A | 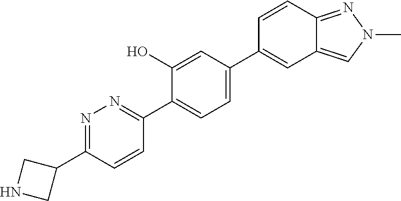 [SMILES: Cn1cc2cc(-c3ccc(-c4ccc(C5CNC5)nn4)c(O)c3)ccc2n1] |
| 17A | 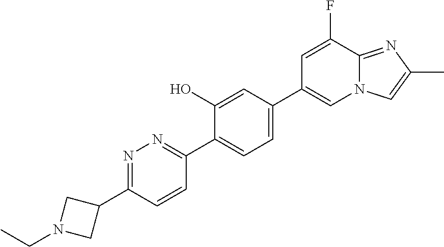 [SMILES: CCN1CC(c2ccc(-c3ccc(-c4cc(F)c5nc(C)cn5c4)cc3O)nn2)C1] |
| 18A | 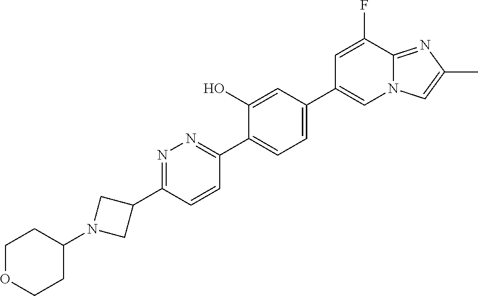 [SMILES: Cc1cn2cc(-c3ccc(-c4ccc(C5CN(C6CCOCC6)C5)nn4)c(O)c3)cc(F)c2n1] |
| 19A | 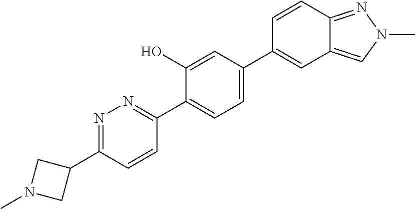 |
| 20A | 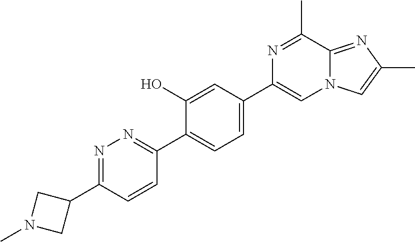 |
| 21A | 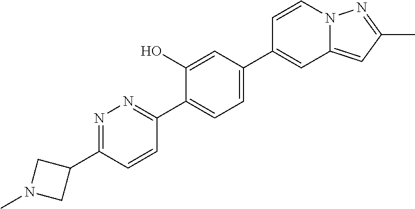 |
| 22A | 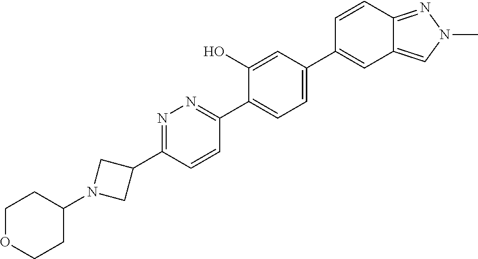 [SMILES: Cn1cc2cc(-c3ccc(-c4ccc(C5CN(C6CCOCC6)C5)nn4)c(O)c3)ccc2n1] |
| 23A | 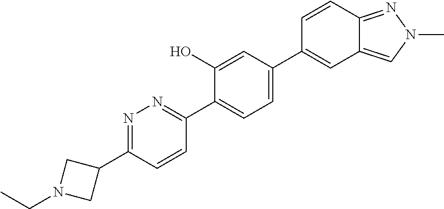 [SMILES: CCN1CC(c2ccc(-c3ccc(-c4ccc5nn(C)cc5c4)cc3O)nn2)C1] |
| 24A | 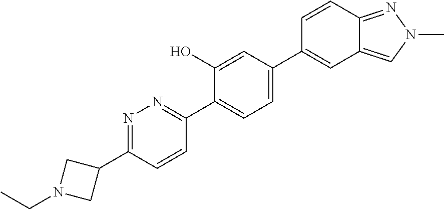 [SMILES: CCN1CC(c2ccc(-c3ccc(-c4ccc5nn(C)cc5c4)cc3O)nn2)C1] |
| 25A | 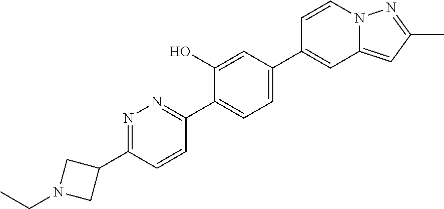 [SMILES: CCN1CC(c2ccc(-c3ccc(-c4ccn5nc(C)cc5c4)cc3O)nn2)C1] |
| 26A | 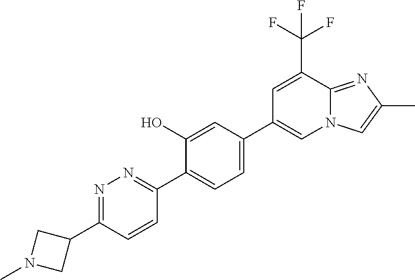 |
| 27A | 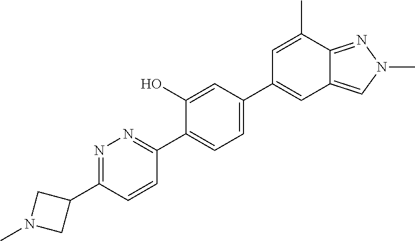 |
| 28A | 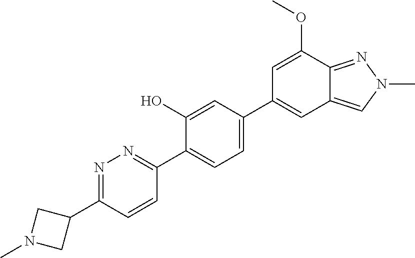 |
| 29A | 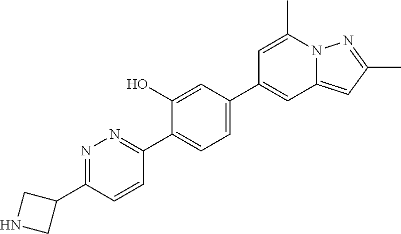 [SMILES: Cc1cc2cc(-c3ccc(-c4ccc(C5CNC5)nn4)c(O)c3)cc(C)n2n1] |
| 30A | 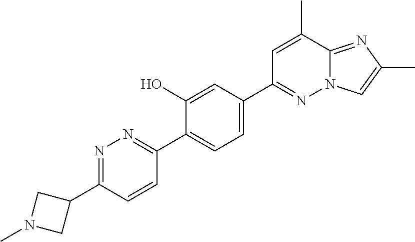 |
| 31A | 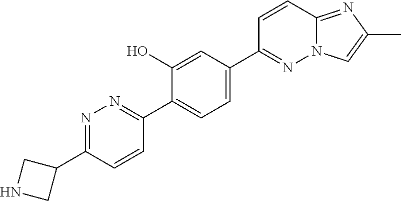 [SMILES: Cc1cn2nc(-c3ccc(-c4ccc(C5CNC5)nn4)c(O)c3)ccc2n1] |
| 32A | 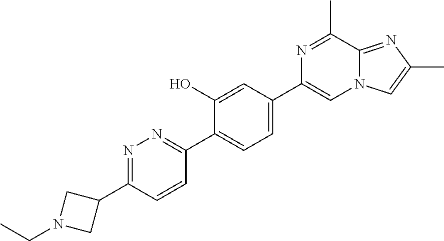 [SMILES: CCN1CC(c2ccc(-c3ccc(-c4cn5cc(C)nc5c(C)n4)cc3O)nn2)C1] |
| 33A | 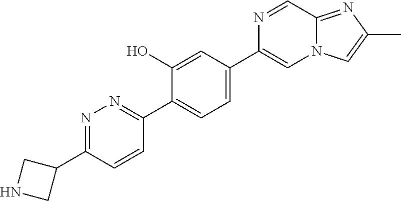 [SMILES: Cc1cn2cc(-c3ccc(-c4ccc(C5CNC5)nn4)c(O)c3)ncc2n1] |
| 34A | 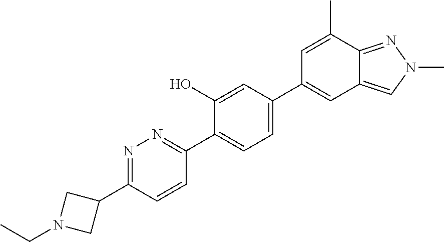 [SMILES: CCN1CC(c2ccc(-c3ccc(-c4cc(C)c5nn(C)cc5c4)cc3O)nn2)C1] |
| 35A | 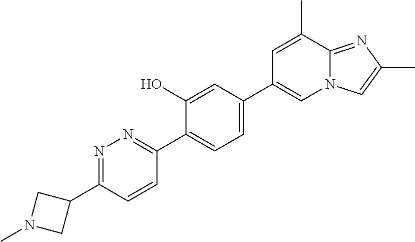 |
| 36A | 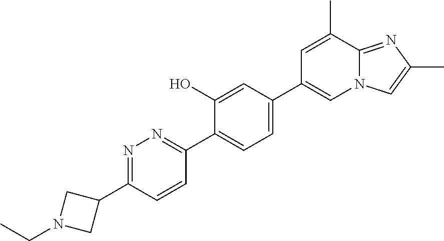 [SMILES: CCN1CC(c2ccc(-c3ccc(-c4cc(C)c5nc(C)cn5c4)cc3O)nn2)C1] |
| 37A | 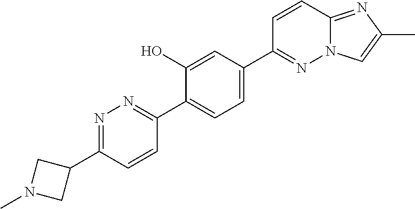 |
| 38A | 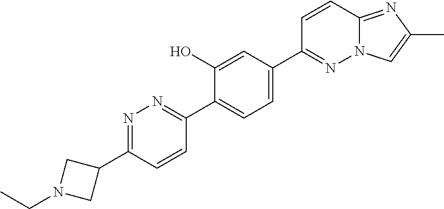 [SMILES: CCN1CC(c2ccc(-c3ccc(-c4ccc5nc(C)cn5n4)cc3O)nn2)C1] |
| 39A | 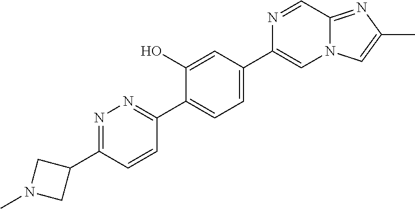 |
| 40A | 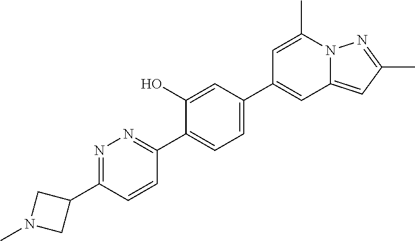 |
| 41A | 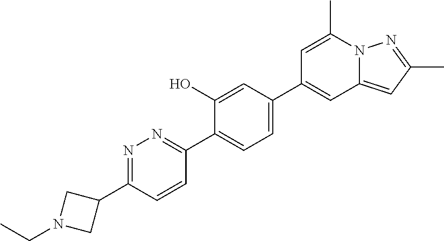 [SMILES: CCN1CC(c2ccc(-c3ccc(-c4cc(C)n5nc(C)cc5c4)cc3O)nn2)C1] |
| 42A | 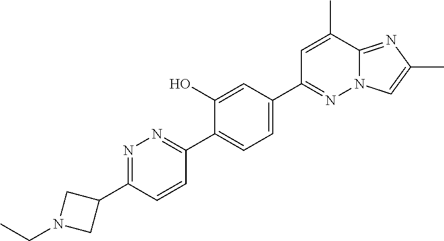 [SMILES: CCN1CC(c2ccc(-c3ccc(-c4cc(C)c5nc(C)cn5n4)cc3O)nn2)C1] |
| 43A | 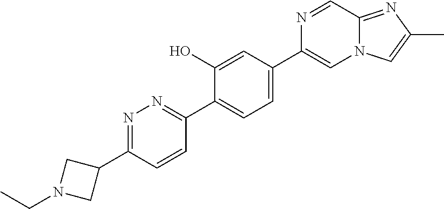 [SMILES: CCN1CC(c2ccc(-c3ccc(-c4cn5cc(C)nc5cn4)cc3O)nn2)C1] |
| 44A | 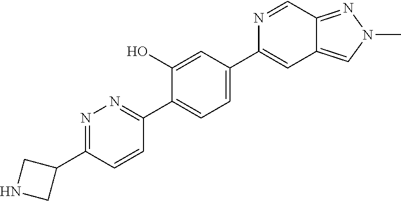 [SMILES: Cn1cc2cc(-c3ccc(-c4ccc(C5CNC5)nn4)c(O)c3)ncc2n1] |
| 45A | 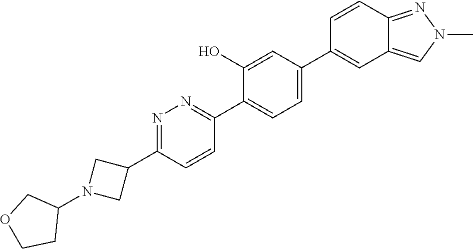 [SMILES: Cn1cc2cc(-c3ccc(-c4ccc(C5CN(C6CCOC6)C5)nn4)c(O)c3)ccc2n1] |
| 46A | 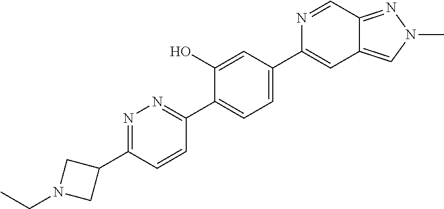 [SMILES: CCN1CC(c2ccc(-c3ccc(-c4cc5cn(C)nc5cn4)cc3O)nn2)C1] |
| 47A | 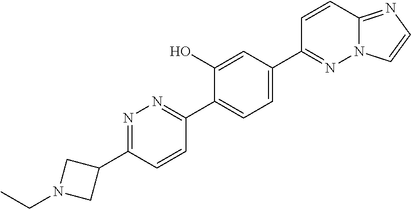 [SMILES: CCN1CC(c2ccc(-c3ccc(-c4ccc5nccn5n4)cc3O)nn2)C1] |
| 48A | 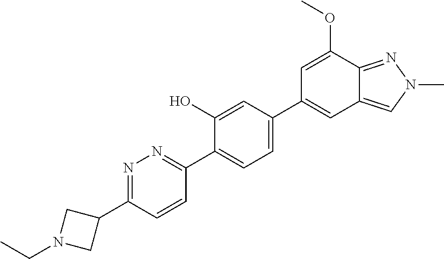 [SMILES: CCN1CC(c2ccc(-c3ccc(-c4cc(OC)c5nn(C)cc5c4)cc3O)nn2)C1] |
| 49A | 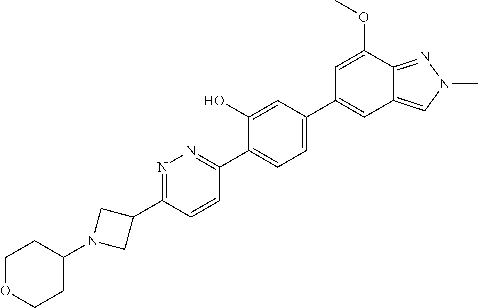 [SMILES: COc1cc(-c2ccc(-c3ccc(C4CN(C5CCOCC5)C4)nn3)c(O)c2)cc2cn(C)nc12] |
| 50A | 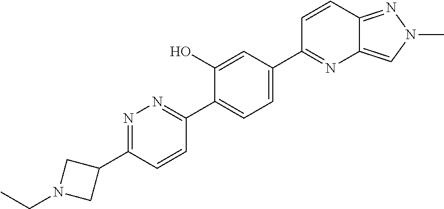 [SMILES: CCN1CC(c2ccc(-c3ccc(-c4ccc5nn(C)cc5n4)cc3O)nn2)C1] |
| 51A | 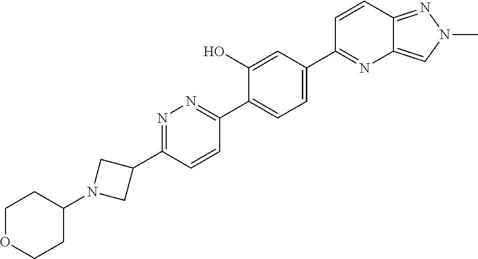 [SMILES: Cn1cc2nc(-c3ccc(-c4ccc(C5CN(C6CCOCC6)C5)nn4)c(O)c3)ccc2n1] |
| 52A | 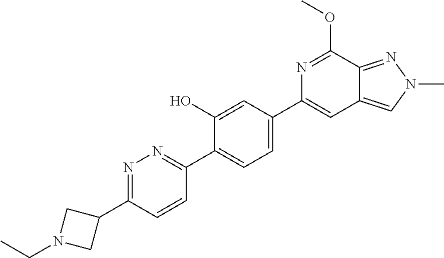 [SMILES: CCN1CC(c2ccc(-c3ccc(-c4cc5cn(C)nc5c(OC)n4)cc3O)nn2)C1] |
| 53A | 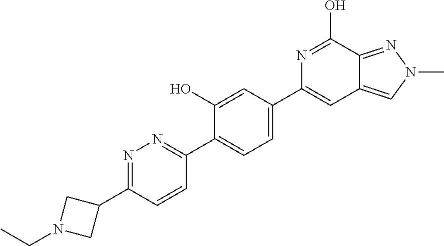 [SMILES: CCN1CC(c2ccc(-c3ccc(-c4cc5cn(C)nc5c(O)n4)cc3O)nn2)C1] |
| 54A | 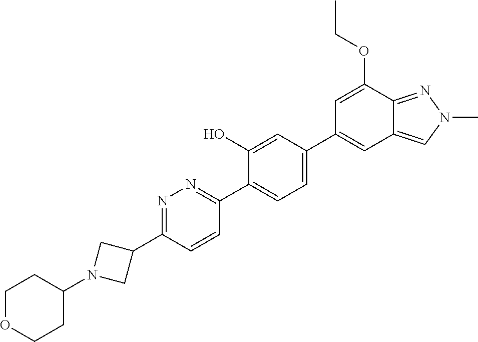 [SMILES: CCOc1cc(-c2ccc(-c3ccc(C4CN(C5CCOCC5)C4)nn3)c(O)c2)cc2cn(C)nc12] |
| 55A | 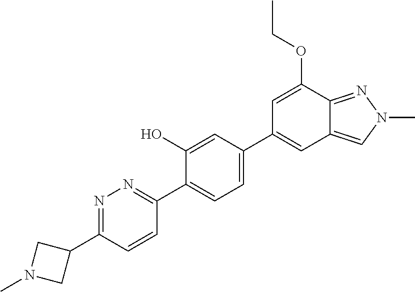 |
| 56A | 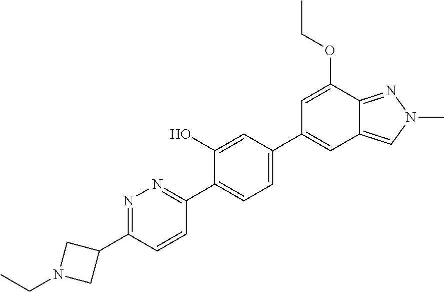 [SMILES: CCOc1cc(-c2ccc(-c3ccc(C4CN(CC)C4)nn3)c(O)c2)cc2cn(C)nc12] |
| 57A | 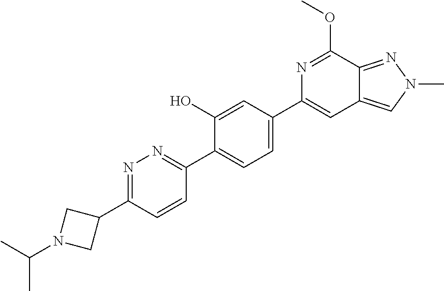 [SMILES: COc1nc(-c2ccc(-c3ccc(C4CN(C(C)C)C4)nn3)c(O)c2)cc2cn(C)nc12] |
| 58A | 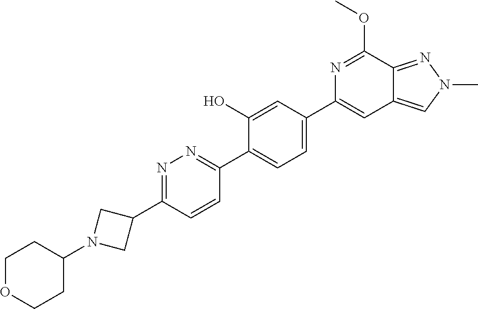 [SMILES: COc1nc(-c2ccc(-c3ccc(C4CN(C5CCOCC5)C4)nn3)c(O)c2)cc2cn(C)nc12] |
| 59A | 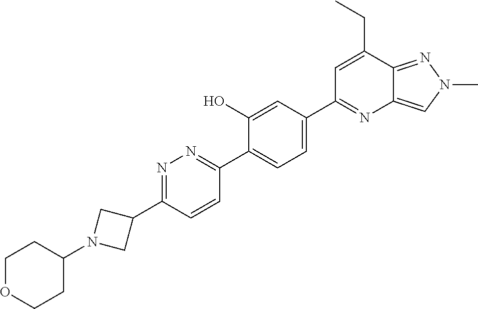 [SMILES: CCc1cc(-c2ccc(-c3ccc(C4CN(C5CCOCC5)C4)nn3)c(O)c2)nc2cn(C)nc12] |
| 60A | 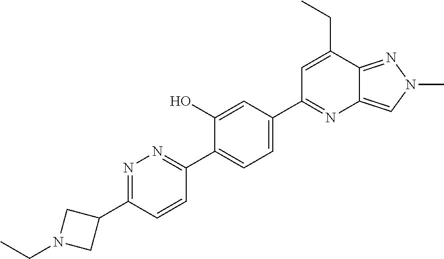 [SMILES: CCc1cc(-c2ccc(-c3ccc(C4CN(CC)C4)nn3)c(O)c2)nc2cn(C)nc12] |
| 61A | 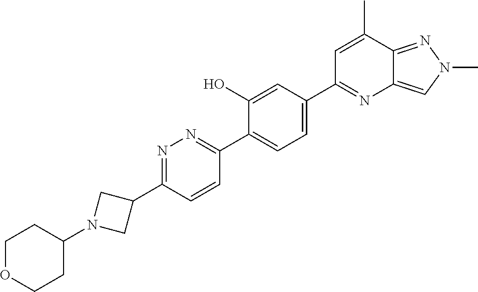 [SMILES: Cc1cc(-c2ccc(-c3ccc(C4CN(C5CCOCC5)C4)nn3)c(O)c2)nc2cn(C)nc12] |
| 62A | 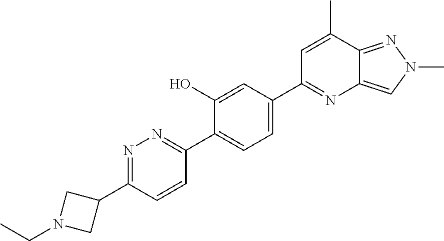 [SMILES: CCN1CC(c2ccc(-c3ccc(-c4cc(C)c5nn(C)cc5n4)cc3O)nn2)C1] |
| 63A | 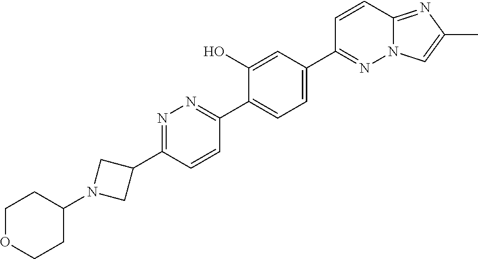 [SMILES: Cc1cn2nc(-c3ccc(-c4ccc(C5CN(C6CCOCC6)C5)nn4)c(O)c3)ccc2n1] |
| 64A | 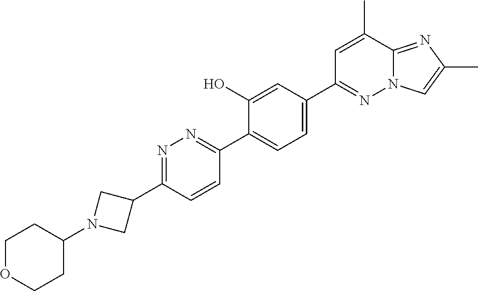 [SMILES: Cc1cn2nc(-c3ccc(-c4ccc(C5CN(C6CCOCC6)C5)nn4)c(O)c3)cc(C)c2n1] |
| 65A | 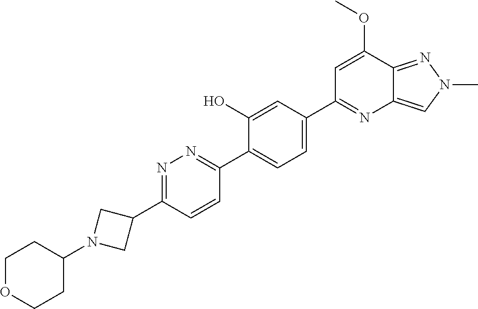 [SMILES: COc1cc(-c2ccc(-c3ccc(C4CN(C5CCOCC5)C4)nn3)c(O)c2)nc2cn(C)nc12] |
| 66A | 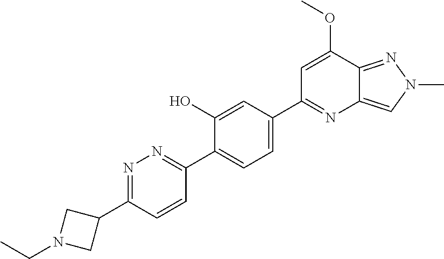 [SMILES: CCN1CC(c2ccc(-c3ccc(-c4cc(OC)c5nn(C)cc5n4)cc3O)nn2)C1] |
| 70A | 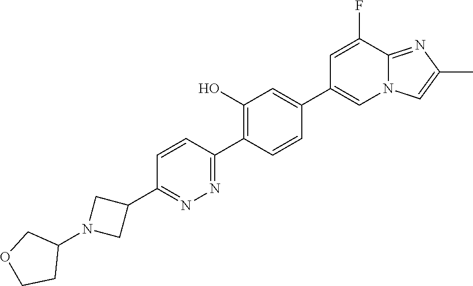 [SMILES: Cc1cn2cc(-c3ccc(-c4ccc(C5CN(C6CCOC6)C5)nn4)c(O)c3)cc(F)c2n1] |
| 71A | 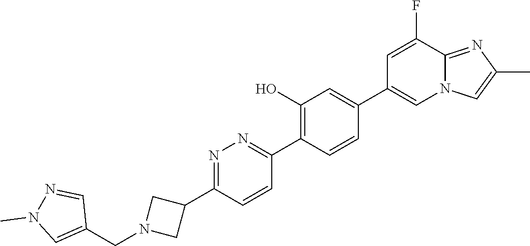 |
| 72A | 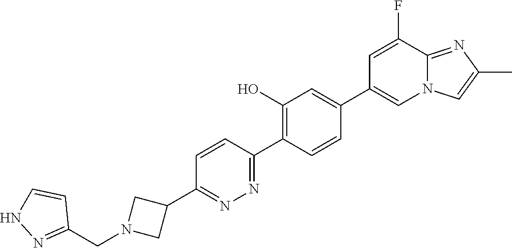 [SMILES: Cc1cn2cc(-c3ccc(-c4ccc(C5CN(Cc6cc[nH]n6)C5)nn4)c(O)c3)cc(F)c2n1] |
| 73A | 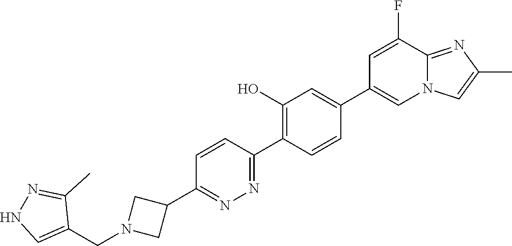 [SMILES: Cc1cn2cc(-c3ccc(-c4ccc(C5CN(Cc6c[nH]nc6C)C5)nn4)c(O)c3)cc(F)c2n1] |
| 74A | 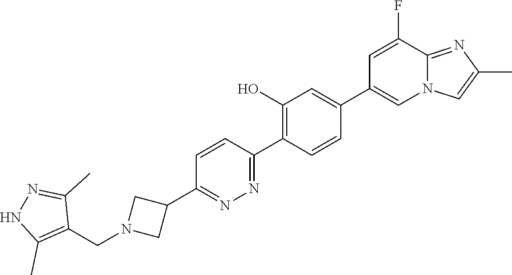 [SMILES: Cc1cn2cc(-c3ccc(-c4ccc(C5CN(Cc6c(C)n[nH]c6C)C5)nn4)c(O)c3)cc(F)c2n1] |
| 75A | 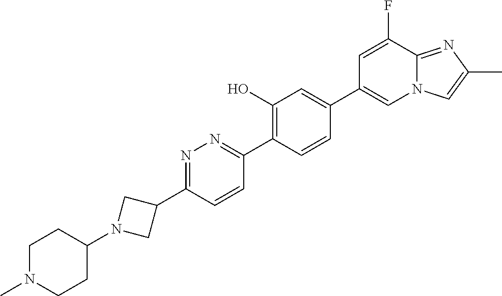 |
| 76A | 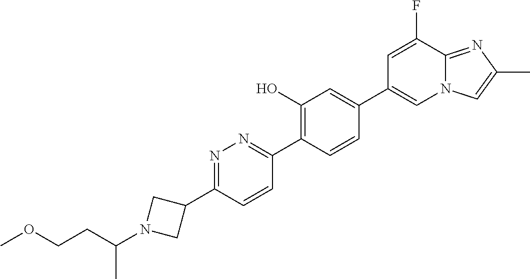 |
| 77A | 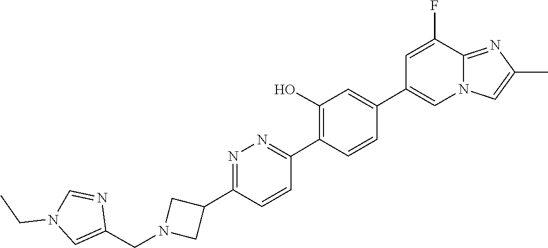 [SMILES: CCn1cnc(CN2CC(c3ccc(-c4ccc(-c5cc(F)c6nc(C)cn6c5)cc4O)nn3)C2)c1] |
| 78A | 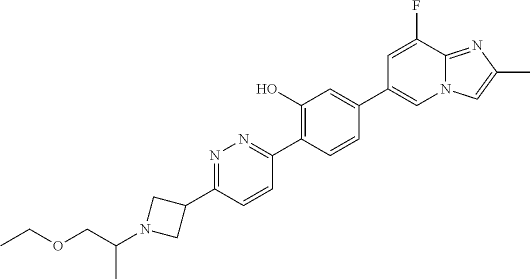 [SMILES: CCOCC(C)N1CC(c2ccc(-c3ccc(-c4cc(F)c5nc(C)cn5c4)cc3O)nn2)C1] |
| 79A | 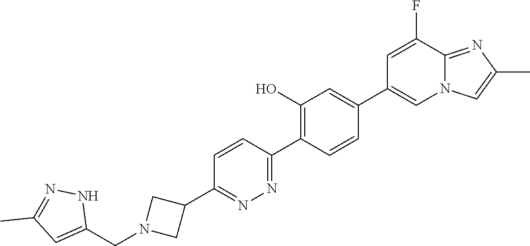 |
| 80A | 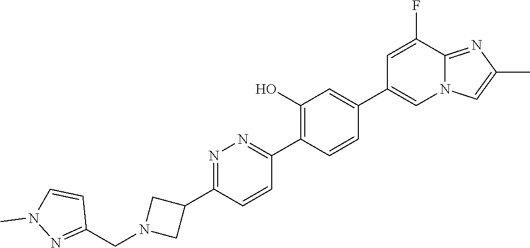 |
| 81A | 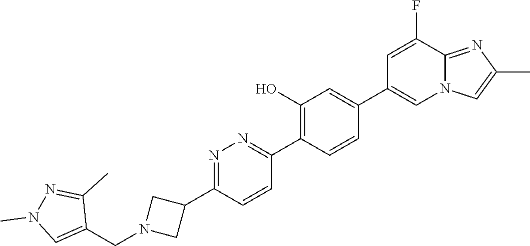 |
| 82A | 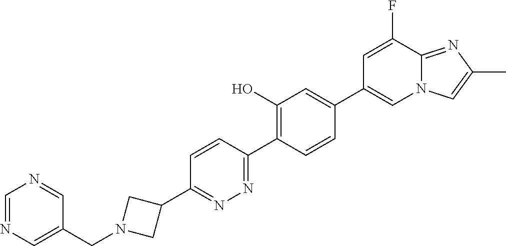 [SMILES: Cc1cn2cc(-c3ccc(-c4ccc(C5CN(Cc6cncnc6)C5)nn4)c(O)c3)cc(F)c2n1] |
| 83A | 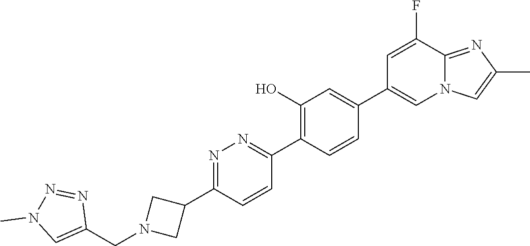 |
| 84A | 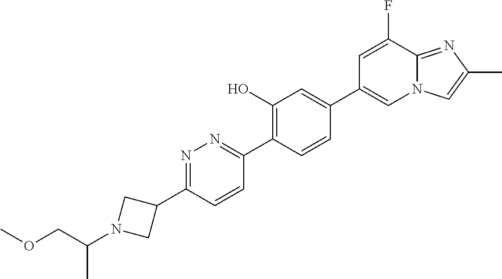 |
| 85A | 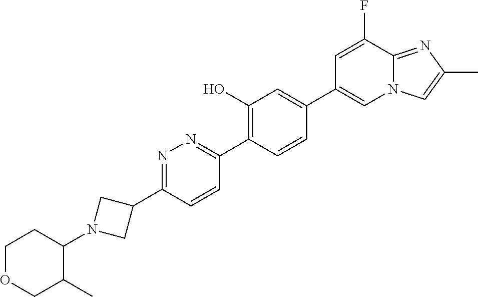 [SMILES: Cc1cn2cc(-c3ccc(-c4ccc(C5CN(C6CCOCC6C)C5)nn4)c(O)c3)cc(F)c2n1] |
| 86A | 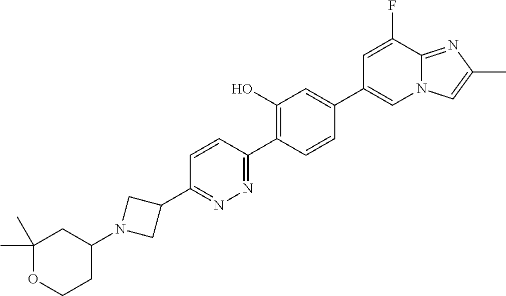 |
| 87A | 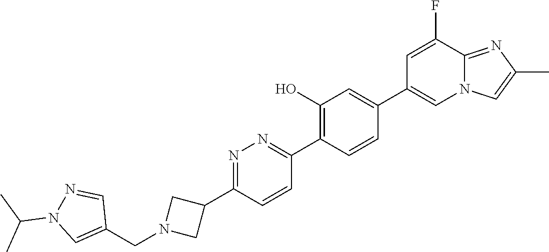 [SMILES: Cc1cn2cc(-c3ccc(-c4ccc(C5CN(Cc6cnn(C(C)C)c6)C5)nn4)c(O)c3)cc(F)c2n1] |
| 88A | 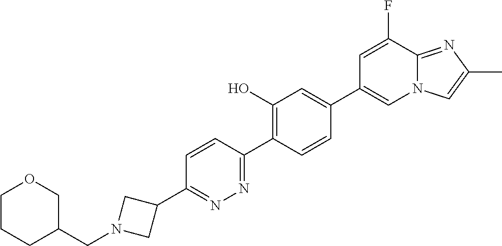 [SMILES: Cc1cn2cc(-c3ccc(-c4ccc(C5CN(CC6CCCOC6)C5)nn4)c(O)c3)cc(F)c2n1] |
| 89A | 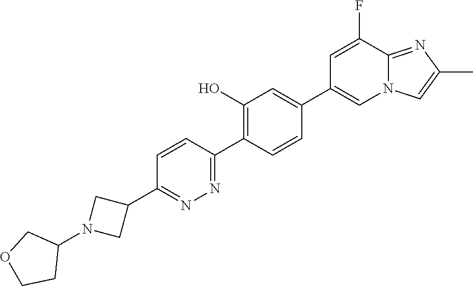 [SMILES: Cc1cn2cc(-c3ccc(-c4ccc(C5CN(C6CCOC6)C5)nn4)c(O)c3)cc(F)c2n1] |
| 90A | 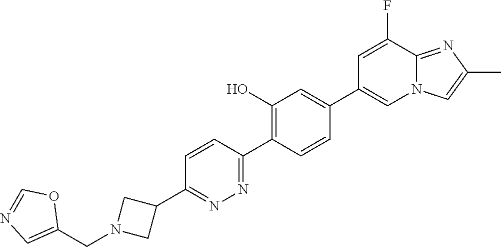 [SMILES: Cc1cn2cc(-c3ccc(-c4ccc(C5CN(Cc6cnco6)C5)nn4)c(O)c3)cc(F)c2n1] |
| 91A | 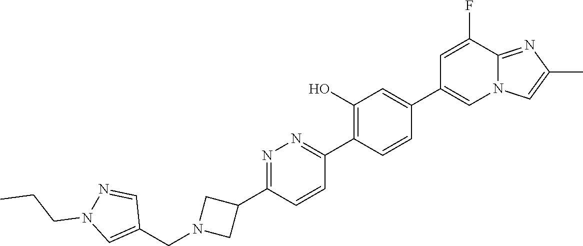 [SMILES: CCCn1cc(CN2CC(c3ccc(-c4ccc(-c5cc(F)c6nc(C)cn6c5)cc4O)nn3)C2)cn1] |
| 92A | 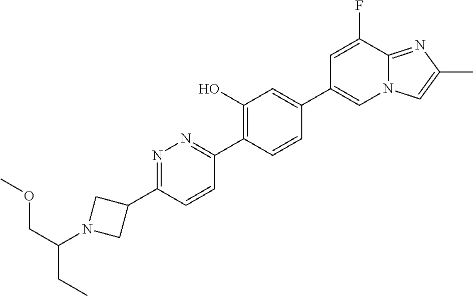 |
| 93A | 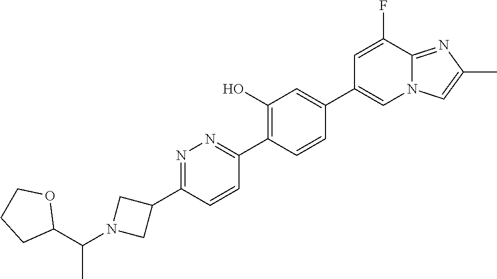 [SMILES: Cc1cn2cc(-c3ccc(-c4ccc(C5CN(C(C)C6CCCO6)C5)nn4)c(O)c3)cc(F)c2n1] |
| 94A | 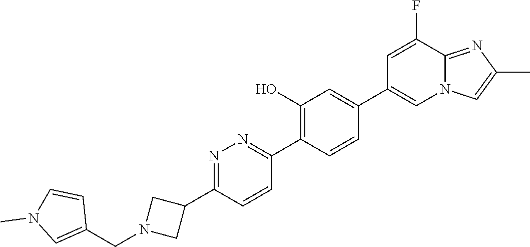 |
| 95A | 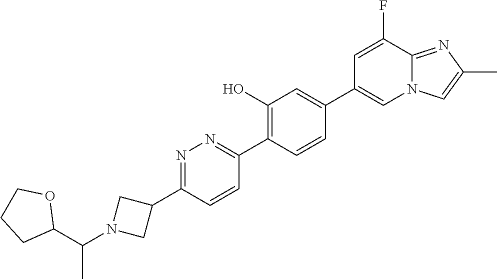 [SMILES: Cc1cn2cc(-c3ccc(-c4ccc(C5CN(C(C)C6CCCO6)C5)nn4)c(O)c3)cc(F)c2n1] |
| 96A | 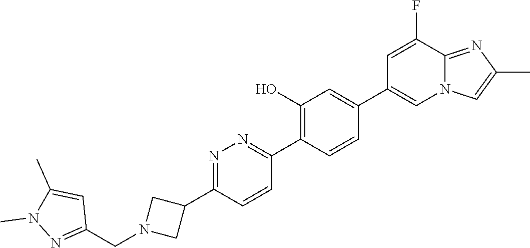 |
| 97A | 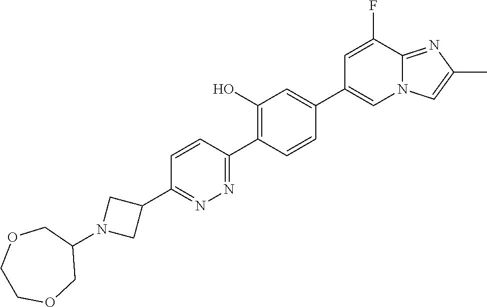 [SMILES: Cc1cn2cc(-c3ccc(-c4ccc(C5CN(C6COCCOC6)C5)nn4)c(O)c3)cc(F)c2n1] |
| 98A | 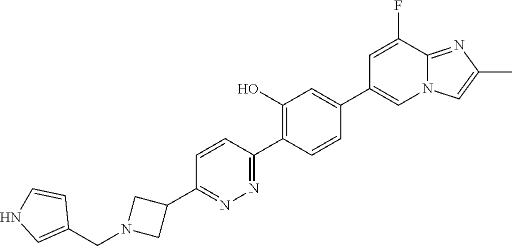 [SMILES: Cc1cn2cc(-c3ccc(-c4ccc(C5CN(Cc6cc[nH]c6)C5)nn4)c(O)c3)cc(F)c2n1] |
| 99A | 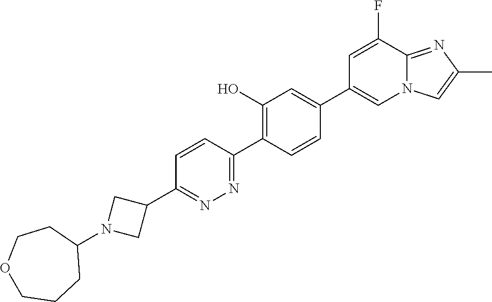 [SMILES: Cc1cn2cc(-c3ccc(-c4ccc(C5CN(C6CCCOCC6)C5)nn4)c(O)c3)cc(F)c2n1] |
| 100A | 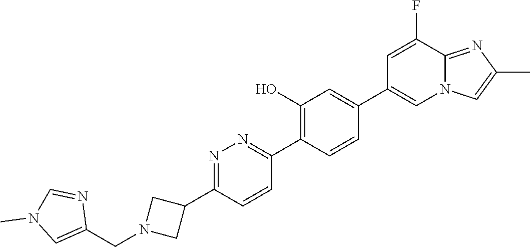 |
| 101A | 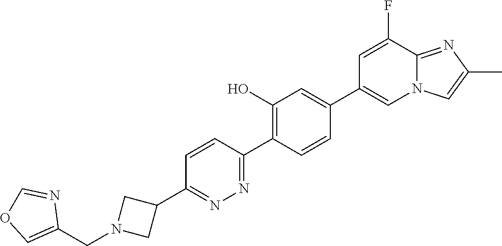 [SMILES: Cc1cn2cc(-c3ccc(-c4ccc(C5CN(Cc6cocn6)C5)nn4)c(O)c3)cc(F)c2n1] |
| 102A | 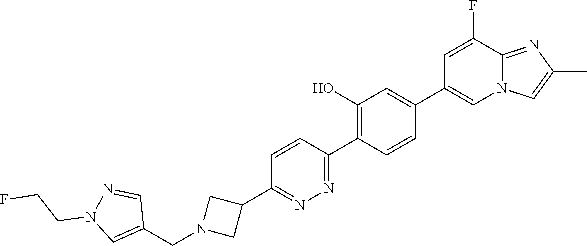 [SMILES: Cc1cn2cc(-c3ccc(-c4ccc(C5CN(Cc6cnn(CCF)c6)C5)nn4)c(O)c3)cc(F)c2n1] |
| 103A | 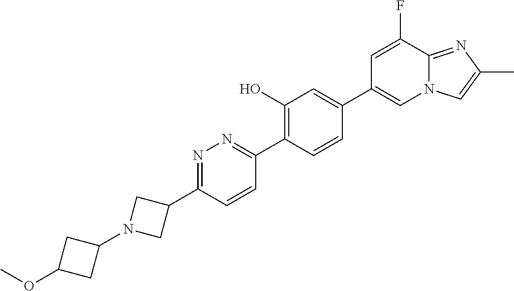 |
| 104A | 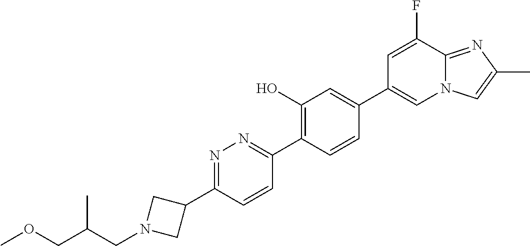 |
| 105A | 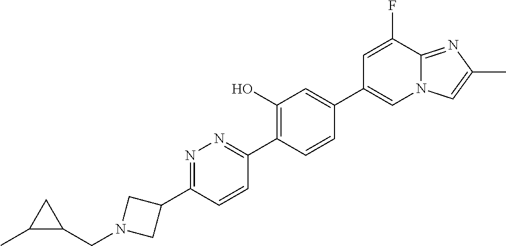 |
| 106A | 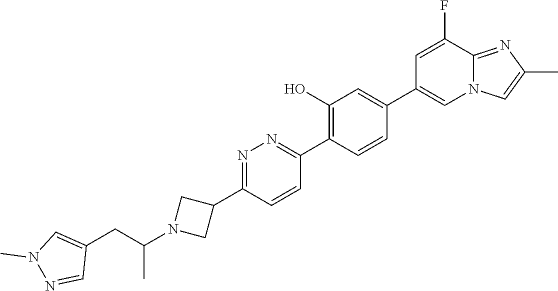 |
| 107A | 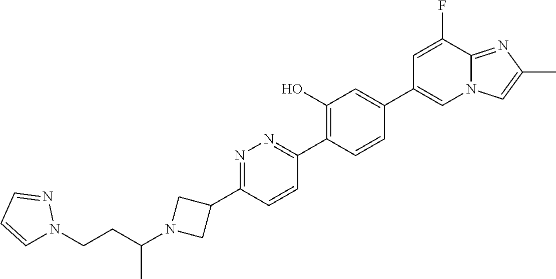 [SMILES: Cc1cn2cc(-c3ccc(-c4ccc(C5CN(C(C)CCn6cccn6)C5)nn4)c(O)c3)cc(F)c2n1] |
| 108A | 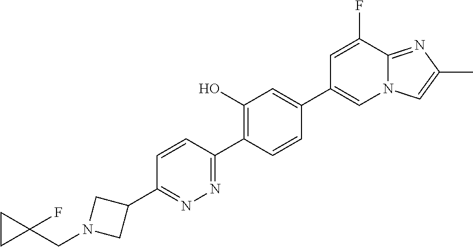 [SMILES: Cc1cn2cc(-c3ccc(-c4ccc(C5CN(CC6(F)CC6)C5)nn4)c(O)c3)cc(F)c2n1] |
| 109A | 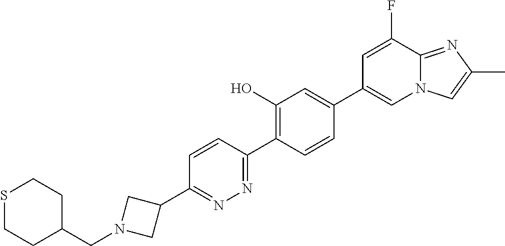 [SMILES: Cc1cn2cc(-c3ccc(-c4ccc(C5CN(CC6CCSCC6)C5)nn4)c(O)c3)cc(F)c2n1] |
| 110A | 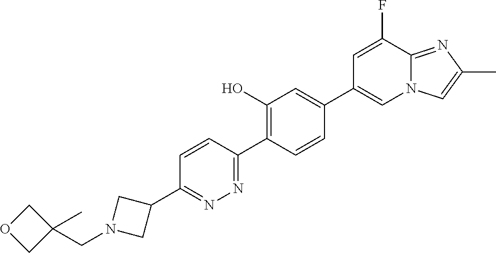 [SMILES: Cc1cn2cc(-c3ccc(-c4ccc(C5CN(CC6(C)COC6)C5)nn4)c(O)c3)cc(F)c2n1] |
| 111A | 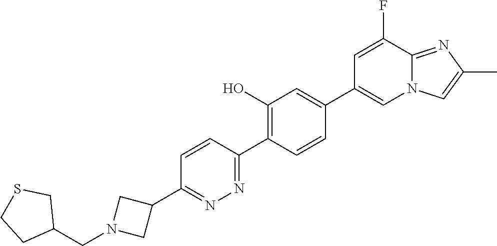 [SMILES: Cc1cn2cc(-c3ccc(-c4ccc(C5CN(CC6CCSC6)C5)nn4)c(O)c3)cc(F)c2n1] |
| 112A | 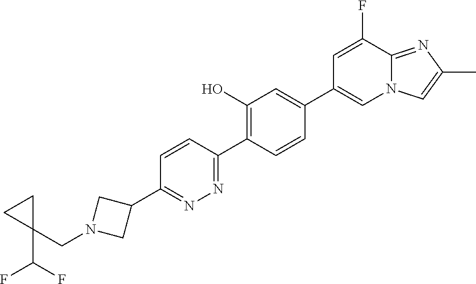 [SMILES: Cc1cn2cc(-c3ccc(-c4ccc(C5CN(CC6(C(F)F)CC6)C5)nn4)c(O)c3)cc(F)c2n1] |
| 113A | 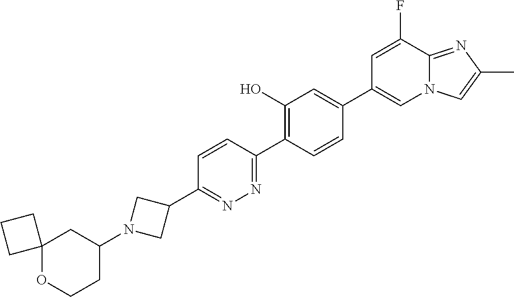 [SMILES: Cc1cn2cc(-c3ccc(-c4ccc(C5CN(C6CCOC7(CCC7)C6)C5)nn4)c(O)c3)cc(F)c2n1] |
| 114A | 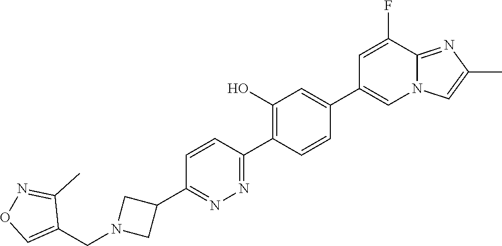 [SMILES: Cc1cn2cc(-c3ccc(-c4ccc(C5CN(Cc6conc6C)C5)nn4)c(O)c3)cc(F)c2n1] |
| 115A | 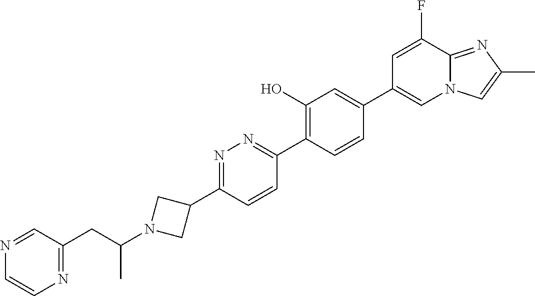 [SMILES: Cc1cn2cc(-c3ccc(-c4ccc(C5CN(C(C)Cc6cnccn6)C5)nn4)c(O)c3)cc(F)c2n1] |
| 116A | 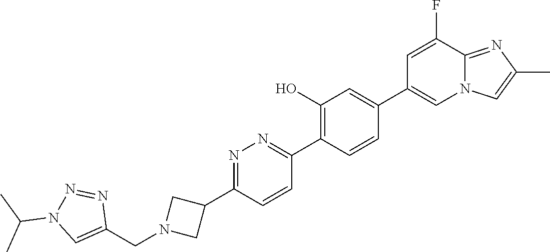 [SMILES: Cc1cn2cc(-c3ccc(-c4ccc(C5CN(Cc6cn(C(C)C)nn6)C5)nn4)c(O)c3)cc(F)c2n1] |
| 117A | 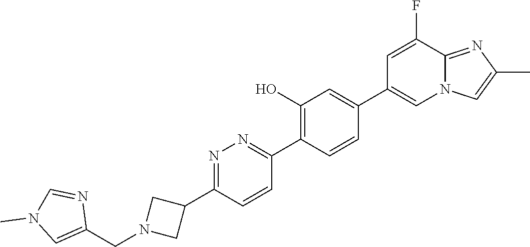 |
| 118A | 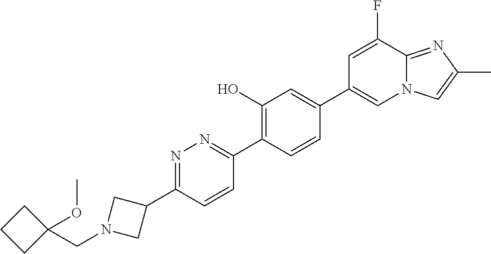 [SMILES: COC1(CN2CC(c3ccc(-c4ccc(-c5cc(F)c6nc(C)cn6c5)cc4O)nn3)C2)CCC1] |
| 119A | 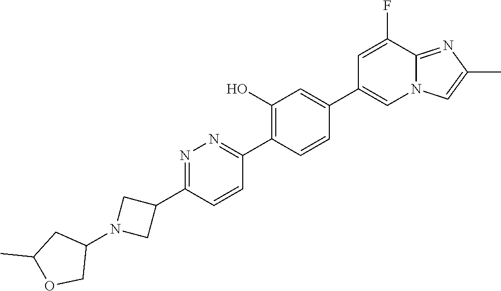 |
| 120A | 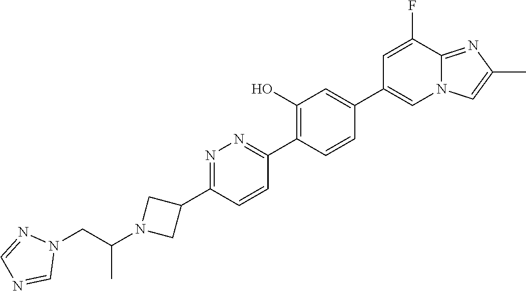 [SMILES: Cc1cn2cc(-c3ccc(-c4ccc(C5CN(C(C)Cn6cncn6)C5)nn4)c(O)c3)cc(F)c2n1] |
| 121A | 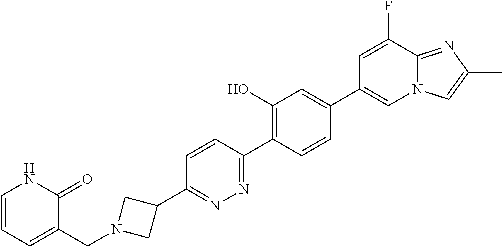 [SMILES: Cc1cn2cc(-c3ccc(-c4ccc(C5CN(Cc6ccc[nH]c6=O)C5)nn4)c(O)c3)cc(F)c2n1] |
| 122A | 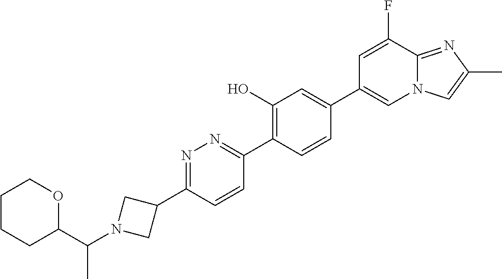 [SMILES: Cc1cn2cc(-c3ccc(-c4ccc(C5CN(C(C)C6CCCCO6)C5)nn4)c(O)c3)cc(F)c2n1] |
| 123A | 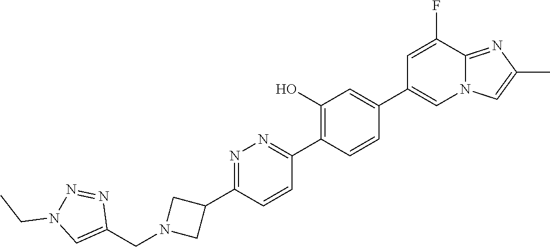 [SMILES: CCn1cc(CN2CC(c3ccc(-c4ccc(-c5cc(F)c6nc(C)cn6c5)cc4O)nn3)C2)nn1] |
| 124A | 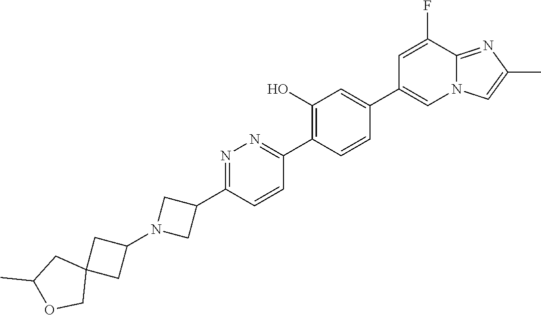 |
| 125A | 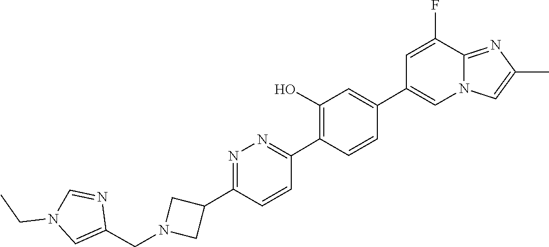 [SMILES: CCn1cnc(CN2CC(c3ccc(-c4ccc(-c5cc(F)c6nc(C)cn6c5)cc4O)nn3)C2)c1] |
| 126A | 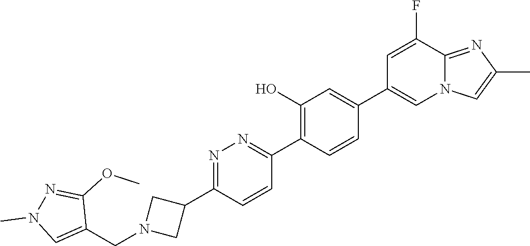 |
| 127A | 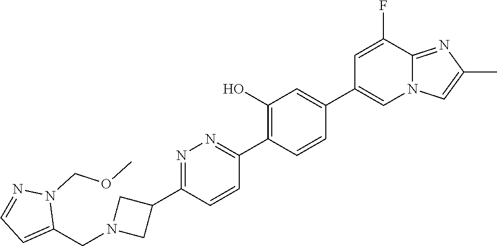 [SMILES: COCn1nccc1CN1CC(c2ccc(-c3ccc(-c4cc(F)c5nc(C)cn5c4)cc3O)nn2)C1] |
| 128A | 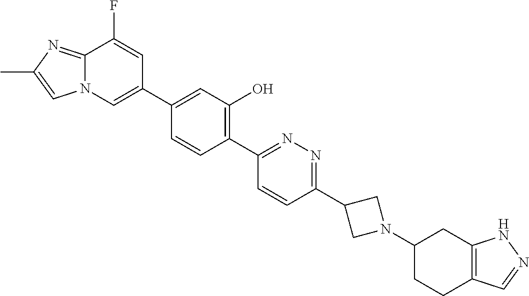 [SMILES: Cc1cn2cc(-c3ccc(-c4ccc(C5CN(C6CCc7cn[nH]c7C6)C5)nn4)c(O)c3)cc(F)c2n1] |
| 129A | 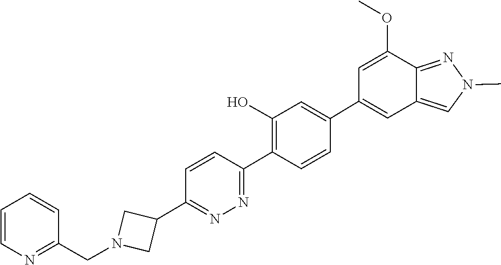 [SMILES: COc1cc(-c2ccc(-c3ccc(C4CN(Cc5ccccn5)C4)nn3)c(O)c2)cc2cn(C)nc12] |
| 130A | 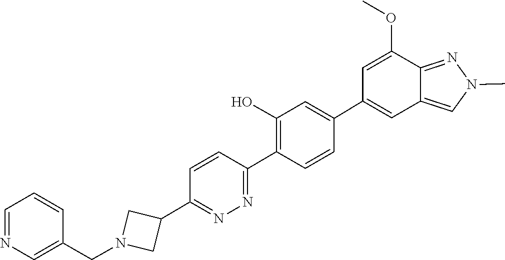 [SMILES: COc1cc(-c2ccc(-c3ccc(C4CN(Cc5cccnc5)C4)nn3)c(O)c2)cc2cn(C)nc12] |
| 131A | 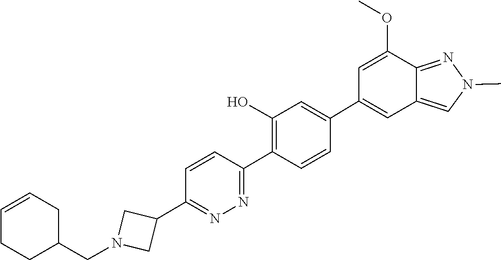 [SMILES: COc1cc(-c2ccc(-c3ccc(C4CN(CC5CC=CCC5)C4)nn3)c(O)c2)cc2cn(C)nc12] |
| 132A | 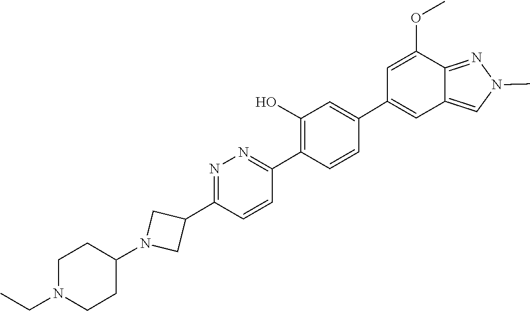 [SMILES: CCN1CCC(N2CC(c3ccc(-c4ccc(-c5cc(OC)c6nn(C)cc6c5)cc4O)nn3)C2)CC1] |
| 133A | 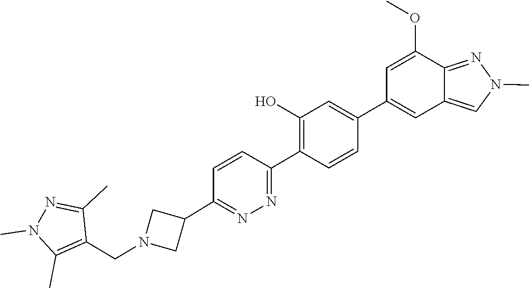 |
| 134A | 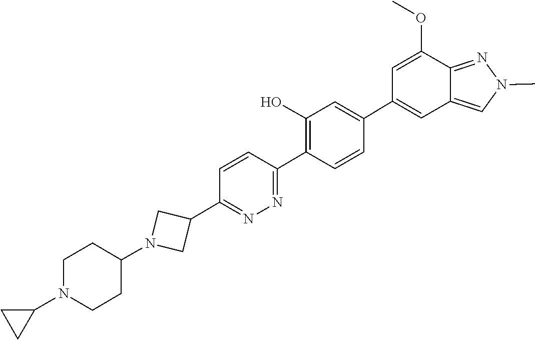 [SMILES: COc1cc(-c2ccc(-c3ccc(C4CN(C5CCN(C6CC6)CC5)C4)nn3)c(O)c2)cc2cn(C)nc12] |
| 135A | 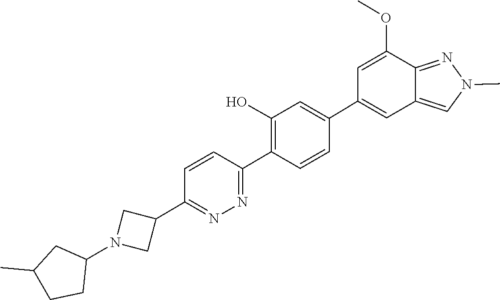 |
| 136A | 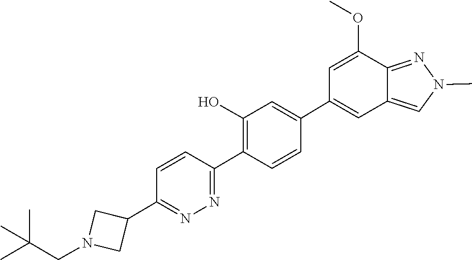 [SMILES: COc1cc(-c2ccc(-c3ccc(C4CN(CC(C)(C)C)C4)nn3)c(O)c2)cc2cn(C)nc12] |
| 137A | 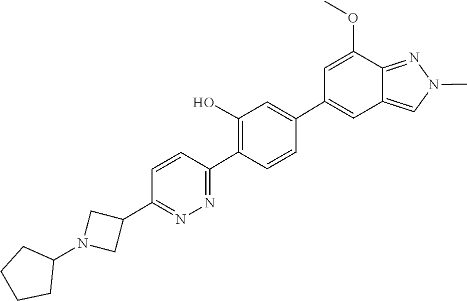 [SMILES: COc1cc(-c2ccc(-c3ccc(C4CN(C5CCCC5)C4)nn3)c(O)c2)cc2cn(C)nc12] |
| 138A | 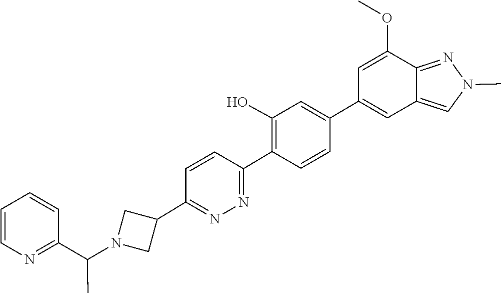 [SMILES: COc1cc(-c2ccc(-c3ccc(C4CN(C(C)c5ccccn5)C4)nn3)c(O)c2)cc2cn(C)nc12] |
| 139A | 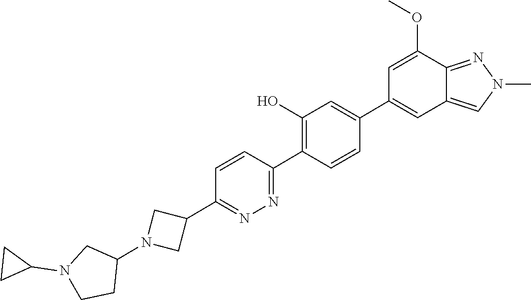 [SMILES: COc1cc(-c2ccc(-c3ccc(C4CN(C5CCN(C6CC6)C5)C4)nn3)c(O)c2)cc2cn(C)nc12] |
| 140A | 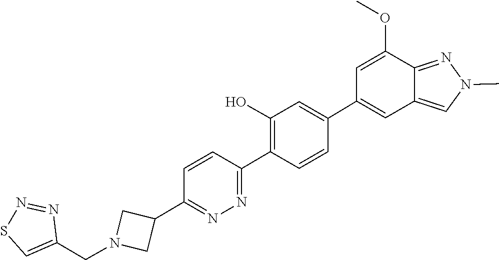 [SMILES: COc1cc(-c2ccc(-c3ccc(C4CN(Cc5csnn5)C4)nn3)c(O)c2)cc2cn(C)nc12] |
| 141A | 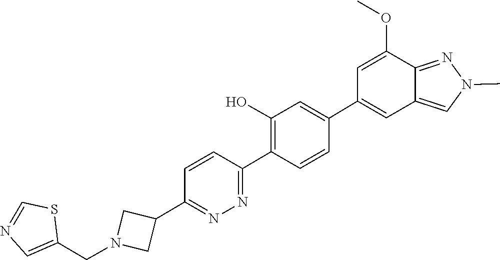 [SMILES: COc1cc(-c2ccc(-c3ccc(C4CN(Cc5cncs5)C4)nn3)c(O)c2)cc2cn(C)nc12] |
| 142A | 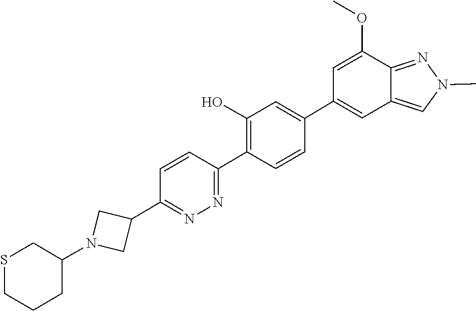 [SMILES: COc1cc(-c2ccc(-c3ccc(C4CN(C5CCCSC5)C4)nn3)c(O)c2)cc2cn(C)nc12] |
| 143A | 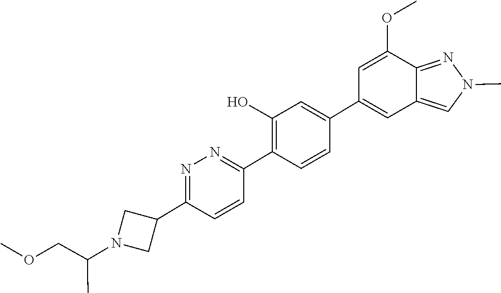 |
| 144A | 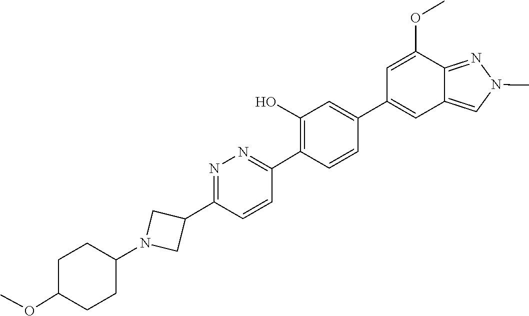 |
| 145A | 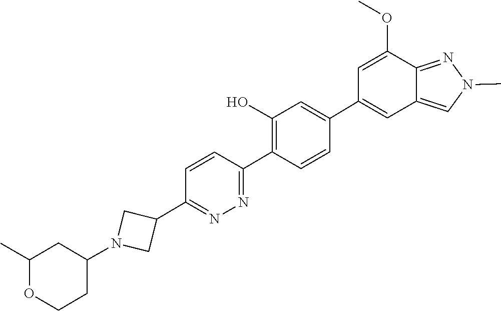 |
| 146A | 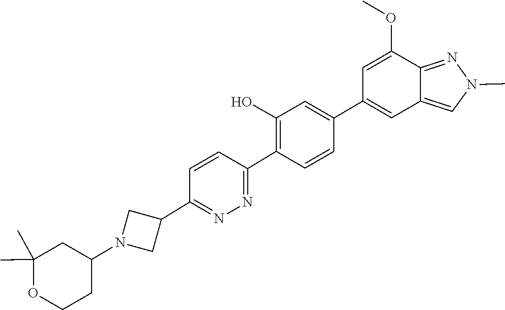 |
| 147A | 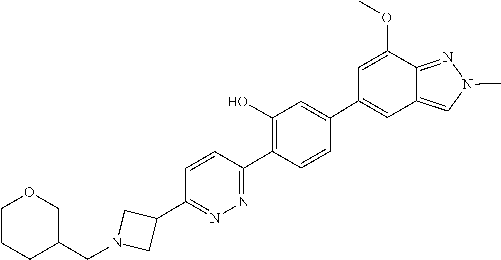 [SMILES: COc1cc(-c2ccc(-c3ccc(C4CN(CC5CCCOC5)C4)nn3)c(O)c2)cc2cn(C)nc12] |
| 148A | 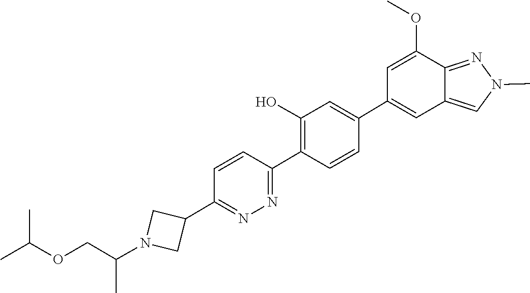 [SMILES: COc1cc(-c2ccc(-c3ccc(C4CN(C(C)COC(C)C)C4)nn3)c(O)c2)cc2cn(C)nc12] |
| 149A | 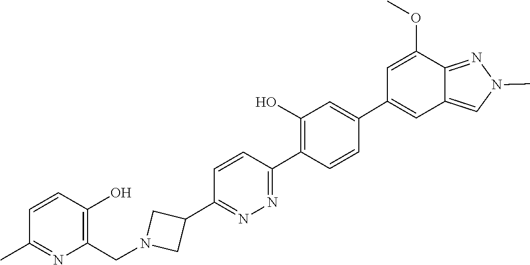 |
| 150A | 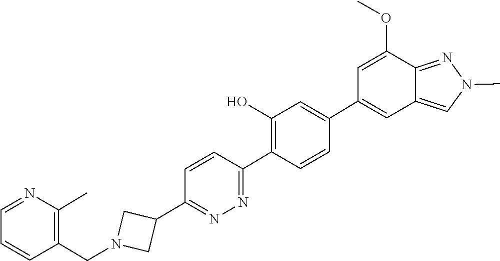 [SMILES: COc1cc(-c2ccc(-c3ccc(C4CN(Cc5cccnc5C)C4)nn3)c(O)c2)cc2cn(C)nc12] |
| 151A | 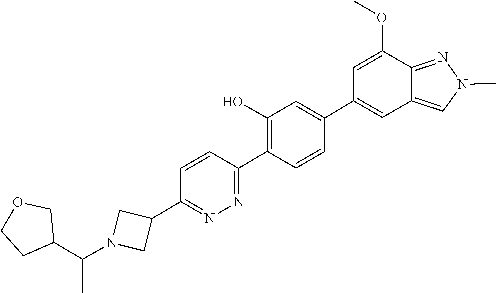 [SMILES: COc1cc(-c2ccc(-c3ccc(C4CN(C(C)C5CCOC5)C4)nn3)c(O)c2)cc2cn(C)nc12] |
| 152A | 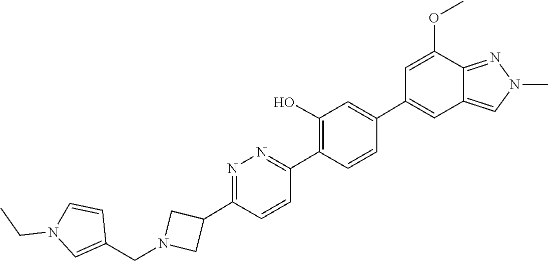 [SMILES: CCn1ccc(CN2CC(c3ccc(-c4ccc(-c5cc(OC)c6nn(C)cc6c5)cc4O)nn3)C2)c1] |
| 153A | 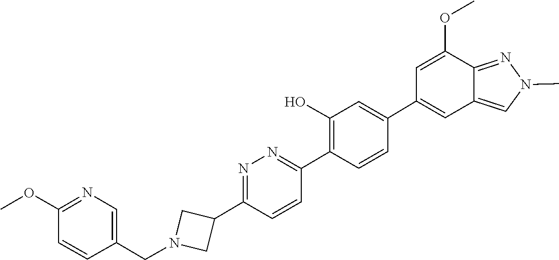 |
| 154A | 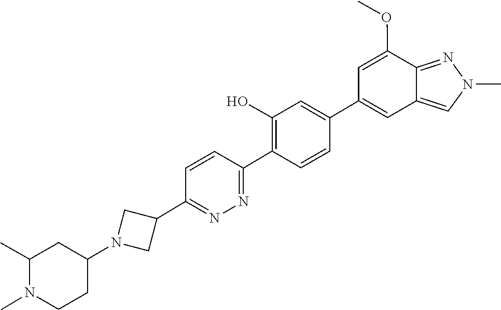 |
| 155A | 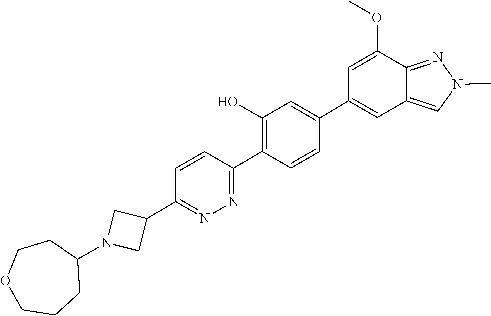 [SMILES: COc1cc(-c2ccc(-c3ccc(C4CN(C5CCCOCC5)C4)nn3)c(O)c2)cc2cn(C)nc12] |
| 156A | 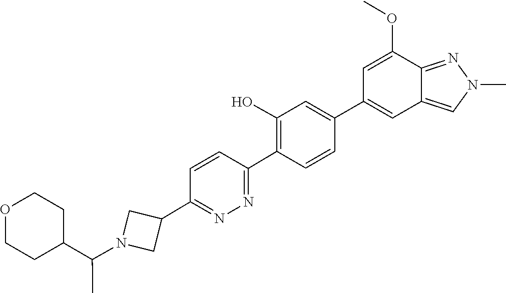 [SMILES: COc1cc(-c2ccc(-c3ccc(C4CN(C(C)C5CCOCC5)C4)nn3)c(O)c2)cc2cn(C)nc12] |
| 157A | 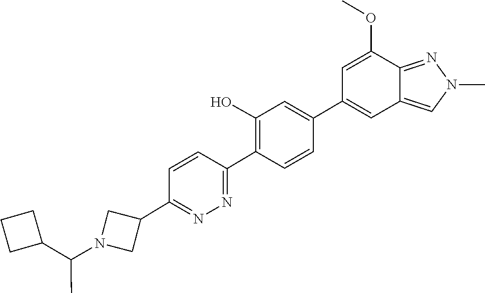 [SMILES: COc1cc(-c2ccc(-c3ccc(C4CN(C(C)C5CCC5)C4)nn3)c(O)c2)cc2cn(C)nc12] |
| 158A | 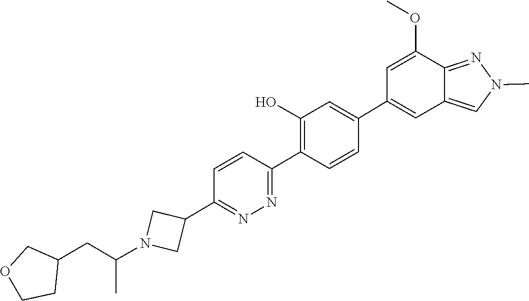 [SMILES: COc1cc(-c2ccc(-c3ccc(C4CN(C(C)CC5CCOC5)C4)nn3)c(O)c2)cc2cn(C)nc12] |
| 159A | 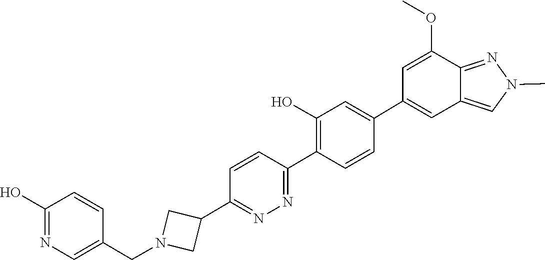 [SMILES: COc1cc(-c2ccc(-c3ccc(C4CN(Cc5ccc(O)nc5)C4)nn3)c(O)c2)cc2cn(C)nc12] |
| 160A | 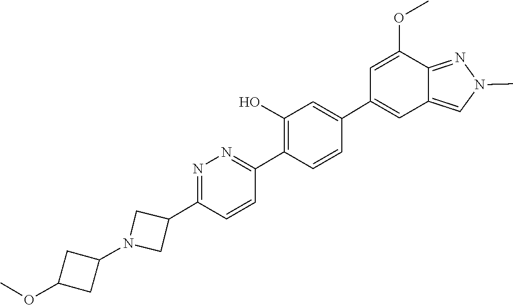 |
| 161A | 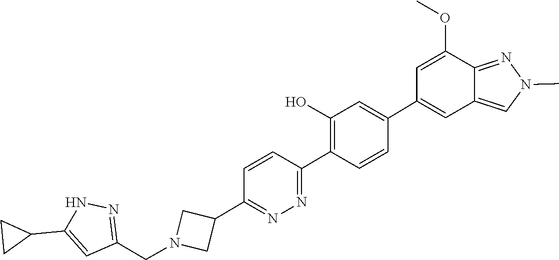 [SMILES: COc1cc(-c2ccc(-c3ccc(C4CN(Cc5cc(C6CC6)[nH]n5)C4)nn3)c(O)c2)cc2cn(C)nc12] |
| 162A | 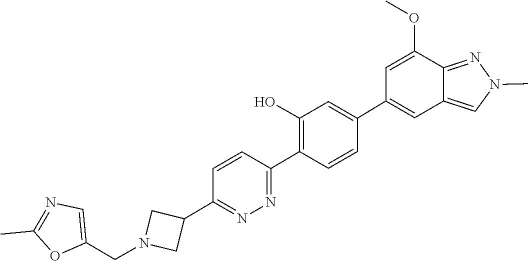 |
| 163A | 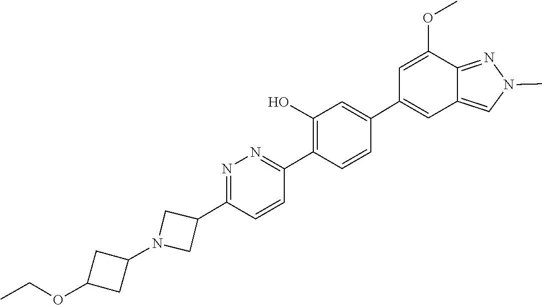 [SMILES: CCOC1CC(N2CC(c3ccc(-c4ccc(-c5cc(OC)c6nn(C)cc6c5)cc4O)nn3)C2)C1] |
| 164A | 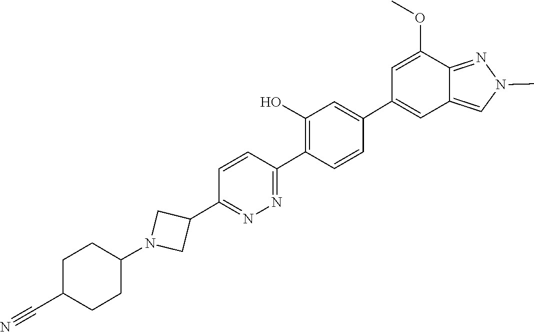 [SMILES: COc1cc(-c2ccc(-c3ccc(C4CN(C5CCC(C#N)CC5)C4)nn3)c(O)c2)cc2cn(C)nc12] |
| 165A | 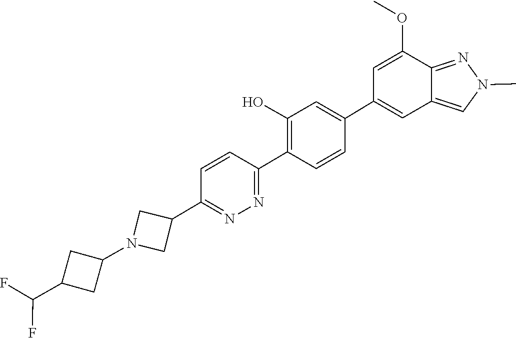 [SMILES: COc1cc(-c2ccc(-c3ccc(C4CN(C5CC(C(F)F)C5)C4)nn3)c(O)c2)cc2cn(C)nc12] |
| 166A | 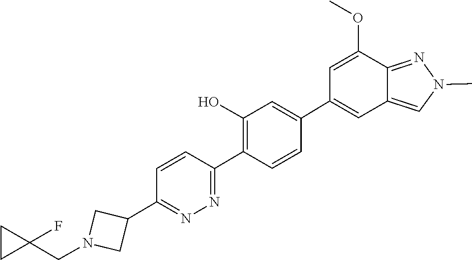 [SMILES: COc1cc(-c2ccc(-c3ccc(C4CN(CC5(F)CC5)C4)nn3)c(O)c2)cc2cn(C)nc12] |
| 167A | 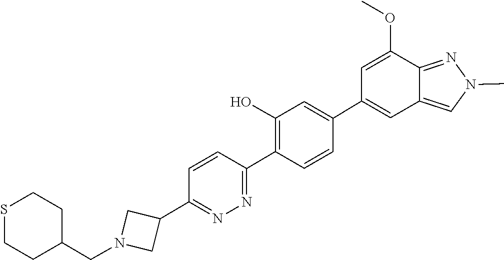 [SMILES: COc1cc(-c2ccc(-c3ccc(C4CN(CC5CCSCC5)C4)nn3)c(O)c2)cc2cn(C)nc12] |
| 168A | 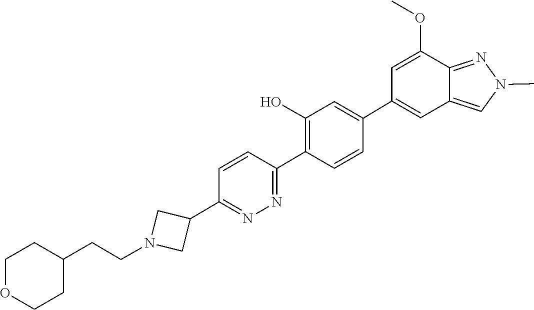 [SMILES: COc1cc(-c2ccc(-c3ccc(C4CN(CCC5CCOCC5)C4)nn3)c(O)c2)cc2cn(C)nc12] |
| 169A | 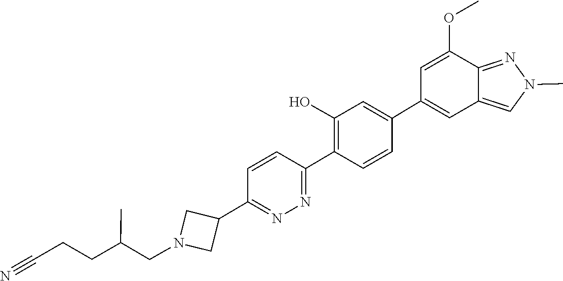 [SMILES: COc1cc(-c2ccc(-c3ccc(C4CN(CC(C)CCC#N)C4)nn3)c(O)c2)cc2cn(C)nc12] |
| 170A | 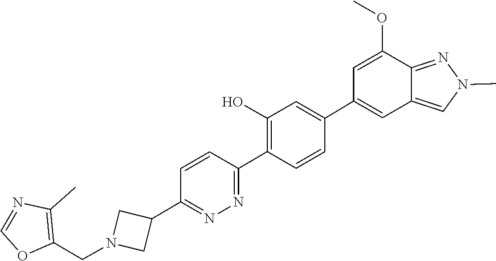 [SMILES: COc1cc(-c2ccc(-c3ccc(C4CN(Cc5ocnc5C)C4)nn3)c(O)c2)cc2cn(C)nc12] |
| 171A | 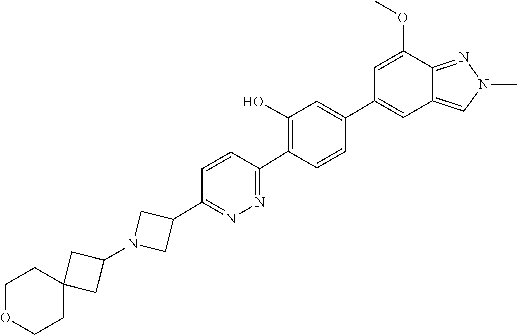 [SMILES: COc1cc(-c2ccc(-c3ccc(C4CN(C5CC6(CCOCC6)C5)C4)nn3)c(O)c2)cc2cn(C)nc12] |
| 172A | 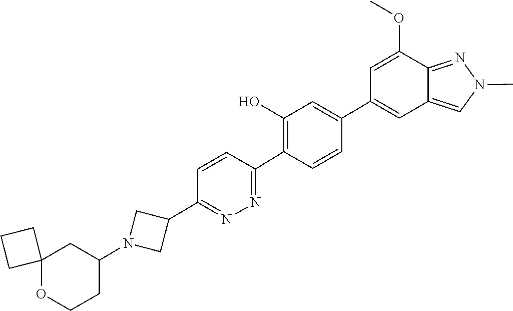 [SMILES: COc1cc(-c2ccc(-c3ccc(C4CN(C5CCOC6(CCC6)C5)C4)nn3)c(O)c2)cc2cn(C)nc12] |
| 173A | 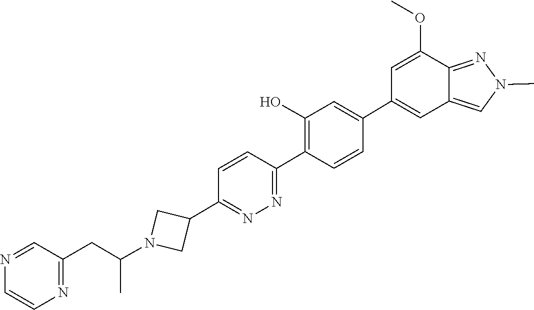 [SMILES: COc1cc(-c2ccc(-c3ccc(C4CN(C(C)Cc5cnccn5)C4)nn3)c(O)c2)cc2cn(C)nc12] |
| 174A | 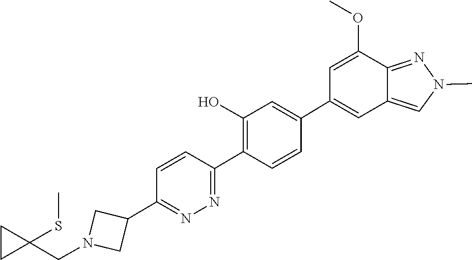 [SMILES: COc1cc(-c2ccc(-c3ccc(C4CN(CC5(SC)CC5)C4)nn3)c(O)c2)cc2cn(C)nc12] |
| 175A | 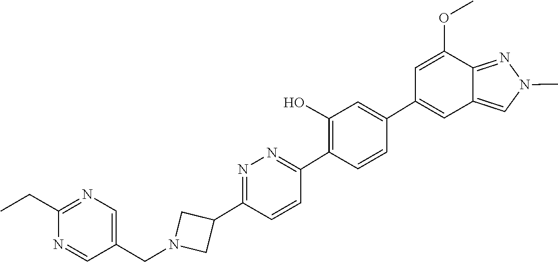 [SMILES: CCc1ncc(CN2CC(c3ccc(-c4ccc(-c5cc(OC)c6nn(C)cc6c5)cc4O)nn3)C2)cn1] |
| 176A | 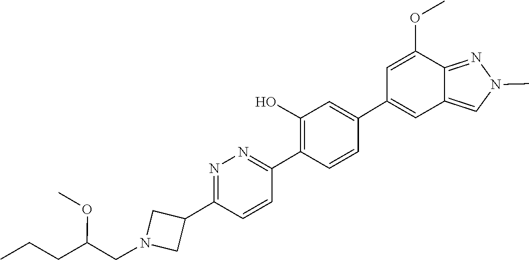 [SMILES: CCCC(CN1CC(c2ccc(-c3ccc(-c4cc(OC)c5nn(C)cc5c4)cc3O)nn2)C1)OC] |
| 177A | 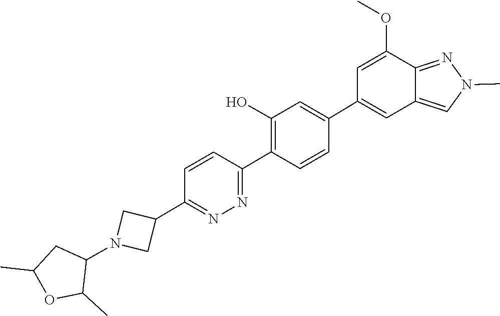 |
| 178A | 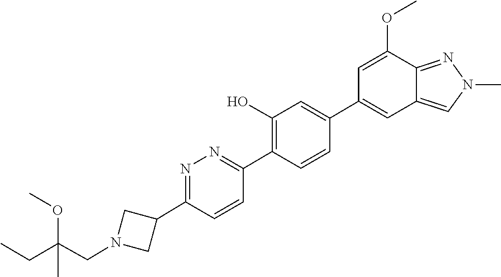 [SMILES: CCC(C)(CN1CC(c2ccc(-c3ccc(-c4cc(OC)c5nn(C)cc5c4)cc3O)nn2)C1)OC] |
| 179A | 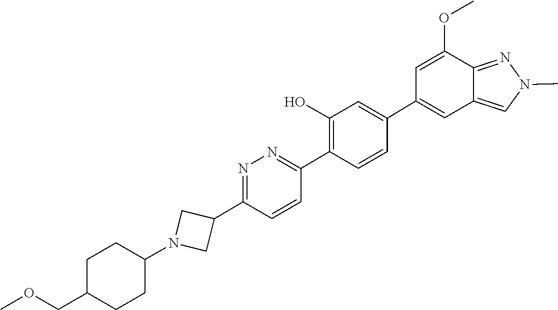 |
| 180A | 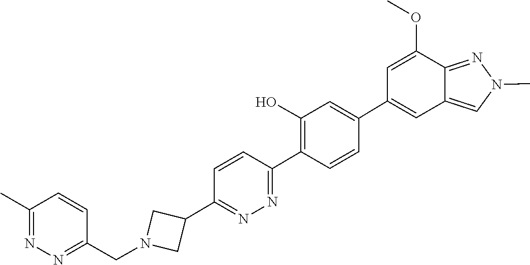 |
| 181A | 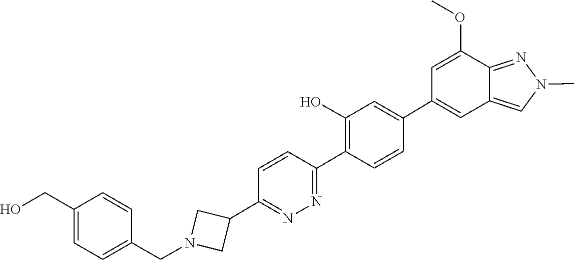 [SMILES: COc1cc(-c2ccc(-c3ccc(C4CN(Cc5ccc(CO)cc5)C4)nn3)c(O)c2)cc2cn(C)nc12] |
| 182A | 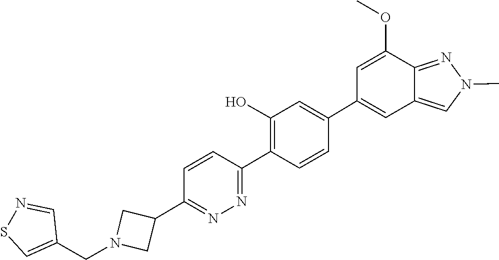 [SMILES: COc1cc(-c2ccc(-c3ccc(C4CN(Cc5cnsc5)C4)nn3)c(O)c2)cc2cn(C)nc12] |
| 183A | 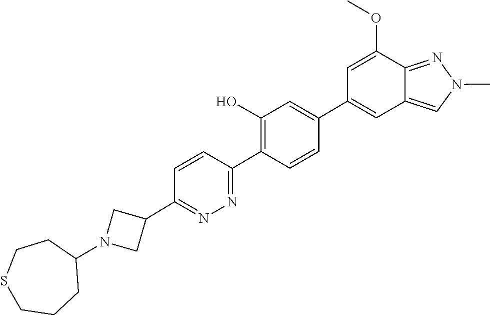 [SMILES: COc1cc(-c2ccc(-c3ccc(C4CN(C5CCCSCC5)C4)nn3)c(O)c2)cc2cn(C)nc12] |
| 184A | 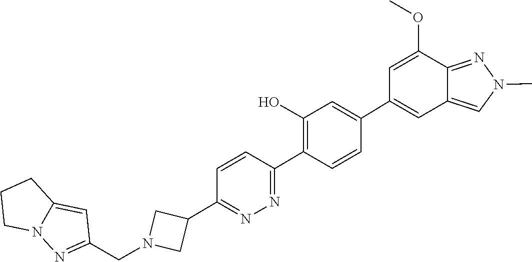 [SMILES: COc1cc(-c2ccc(-c3ccc(C4CN(Cc5cc6n(n5)CCC6)C4)nn3)c(O)c2)cc2cn(C)nc12] |
| 185A | 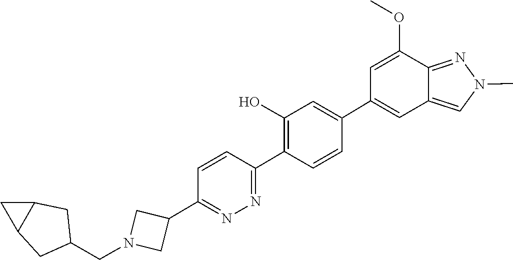 [SMILES: COc1cc(-c2ccc(-c3ccc(C4CN(CC5CC6CC6C5)C4)nn3)c(O)c2)cc2cn(C)nc12] |
| 186A | 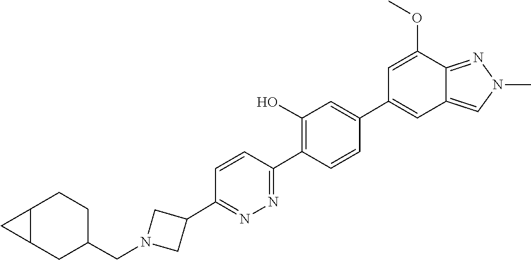 [SMILES: COc1cc(-c2ccc(-c3ccc(C4CN(CC5CCC6CC6C5)C4)nn3)c(O)c2)cc2cn(C)nc12] |
| 187A | 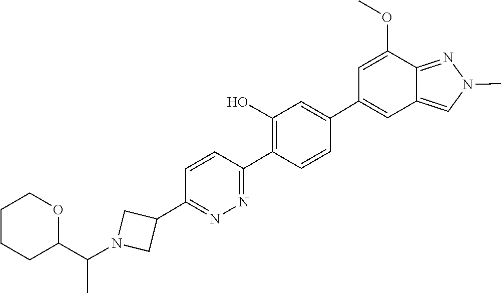 [SMILES: COc1cc(-c2ccc(-c3ccc(C4CN(C(C)C5CCCCO5)C4)nn3)c(O)c2)cc2cn(C)nc12] |
| 188A | 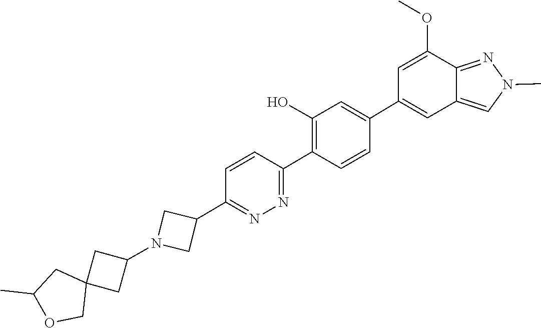 |
| 189A | 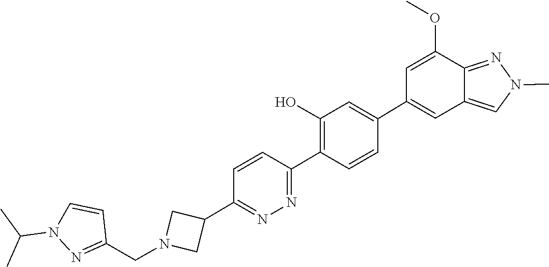 [SMILES: COc1cc(-c2ccc(-c3ccc(C4CN(Cc5ccn(C(C)C)n5)C4)nn3)c(O)c2)cc2cn(C)nc12] |
| 190A | 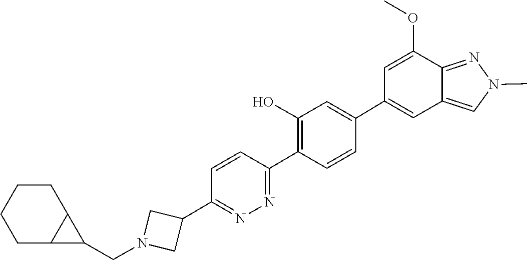 [SMILES: COc1cc(-c2ccc(-c3ccc(C4CN(CC5C6CCCCC65)C4)nn3)c(O)c2)cc2cn(C)nc12] |
| 191A | 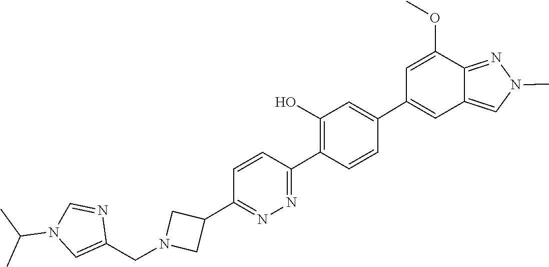 [SMILES: COc1cc(-c2ccc(-c3ccc(C4CN(Cc5cn(C(C)C)cn5)C4)nn3)c(O)c2)cc2cn(C)nc12] |
| 192A | 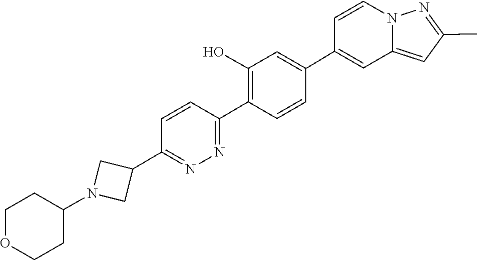 [SMILES: Cc1cc2cc(-c3ccc(-c4ccc(C5CN(C6CCOCC6)C5)nn4)c(O)c3)ccn2n1] |
| 100B | 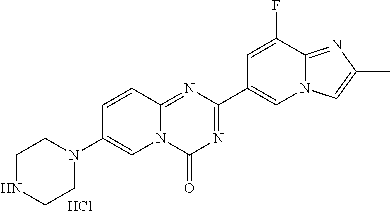 [SMILES: Cc1cn2cc(-c3nc(=O)n4cc(N5CCNCC5)ccc4n3)cc(F)c2n1 ; Cl] |
| 101B | 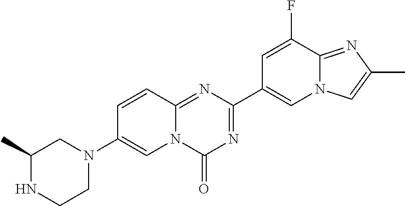 |
| 102B | 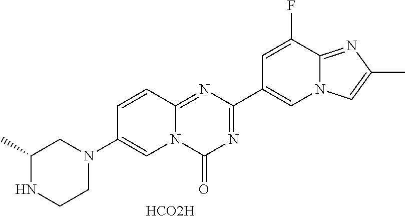 |
| 103B | 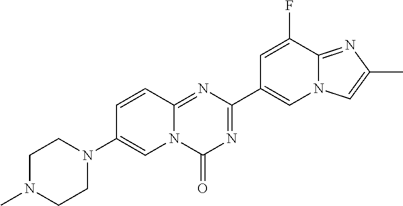 |
| 104B | 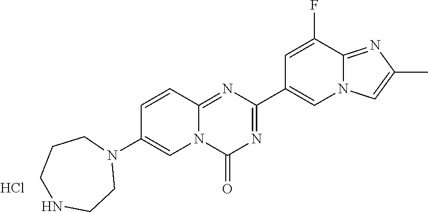 [SMILES: Cl ; Cc1cn2cc(-c3nc(=O)n4cc(N5CCCNCC5)ccc4n3)cc(F)c2n1] |
| 105B | 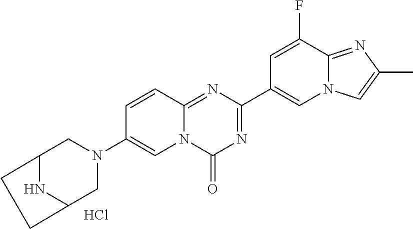 [SMILES: Cc1cn2cc(-c3nc(=O)n4cc(N5CC6CCC(C5)N6)ccc4n3)cc(F)c2n1 ; Cl] |
| 106B | 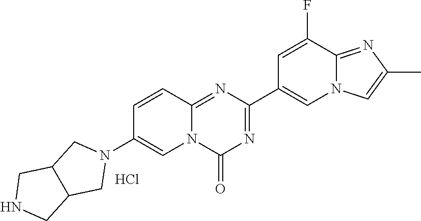 [SMILES: Cc1cn2cc(-c3nc(=O)n4cc(N5CC6CNCC6C5)ccc4n3)cc(F)c2n1 ; Cl] |
| 107B | 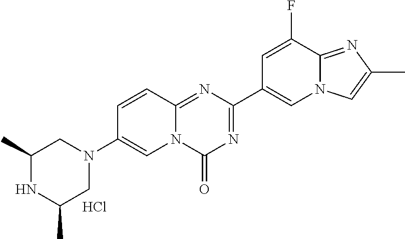 |
| 108B | 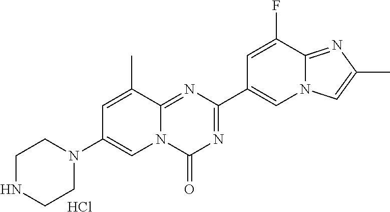 [SMILES: Cc1cn2cc(-c3nc(=O)n4cc(N5CCNCC5)cc(C)c4n3)cc(F)c2n1 ; Cl] |
| 109B | 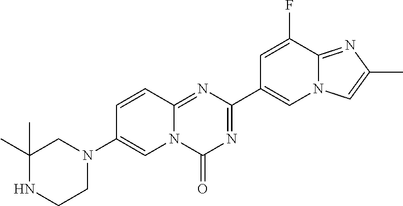 |
| 110B | 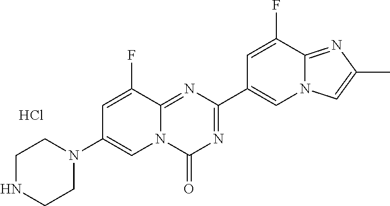 [SMILES: Cc1cn2cc(-c3nc(=O)n4cc(N5CCNCC5)cc(F)c4n3)cc(F)c2n1 ; Cl] |
| 111B | 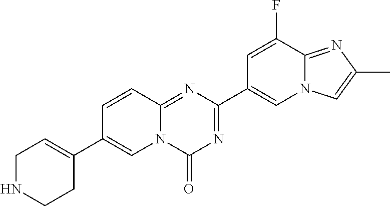 [SMILES: Cc1cn2cc(-c3nc(=O)n4cc(C5=CCNCC5)ccc4n3)cc(F)c2n1] |
| 112B | 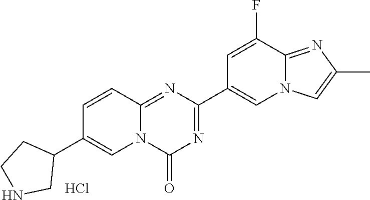 [SMILES: Cc1cn2cc(-c3nc(=O)n4cc(C5CCNC5)ccc4n3)cc(F)c2n1 ; Cl] |
| 113B | 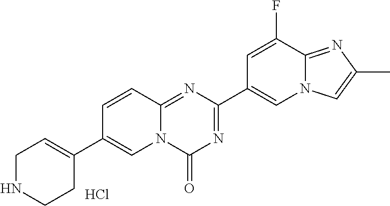 [SMILES: Cc1cn2cc(-c3nc(=O)n4cc(C5=CCNCC5)ccc4n3)cc(F)c2n1 ; Cl] |
| 114B | 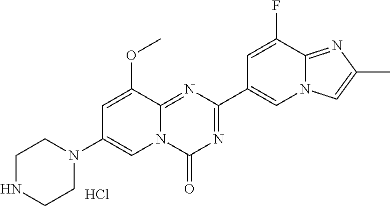 [SMILES: COc1cc(N2CCNCC2)cn2c(=O)nc(-c3cc(F)c4nc(C)cn4c3)nc12 ; Cl] |
| 115B | 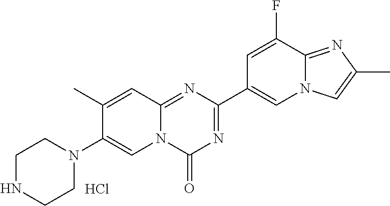 [SMILES: Cc1cn2cc(-c3nc(=O)n4cc(N5CCNCC5)c(C)cc4n3)cc(F)c2n1 ; Cl] |
| 116B | 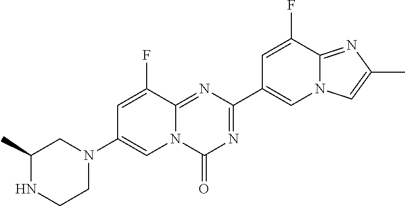 |
| 117B | 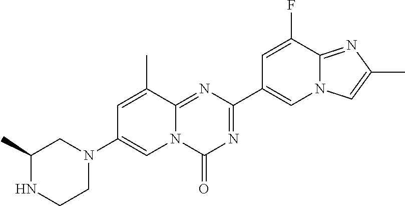 |
| 118B | 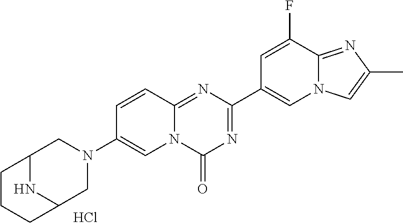 [SMILES: Cc1cn2cc(-c3nc(=O)n4cc(N5CC6CCCC(C5)N6)ccc4n3)cc(F)c2n1 ; Cl] |
| 119B | 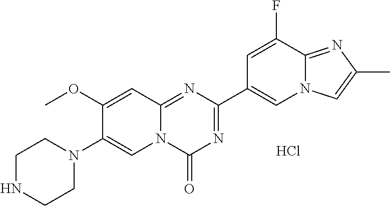 [SMILES: COc1cc2nc(-c3cc(F)c4nc(C)cn4c3)nc(=O)n2cc1N1CCNCC1 ; Cl] |
| 120B | 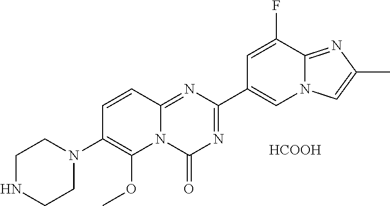 [SMILES: COc1c(N2CCNCC2)ccc2nc(-c3cc(F)c4nc(C)cn4c3)nc(=O)n12 ; "HCOOH"] |
| 121B | 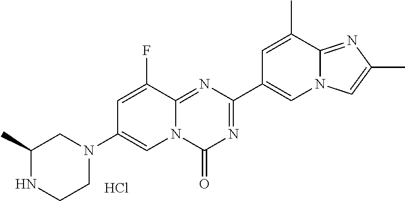 |
| 122B | 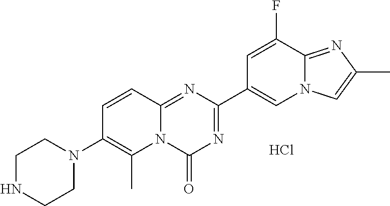 [SMILES: Cc1cn2cc(-c3nc(=O)n4c(C)c(N5CCNCC5)ccc4n3)cc(F)c2n1 ; Cl] |
| 123B | 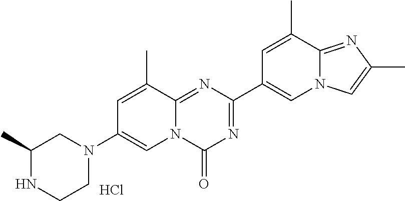 |
| 124B | 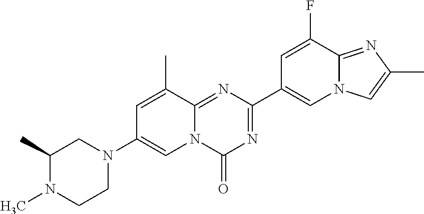 |
| 125B | 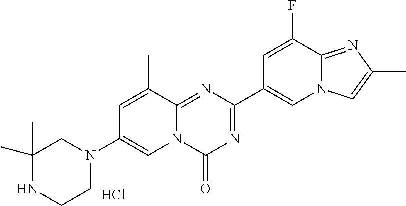 |
| 126B | 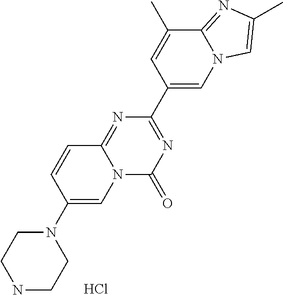 [SMILES: Cc1cn2cc(-c3nc(=O)n4cc(N5CCNCC5)ccc4n3)cc(C)c2n1 ; Cl] |
| 127B | 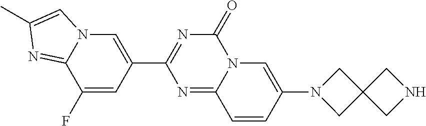 [SMILES: Cc1cn2cc(-c3nc(=O)n4cc(N5CC6(CNC6)C5)ccc4n3)cc(F)c2n1] |
| | 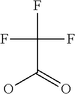 |
| 128B | 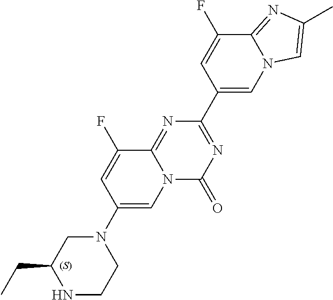 |
| 129B | 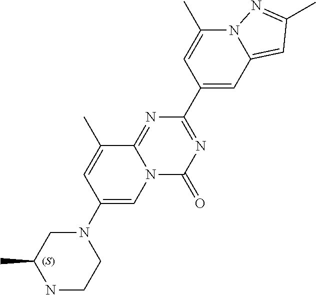 |
| 130B | 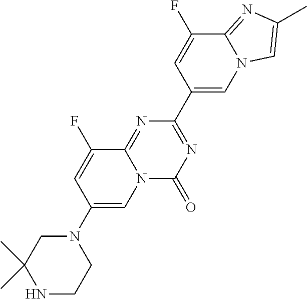 |
| 131B | 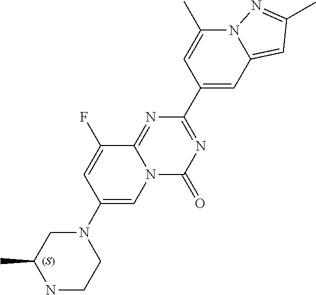 |
| 132B | 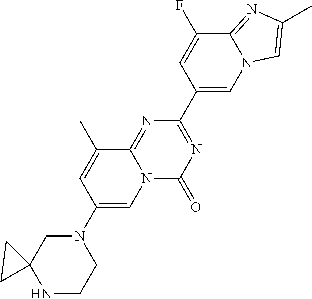 [SMILES: Cc1cn2cc(-c3nc(=O)n4cc(N5CCNC6(CC6)C5)cc(C)c4n3)cc(F)c2n1] |
| 133B | 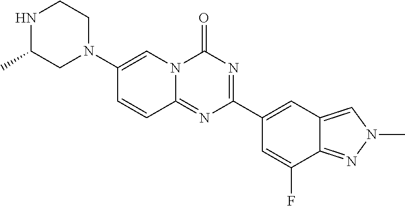 |
| 134B | 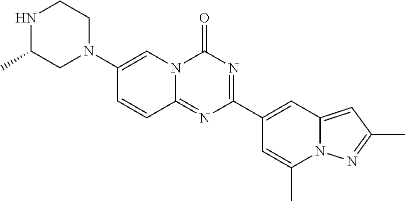 |
| 135B | 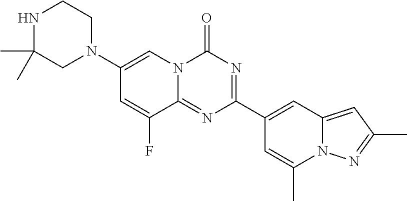 |

In some embodiments, the compounds is selected from the group consisting of 1A-192A and 100B-135B. In some embodiments, the compound is selected from the group comprising 3A, 6A, 8A, 10A, 15A, 24A, 86A, 1001B, 1111B, 1171B, 1211B, 1351W and 192 A. In some embodiments, the compound is Compound 116B. In some embodiments, the compound is Compound 100 GB. In some embodiments, the compound is Compound I A. In some embodiments, the compound is Compound 24 A. In some embodiments, the compound is Compound 2 A. In some embodiments, the compound is Compound 22 A. In some embodiments, the compound is Compound 34 A.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I), are typically chosen such that the molecular weight of the compound does not exceed 1000 daltons. More usually, the molecular weight of the compound will be less than 900, for example less than 800, or less than 750, or less than 700, or less than 650 daltons. More conveniently, the molecular weight is less than 600 and, for example, is 550 daltons or less.

It will be understood that the compounds of any one of the Formula disclosed herein and any pharmaceutically acceptable salts thereof, comprise stereoisomers, or mixtures of stereoisomers of all isomeric forms of said compounds.

It is to be understood that the compounds of any Formula described herein include the compounds themselves, as well as their salts, and their solvates, if applicable.

The in vivo effects of a compound of any one of the Formula disclosed herein may be exerted in part by one or more metabolites that are formed within the human or animal body after administration of a compound of any one of the Formula disclosed herein. As stated hereinbefore, the in vivo effects of a compound of any one of the Formula disclosed herein may also be exerted by way of metabolism of a precursor compound (a prodrug).

Suitably, the present disclosure excludes any individual compounds not possessing the biological activity defined herein.

In some embodiments, a protein, RNA, miRNA, or shRNA of interest (e.g., a protein, RNA, miRNA, or shRNA encoded by one of the above-described genes) is expressed following the co-administration of (i) a nucleic acid molecule or vector encoding the same and (ii) a splice modulator described herein.

A splice modulator is administered, for example, to a subject in a therapeutically effective amount. In some embodiments, administering to a subject a therapeutically effective amount of the splice modulator causes an inclusion of one of the two or more exons, one of the one or more exons, the first exon, or the second exon. In some embodiments, one of the two or more exons or one of the one or more exons is the second exon.

In some embodiments, a nucleic acid molecule of the disclosure comprises a regulatory site capable of being recognized by a spliceosome complex, such that, in some embodiments, administering to a subject a therapeutically effective amount of the splice modulator causes the stabilization of an interaction between a spliceosome complex and the regulatory site capable of being recognized by the spliceosome complex.

In some embodiments, a splice modulator is tested for their ability to alter splicing such that a net increase in inclusion of a bipartite stop codon and transgene expression is generated. The screen may utilize a DNA construct including a minigene, assembled through DNA synthesis and molecular cloning techniques known in the art, and inserted into mammalian cells using electroporation, chemical transfection, viral-mediated integration, or viral mediated episomal introduction. In some embodiments, assessment of the candidate splicing modulator is assessed, for example, by fusing a reporter gene such as a luminescent enzyme (e.g., firefly luciferase, nanoluc luciferase, renilla luciferase, or gaussia luciferase,), fluorescent protein (e.g., green fluorescent protein, blue fluorescent protein, or red fluorescent protein), or colorimetric enzyme (e.g., beta lactamase, or secreted embryonic alkaline phosphatase,) to the DNA construct such that the construct generates a quantifiable signal proportional to the splicing of an exon (e.g., the second exon), readable, for example, by flow cytometry, microscopy, or multi-modal microplate readers using photo-multiplier tubes. Alternatively, for example, the candidate splice modulator-dependent biological activity is assessed by sequencing the mRNA transcripts produced by the DNA construct with RNASeq or by quantitative reverse transcription PCR. In some embodiments, following such an exemplary screen, candidate splice modulators are applied to cells containing the DNA construct for up to 6, 12, 24, or 72 hours and then assessed for start-codon inclusion activity.

In some embodiments, a splice modulator is tested by a screen of various substances and/or chemical derivations of a substance for their ability to regulate the expression of a reporter gene linked to a minigene construct in mammalian cells. The minigene template may derived from a naturally occurring mammalian gene or a synthetic intron-containing construct with canonical 5′ and 3′ splice site sequences. In some embodiments, the minigene template is linked to a reporter gene such as, for example, firefly luciferase to enable detection of gene expression changes resulting from chemical substances that can act as splice modulators to mediate alternative splicing of the minigene.

In some embodiments, a splice modulator is tested for their ability to bind segments of RNA that correspond to segments of a transcribed minigene. In some embodiments, the RNA segments screened are regulatory elements or a splice modulator binding site that control the splicing of an exon (e.g., the second exon) in the minigene. In some embodiments, RNA segments are generated, for example, by in vitro transcription, by chemical RNA synthesis, or by transient or stable expression in cells. In some embodiments, assessment of the splice modulator binding to regulatory RNA segments is assessed by a biophysical method, such as affinity selection mass spectrometry, affinity chromatography, or a similar technique where a difference in liquid phase mobility or migration of the splice modulator is related to the detectable association of the splice modulator with an RNA or RNA-protein complex. In one embodiment, the biophysical method includes assessing a liquid phase mobility or migration of the splice modulator. In some embodiments, assessment of the splice modulator binding to regulatory RNA segments is also assessed by functional methods. In one embodiment, the functional method comprises assessing minigene reporter expression affected by the splice modulator. In one embodiment, the splice modulator is modified in a detectable manner.

In some embodiments, a splice modulator binding site is tested in a screen of various nucleic acid modifications (e.g., single and/or multiple point mutations, insertions, and/or deletions) to a minigene template. The minigene template may be derived from a naturally occurring mammalian gene or a synthetic intron-containing construct with canonical 5′ and 3′ splice site sequences. In some embodiments, the minigene template is linked to a reporter gene such as, for example, firefly luciferase to enable detection of gene expression changes resulting from alternative splicing of the minigene by a splice modulator. In some embodiments, single and/or multiple point mutations, insertions, and/or deletions are introduced into the minigene template to generate combinatorial libraries that are assayed in mammalian cells to select one or more minigene sequence(s) that are optimally responsive to the splice modulator to control the expression of the reporter gene.

In some embodiments, the splice modulator binds to an RNA binding protein and/or a segment of the splice modulator binding site of the nucleic acid of any one of embodiments.

In some embodiments, the splice modulator binds to an RNA binding protein and/or a segment of the splice modulator binding site of the nucleic acid of any one of embodiments.

In some embodiments, the splice modulator binds to an RBP. In some embodiments, the expression of such an RBP is tissue-specific. In some embodiments, an RBP is exclusively expressed in the brain or any other specific tissue of interest. In accordance with this principle, in some embodiments, a nucleic acid molecule of the disclosure operates by an exon-inclusion principle by using a splice modulator that targets an RBP with tissue-specific expression. For example, the nucleic acid molecule is linked to a tissue-specific promoter that is concordant with the expression profile of the RBP of interest.

In some embodiments, the RBP is a splicing enhancer (e.g.; a serine and arginine-rich (SR) protein). In some embodiments, the RBP is a splicing repressor (e.g.; a heterogeneous nuclear ribonucleoprotein (hnRNP)).

### Methods of Synthesis

By way of example only, provided is a scheme for preparing a splicing modulator described herein.

In some embodiments, a scheme for preparing a splicing modulator is described herein in Scheme 1:

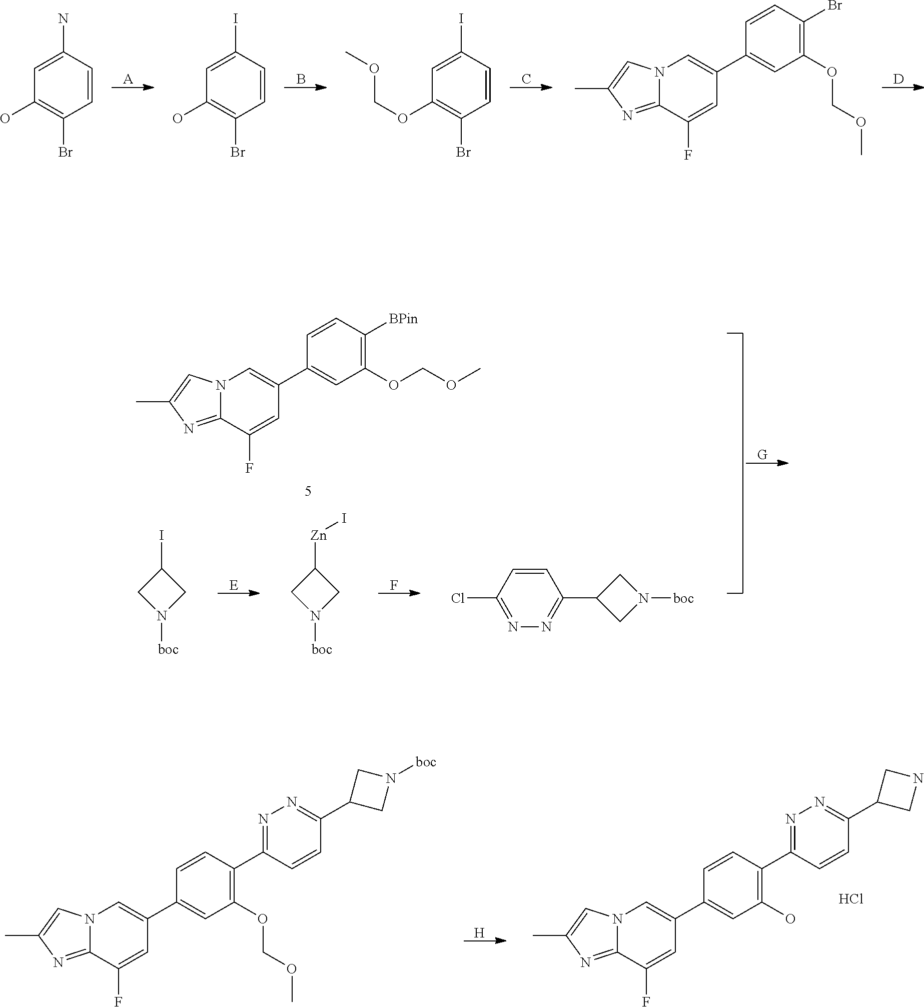

In some embodiments, a scheme for preparing a splicing modulator is described herein in Scheme 2:

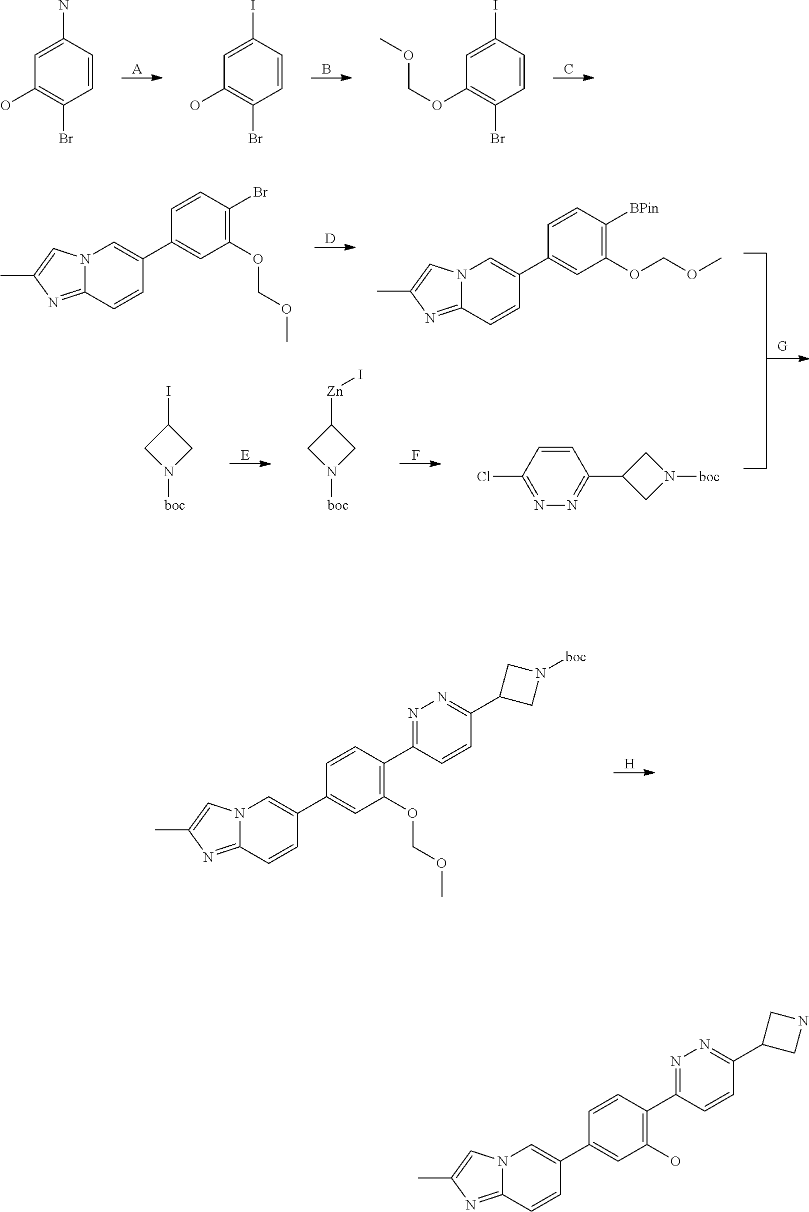

The compounds of the present disclosure can be prepared by any suitable technique known in the art. Particular processes for the preparation of these compounds are described further in the accompanying examples.

In some embodiments, the synthesis of any of the compounds disclosed herein, compounds 1A-192 A and 100B-135B are described in PCT/US2022/080352 (WO2023092149) or PCT/US2022/079748 (WO2023086959), the contents of which are hereby incorporated by reference.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a structure of Formula (I) has been synthesized by any one of the processes defined herein, the processes may then further comprise the additional steps of: (i) removing any protecting groups present; (ii) converting the structure of Formula (I) into another structure of Formula (I) forming a pharmaceutically acceptable salt, hydrate or solvate thereof; and/or (iv) forming a prodrug thereof.

In some embodiments, the resultant compounds of Formula (I) are isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol: ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP): nitriles, such as acetonitrile; sulphoxides, such as dimethyl sulphoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, in some embodiments, by utilizing the procedures described herein, in conjunction with ordinary skills in the art, additional compounds of the present disclosure are readily prepared. In some embodiments, those skilled in the art will readily understand that known variations of the conditions and processes of the following preparative procedures is used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, in some embodiments, some of the compounds of the present disclosure are readily synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P.G.M. Wuts, T.W. Greene, “Greene's Protective Groups in Organic Synthesis”, 4th edition (2006) (John Wiley & Sons).

General routes for the preparation of a compound of the application are described herein.

### Nucleic Acid Vectors

In some embodiments, effective intracellular concentrations of a nucleic acid molecule disclosed herein is also achieved via the stable expression of a vector encoding a nucleic acid molecule (e.g., by integration into the nuclear or mitochondrial genome of a mammalian cell), such as a nucleic acid molecule that contains a minigene linked to a transgene, in which the minigene comprises a first exon, a first intron, a second exon, a start codon, and a second intron, as described herein. In order to introduce such a nucleic acid molecule into a mammalian cell, the nucleic acid molecule can be incorporated into a vector.

Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)).

In some embodiments, the nucleic acid molecules disclosed herein are introduced into a mammalian cell by targeting a vector containing a polynucleotide encoding such a nucleic acid molecule to cell membrane phospholipids. For example, in some embodiments, vectors are targeted to the phospholipids on the extracellular surface of the cell membrane by linking the vector molecule to a VSV-G protein, a viral protein with affinity for all cell membrane phospholipids. In some embodiments, a construct is produced using conventional and routine methods of the art. In addition to achieving high rates of transcription and translation, stable expression of an exogenous polynucleotide in a mammalian cell can be achieved by integration of the polynucleotide containing the gene into the nuclear genome of the mammalian cell. A variety of vectors for the delivery and integration of polynucleotides encoding exogenous proteins or RNA products (e.g., miRNA or shRNA) into the nuclear DNA of a mammalian cell have been developed. Examples of expression vectors are disclosed in, e.g., WO 1994/011026. Expression vectors for use in the compositions and methods described herein contain a polynucleotide sequence that encodes a nucleic acid molecule as well as, e.g., additional sequence elements used for the expression of these nucleic acid molecules and/or the integration of these polynucleotide sequences into the genome of a mammalian cell. In some embodiments, certain vectors that are used include plasmids that contain regulatory sequences, such as promoter and enhancer regions, which direct gene transcription. Other useful vectors contain polynucleotide sequences that enhance the rate of translation of these genes or improve the stability or nuclear export of the mRNA that results from gene transcription. These sequence elements include, e.g., 5′ and 3′ UTR regions, an internal ribosomal entry site (IRES), and polyA in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin.

### Viral Vectors

Viral genomes provide a rich source of vectors that can be used for the efficient delivery of exogenous polynucleotides into a mammalian cell. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a parvovirus (e.g., adeno-associated viruses (AAV)), retrovirus (e.g., Retroviridae family viral vector), adenovirus (e.g., Ad5, Ad26, Ad34. Ad35, and Ad48), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picomavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven. Philadelphia. (1996))). Other examples are murine leukemia viruses, murine sarcoma viruses, murine mammary tumor virus, bovine leukemia virus, feline leukemia virus, feline sarcoma virus, avian leukemia virus, human T-cell leukemia virus, baboon endogenous virus. Gibbon ape leukemia virus, nucleic acid molecule Pfizer monkey virus, simian immunodeficiency virus, simian sarcoma virus, Simian virus 40 (SV40), Rous sarcoma virus and lentiviruses. Other examples of vectors are described, for example, in McVey et al., (U.S. Pat. No. 5,801,030).

### Retroviral Vectors

The delivery vector used in the methods and compositions described herein may be a retroviral vector. Retroviruses may be chosen as gene delivery vectors due to their ability to integrate their genes into the host genome, transferring a large amount of foreign genetic material, infecting a broad spectrum of species and cell types and for being packaged in special cell-lines. Furthermore, retroviral vectors are able to infect a broad variety of cell types.

One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the polynucleotide. An overview of optimization strategies for packaging and transducing LVs is provided in Delenda, The Journal of Gene Medicine 6: S125 (2004). The use of lentivirus-based gene transfer techniques relies on the in vitro production of recombinant lentiviral particles carrying a highly deleted viral genome in which the polynucleotide of interest is accommodated. In particular, the recombinant lentivirus are recovered through the in trans co-expression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, encapsidation, and expression, in which the sequences to be expressed are inserted. A LV used in the methods and compositions described herein may include one or more of a 5′-Long terminal repeat (LTR), HIV signal sequence, HIV Psi signal 5′-splice site (SD), delta-GAG element, Rev Responsive Element (RRE). 3′-splice site (SA), elongation factor (EF) 1-alpha promoter, and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597. The lentiviral vector may further include a pHR′ backbone, which may include for example as provided herein. The Lentigen LV described in Lu et al., Journal of Gene Medicine 6:963 (2004) may be used to express the DNA molecules and/or transduce cells.

A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). Optionally, one or more of these regions is substituted with another region performing a similar function. Enhancer elements can be used to increase expression of modified DNA molecules or increase the lentiviral integration efficiency. The LV used in the methods and compositions described herein may include a nef sequence.

The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells.

The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of polynucleotide expression from several different promoters, both in vitro and in vivo. The LV used in the methods and compositions described herein may include both a cPPT sequence and WPRE sequence.

The vector may also include an IRES sequence that permits the expression of multiple polypeptides from a single promoter. In addition to IRES sequences, other elements which permit expression of multiple polynucleotides are useful. The vector used in the methods and compositions described herein may include multiple promoters that permit expression more than one polynucleotide.

Other elements that permit expression of multiple polynucleotides identified in the future are useful and may be utilized in the vectors suitable for use with the compositions and methods described herein.

The vector used in the methods and compositions described herein may, be a clinical grade vector. Accordingly, retroviral vectors may be employed in conjunction with the disclosed methods and compositions.

In order to construct a retroviral vector, a nucleic acid encoding a gene of interest is inserted into the viral genome in the place of specific viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (e.g., by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer.

### AAV Vectors

In some embodiments, nucleic acid molecules described herein are incorporated into recombinant AAV (rAAV) vectors in order to facilitate their introduction into a cell, such as a target cell, rAAV vectors useful in the conjunction with the compositions and methods described herein include recombinant nucleic acid constructs that contain (1) a nucleic acid molecule and (2) nucleic acids that facilitate and expression of the heterologous genes. The viral nucleic acids may include those sequences of AAV that are required in cis for replication and packaging (e.g., functional ITRs) of the DNA into a virion. Such rAAV vectors may also contain marker or reporter genes.

Useful rAAV vectors include those having one or more of the naturally-occurring AAV genes deleted in whole or in part, but retain functional flanking ITR sequences. The AAV ITRs may be of any serotype (e.g., derived from serotype 2 or 5) suitable for a particular application. Methods for using rAAV vectors are described, for example, in Tal et al., J. Biomed. Sci. 7:279-291 (2000), and Monahan and Samulski. Gene Delivery 7:24-30 (2000), the disclosures of each of which are incorporated herein by reference as they pertain to AAV vectors for gene delivery.

In some embodiments, the nucleic acids and vectors described herein are incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J Virol. 76:791-801 (2002) and Bowles et al., J Virol. 77:423-432 (2003).

rAAV virions useful in conjunction with the compositions and methods described herein include those derived from a variety of AAV serotypes including AAV 1, 2, 3, 4, 5, 6, 7, 8 and 9. Construction and use of AAV vectors and AAV proteins of different serotypes are described, for example, in Chao et al., Mol. Ther. 2:619-623 (2000); Davidson et al., Proc. Natl. Acad. Sci. USA 97:3428-3432 (2000); Xiao et al., J. Virol. 72:2224-2232 (1998); Halbert et al., J. Virol. 74:1524-1532 (2000): Halbert et al., J. Virol. 75:6615-6624 (2001); and Auricchio et al., Hum. Molec. Genet. 10:3075-3081 (2001).

Also useful in conjunction with the compositions and methods described herein are pseudotyped rAAV vectors. Pseudotyped vectors include AAV vectors of a given serotype pseudotyped with a capsid gene derived from a serotype other than the given serotype (e.g., AAV1, AAV3, AAV4, AAV5, AAV6, AAV7, AAV8, or AAV9, among others). For example, a representative pseudotyped vector is an AAV2 vector encoding a therapeutic protein pseudotyped with a capsid gene derived from AAV serotype 8 or AAV serotype 9. Techniques involving the construction and use of pseudotyped rAAV virions are known in the art and are described, for example, in Duan et al., J. Virol. 75:7662-7671 (2001); Halbert et al., J Virol. 74:1524-1532 (2000); Zolotukhin et al., Methods, 28:158-167 (2002); and Auricchio et al., Hum. Molec. Genet., 10:3075-3081 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635-45 (2000).

Other rAAV virions that can be used in methods of the disclosure include those capsid hybrids that are generated by molecular breeding of viruses as well as by exon shuffling. See, e.g., Soong et al., Nat. Genet., 25:436-439 (2000) and Kolman and Stemmer, Nat. Biotechnol. 19:423-428 (2001).

### Methods for the Delivery of Exogenous Nucleic Acids to Target Cells

In some embodiments, techniques that are used to introduce a nucleic acid molecule into a mammalian cell are well known in the art. For example, electroporation is used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987). A similar technique, Nucleofection™, utilizes an applied electric field in order to stimulate the uptake of exogenous polynucleotides into the nucleus of a eukaryotic cell.

Nucleofection™ and protocols useful for performing this technique are described in detail, e.g., in Distler et al., Experimental Dermatology 14:315 (2005), as well as in US 2010/0317114.

Additional techniques useful for the transfection of target cells are the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., JoVE 81:e50980 (2013).

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386.

Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003)) polyethylenimine, and diethylaminoethyl (DEAE)-dextran, the use of which as a transfection agent is described in detail, for example, in Gulick et al., Current Protocols in Molecular Biology 40:1:9.2:9.2.1 (1997). Magnetic beads are another tool that can be used to transfect target cells in a mild and efficient manner, as this methodology utilizes an applied magnetic field in order to direct the uptake of nucleic acids. This technology is described in detail, for example, in US 2010/0227406.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires.

Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:1870 (2010).

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002).

Another useful tool for inducing the uptake of exogenous nucleic acids by target cells is sonoporation, a technique that involves the use of sound (typically ultrnucleic acid moleculenic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007).

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13. Abstract No. 122.

### Nucleic Acid and Protein Detection

A splicing event mediated by one or more compounds described above can be characterized using a variety of assays known to those skilled in the art to determine whether a transgene has been transcribed. For example, in some embodiments, a transgene is characterized by assays, including but not limited to those assays described herein, to determine whether a nucleic acid thereof is expressed. In some embodiments, nucleic acid levels are determined using Northern blotting, Southern blotting, nuclease protection assays (NPA), in situ hybridization (ISH), reverse transcription-polymerase chain reaction (RT-PCR), or RNA sequencing (RNA-Seq). In some embodiments, RNA sequencing is performed using Sanger sequencing, Illumina sequencing, Ion Torrent sequencing, 454 sequencing, SOLiD sequencing, or nanopore sequencing.

In some embodiments, gene expression levels are determined using microarray-based platforms

In some embodiments, amplification-based assays, such as PCR or qPCR, also are used to measure the expression level of one or more markers (e.g., genes).

In some embodiments, a splicing event mediated by one or more compounds described above is characterized using a variety of assays known to those skilled in the art to determine whether a transgene has been translated. In some embodiments, a transgene is characterized by assays, including but not limited to, Western blotting, immunoblotting, enzyme-linked immunosorbent assay (ELISA), radioimmunoassay (RIA), immunoprecipitation, immunofluorescence, surface plasmon resonance, chemiluminescence, florescent polarization, phosphorescence, immunohistochemical analyses, matrix-associated laser desorption/ionization time of light (MALDI-TOF) mass spectrometry, liquid chromatography (LC)-mass spectrometry, microcytometry, microscopy, florescence activated cell coating (FACs), mass spectrometry (MS), and flow cytometry.

### Pharmaceutical Compositions and Routes of Administration

Any one of the compositions described herein, such as a nucleic acid molecule, a nucleic acid vector encoding the same, a composition, or a splice modulator may be formulated into pharmaceutical compositions for administration to a mammalian (e.g., a human) subject in a biologically compatible form suitable for administration in vivo.

The pharmaceutical compositions may be manufactured in a manner that is generally known, e.g., by means of mixing, dissolving, granulating, levigating, emulsifying, encapsulating, entrapping, or lyophilizing processes. Pharmaceutical compositions may be formulated in a manner using one or more pharmaceutically acceptable carriers including excipients and/or auxiliaries that facilitate processing of the active compounds into preparations that can be used pharmaceutically, with the appropriate formulation being dependent upon the route of administration chosen.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid.

It is to be understood that a pharmaceutical composition of the disclosure is formulated to be compatible with its intended route of administration. Examples of routes of administration include systemic, parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulphite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that the pharmaceutical compositions can be included in a container, pack, or dispenser together with instructions for administration.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the subject, and the route of administration.

Dosage and administration are adjusted to provide sufficient levels of the active agent(s) or to maintain the desired effect. Factors which may be considered include the severity of the disease state, general health of the subject, age, weight, and gender of the subject, diet, time and frequency of administration, drug combination(s), reaction sensitivities, and tolerance/response to therapy.

Accordingly, the present disclosure relates to a pharmaceutical composition containing a nucleic acid molecule disclosed herein. In some embodiments, the present disclosure relates to a composition comprising a vector comprising a nucleic acid molecule of the disclosure. In a particular example, the disclosure provides a pharmaceutical composition containing a vector (e.g., lentiviral or AAV vector) comprising a nucleic acid molecule of the disclosure linked to a promoter, as is disclosed herein. Such composition may be co-administered with a pharmaceutically acceptable splice modulator. Following, the pharmaceutical composition may include an AAV vector comprising (a) a viral capsid; and (b) an artificial polynucleotide comprising an expression cassette flanked by AAV ITRs, wherein the expression cassette comprises a polynucleotide encoding a bipartite start codon that when bound by the splice modulator regulates the expression of a transgene.

### Methods of Treatment

Diseases associated with changes to RNA transcript amount are often treated with a focus on the aberrant protein expression. However, if the processes responsible for the aberrant changes in RNA levels, such as components of the splicing process or associated transcription factors or associated stability factors, could be targeted by treatment with a small molecule, it would be possible to restore protein expression levels such that the unwanted effects of the expression of aberrant levels of RNA transcripts or associated proteins. Therefore, there is a need for methods of modulating the amount of RNA transcripts encoded by certain genes as a way to prevent or treat diseases associated with aberrant expression of the RNA transcripts or associated proteins.

Aberrant splicing of mRNA, such as pre-mRNA, can result in a defective protein and can cause a disease or a disorder in a subject. In some embodiments, the compositions and methods described herein reduce this aberrant splicing of mRNA, such as pre-mRNA, and treat a disease or a disorder caused by this aberrant splicing.

Any of the compositions described herein may be used in a method of treatment, for example, in a method of modulating the expression level of a protein in a subject in need thereof. Such treatment may, for example, obtain a desired pharmacological and/or physiological effect. The effect may be therapeutic in terms of partially or completely curing a disease and/or adverse effect attributed to the disease.

### Definitions

The features and other details of the disclosure will now be more particularly described. Certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The term “administering,” or a grammatical derivative thereof, as described herein, refers to the delivery of a nucleic acid molecule or a vector including the nucleic acid molecule thereof, to a subject in need thereof. Any suitable method of administration can be selected by one of skill in the art, in view of this disclosure.

The term “aptamer” as used herein refers to the term as commonly used and understood in the art. Without being limiting, the present disclosure includes that an “aptamer” is a fragment (or a domain) of nucleic acid that selectively binds to a ligand or molecule. The introduction of a ligand to a ligand-specific aptamer causes conformational changes within the aptamer and influences nucleic acids adjacent to the aptamer. In some embodiments, such a conformational change may contribute to a splicing event.

The term “binding,” as used herein, refers to the term as commonly used and understood in the art. Without being limiting, the present disclosure includes that “binding” can be between a splice modulator binding site (e.g., a nucleic acid molecule or a protein) and a splice modulator, and can refer to molecular interactions (e.g., hydrogen bonding, hydrophobic interactions, electrostatic interactions, van der Waals interactions) in a degree sufficient to mediate or prevent splicing (e.g., alternative splicing) by a spliceosome at a 5′ splice site or 3′ splice site or, more generally, a splicing domain or a RNA binding protein (RBP) into association with its regulatory sequence on pre-mRNA. Additionally, the splice modulator binding site may include additional modifications.

The term “bipartite” as used herein refers to a noncontiguous (e.g., nucleotides of the codon are split) start codon that may be reconstituted upon a splicing event of a nucleic acid molecule.

The term “codon” as used herein refers to any group of three nucleotide bases in a given messenger RNA molecule, or coding strand of DNA, that specifies a particular amino acid or a starting or stopping signal for translation. The term codon also refers to base triplets in a DNA strand. Such nucleotide bases may be contiguous or noncontiguous (e.g., in the case of a bipartite codon).

Throughout the specification and claims, the word “comprise.” or variations such as “comprises” or “comprising.” will be understood to imply the inclusion of a stated word or group of words but not the exclusion of any other word or group of words.

As used herein, the terms “effective amount,” “therapeutically effective amount,” and the like, when used in reference to a composition described herein, such as a nucleic acid molecule or a vector encoding such a nucleic acid molecule, refer to a quantity sufficient to, when administered to a subject, including a mammal (e.g., a human), effect beneficial or desired results (e.g., expression of a protein, RNA, miRNA, or shRNA of interest), which may include clinical results. For example, an effective amount of one or more composition described herein (e.g., a nucleic acid molecule and a splice modulator) may achieve expression of a protein, miRNA, or shRNA of interest as compared to the expression of said protein. RNA, miRNA, or shRNA without administration of the composition of interest. In some embodiments, the protein, RNA, miRNA, or shRNA is expressed in the presence of the splice modulator. An “effective amount,” “therapeutically effective amount,” and the like, of a composition, such as a nucleic acid molecule or a vector encoding such a nucleic acid molecule, also include an amount that results in a beneficial or desired result in a subject as compared to a control.

As used herein, the term “exon” refers to a region within the coding or noncoding region of a gene, the nucleotide sequence of which determines the nucleotide sequence of the corresponding mRNA and/or amino acid sequence of the corresponding protein. The term exon also refers to the corresponding region of the RNA transcribed from a gene. A gene, as outlined above, may contain several exons separated by intervening introns. Exons are transcribed into pre-mRNA and may be included in the mRNA depending on the alternative splicing of the gene.

As used herein, the term “intron” refers to a region within the noncoding region of a gene, the nucleotide sequence of which is not incorporated into the mRNA of the corresponding gene. The term intron also refers to the corresponding region of the RNA transcribed from a gene. In some embodiments, a gene, for example, may contain several introns, each of which forms the intervening sequence between two exons. Introns are transcribed into pre-mRNA, but are removed during processing, and are not included in the mature mRNA.

As used herein, the terms “excise,” “excised,” “excision,” and “excising,” refer to the exclusion (e.g., ‘splicing out’) of an exon(s) and/or intron(s) from a nucleic acid molecule that occurs during splicing.

The term “exon-inclusion” as used herein refers to a mechanism which depends on a ligand-responsive riboswitch that promotes the binding of the spliceosome to 5′- or 3′-splice sites or a RNA binding protein (RBP) to its regulatory target site.

“Linked” refers to the term as commonly used and understood in the art. Without being limiting, the present disclosure includes that “linked” refers to a juxtaposition wherein the components so described are in a relationship permitting them to function in their intended manner. For embodiment, a promoter is linked to a coding sequence if the promoter affects its transcription or expression.

As used herein, the term “minigene” refers to an isolated nucleic acid sequence encoding a recombinant protein, RNA, miRNA, or shRNA where one or more elements of the corresponding gene encoding the naturally-occurring protein, miRNA, or shRNA have been removed and where the protein, RNA, miRNA, or shRNA encoded by the minigene retains certain segments of the corresponding naturally-occurring or synthetic protein, RNA, miRNA, or shRNA.

As used herein, the terms “nucleic acid molecule” and “nucleic acid” refer to polymers of any length composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), short-hairpin RNA (shRNA), and microRNAs (miRNA).

As used herein, “nucleotide” means a nucleoside having a phosphate group covalently linked to the sugar portion of the nucleoside.

The term “pharmaceutically acceptable” means safe for administration to a mammal, such as a human. In some embodiments, a pharmaceutically acceptable composition is approved by a regulatory agency of the Federal government or a state government or is listed in the U.S. Pharmacopeia or any other generally recognized pharmacopeia for use in animals (e.g., humans). As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “pharmaceutically acceptable carrier” or “pharmaceutically acceptable excipient,” as used herein, refer interchangeably to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions together with one or more pharmaceutically acceptable excipients.

It is to be understood that the present disclosure also provides pharmaceutical compositions including any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the subject. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

The terms “poly adenylation signal” (polyA) or “poly adenylation site” are used herein to mean a nucleic acid sequence sufficient to direct the addition of polyadenosine ribonucleic acid(s) to an RNA molecule expressed in a cell.

As used herein, the term “promoter” refers to a recognition site on DNA that is bound by an RNA polymerase. The polymerase drives transcription of a transgene. Exemplary promoters suitable for use with the compositions and methods described herein are described herein. Additionally, the term “promoter” may refer to a synthetic promoter, such as a regulatory DNA sequence that doe does not occur naturally in a biological system. Synthetic promoters contain parts of naturally occurring promoters combined with polynucleotide sequences that do not occur in nature and can be optimized to express recombinant DNA.

The terms “recipient,” “individual,” “subject,” “host,” and “patient,” are used interchangeably herein and in some embodiments, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. In some embodiments, the mammal is human. None of these terms require the supervision of medical personnel.

“Splicing” refers to the process by which intron sequences are removed from nascent premessenger RNA (pre-mRNA) and exons are bound together to form mRNA. The splice site is at the junction between the exon and the intron and is defined by different consensus sequences at the 5′- and 3′-ends of the intron (i.e., the splice donor site and the splice acceptor site, respectively). Alternative pre-mRNA splicing, or alternative splicing, is a widespread process that occurs in many genes, including multiple exons. It is carried out by many multi-component structures called spliceosomes, which are aggregates of micronucleus ribonuclear proteins (snRNPs) and a wide variety of co-proteins. By recognizing various cis regulatory sequences, spliceosomes define exon/intron boundaries, remove intron sequences, and splice exons into the final translatable message (e.g., mRNA). In the case of alternative splicing, certain exons may or may not be included in order to ultimately alter the encoding message, thereby altering the resulting expressed protein.

A “splicing domain,” as used herein, refers to a nucleic acid sequence having motifs that are recognized by a spliceosome and which mediate splicing (e.g., by alternative splicing). A splicing domain includes a splice site. A splice site may also include other regulatory elements. For example, in some embodiments, a splicing domain includes splicing enhancers (e.g., exonic splicing enhancers or intronic splicing enhancers). In some embodiments, a splicing domain includes a branch point (e.g., a strong conserved branch point), a branch point sequence, or a polypyrimidine tract. In some embodiments, a splicing domain includes a splice acceptor or splice donor.

As used herein, “splice donor site” refers to a splice site found at the 5′-end of an intron or the 3′-end of an exon. The splice donor site is used interchangeably with the “5′ splice site.” As used herein, the term “splice acceptor site” refers to a splice site found at the 3′-end of an intron or the 5′-end of an exon. The splice acceptor site is used interchangeably with the 3′ splice site.

As used herein, the terms “transduction” and “transduce” refer to a method of introducing a viral vector construct or a part thereof into a cell and subsequent expression of a transgene encoded by the vector construct or part thereof in the cell.

As used herein. “transfection” refers to any of a wide variety of techniques commonly used for the introduction of exogenous DNA into a prokaryotic or eukaryotic host cell e.g., electroporation, lipofection, calcium-phosphate precipitation, diethylaminoethyl (DEAE)-dextran transfection, NUCLEOFECTION™, squeeze-poration, sonoporation, optical transfection, MAGNETOFECTION™, impalefection, and the like.

As used herein, the term “transgene” refers to a recombinant nucleic acid (e.g., DNA or cDNA) encoding a gene product (e.g., a protein, RNA, miRNA, or shRNA of interest described herein). The gene product may be an RNA (e.g., an miRNA or shRNA), peptide, or protein. In addition to the coding region for the gene product, the transgene may include or be linked to one or more elements to facilitate or enhance expression, such as a promoter, enhancer(s), destabilizing domains(s), response element(s), reporter element(s), insulator element(s), polyadenylation signal(s), and other functional elements. Embodiments of the disclosure may utilize any known suitable promoter, enhancer(s), destabilizing domains(s), response element(s), reporter element(s), insulator element(s), polyadenylation signal(s), and/or other functional elements.

As used herein, the term “vector” includes a nucleic acid vector, e.g., a DNA vector, such as a plasmid, an RNA vector, or another suitable replicon (e.g., viral vector). A variety of vectors have been developed for the delivery of polynucleotides encoding exogenous polynucleotides or proteins into a prokaryotic or eukaryotic cell. Examples of such expression vectors are disclosed in, e.g., WO 1994/011026. Expression vectors suitable for use with the compositions and methods described herein contain a polynucleotide sequence as well as, e.g., additional sequence elements used for the expression of heterologous nucleic acid materials (e.g., a nucleic acid molecule) in a mammalian cell. Certain vectors that can be used for the expression of the nucleic acid molecules described herein include plasmids that contain regulatory sequences, such as promoter and enhancer regions, which direct gene transcription. Other useful vectors for expression of nucleic acid molecule agents disclosed herein contain polynucleotide sequences that enhance the rate of translation of these polynucleotides or improve the stability or nuclear export of the RNA that results from gene transcription. These sequence elements include, e.g., 5′ and 3′ untranslated regions, an IRES, 2A ribosomal skipping peptides, and polyA sequences in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

As use herein, the phrase “compound of the disclosure” refers to those compounds which are disclosed herein, generically.

As used herein, “alkyl”, “Cl, C₂, C₃, C₄, C₅ or C₆ alkyl” or “C₁-C₆ alkyl” is intended to include Cl, C₂, C₃, C₄, C₅ or C₆ straight chain (linear) saturated aliphatic hydrocarbon groups and C₃, C₄, Cs or C₆ branched saturated aliphatic hydrocarbon groups. For example, C₁-C₆ alkyl is intends to include Cl, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, “alkenyl” is intended to include straight-chain or branched hydrocarbon groups having from 2 to 6 carbon atoms, one or more carbon-carbon double bonds, and no triple bonds (“C₂-C₆alkenyl”). The one or more carbon-carbon double bonds can be internal (such as in 2-butenyl) or terminal (such as in 1-butenyl). Examples of C2-C₆alkenyl groups include ethenyl (C₂), 1-propenyl (C₃), 2-propenyl (C₃), 1-butenyl (C₄), 2-butenyl (C₄), butadienyl (C₄), and the like.

As used herein, “alkynyl” is intended to include straight-chain or branched hydrocarbon groups having from 2 to 6 carbon atoms, one or more carbon-carbon triple bonds, and optionally one or more double bonds (“C₂-C₆ alkynyl”). The one or more carbon-carbon triple bonds can be internal (such as in 2-butynyl) or terminal (such as in 1-butynyl). Examples of C₂-C₄alkynyl groups include, without limitation, ethynyl (C₂), 1-propynyl (C₃), 2-propynyl (C₃), 1-butynyl (C₄), 2-butynyl (C₄), and the like.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2.2.6.6-tetramethyl-1,2,3,6-tetrahydropyridinyl.

As used herein, the term “alkoxy” or “alkoxyl” refers to the group —OR where R is alkyl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e., with between 1 and 6 carbon atoms.

As used herein, “heteroalkyl”, “Cl, C₂, C₃, C₄, C₅, or C₆ heteroalkyl” or “C₁-C₆ heteroalkyl” is intended to include Cl, C₂, C₃, C₄, C₅, or C₆ straight chain (linear) saturated aliphatic hydrocarbon groups and C₃, C₄, C₅, or C₆ branched saturated aliphatic hydrocarbon groups, in which at least one of the carbons has been replaced with N, O, or S. The heteroatom will be bonded to any required hydrogens to complete the heteroatom's valence (e.g., a CH₂ may be replaced with an “O” or a “NH”, a CH may be replaced with an N, etc.)). Such substituents can include, for example, —O—CH(CH₃)₂, —CH₂—N(CH₃)—CH₂CH₂OCH₃, —S—CH₂CH₂—O—CH₂CH₃, and so forth.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” or “heterocyclyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g. 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl. 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1.4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3.4-c]pyridinyl. 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system. Examples of aryl groups include, but are not limited to, phenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur. The nitrogen atom may be substituted or unsubstituted (i.e., N or NR wherein R is H or other substituents, as defined). The nitrogen and sulphur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O—.

As used herein, the term “cyano” refers to the group —CN.

As used herein, the term “nitro” refers to the radical —NO₂.

As used herein, the term “oxo” refers to ═O.

As used herein, the term “halo” or “halogen” refers to fluoro, chloro, bromo and iodo.

As used herein, the term “haloalkyl” refers to a branched or unbranched alkyl substituted with one or more halogens. For example, a C₁₋₆haloalkyl is an alkyl group of from one to seven carbons wherein at least one H is substituted by a halogen. Examples of haloalkyl include but are not limited to CFH₂, CF₂H, CF₃, CH₂CF₃, CF₂CF₃, C(F)(CH₃)₂, CH₂CH₂Br, CH(I)CH₂F, and CH₂Cl.

As used herein, the term “haloalkoxy” refers to alkoxy structures that are substituted with one or more halo groups or with combinations thereof. For example, the terms “fluoroalkyl” and “fluoro alkoxy” include haloalkyl and haloalkoxy groups, respectively, in which the halo is fluorine.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety.

As used herein, the expressions “one or more of A, B, or C,” “one or more A. B, or C,” “one or more of A. B, and C,” “one or more A, B, and C.” “selected from the group consisting of A. B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formula described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the disclosure remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March 's Advanced Organic Chemistry: Reactions, Mechanisms, and Structure, 5^{th} edition, John Wiley & Sons: New York, 2001; Greene, T.W., Wuts, P.G.M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser 's Reagents for Organic Synthesis, John Wiley and Sons (1994); and L. Paquette, ed., Encyclopedia of Reagents for Organic Synthesis, John Wiley and Sons (1995) are useful and recognized reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene. T.W., Wuts, P.G. M., Protective Groups in Organic Synthesis, 3^{rd} edition, John Wiley & Sons: New York. 1999.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulphonic, acetic, ascorbic, benzene sulphonic, benzoic, bicarbonic, carbonic, citric, edetic, ethane disulphonic, 1,2-ethane sulphonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulphonic, maleic, malic, mandelic, methane sulphonic, napsylic, nitric, oxalic, pamoic, pantothenic, phenylacetic, phosphoric, polygalacturonic, propionic, salicylic, stearic, subacetic, succinic, sulphamic, sulphanilic, sulphuric, tannic, tartaric, toluene sulphonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulphonic acid, 2-naphthalenesulphonic acid, 4-toluenesulphonic acid, camphorsulphonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognise the advantages of certain routes of administration.

A salt, for example, can be formed between an anion and a positively charged group (e.g., amino) on a substituted compound disclosed herein. Suitable anions include chloride, bromide, iodide, sulphate, bisulphate, sulphamate, nitrate, phosphate, citrate, methanesulphonate, trifluoroacetate, glutamate, glucuronate, glutarate, malate, maleate, succinate, fumarate, tartrate, tosylate, salicylate, lactate, naphthalenesulphonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create anew compound with similar biological properties to the parent compound. The bioisosteric replacement may be physicochemically or topologically based. Examples of carboxylic acid bioisosteres include, but are not limited to, acyl sulphonamides, tetrazoles, sulphonates and phosphonates. See, e.g., Patani and LaVoie, Chem. Rev. 96, 3147-3176, 1996.

It is also to be understood that certain compounds of any one of the Formula disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate.

Compounds of any one of the Formula disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I).

Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

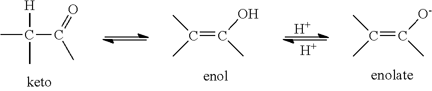

Compounds of any one of the Formula disclosed herein containing an amine function may also form N-oxides. A reference herein to a structure of Formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g., a peroxycarboxylic acid), see for example Advanced Organic Chemistry, by Jerry March, 4th Edition. Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formula disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formula disclosed herein.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterised by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterised by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers).

Accordingly, the present disclosure includes those compounds of any one of the Formula disclosed herein as defined hereinbefore when made available by organic synthesis and when made available within the human or animal body by way of cleavage of a prodrug thereof. Accordingly, the present disclosure includes those compounds of any one of the Formula disclosed herein that are produced by organic synthetic means and also such compounds that are produced in the human or animal body by way of metabolism of a precursor compound, that is a compound of any one of the Formula disclosed herein may be a synthetically-produced compound or a metabolically-produced compound.

A suitable pharmaceutically acceptable prodrug of a compound of any one of the Formula disclosed herein is one that is based on reasonable medical judgment as being suitable for administration to the subject without undesirable pharmacological activities and without undue toxicity.

A suitable pharmaceutically acceptable prodrug of a compound of any one of the Formula disclosed herein that possesses a hydroxy group is, for example, an in vivo cleavable ester or ether thereof. An in vivo cleavable ester or ether of a compound of any one of the Formula disclosed herein containing a hydroxy group is, for example, a pharmaceutically acceptable ester or ether which is cleaved in the subject to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N-(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formula disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formula disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

The dosage regimen utilizing the compounds is selected in accordance with a variety of factors including type, species, age, weight, sex and medical condition of the subject; the severity of the condition to be treated; the route of administration; the renal and hepatic function of the subject; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

Citation of publications and patent documents is not intended as an admission that any is pertinent prior art, nor does it constitute any admission as to the contents or date of the same. The disclosure having now been described by way of written description, those of skill in the art will recognize that the disclosure can be practiced in a variety of embodiments and that the foregoing description and examples herein are for purposes of illustration and not limitation of the claims that follow.

### EXAMPLES

The disclosure is further illustrated by the following examples. The examples are provided for illustrative purposes only, and are not to be construed as limiting the scope or content of the disclosure in any way.

### Example 1: Selection of Switch Sequences Containing Splice Modulator Binding Sites

This Example shows identification of minigene sequences using a library of variant switch sequences with unique barcodes and a splice modulator.

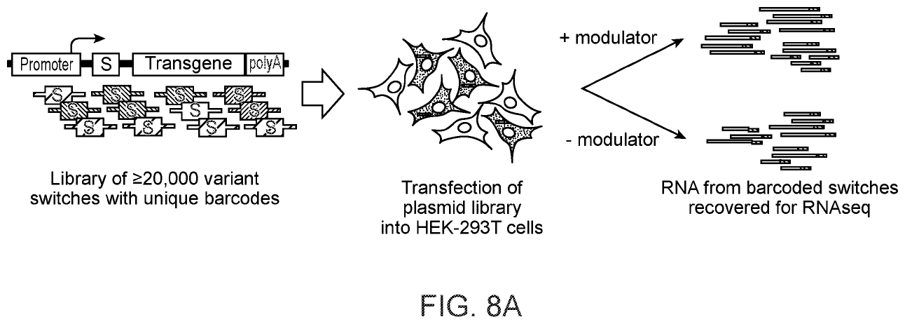

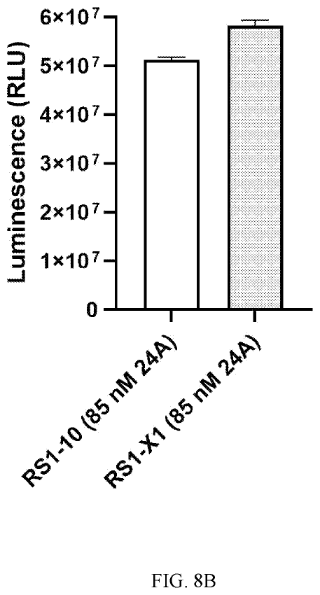

A splice modulator binding site was designed by screening greater than 20,000 switch sequence variants, each labeled with a unique nucleotide barcode. Sequence variants included nucleotide changes, such as insertions, deletions, and mutagenesis in various patterns. The oligonucleotide library was then cloned into expression plasmids, resulting in a plasmid pool. The plasmid pool was transfected into cells, such as HEK-293T, using nucleofection. After transfection, cells were treated with a splice modulator compound at specific concentrations. After 24-72 hours of incubation with the compound, the cells were harvested, total RNA was purified, and cDNA was synthesized from the purified RNA. FIG. 8A displays a scheme of this assay. Targeted RNA-seq libraries were generated by amplifying the switch transcript sequences from the cDNA pool. Paired end RNA sequencing identified for each switch transcript: a variant-identifying barcode and whether the target exon was spliced into the given transcript. For each barcoded variant, the percent-spliced-in (spliced reads/total reads) was calculated for each vehicle or drug condition utilized in the screen. Variants were selected for further analysis if the percent-spliced-in for the vehicle condition was less than the percent for the wild-type control, and the fold change increase in percent-spliced-in following compound treatment was above a desired cut off, such as the 95^{th} percentile.

A plasmid pool that was transfected into HEK-293T cells was treated for 24 hours with splice modulator 24 A at a concentration of 85 nM. RS1-10 was the starting minigene sequence and a variant switch sequence was identified from the switch sequence screen containing a 28 nucleotide intronic deletion compared to the RS1-10 sequence. RSI-X10. RS1-10 controlling a firefly luciferase gene or RS1-X1 controlling a firefly luciferase gene were cloned into individual expression plasmids containing a CMV promoter. HEK 293T cells were transfected with either the RS1-10 or RST-XT expression plasmid and were then treated for 24 hours with 85 nM 24 A while incubated in a tissue culture incubator (37° C., 5% CO₂). The luminescence was measured as an endpoint analysis by adding 100 μL of a Luciferase substrate, such as Steady-Luc Firefly Luciferase substrate, to each well of the assay plate. The liquid contents of each well was transferred to a 96-well plate, mixed again for 2 min on an orbital shaker, and incubated at room temperature for 5 min. Finally, the luminescent signal from each well was measured with a plate reader (FIG. 8B). RS1-X1 produced a higher luminescence (RLU) than RS1-10.

### Example 2: Selection of a Splice Modulator

This Example describes an assay for identifying candidate splice modulators.

A splice modulator is generated by screening greater than 100 (e.g., greater than 1,000, greater than 10,000, greater than 100,000, or greater than 1,000,000) candidate splice modulators for their ability to alter splicing of a minigene from Example 1 to increase inclusion of a bipartite start codon and allow transgene expression. The screen utilizes a DNA construct including a minigene linked to a reporter gene, which is assembled through DNA synthesis and molecular cloning techniques known in the art, and inserted into mammalian cells using electroporation, chemical transfection, viral-mediated integration, or viral mediated episomal introduction. A reporter gene is a luminescent enzyme (e.g., firefly luciferase, nanoluc luciferase, renilla luciferase, or gaussia luciferase,), fluorescent protein (e.g., green fluorescent protein, blue fluorescent protein, or red fluorescent protein), or colorimetric enzyme (e.g., beta lactamase or secreted embryonic alkaline phosphatase). The construct generates a quantifiable signal proportional to a splicing event that leads to the inclusion of the start codon, which is readable by flow cytometry, microscopy, or multi-modal microplate readers using photo-multiplier tubes. Alternatively, the candidate splice modulator-dependent biological activity is assessed by sequencing the alternatively spliced mRNA transcripts produced by the DNA construct with RNASeq or by quantitative reverse transcription PCR (RT-qPCR) in singleton or multiplexed formats. After the screen, candidate splice modulators are applied to cells containing the DNA construct for up to 6, 12, 24, or 72 hours and then assessed for start-codon inclusion activity.

### Example 3: Nucleic Acid Molecules Including a Bipartite Start Codon for Selective Expression of a Transgene

This example demonstrates the ability of a nucleic acid molecule described herein to selectively control transgene expression in vitro.

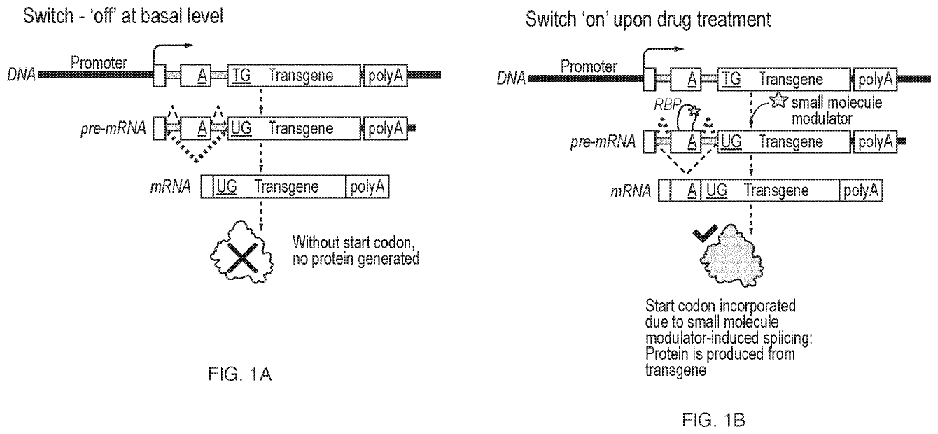

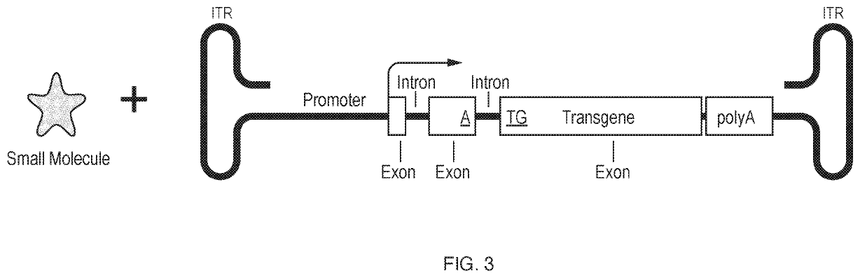

In this Example, a nucleic acid molecule of the disclosure (e.g., FIG. 2, FIGS. 6A-6L, and FIGS. 7A-7H) incorporated into a vector (FIG. 3) is designed, as described in Example 1, and generated using methods known in the art. The designed nucleic acid molecules function by exon-inclusion mechanisms, with five exemplary mechanisms provided in FIGS. 4A-4E. Cultured cells (e.g., HEK293 cells) are transfected with the described vector, which includes a bipartite start codon (e.g., at least one nucleotide of the start codon is located in the second exon and at least one nucleotide of the start codon is located in a transgene of the respective nucleic acid molecule) and a transgene under the control of splice modulator-dependent switch. After 24 hours of culture at 370 C and 5% CO₂, cells are treated with and without a splice modulator (e.g., a small molecule). In the presence of the splice modulator, the bipartite start codon is incorporated into the 5′-end of the transgene mRNA transcript to create a full start codon (“on;” FIG. 1B), thereby enabling transgene translation. Alternatively, when the splice modulator (e.g., a small molecule) is not present, the start codon is omitted by splicing (FIG. 1A) and the transgene is not expressed. After 24 hours of continuous culture, cells are fixed in 4% paraformaldehyde for 15 minutes at room temperature, blocked and permeabilized for 45 minutes with 10% FBS in PBS with 0.2% detergent. Samples are then incubated with primary antibodies against the reporter gene and secondary antibodies conjugated with Alexa 488 in PBS with 10% PBS and 0.1% detergent for 2 hours at room temperature or overnight at 4 C. Cells are then washed with PBS and subsequently analyzed using a fluorescent microscope for detection of expression of the reporter gene. The reporter gene is expressed when the splice modulator (e.g., a small molecule) is administered in combination with the above-described vector. In the absence of the splice modulator, no expression of the reporter gene is observed.

### Example 4: Modulation of a Protein in a Subject in Need Thereof by Administration of a Vector Containing a Nucleic Acid Molecule Including a Bipartite Start Codon

Using conventional molecular biology techniques known in the art, a nucleic acid molecule including a bipartite start codon (e.g., at least one nucleotide of the start codon is located in the second exon and at least one nucleotide of the start codon is located in a transgene of the respective nucleic acid molecule) and a transgene encoding a protein. RNA, miRNA, or shRNA of interest (e.g., a therapeutic protein or RNA product), is generated. The nucleic acid molecule(s) is subsequently incorporated into a vector, such as a viral vector, and administered to a subject in need thereof, e.g., a subject suffering from a disease associated with a deficiency in the protein, RNA, miRNA, or shRNA of interest. A subject is administered a viral vector encoding a protein. RNA, miRNA, or shRNA of interest under the control of a splice modulator-dependent switch that, in the presence of a splice modulator, promotes the incorporation of a bipartite start codon (e.g., at least one nucleotide of the start codon is located in the second exon and at least one nucleotide of the start codon is located in the transgene) by an alternative splicing event. An AAV vector, such as a pseudotyped AAV2/8 or AAV2/9 vector, is generated that incorporates a nucleic acid molecule described herein between 5′ and 3′ inverted terminal repeats of the vector, and a bipartite start codon (e.g., at least one nucleotide of the start codon is located in the second exon and at least one nucleotide of the start codon is located in the transgene) is placed under control of the splice modulator-dependent switch described in Examples 3 and 4, above. The AAV vector is administered to the subject by a variety of routes, including intravenously, intramuscularly, or subcutaneously, among others.

Following administration of the vector to a subject, a practitioner of skill in the art monitors the expression of the transgene by a variety of methods.

### Example 5: Screening of RS1-1 and Derivatives with Splice Modulator Molecules

This Example shows identification of RS1-1 variant constructs that result in expression of a gene in response to the presence of a splice modulator.

### Luciferase Induction Assay

HEK-293T cells were cultured in DMEM+10% FBS, collected after trypsinization, and dispensed into 96-well plates (cell culture-treated). Each well of the plate was seeded with 1.0×10⁵ cells in 100 μL culture medium. After a 1-hour incubation in the tissue culture incubator (5% CO2, 37° C.), plasmid DNA was complexed with a transfection reagent, such as Lipofectamine 3000, and added to respective wells on the plate. Each well received 100 ng of plasmid DNA complexed with 0.15 μL Lipofectamine 3000. The plasmids utilized in the experiment encoded a CMV promoter upstream of the RS1-1 variant that controlled the expression of a Firefly Luciferase gene followed by a SV40 polyadenylation signal. After a 10-minute incubation at room temperature, either DMSO vehicle or a splice modulator compound at specified doses was added to the wells of the plate. Plates were then incubated for 18 hours in a tissue culture incubator. Endpoint analysis was performed by adding 100 μL of a Luciferase substrate, such as Steady-Luc Firefly Luciferase substrate, to each well of the assay plate. The liquid contents of each was transferred to a 96-well plate, mixed again for 2 min on an orbital shaker, and incubated at room temperature for 5 min. Finally, the luminescent signal from each well was measured with a plate reader.

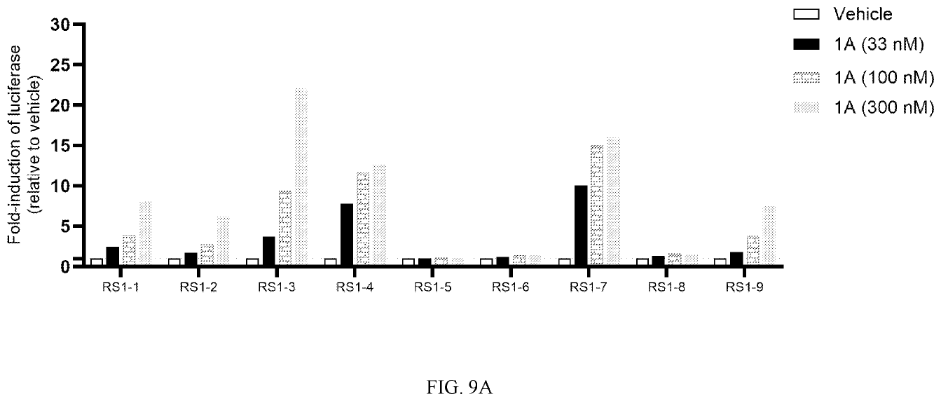

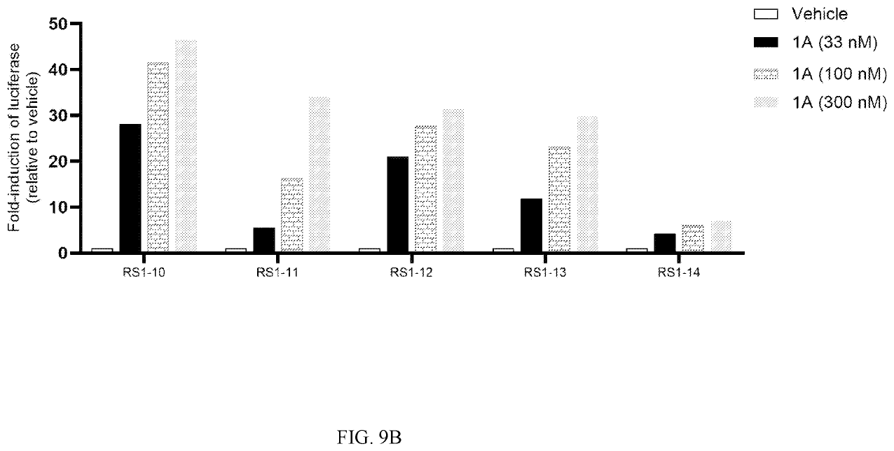

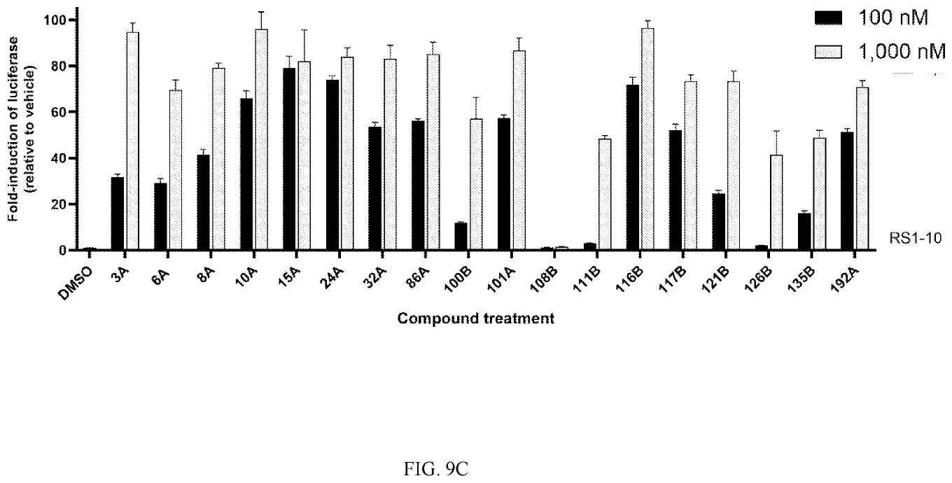

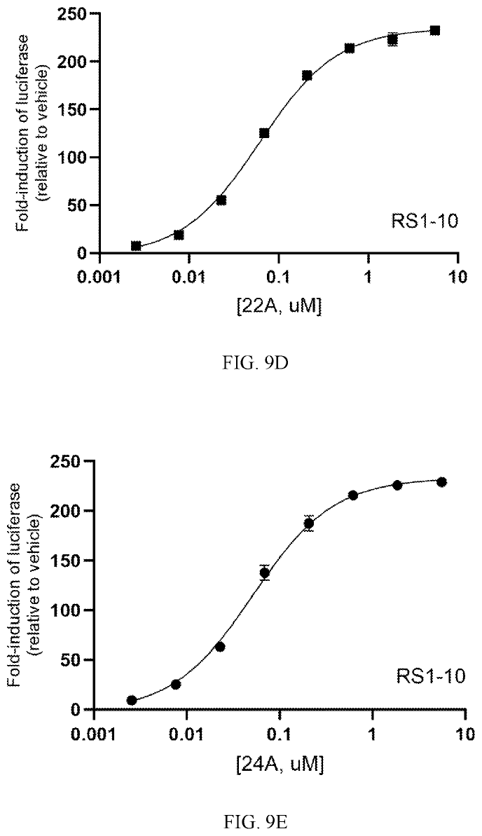

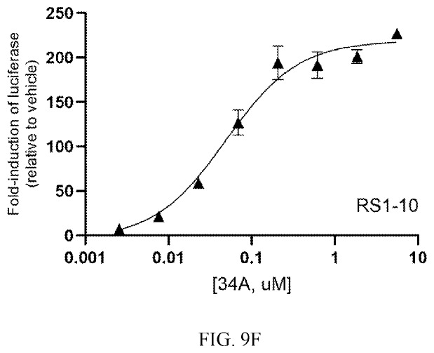

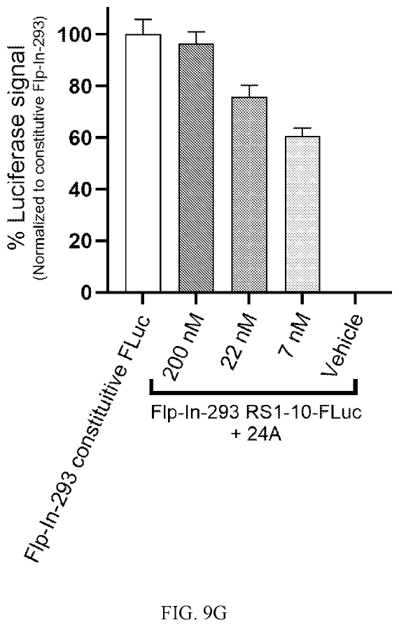

RS1-1 and variants were screened for induction of luminescent signal with either DMSO vehicle or 1 A (FIG. 9A) with this assay. RS1-1, RS1-2, RS1-3. RS1-4. RS1-7, and RS1-9 show increasing fold induction of luciferase with increasing concentration of splice modulator.

RS1-1 variants were screened for induction of luminescent signal with either DMSO vehicle or IA (FIG. 9B) with this assay. The constructs show increasing fold induction of luciferase with increasing concentration of splice modulator.

Screening Exemplary RSI-10 with Splice Modulator Molecules

HEK-293T cells were cultured in DMEM+10% FBS, collected after trypsinization, and then dispensed into 96-well plates (cell culture-treated). Each well of the plate was seeded with 1.0×10⁵ cells in 100 μL culture medium. After a 1-hour incubation in a tissue culture incubator (5% CO₂, 37 degrees C.), plasmid DNA was complexed with a transfection reagent, such as Lipofectamine 3000, and added to respective wells on the plate. Each well received an equivalent of 100 ng of plasmid DNA complexed with 0.15 μL Lipofectamine. Triplicate wells were used for each condition tested in the experiment. The plasmid utilized in the experiment encoded a CMV promoter upstream of the RS I-10 variant that controlled the expression of a Firefly Luciferase gene followed by a SV40 polyadenylation signal. After a 10-minute incubation at room temperature, either DMSO vehicle or specified compound at specified dose was added to the wells of the plate. Plates were then incubated for 18 hours in a tissue culture incubator. Endpoint analysis was performed by adding 100 uL Steady-Luc Firefly Luciferase substrate to each well of the assay plate. After mixing, the liquid contents of each was transferred to a 96-well plate, mixed again for 2 min on an orbital shaker, and incubated at room temperature for 5 min. Finally, the luminescent signal from each well was measured with a with PerkinElmer Envision plate reader. The results of this assay show RS1-10 responded to the majority of the splice modulator compounds used in a dose responsive manner (FIG. 9C). Bars represent mean+/−standard deviation of 3 wells normalized to vehicle controls. The mean fold-induction of luciferase (relative to vehicle control) for the various compounds tested is summarized in Table 4.

### Dose-Response Profiling Assay

Single-copy insertion of an switch construct and subsequent cell-line generation was performed in Flp-In-293 cells according to manufacturer's protocols. The RS1-10 sequence fused to a Firefly Luciferase gene was cloned into a Flp-In compatible pcDNA5/FRT expression vector with a CMV promoter and BGH polyadenylation signal. Resulting Flp-In cell line carried a single-copy expression cassette (RSwitch-controlled) integrated into the genome. The RS1-10 Flp-In cell line was cultured in DMEM+10% FBS, collected after trypsinization, and then dispensed into 96-well plates (cell culture-treated). Each well of the plate was seeded with 1.0×10⁵ cells in 100 μL culture medium. Subsequently, either DMSO vehicle or a splice modulator compound at specified doses was added to the wells of the plate. Triplicate wells were used for each condition tested in the experiment. Plates were then incubated for 18 hours in a tissue culture incubator (5% CO2, 37° C.). Endpoint analysis was performed by adding 100 uL of a Luciferase substrate, such as Steady-Luc Firefly Luciferase substrate, to each well of the assay plate. The liquid contents of each was transferred to a 96-well plate, mixed again for 2 min on an orbital shaker, and incubated at room temperature for 5 min. Finally, the luminescent signal from each well was measured with a plate reader. Each plotted data point on a graph in a figure represents a mean of 3 wells+/−standard deviation normalized to vehicle controls. A nonlinear regression curve fit of the plotted data points was generated in graphing software.

The dose response of RSI-10 to specified concentrations of 22 A (FIG. 9D), 24A(FIG. 9E), or 34 A (FIG. 9F) was measured. RSI-10 shows an increase in fold-induction of luciferase signal with increasing concentrations of 22 A (FIG. 9D), 24 A (FIG. 9E), or 34 A (FIG. 9F).

Comparison of RSI-10 Flp-In Cell Line with Constitutive Flp-In Cell Line

Single-copy insertion of RSI-10 sequence and subsequent cell-line generation was performed in Flp-In-293 cells according to manufacturer's protocols. The RS1-10 sequence fused to a Firefly Luciferase gene was cloned into a Flp-In compatible pcDNA5/FRT expression vector with a CMV promoter and BGH polyadenylation signal. The resulting Flp-In cell line carried a single-copy expression cassette of RS1-10 integrated into the genome. Similarly, a cell line constitutively expressing Firefly Luciferase from a single-copy genomic insertion was generated in Flp-In-293 cells. For the constitutive cell line, only the Firefly Luciferase gene was cloned into the pcDNA5/FRT expression vector that was used to generate the single-copy genomic insertion. Both cell lines were cultured in DMEM+10% FBS, collected after trypsinization, and then dispensed into 96-well plates (cell culture-treated). Each well of the plate was seeded with 1.0×10⁵ cells in 100 μL culture medium. Subsequently, either DMSO vehicle or 24 A at specified doses was added to the wells of the plate containing the RS1-10 Flp-In 293 cells. Triplicate wells were used for each condition tested in the experiment. Plates were then incubated for 18 hours in a tissue culture incubator (5% CO2, 37 degrees C.). Endpoint analysis was performed by adding 100 uL of a Luciferase substrate, such as Steady-Luc Firefly Luciferase substrate, to each well of the assay plate. The liquid contents of each was transferred to a 96-well plate, mixed again for 2 min on an orbital shaker, and incubated at room temperature for 5 min. The results are shown in FIG. 9G. Bars on graph represent mean of 3 wells+/−standard deviation normalized to the constitutively expressed Firefly Luciferase cell line as 100%. RS1-10 Flp-In-293 cells showed at least 60% luciferase signal in the presence of 24 A at 7 nM, with 200 nM 24 A resulting in similar luciferase expression as the Flp-In 293 cell line constitutively expressing Firefly Luciferase.

### Example 6: RS1-10 Performance in Various Cell Lines

This Example shows the response of RS1-10 to the presence of a splice modulator in various cell lines.

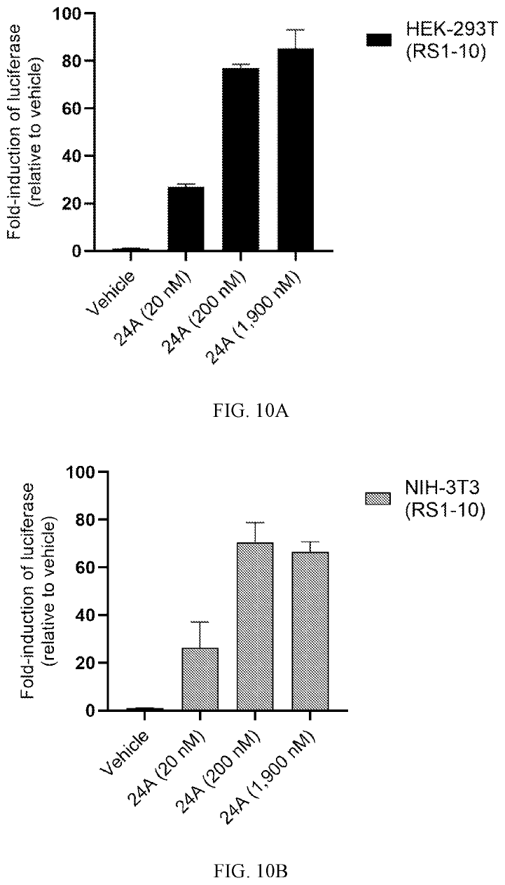

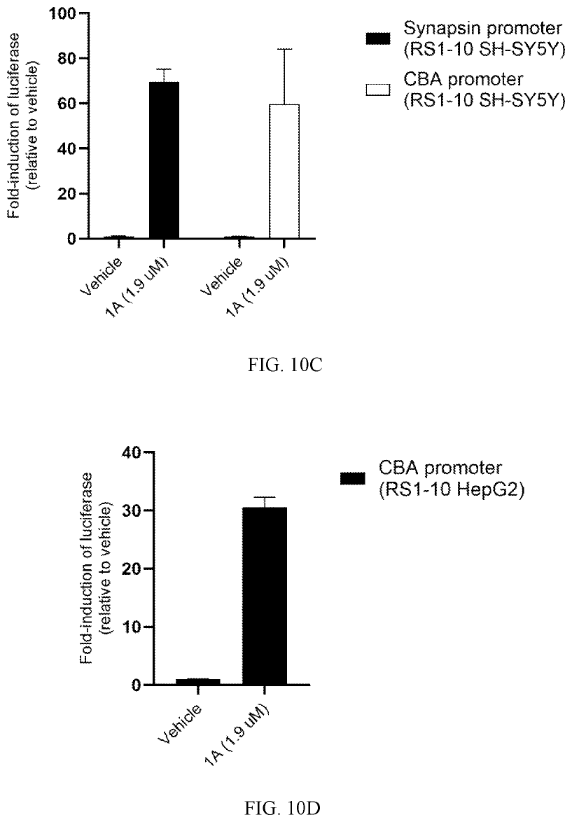

Human cell line HEK-293T cells or NIH-3T3 mouse fibroblast cell line cells were cultured in DMEM+10% FBS, collected after trypsinization, and then dispensed into 96-well plates (cell culture-treated). Each well of the plate was seeded with 1.0×10⁵ cells in 100 μL culture medium. After a 1-hour incubation in the tissue culture incubator (5% CO2, 37 degrees C.), plasmid DNA was complexed with a transfection reagent, such as Lipofectamine 3000, and added to respective wells on the plate. Each well received an equivalent of 100 ng of plasmid DNA complexed with 0.15 μL Lipofectamine 3000. Triplicate wells were used for each condition tested in the experiment. The plasmid utilized in the experiments for HEK-293T cells and NIH-3T3 mouse fibroblast cells encoded a CMV promoter upstream of the RS1-10 construct that controlled the expression of a Firefly Luciferase gene followed by a SV40 polyadenylation signal. After a 10-minute incubation at room temperature, either DMSO vehicle or 24 A at specified doses was added to the wells of the plate. Plates were then incubated for 18 hours in a tissue culture incubator. Endpoint analysis was performed by adding 100 uL of a Luciferase substrate, such as Steady-Luc Firefly Luciferase substrate, to each well of the assay plate. The liquid contents of each was transferred to a 96-well plate, mixed again for 2 min on an orbital shaker, and incubated at room temperature for 5 min. Finally, the luminescent signal from each well was measured with a plate reader. The results are shown in FIG. 10A and FIG. 10B. RS-10 shows increasing fold-induction of luciferase with increasing concentrations of 24 A in HEK-293T cells (FIG. 10A) and NIH-373 cells (FIG. 10B). Bars represent mean+/−standard deviation of 3 wells normalized to vehicle controls. RST-10 showed increasing fold-induction of luciferase with increasing concentrations of 24 A.

The human SH-SY5Y neuroblastoma cell line or the human hepatic HepG2 cell line was cultured in EMEM+10% FBS, collected after trypsinization, and then dispensed into 96-well plates (cell culture-treated). Each well of the plate was seeded with 1.0×10⁵ cells in 100 μL culture medium. After a 1-hour incubation in the tissue culture incubator (5% CO2, 37 degrees C.), plasmid DNA was complexed with a transfection reagent, such as Lipofectamine 3000, and added to respective wells on the plate. Each well received an equivalent of 100 ng of plasmid DNA complexed with 0.15 μL Lipofectamine 3000 (Opti-MEM I was used as diluent). Triplicate wells were used for each condition tested in the experiment. The plasmids utilized in the human SH-SY5Y neuroblastoma cell line encoded either a CBA promoter or cloned human Synapsin promoter upstream of the RS1-10 construct that controlled the expression of a Firefly Luciferase gene followed by a SV40 polyadenylation signal. The plasmids utilized in the HepG2 cells encoded a CBA promoter upstream of the RS1-10 construct that controlled the expression of a Firefly Luciferase gene followed by a SV40 polyadenylation signal.

After a 10-minute incubation at room temperature, either DMSO vehicle or TA was added to the wells of the plate. Plates were then incubated for 18 hours in a tissue culture incubator. Endpoint analysis was performed by adding 100 uL of a Luciferase substrate, such as Steady-Luc Firefly Luciferase substrate, to each well of the assay plate. The liquid contents of each was transferred to a 96-well plate, mixed again for 2 min on an orbital shaker, and incubated at room temperature for 5 min. Finally, the luminescent signal from each well was measured with a plate reader. RS-10 shows fold-induction of luciferase in the presence of TA in SH-SY5Y cells with either a CBA promoter or Synapsin promoter (FIG. 10C). Bars represent mean+/−standard deviation of 3 wells normalized to vehicle controls. RS-10 showed fold-induction of luciferase in the presence of TA in HepG2 cells with a CBA promoter (FIG. 10D). Bars represent mean+/−standard deviation of 3 wells normalized to vehicle controls.

### Example 7: RS1-10 Performance in Mice

This Example shows a design of an AAV cassette comprising RS1-10, showing exemplary switch, RS1-10, expresses luciferase promptly after treatment with a splice modulator and the expression is not constitutive.

In Vivo Mouse Study to Demonstrate Viral (AAV) Gene Therapy Proof of Concept with RSI-10

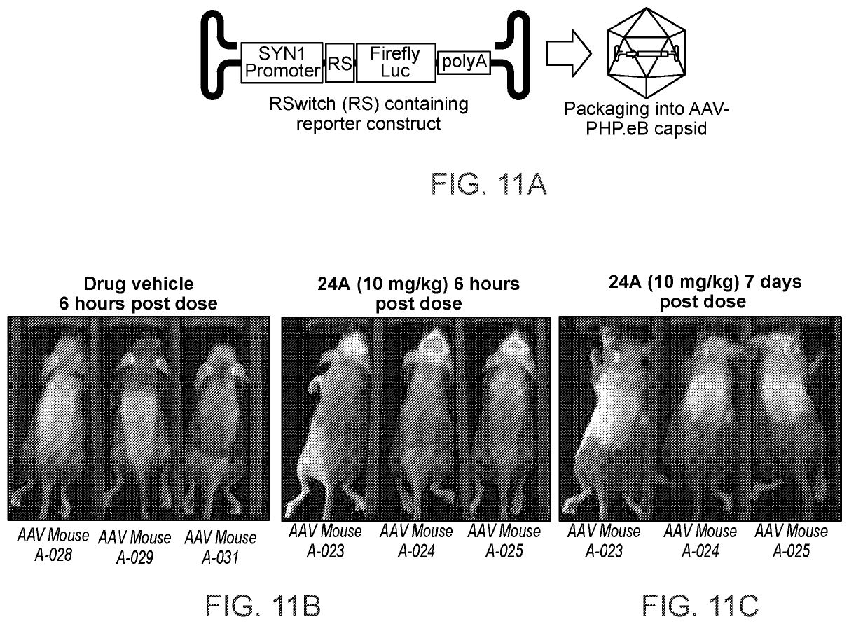

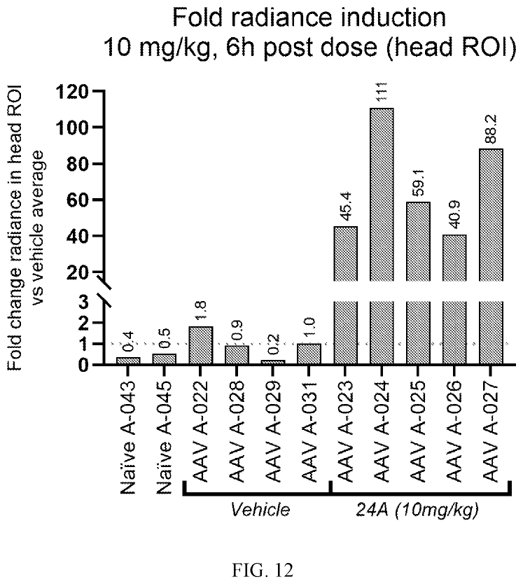

C57BL/6 mice underwent tail vein injection of 3.00E+11 vector (depicted in FIG. 11A) genomes/mouse AAV vector composed of a genome containing a cloned human Synapsin promoter driving the expression of a Firefly Luciferase gene. Expression of the luciferase gene was gated by the RS1-10 switch. The AAV vector utilized a brain-penetrant AAV-PHP.eB capsid (FIG. 11A). At approximately 3 weeks after AAV delivery, mice were either dosed with compound vehicle or 10 mg/kg 24 A as a single oral gavage. 6 hours after the oral dosing, mice were intraperitoneally administered D-Luciferin (150 mg/kg), anesthetized, and imaged with a bioluminescent imaging (BLI) system to analyze activation of RS1-10. Light flux from expressed Firefly Luciferase was measured from the whole body of each mouse as radiance and a brightfield image was also taken as an anatomical reference (FIG. 11B). Significant increases in light flux was observed from the heads of the 24 A treated mice while the vehicle treated mice only displayed background levels of luminescent signal from their heads (FIG. 12). Then, 7 days post 24 A administration, mice were once again dosed with D-Luciferin and imaged as before to characterize RS1-10 switching off transgene expression after drug washout (FIG. 11C). Images displayed are white light images with BLI signal overlay. No significant signal was detected in the mice 7 days post dose of 24 A.

Quantification of the RSI-10 Activation Obtained from In Vivo Mouse Study to Demonstrate a Viral (AAV) Gene Therapy Proof of Concept with RS1

C57BL/6 mice underwent tail vein injection of 3.00E+11 vector genomes/mouse AAV vector composed of a genome containing a cloned human Synapsin promoter driving the expression of a Firefly Luciferase gene. Expression of the luciferase gene was gated by the RS1-10 construct. The AAV vector utilized a brain-penetrant AAV-PHP.eB capsid. Approximately 3 weeks after AAV injection, mice were either dosed with compound vehicle or 10 mg/kg 24 A as a single oral gavage. 6 hours after the oral dosing, mice were intraperitoneally administered D-Luciferin (150 mg/kg), anesthetized, and imaged with a bioluminescent imaging system. Light flux from expressed Firefly Luciferase was measured from the whole body of each mouse as radiance and a brightfield image was also taken as an anatomical reference. Naive (no AAV, no drug) mice were included for background radiance comparison. The results are shown in FIG. 12, which represents the luciferase radiance quantification of a region encompassing the head of each mouse normalized to the average radiance of the vehicle mice. The mice that injected with an AAV vector comprising RS1-10 show greater fold change radiance than the naïve mice. Analysis of each mouse's brain (cortex) by digital PCR indicated AAV transduction range was between 0.8-2.1 vector genomes per diploid cell.

### Example 8: Screening of RS2-1 and Derivatives with Splice Modulator Molecules

This Example shows RS2-1 variant constructs that result in expression of a gene in response to the presence of a splice modulator.

### Luciferase Induction Assay

HEK-293T cells were cultured in DMEM+10% FBS, collected after trypsinization, and dispensed into 96-well plates (cell culture-treated). Each well of the plate was seeded with 1.0×10⁵ cells in 100 μL culture medium. After a 1-hour incubation in the tissue culture incubator (5% CO2, 37° C.), plasmid DNA was complexed with a transfection reagent, such as Lipofectamine 3000, and added to respective wells on the plate. Each well received 100 ng of plasmid DNA complexed with 0.15 μL Lipofectamine 3000. The plasmids utilized in the experiment encoded a CMV promoter upstream of the RS2-1 construct and variants that controlled the expression of a Firefly Luciferase gene followed by a SV40 polyadenylation signal. After a 10-minute incubation at room temperature, either DMSO vehicle or 100B at specified doses was added to the wells of the plate. Plates were then incubated for 18 hours in a tissue culture incubator. Endpoint analysis was performed by adding 100 μL of a Luciferase substrate, such as Steady-Luc Firefly Luciferase substrate, to each well of the assay plate. The liquid contents of each was transferred to a 96-well plate, mixed again for 2 min on an orbital shaker, and incubated at room temperature for 5 min. Finally, the luminescent signal from each well was measured with a plate reader.

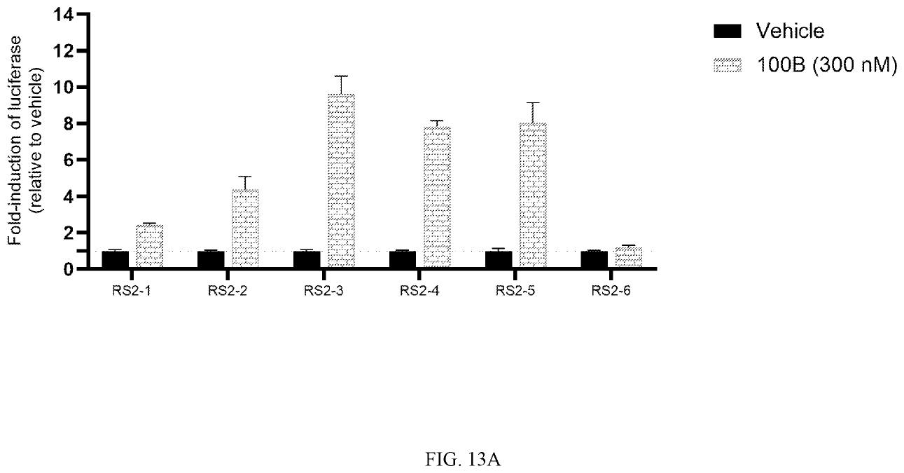

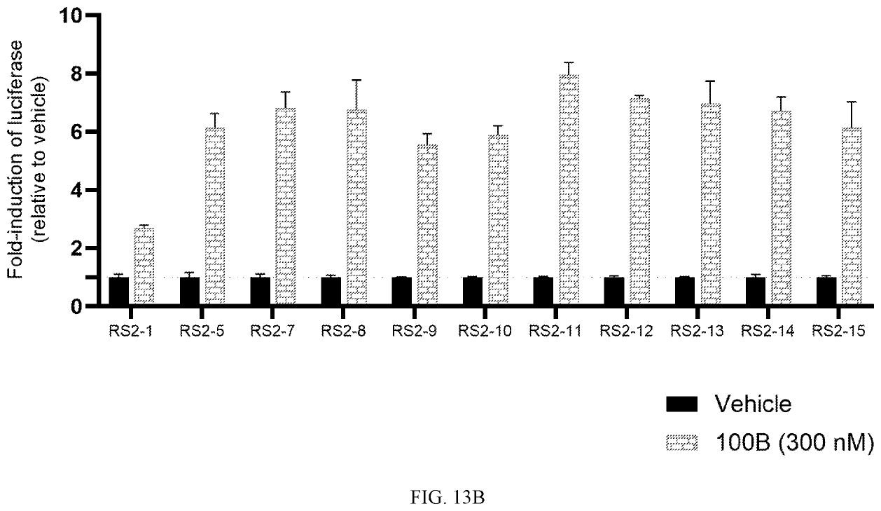

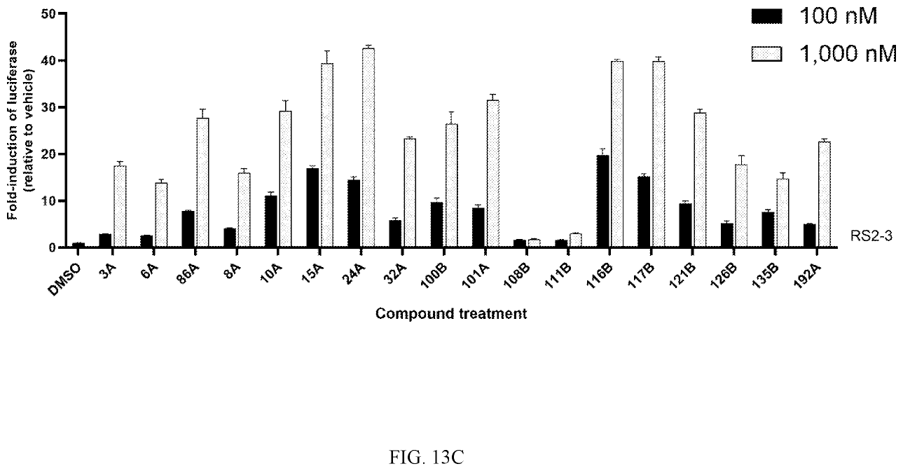

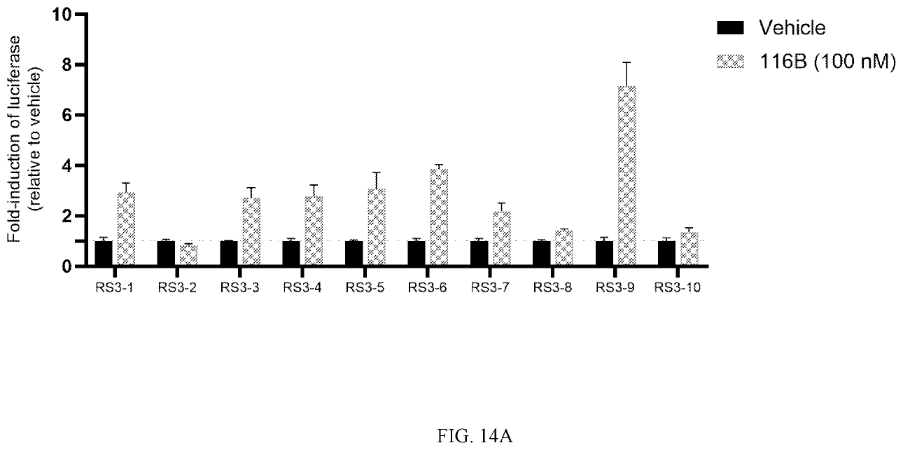

RS2-1 and variants were screened for induction of luminescent signal with either DMSO vehicle or 300 nM 100B with this assay, the results of which are shown in FIG. 13A. Additional RS2-1 variants were screened for induction of luminescent signal (FIG. 13B). The majority of the constructs show higher fold induction of luciferase in the presence of 1OOB compared to vehicle than RS2-1.

Screening Exemplary RS2-3 with Splice Modulator Molecules

HEK-293T cells were cultured in DMEM+10% FBS, collected after trypsinization, and then dispensed into 96-well plates (cell culture-treated). Each well of the plate was seeded with 1.0×10⁵ cells in 100 μL culture medium. After a 1-hour incubation in a tissue culture incubator (5% CO2, 37 degrees C.), plasmid DNA was complexed with a transfection reagent, such as Lipofectamine 3000, and added to respective wells on the plate. Each well received an equivalent of 100 ng of plasmid DNA complexed with 0.15 μL Lipofectamine. Triplicate wells were used for each condition tested in the experiment. The plasmid utilized in the experiment encoded a CMV promoter upstream of the RS2-3 variant that controlled the expression of a Firefly Luciferase gene followed by a SV40 polyadenylation signal. After a 10-minute incubation at room temperature, either DMSO vehicle or specified compound at specified dose was added to the wells of the plate. Plates were then incubated for 18 hours in a tissue culture incubator. Endpoint analysis was performed by adding 100 uL Steady-Luc Firefly Luciferase substrate to each well of the assay plate. After mixing, the liquid contents of each was transferred to a 96-well plate, mixed again for 2 min on an orbital shaker, and incubated at room temperature for 5 min. Finally, the luminescent signal from each well was measured with a with PerkinElmer Envision plate reader. The results of this assay show RS2-3 responded to the majority of the splice modulator compounds used (FIG. 13C) Bars represent mean+/−standard deviation of 3 wells normalized to vehicle controls. The mean fold-induction of luciferase (relative to vehicle control) for the various compounds tested is summarized in Table 4.

### Example 9: Screening of RS3-1 and Derivatives with Splice Modulator Molecules

This Example shows RS3-1 variant constructs that result in expression of a gene in response to the presence of a splice modulator.

### Luciferase Induction Assay

HEK-293T cells were cultured in DMEM+10% FBS, collected after trypsinization, and dispensed into 96-well plates (cell culture-treated). Each well of the plate was seeded with 1.0×10⁵ cells in 100 μL culture medium. After a 1-hour incubation in the tissue culture incubator (5% CO2, 37° C.), plasmid DNA was complexed with a transfection reagent, such as Lipofectamine 3000, and added to respective wells on the plate. Each well received 100 ng of plasmid DNA complexed with 0.15 μL Lipofectamine 3000. The plasmids utilized in the experiment encoded a CMV promoter upstream of the RS3-1 construct and variants that controlled the expression of a Firefly Luciferase gene followed by a SV40 polyadenylation signal. After a 10-minute incubation at room temperature, either DMSO vehicle or 116B at specified doses was added to the wells of the plate. Plates were then incubated for 18 hours in a tissue culture incubator. Endpoint analysis was performed by adding 100 μL of a Luciferase substrate, such as Steady-Luc Firefly Luciferase substrate, to each well of the assay plate. The liquid contents of each was transferred to a 96-well plate, mixed again for 2 min on an orbital shaker, and incubated at room temperature for 5 min. Finally, the luminescent signal from each well was measured with a plate reader.

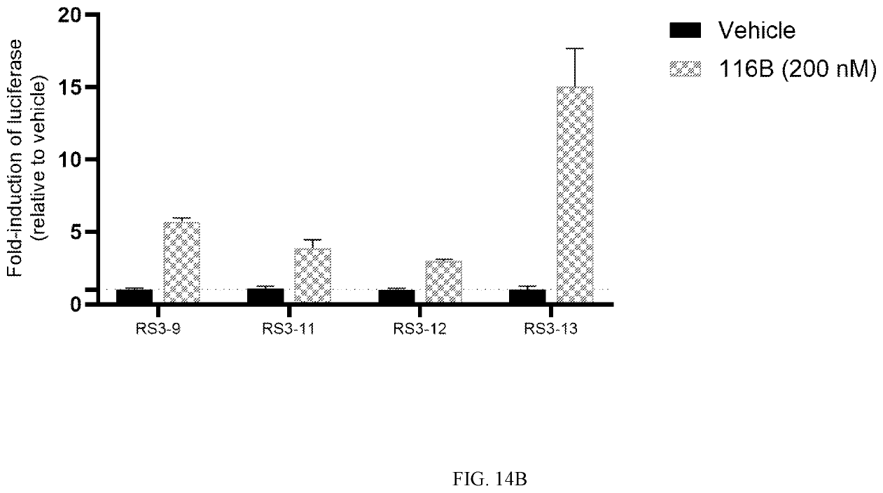

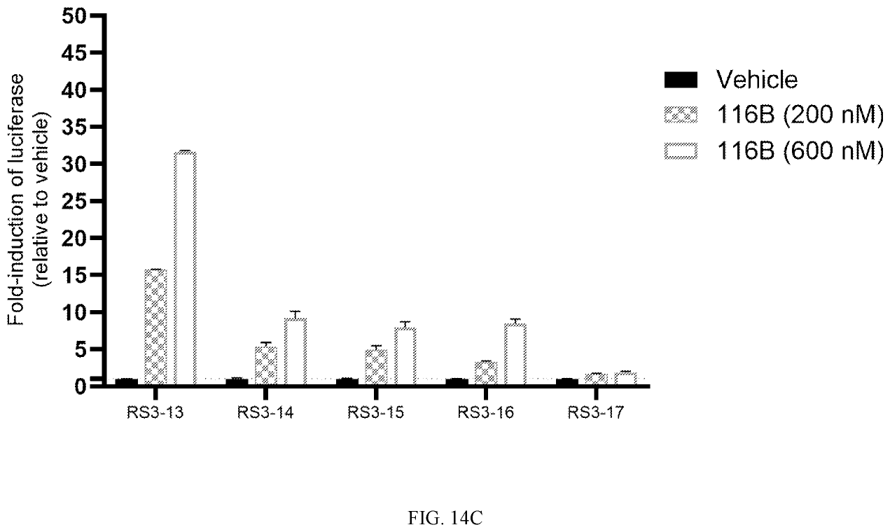

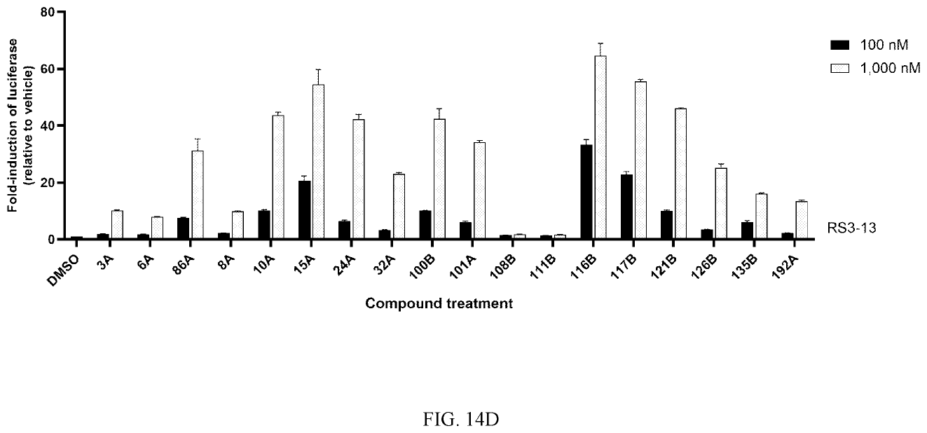

RS3-1 and variants were screened for induction of luminescent signal with either DMSO vehicle or 100 nM 116B with this assay (FIG. 14A, FIG. 14B). Additional RS3-1 variants were screened for induction of luminescent signal with either DMSO vehicle, 200 nM 116B, or 600 nM 116B (FIG. 14C). Some of the constructs show higher fold induction of luciferase in the presence of 116B compared to vehicle than RS3-1, and higher fold induction of luciferase with higher concentrations of 116B.]

Screening Exemplary RS3-13 with Splice Modulator Molecules

HEK-293T cells were cultured in DMEM+10% FBS, collected after trypsinization, and then dispensed into 96-well plates (cell culture-treated). Each well of the plate was seeded with 1.0×10⁵ cells in 100 μL culture medium. After a 1-hour incubation in a tissue culture incubator (5% CO2, 37 degrees C.), plasmid DNA was complexed with a transfection reagent, such as Lipofectamine 3000, and added to respective wells on the plate. Each well received an equivalent of 100 ng of plasmid DNA complexed with 0.15 μL Lipofectamine. Triplicate wells were used for each condition tested in the experiment. The plasmid utilized in the experiment encoded a CMV promoter upstream of RS3-13 that controlled the expression of a Firefly Luciferase gene followed by a SV40 polyadenylation signal. After a 10-minute incubation at room temperature, either DMSO vehicle or specified compound at specified dose was added to the wells of the plate. Plates were then incubated for 18 hours in a tissue culture incubator. Endpoint analysis was performed by adding 100 uL Steady-Luc Firefly Luciferase substrate to each well of the assay plate. After mixing, the liquid contents of each was transferred to a 96-well plate, mixed again for 2 min on an orbital shaker, and incubated at room temperature for 5 min. Finally, the luminescent signal from each well was measured with a with PerkinElmer Envision plate reader. The results of this assay show RS3-13 responded to the majority of the splice modulator compounds used (FIG. 14D) Bars represent mean+/−standard deviation of 3 wells normalized to vehicle controls. The mean fold-induction of luciferase (relative to vehicle control) for the various compounds tested is summarized in Table 4.

TABLE 4
Summary of mean fold-induction of luciferase of minigene

| | RS1-10 | | | | RS2-3 | | | | RS3-13 | | | |
|---|---|---|---|---|---|---|---|---|---|---|---|---|
| | 100 nM | | 1,000 nM | | 100 nM | | 1,000 nM | | 100 nM | | 1,000 nM | |
| Compound | Mean | S.D. | Mean | S.D. | Mean | S.D. | Mean | S.D. | Mean | S.D. | Mean | S.D. |
| 3A | 31.71 | 1.27 | 94.69 | 3.94 | 2.86 | 0.12 | 17.55 | 0.79 | 1.81 | 0.04 | 10.16 | 0.18 |
| 6A | 29.42 | 1.74 | 69.62 | 4.34 | 2.54 | 0.09 | 13.95 | 0.62 | 1.69 | 0.05 | 7.94 | 0.09 |
| 8A | 41.50 | 2.17 | 79.19 | 2.05 | 4.10 | 0.08 | 15.97 | 0.90 | 2.11 | 0.08 | 9.79 | 0.25 |
| 10A | 65.94 | 3.31 | 96.02 | 7.37 | 11.14 | 0.71 | 29.17 | 2.20 | 10.14 | 0.25 | 43.62 | 1.08 |
| 15A | 79.03 | 5.18 | 81.98 | 13.70 | 16.96 | 0.47 | 39.37 | 2.64 | 20.64 | 1.63 | 54.52 | 5.23 |
| 24A | 74.09 | 1.48 | 84.05 | 3.81 | 14.54 | 0.57 | 42.63 | 0.61 | 6.27 | 0.48 | 42.35 | 1.51 |
| 32A | 53.46 | 2.06 | 83.10 | 5.89 | 5.90 | 0.43 | 23.32 | 0.29 | 3.24 | 0.12 | 23.00 | 0.46 |
| 86A | 56.15 | 0.99 | 85.06 | 5.25 | 7.86 | 0.11 | 27.73 | 1.88 | 7.54 | 0.16 | 31.27 | 4.10 |
| 100B | 11.89 | 0.48 | 57.14 | 9.11 | 9.79 | 0.80 | 26.44 | 2.55 | 10.04 | 0.15 | 42.36 | 3.55 |
| 101A | 57.48 | 1.28 | 86.80 | 5.33 | 8.48 | 0.64 | 31.54 | 1.23 | 6.00 | 0.40 | 34.21 | 0.60 |
| 108B | 1.16 | 0.09 | 1.43 | 0.13 | 1.65 | 0.09 | 1.76 | 0.07 | 1.41 | 0.02 | 1.66 | 0.13 |
| 111B | 3.06 | 0.11 | 48.36 | 1.40 | 1.60 | 0.07 | 2.97 | 0.17 | 1.32 | 0.04 | 1.63 | 0.04 |
| 116B | 71.75 | 3.34 | 96.58 | 3.11 | 19.77 | 1.27 | 39.87 | 0.38 | 33.37 | 1.73 | 64.60 | 4.38 |
| 117B | 52.12 | 2.62 | 73.54 | 2.67 | 15.18 | 0.60 | 39.85 | 0.89 | 22.76 | 1.03 | 55.61 | 0.67 |
| 121B | 24.65 | 1.39 | 73.47 | 4.33 | 9.41 | 0.56 | 28.84 | 0.75 | 9.95 | 0.35 | 45.98 | 0.33 |
| 126B | 2.06 | 0.03 | 41.45 | 10.37 | 5.18 | 0.51 | 17.79 | 1.90 | 3.46 | 0.10 | 25.21 | 1.2 [symbol] |
| 135B | 16.07 | 1.00 | 48.79 | 3.30 | 7.61 | 0.45 | 14.73 | 1.23 | 5.99 | 0.57 | 16.12 | 0.2 [symbol] |
| 192A | 51.29 | 1.65 | 70.80 | 2.92 | 4.97 | 0.16 | 22.66 | 0.56 | 2.18 | 0.08 | 13.42 | 0.4 [symbol] |

Mean = Mean fold-induction of luciferase (relative to vehicle control); S.D. = Standard Deviation; all experiments were done with an n of 3. [symbol]
indicates data missing or illegible when filed

### Example 10: Screening of RS4-1 and Derivatives with Splice Modulator Molecules

This Example shows RS4-1 variants that result in expression of a gene in response to the presence of a splice modulator.

### Luciferase Induction Assay

HEK-293T cells were cultured in DMEM+10% FBS, collected after trypsinization, and dispensed into 96-well plates (cell culture-treated). Each well of the plate was seeded with 1.0×10⁵ cells in 100 μL culture medium. After a 1-hour incubation in the tissue culture incubator (5% CO2, 37° C.), plasmid DNA was complexed with a transfection reagent, such as Lipofectamine 3000, and added to respective wells on the plate. Each well received 100 ng of plasmid DNA complexed with 0.15 μL Lipofectamine 3000. The plasmids utilized in the experiment encoded a CMV promoter upstream of the RS4-1 construct and variants that controlled the expression of a Firefly Luciferase gene followed by a SV40 polyadenylation signal. After a 10-minute incubation at room temperature, either DMSO vehicle or 24 A at specified doses was added to the wells of the plate. Plates were then incubated for 18 hours in a tissue culture incubator. Endpoint analysis was performed by adding 100 uL of a Luciferase substrate, such as Steady-Luc Firefly Luciferase substrate, to each well of the assay plate. The liquid contents of each was transferred to a 96-well plate, mixed again for 2 min on an orbital shaker, and incubated at room temperature for 5 min. Finally, the luminescent signal from each well was measured with a plate reader.

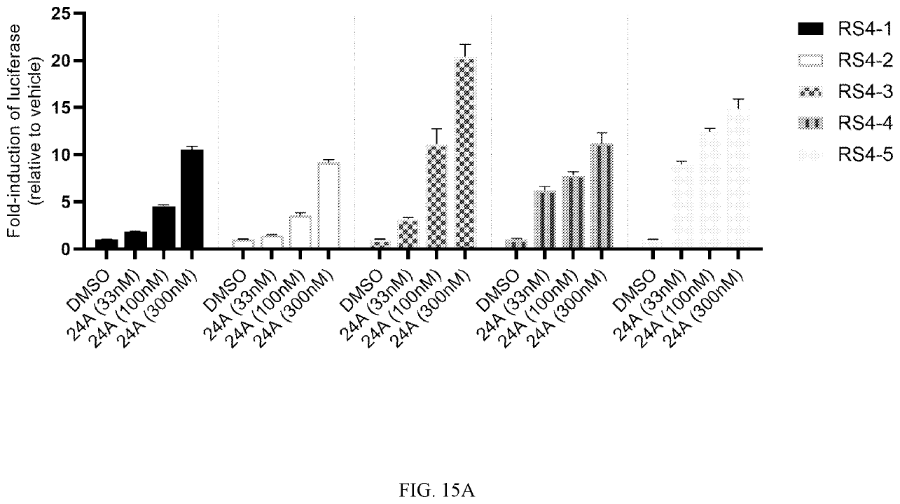

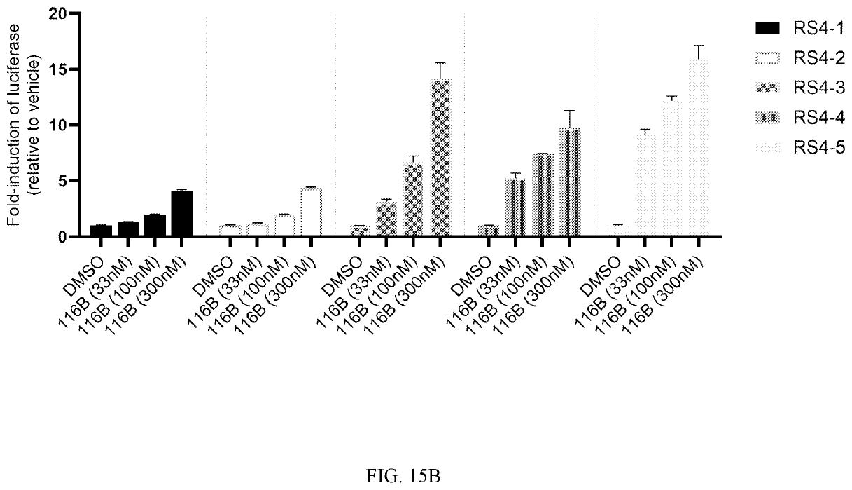

RS4-1 and variants were screened for induction of luminescent signal with either DMSO vehicle or a specified dose of 24 A or 116B with this assay. The RS4-1 construct and variants show increasing fold-induction of luciferase with increasing concentrations of 24 A (FIG. A) or 116B (FIG. 15B).

### Example 11. Synthesis of (S)-9-fluoro-2-(8-fluoro-2-methylimidazo[1,2-a]pyridine-6-yl)-7-(3-methylpiperazin-1-yl)-4H-pyrido[1,2-a][1,3,5]triazin-4-one (Compound 116B)

This Example shows the synthesis scheme for Compound 116B.

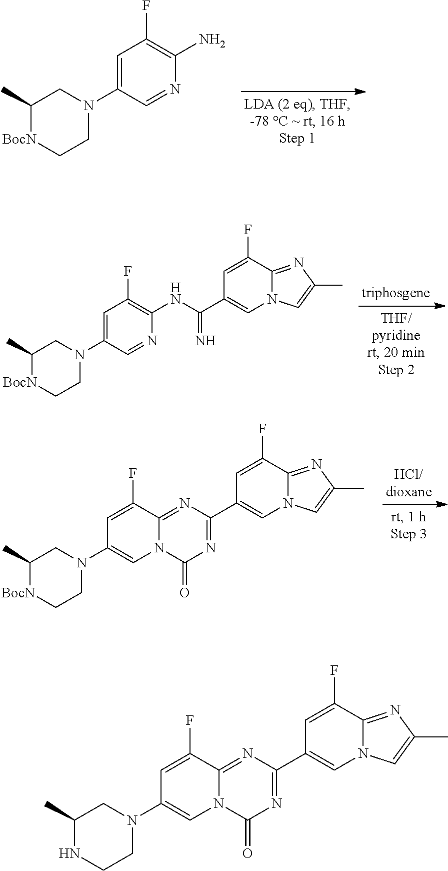

### Step 1: tert-butyl (S)-4-(5-fluoro-6-(8-fluoro-2-methylimidazo[1,2-a]pyridine-6-carboximidamido)pyridin-3-yl)-2-methylpiperazine-1-carboxylate

To a mixture of diisopropylamine (0.3 mL, 2.2 mmol) in dry THF (10 mL) was added n-BuLi (1.5 mL, 2.4 mmol, 1.6M) dropwise at −65° C. The mixture was stirred at this temperature for 1 hour, then tert-butyl (S)-4-(6-amino-5-fluoropyridin-3-yl)-2-methylpiperazine-1-carboxylate (465 mg, 1.56 mmol) in THF (2 mL) was added, the mixture was allowed to warm to rt and stirred for 1.5 h. The mixture was cooling down to −60° C. again. Then 8-fluoro-2-methylimidazo[1,2-a]pyridine-6-carbonitrile (175 mg, 1 mmol) in THF (5 mL) was added slowly, the mixture was stirred overnight at rt. The reaction mixture was diluted with water (30 mL) and extracted with EtOAc (40 mL×3). The organic layer was washed with brine, dried with Na₂SO₄ and evaporated to give crude title compound. The residue was purified by C18 column chromatography to give title compound (150 mg, yield: 30%) as a grey solid. ESI-MS (M+H)⁺: 486.3. ¹H NMR (400 MHz, CDCl₃): δ 8.65 (d, J=1.3 Hz, 1H), 7.78 (d, J=2.6 Hz, 1H), 7.48 (d, J=2.4 Hz, 1H), 7.33 (dd, J=11.1, 1.3 Hz, 1H), 7.04 (dd, J=12.4, 2.6 Hz, 1H), 4.44-4.34 (m, 1H), 3.98 (d, J=13.8 Hz, 1H), 3.48 (d, J=11.9 Hz, 1H), 3.38-3.21 (m, 2H), 3.02-3.00 (m, 1H), 2.83-2.80 (m, 1H), 2.50 (s, 3H), 1.49 (s, 9H). 1.31 (d, J=6.7 Hz, 3H).

### Step 2: tert-butyl (S)-4-(9-fluoro-2-(8-fluoro-2-methylimidazo[1,2-a]pyridin-6-yl)-4-oxo-4H-pyrido[1,2-a][1, 3,5]triazin-7-yl)-2-methylpiperazine-1-carboxylate

To a solution of tert-butyl (S)-4-(5-fluoro-6-(8-fluoro-2-methylimidazo[1,2-a]pyridine-6-carboximidamido)pyridin-3-yl)-2-methylpiperazine-1-carboxylate (1.0 g, 2.06 mmol) in THF (100 mL) and pyridine(10 mL) was added triphosgene (1.83 g, 6.18 mmol) at 0° C., the mixture was stirred for 20 min at 0° C. The reaction mixture was diluted with water (50 mL) and extracted with EtOAc (100 mL×3). The organic layer was washed with brine, dried with Na₂SO₄ and evaporated to give crude title compound. The residue was purified by prep-HPLC (0.05% HCl in water/CH₃CN) to give title compound (550 mg yield: 52.6%) as a yellow solid. ESI-MS (M+H)+:512.0. ¹H NMR (400 MHz, CDCl₃) δ 9.22 (d, J=1.2 Hz, 1H), 8.31 (d, J=2.0 Hz, 1H), 7.97 (dd, J=11.6, 1.2 Hz, 1H), 7.63 (dd, J=10.5, 2.6 Hz, 1H), 7.49 (d, J=2.1 Hz, 1H), 4.44-4.40 (m, 1H), 4.04 (d, J=13.6 Hz, 1H), 3.59 (d, J=12.0 Hz, 1H), 3.44-3.40 (m, 1H), 3.36-3.29 (m, 1H), 3.21-3.16 (m, 1H), 3.03-2.96 (m, 1H), 2.51 (s, 3H), 1.50 (s, 9H), 1.30 (d, J=6.8 Hz, 3H).

### Step 3: (S)-9-fluoro-2-(8-fluoro-2-methylimidazo[1,2-a]pyridine-6-yl)-7-(3-methylpiperazin-1-yl)-4H-pyrido[1,2-a][1,3,5]triazin-4-one

To a solution of tert-butyl (S)-4-(9-fluoro-2-(8-fluoro-2-methylimidazo[1,2-a]pyridin-6-yl)-4-oxo-4H-pyrido [1,2-a][1,3,5]triazin-7-yl)-2-methylpiperazine-1-carboxylate (1.1 g, 2.15 mmol) in EtOAc (10 mL) was added 3M HCl/EtOAc (15 ml) at room temperature. The reaction mixture was stirred for 1 h. The mixture was concentrated in vacuo, the residue was purified by prep-HPLC (0.05% NH4OH in water/CH₃CN) to afford title compound (520 mg, 58%) as a yellow solid. ESI-MS (M+H)⁺:412.1. ¹H NMR (400 MHz, DMSO-d6) δ 9.43 (d, J=1.1 Hz, 1H), 8.43 (dd, J=12.4, 2.5 Hz, 1H), 8.10 (d, J=2.0 Hz, 1H), 8.06 (d, J=2.4 Hz, 1H), 7.78 (dd, J=12.3, 1.0 Hz, 1H), 3.70-3.65 (m, 2H), 3.02-2.99 (m, 1H), 2.81-2.66 (m, 3H), 2.39 (s), 2.37-2.33 (m, 1H), 1.06 (d, J=6.0 Hz, 3H)

### Example 12. Synthesis of 2-(8-fluoro-2-methylimidazo[1,2-a]pyridin-6-yl)-7-(piperazin-1-yl)-4H-pyrido[1,2-a][1,3,5]triazin-4-one hydrochloride (Compound 100B)

This Example shows the synthesis scheme for Compound 100B.

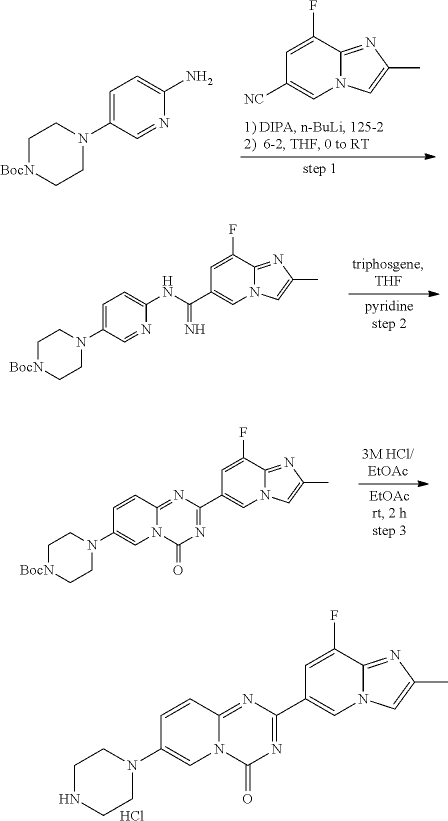 [SMILES: CC(C)(C)OC(=O)N1CCN(c2ccc(N)nc2)CC1 ; Cc1cn2cc(C#N)cc(F)c2n1 ; Cc1cn2cc(C(=N)Nc3ccc(N4CCN(C(=O)OC(C)(C)C)CC4)cn3)cc(F)c2n1 ; Cc1cn2cc(-c3nc(=O)n4cc(N5CCN(C(=O)OC(C)(C)C)CC5)ccc4n3)cc(F)c2n1 ; Cc1cn2cc(-c3nc(=O)n4cc(N5CCNCC5)ccc4n3)cc(F)c2n1 ; Cl]

### Step 1: tert-butyl 4-(6-(8-fluoro-2-methylimidazo[1.2-a]pyridine-6-carboximidamido)pyridin-3-yl)piperazine-1-carboxylate

To a mixture of diisopropylamine (0.6 mL, 4.4 mmol) in dry THF (15 mL) was added n-BuLi (3 mL, 4.8 mmol, 1.6M) dropwise at −65° C. The mixture was stirred at this temperature for 1 hour. Tert-butyl 4-(6-aminopyridin-3-yl)piperazine-1-carboxylate (1.12 g, 4.0 mmol) in dry THF (5 mL) was added. The mixture was allowed to warm to room temperature and stirred for 1 h. 8-fluoro-2-methylimidazo[1,2-a]pyridine-6-carbonitrile (350 mg, 2.0 mmol) in dry THF (10 mL) was added to the mixture at −40° C. and allowed to warm to room temperature and stirred for 16 h. The mixture was diluted with water (30 mL), extracted with EtOAc (30 mL×2). The combined organic layer was washed with brine (50 mL), dried over sodium sulfate, filtered and concentrated in vacuo. The residue was purified by column chromatography (PE/EA=1:1 to 0:1), then slurried with MeOH (30 mL) to give title product (600 mg, yield: 44.4%, three batches) as a yellow solid. ¹H NMR (400 MHz, CDCl₃) δ 8.57 (s, 1H), 8.01 (d, J=2.8 Hz, 1H), 7.47 (d, J=2.0 Hz, 1H), 7.36-7.31 (m, 2H), 7.21 (d, J=8.8 Hz, 1H), 3.62-3.58 (m, 4H), 3.15-3.12 (m, 4H), 2.50 (s. 3H), 1.49 (s, 9H). ESI-MS: [M+H]⁺: 454.3.

### Step 2: tert-butyl 4-(2-(8-fluoro-2-methylimidazo[1.2-a]pyridin-6-yl)-4-oxo-4H-pyrido[1,2-a][J, 3,5]triazin-7-yl)piperazine-1-carboxylate

To a solution of tert-butyl 4-(6-(8-fluoro-2-methylimidazo[1,2-a]pyridine-6-carboximidamido) pyridin-3-yl)piperazine-1-carboxylate (600 mg, 1.32 mmol) in THF (60 mL) and pyridine (6 mL) was added triphosgene (1.18 g. 3.97 mmol). The reaction mixture was stirred for I h at rt. The mixture was diluted with water (30 mL), extracted with EtOAc (30 mL×2). The combined organic layer was washed with brine (50 mL), dried over sodium sulfate, filtered and concentrated in vacuo. The residue was diluted with MeOH (40 mL) and stirred for 1 hour at rt. The precipitate was filtered and dried in vacuo to give title product (600 mg, yield: 97%). ¹H NMR (400 MHz, CDCl₃) δ 9.21 (s, 1H). 8.48 (d, J=2.4 Hz, 1H). 7.95-7.90 (m, 2H). 7.66 (d, J=9.6 Hz, 1H), 7.48 (d, J=2.0 Hz, 1H), 3.67-3.63 (m, 4H), 3.29-3.26 (m, 4H), 2.50 (s, 3H), 1.50 (s, 9H). ESI-MS: [M+H]⁺: 480.0.

### Step 3: 2-(8-fluoro-2-methylimidazo[1,2-a]pyridin-6-yl)-7-(piperazin-1-yl)-4H-pyrido[1,2-a][1,3,5]triazin-4-one hydrochloride

To a solution of tert-butyl 4-(2-(8-fluoro-2-methylimidazo[1,2-a]pyridin-6-yl)-4-oxo-4H-pyrido[1,2-a][1,3,5]triazin-7-yl)piperazine-1-carboxylate (1.25 g, 2.61 mmol) in EtOAc (15 mL) was added 3M HCl in EtOAc (15 ml) at room temperature. The reaction mixture was stirred for 2 h. The precipitate was filtered and lyophilized to give title product as HCl salt (1.0 g, yield: 86.2%) as a yellow solid. ¹H NMR (400 MHz, MeOD-d4) δ 9.66 (s, 1H). 8.66 (d, J=11.2 Hz. 1H), 8.60 (d, J=2.4 Hz, 1H), 8.41 (dd, J=9.6, 2.8 Hz, 1H), 8.25 (s, 1H), 7.91 (d, J=9.6 Hz, 1H), 3.71-3.67 (m, 4H), 3.49-3.46 (m, 4H), 2.62 (s, 3H). ESI-MS: [M+H]⁺ 380.2.

### Example 13. 2-(6-(azetidin-3-yl)pyridazin-3-yl)-5-(8-fluoro-2-methylimidazo[1,2-a]pyridin-6-yl)phenol, HCl. (Compound 1A)

This Example shows the synthesis scheme for Compound 1 A.

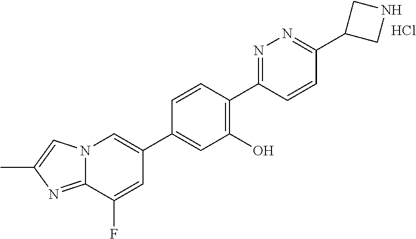 [SMILES: Cc1cn2cc(-c3ccc(-c4ccc(C5CNC5)nn4)c(O)c3)cc(F)c2n1 ; Cl]

### Step 1: Synthesis of 2-bromo-5-iodophenol

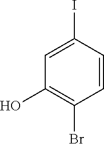

To a solution of 5-amino-2-bromophenol (50.0 g, 265.9 mmol) in 400 mL of 2.5 M hydrochloric acid, sodium nitrite (18.78 g, 272.2 mmol) in water (120 mL) was added dropwise at 0° C. After 15 min a solution of potassium iodide (49.3 g, 2967 mmol) in water (100 mL) was added dropwise and the whole mixture was heated at 60° C. for 1 h. The resulting mixture was extracted with MTBE (2×300 mL). The organic phase was dried over Na₂SO₄ and concentrated under reduced pressure, to give a brown liquid, which was purified by flash chromatography to give 2-bromo-5-iodophenol (60.0 g, 172.6 mmol, 75% yield).

### Step 2: Synthesis of 1-bromo-4-iodo-2-(methoxymethoxy)benzene

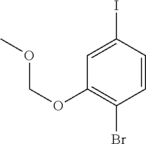

A mixture of 2-bromo-5-iodophenol (30 g. 100 mmol), potassium carbonate (41.4 g, 300 mmol) and MOM-Cl (24.15, 300 mmol) in DMF (400 mL) was stirred at rt for 48 h. Next, the reaction mixture was diluted with water (1000 mL) and extracted with MTBE (2×600 mL). The organic phases were washed with water and brine, dried over Na₂SO₄ and concentrated under reduced pressure, to give a brown liquid, which was purified by flash chromatography to give 1-bromo-4-iodo-2-(methoxymethoxy)benzene (21 g, 61 mmol, 61% yield).

### Step 3: Synthesis of 6-(4-bromo-3-(methoxymethoxy)phenyl)-8-fluoro-2-methylimidazo[1,2-a]pyridine

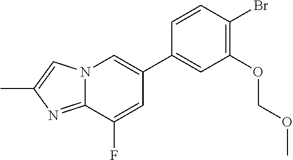

A portion of Pd(dppf)Cl₂ (950 mg, 1.2 mmol) was added to a suspension of 1-bromo-4-iodo-2-(methoxymethoxy)benzene (4 g. 11.7 mmol), 8-fluoro-2-methyl-6-(4,4,5,5-tetramethyl-1,3,2-dioxaborolan-2-yl)imidazo[1,2-a]pyridine (3.87 g, 14 mmol) and potassium carbonate (3.31 g, 24 mmol) in 100 mL of dioxane and 2 mL of water. The mixture was stirred at 80° C. for 12 h. After cooling, the solid was collected by filtration and washed with EtOAc. The filtrate was concentrated and partitioned between EtOAc and water. The organic layer was washed with brine, dried over Na₂SO₄, and concentrated to give crude product, which was purified by column chromatography to give 6-(4-bromo-3-(methoxymethoxy)phenyl)-8-fluoro-2-methylimidazo[1,2-a]pyridine (2 g, 5.5 mmol, 47% yield).

### Step 4:Synthesis of 8-fluoro-6-(3-(methoxymethoxy)-4-(4,4,5,5-tetramethyl-1,3,2-dioxaborolan-2-yl)phenyl)-2-methylimidazo[1,2-a]pyridine

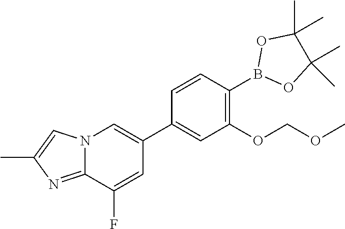

A mixture of 6-(4-bromo-3-(methoxymethoxy)phenyl)-8-fluoro-2-methylimidazo[1,2-a]pyridine (2 g, 5.5 mmol), bis(pinacolato)diboron (1.39 g, 5.5 mmol), potassium acetate (1.61 g, 16 mmol) and Pd(dppf)Cl₂ (450 mg. 0.55 mmol) in dioxane (20 mL) was stirred at 80° C. (under Ar atmosphere) for 12 h. Next it was concentrated, worked-up with EtOAc/water. The extract was dried and evaporated to give crude material, which was purified by column chromatography to afford 8-fluoro-6-(3-(methoxymethoxy)-4-(4,4,5,5-tetramethyl-1,3,2-dioxaborolan-2-yl)phenyl)-2-methylimidazo[1,2-a]pyridine (1.1 g, 2.6 mmol, 46% yield).

### Step 5: Synthesis of tert-butyl 3-(6-(4-(8-fluoro-2-methylimidazo[1,2-a]pyridin-6-yl)-2-(methoxymethoxy)phenyl)pyridazin-3-yl)azetidine-1-carboxylate

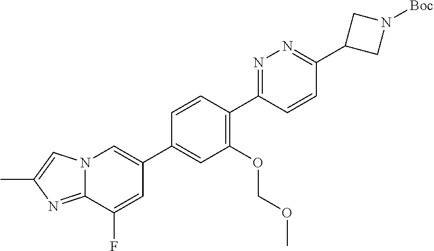 [SMILES: COCOc1cc(-c2cc(F)c3nc(C)cn3c2)ccc1-c1ccc(C2CN(C(=O)OC(C)(C)C)C2)nn1]

A portion of Pd(dppf)Cl₂ (70 mg, 85.77 μmol) was added to a suspension of 8-fluoro-6-(3-(methoxymethoxy)-4-(4,4,5,5-tetramethyl-1,3,2-dioxaborolan-2-yl)phenyl)-2-methylimidazo[1,2-a]pyridine (350 mg, 0.82 mmol), tert-butyl 3-(6-chloropyridazin-3-yl)azetidine-1-carboxylate (170 mg, 0.63 mmol) and potassium carbonate (355.6 mg, 2.57 mmol) in dioxane (10 mL) and water (1 mL). The mixture was stirred at 80° C. for 12 h. After cooling, the solid was collected by filtration and washed with EtOAc. The filtrate was concentrated and partitioned between EtOAc and water. The organic layer was washed with brine, dried over Na₂SO₄, and concentrated to give crude product, which was purified by column chromatography to give tert-butyl 3-(6-(4-(8-fluoro-2-methylimidazo[1,2-a]pyridin-6-yl)-2-(methoxymethoxy)phenyl)pyridazin-3-yl)azetidine-1-carboxylate (50.0 mg, 0.096 mmol, 15% yield).

### Step 6: 2-(6-(azetidin-3-yl)pyridazin-3-yl)-5-(8-fluoro-2-methylimidazo[1,2-a]pyridin-6-yl)phenol, HCl

Tert-butyl 3-(6-(4-(8-fluoro-2-methylimidazo[1.2-a]pyridin-6-yl)-2-(methoxymethoxy)phenyl)pyridazin-3-yl)azetidine-1-carboxylate (45 mg, 86.5 μmol) was suspended in 0.5 mL of dioxane/HCl (10%) and stirred for 12 h. Next, it was evaporated to dryness to give 2-(6-(azetidin-3-yl)pyridazin-3-yl)-5-(8-fluoro-2-methylimidazo[1,2-a]pyridin-6-yl)phenol (26 mg, 65.5 μmol, 76%). ¹H NMR (400 MHz, DMSO-d6) δ 2.44 (s, 3H), 4.35 (m, 4H), 4.44 (m, 1H), 7.44 (m, 2H), 7.96 (d, 1H), 8.06 (s, 1H), 8.11 (d, 1H), 8.20 (d, 1H), 8.60 (d, 1H), 9.04 (s, 1H), 9.14 (s, 1H), 9.30 (s, 1H). LC-HRMS calculated for C₂₁H₁₈FN₅O: m/z=375.15, found 374.1.0 (M−1).

### Example 14. 2-[6-(1-ethylazetidin-3-yl)pyridazin-3-yl]-5-(2-methyl-2H-indazol-5-yl)phenol (Compound 24A)

This Example shows the synthesis scheme for Compound 24 A.

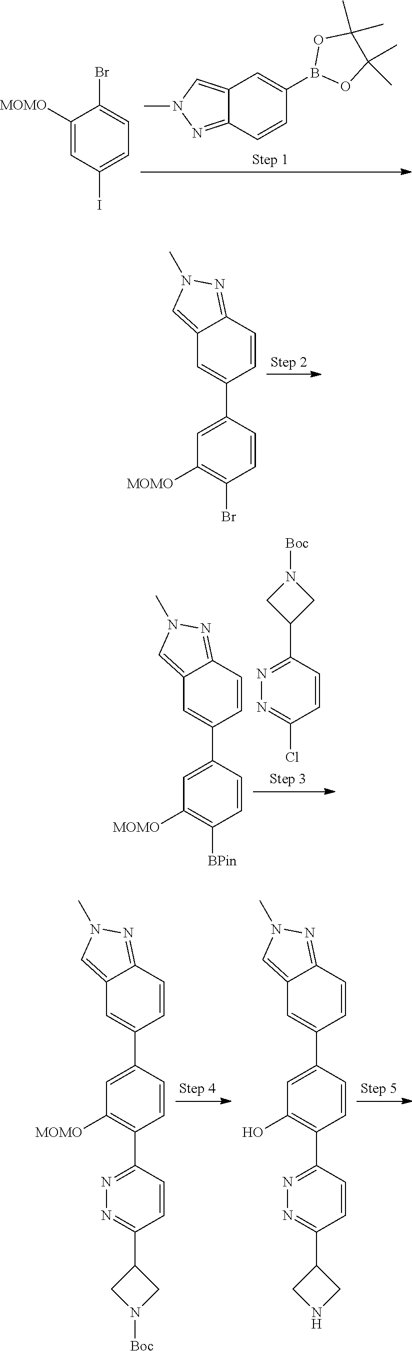

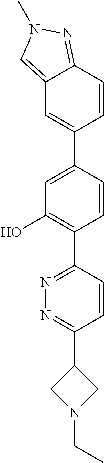 [SMILES: CCN1CC(c2ccc(-c3ccc(-c4ccc5nn(C)cc5c4)cc3O)nn2)C1]

### Step 1: Preparation of 5-(4-bromo-3-(methoxymethoxy)phenyl)-2-methyl-2H-indazole

A solution of 2-methyl-5-(tetramethyl-1,3,2-dioxaborolan-2-yl)-2H-indazole (500.33 mg, 1.94 mmol), 1-bromo-4-iodo-2-(methoxymethoxy)benzene (664.76 mg, 1.94 mmol), [1,1′-bis(diphenylphosphino)ferrocene]dichloropalladium (II) dichloromethane adduct (79.15 mg, 96.92 μmol), and potassium carbonate (535.78 mg, 3.88 mmol) in dioxane (10 mL) and water (2 nil) was degassed and purged with Ar. The resulting mixture was heated at 90° C. overnight. The reaction mixture was cooled to rt EtOAc (15 mL) was added and the mixture was filtered through a pad of Celite. The filtrate was concentrated in vacuo to give crude which was purified by flash column chromatography (Hex/EtOAc 3/1) to give 5-[4-bromo-3-(methoxymethoxy)phenyl]-2-methyl-2H-indazole (380.0 mg, Y: 50.8%). ESI-MS (M+H)+: 347.0 ¹H NMR (400 MHz, DMSO-d₆) δ 8.41 (s, 1H), 7.99 (s, 1H), 7.66 (dd, J=13.2, 8.6 Hz, 2H), 7.58-7.41 (m, 2H), 7.27 (dd, J=8.3, 2.1 Hz, 1H), 5.42 (s, 2H), 4.19 (s, 3H), 3.45 (s, 3H).

### Step 2: Preparation of 5-[3-(methoxymethoxy)-4-(4,4,5,5-tetramethyl-1,3,2-dioxaborolan-2-yl)phenyl]-2-methyl-2H-indazole

A solution of 5-[4-bromo-3-(methoxymethoxy)phenyl]-2-methyl-2H-indazole (380.0 mg, 1.09 mmol), 4,4,5,5-tetramethyl-2-(4,4,5,5-tetramethyl-1,3,2-dioxaborolan-2-yl)-1,3,2-dioxaborolane (278.24 mg, 1.1 mmol), [1,1′-bis(diphenylphosphino)ferrocene]dichloropalladium (II) dichloromethane adduct (89.48 mg, 109.57 mol), and potassium acetate (215.07 mg, 2.19 mmol) in TolH (10 mL) was degassed and purged with Ar. The resulting mixture was heated at 110° C. overnight. The reaction mixture was diluted with EtOAc (10 mL) and filtered. The filtrate was evaporated to give crude 5-[3-(methoxymethoxy)-4-(4.4.5.5-tetramethyl-1,3,2-dioxaborolan-2-yl)phenyl]-2-methyl-2H-indazole (430.0 mg, Y: 51.8%) which was used in the next step without purification. ESI-MS (M+H)+: 395.2.

### Step 3: Preparation of tert-butyl 3-6-[2-(methoxymethoxy)-4-(2-methyl-2H-indazol-5-yl)phenyl]pyridazin-3-ylazetidine-1-carboxylate

A solution of 5-[3-(methoxymethoxy)-4-(4,4,5,5-tetramethyl-1.3.2-dioxaborolan-2-yl)phenyl]-2-methyl-2H-indazole (432.0 mg, 1.1 mmol), tert-butyl 3-(6-chloropyridazin-3-yl)azetidine-1-carboxylate (295.77 mg, 1.1 mmol), [1,1′-bis(diphenylphosphino)ferrocene]dichloropalladium (II) dichloromethane adduct (44.77 mg, 54.83 μmol), and potassium carbonate (303.1 mg. 2.19 mmol) in dioxane (10 mL) and water (2 ml) was degassed and purged with Ar. The resulting mixture was heated at 90° C. overnight. The reaction mixture was cooled to rt diluted with MTBE (15 ml) and filtered through a pad of Celite. The filtrate was concentrated in vacuo to give crude, which was purified by preparative HPLC to give tert-butyl 3-6-[2-(methoxymethoxy)-4-(2-methyl-2H-indazol-5-yl)phenyl]pyridazin-3-ylazetidine-1-carboxylate (207.0 mg, Y: 37.6%). ESI-MS (M+H)+: 502.2.

### Step 4: Preparation of 2-[6-(azetidin-3-yl)pyridazin-3-yl]-5-(2-methyl-2H-indazol-5-yl)phenol trifluoroacetate

To a solution of tert-butyl 3-6-[2-(methoxymethoxy)-4-(2-methyl-2H-indazol-5-yl)phenyl]pyridazin-3-ylazetidine-1-carboxylate (207.47 mg, 413.63 μmol) in dichloromethane (5 mL) 2,2,2-trifluoroacetic acid (472.64 mg, 4.15 mmol, 320.0 pl) was added and the mixture was stirred overnight. Then the mixture was evaporated to dryness under reduced pressure to give crude 2-[6-(azetidin-3-yl)pyridazin-3-yl]-5-(2-methyl-2H-indazol-5-yl)phenol trifluoroacetate (135.0 mg, Y: 69.2%) which was used in the next step without purification. ESI-MS (M+H)+: 358.2

### Step 5: Preparation of 2-[6-(1-ethylazetidin-3-yl)pyridazin-3-yl]-5-(2-methyl-2H-indazol-5-yl)phenol trifluoroacetate

2-[6-(azetidin-3-yl)pyridazin-3-yl]-5-(2-methyl-2H-indazol-5-yl)phenol trifluoroacetate (134.02 mg, 284.29 mol) and acetaldehyde (124.8 mg, 2.83 mmol, 160.0 pl) in methanol was stirred at ambient temperature for 2 h. Sodium cyanoborohydride (89.33 mg, 1.42 mmol) was added, and the mixture was stirred at ambient temperature for 16 h. The reaction mixture was diluted with water and precipitate formed was filtered and purified by HPLC to afford the 2-[6-(1-ethylazetidin-3-yl)pyridazin-3-yl]-5-(2-methyl-2H-indazol-5-yl)phenol trifluoroacetate (2.2 mg, Y: 1.4%). ESI-MS (M+H)+: 386.2.

### Example 15. 5-(2-methyl-2H-indazol-5-yl)-2-{6-[1-(oxan-4-yl)azetidin-3-yl]pyridazin-3-yl}phenol (Compound 22A)

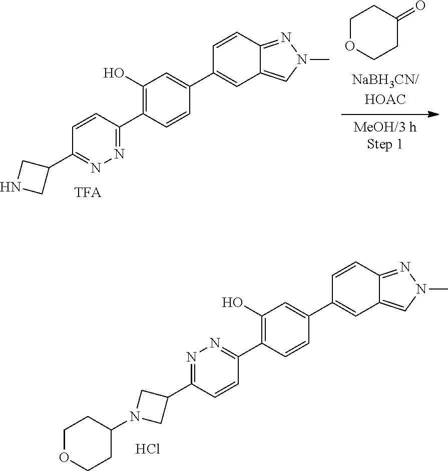

### Step 1: Preparation of 5-(2-methyl-2H-indazol-5-yl)-2-(6-(1-(tetrahydro-2H-pyran-4-yl)azetidin-3-yl)pyridazin-3-yl)phenol hydrochloride

To a solution of 2-(6-(azetidin-3-yl)pyridazin-3-yl)-5-(2-methyl-2H-indazol-5-yl)phenol (1.2 g, 3.36 mmol) in MeOH (100 mL) was added tetrahydro-4H-pyran-4-one (1.68 g, 16.807 mmol) and acetic acid (1 g, 16.807 mmol). The mixture was stirred at RT for 1 h, then sodium cyanoborohydride(635 mg, 10.08 mmol) was added under ice bath and the mixture was stirred at rt for 2 h, then water(10 mL) was added and the solution was concentrated in vacuo. The crude was purified by prep-HPLC (0.05% HCl in water/CH₃CN) to give title compound (657 mg, yield: 44.3%) as a solid. ESI-MS (M+H)+: 442.2, ¹H NMR (400 MHz, DMSO-d₆) δ 12.13-11.31 (m, 1H), 8.64-8.56 (m, 1H), 8.45 (s, 1H), 8.13-8.07 (m, 2H), 7.99 (d, J=9.1 Hz, 1H), 7.71 (d, J=9.0 Hz, 1H), 7.65-7.60 (m, 1H), 7.39-7.36 (m, 2H), 4.64-4.31 (m, 5H), 4.21 (s, 3H), 4.02-3.92 (m, 2H), 3.67-3.44 (m, 1H), 3.34-3.23 (m, 2H). 1.98-1.88 (m, 2H). 1.63-1.49 (m, 2H).

### Example 36. 5-(2,7-dimethyl-2H-indazol-5-yl)-2-[6-(1-ethylazetidin-3-yl)pyridazin-3-yl]phenol (Compound 34A)

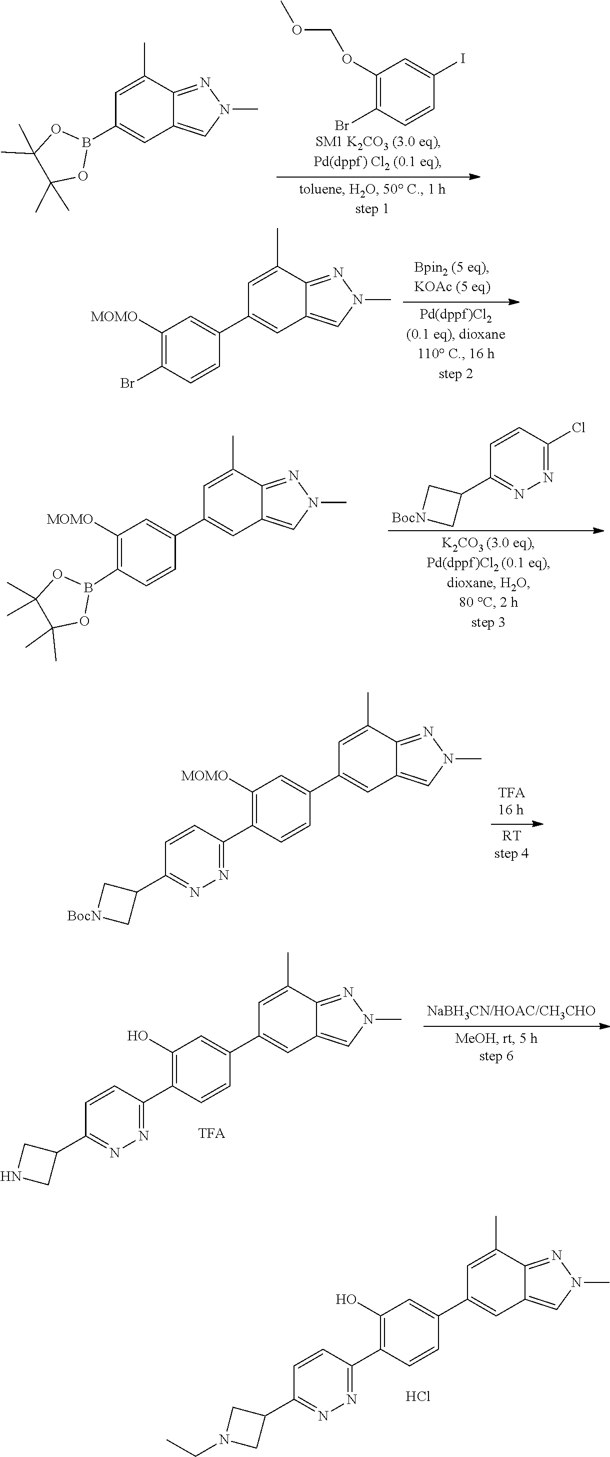

### Step 1: Preparation of 5-(4-bromo-3-(methoxymethoxy)phenyl)-2.7-dimethyl-2H-indazole

To a mixture of 2,7-dimethyl-5-(4,4,5,5-tetramethyl-1,3,2-dioxaborolan-2-yl)-2H-indazole (5.30 g, 19.48 mmol) and 1-bromo-4-iodo-2-(methoxymethoxy) benzene (6.60 g, 19.48 mmol) in dioxane: H2O (90 mL: 15 mL) were added Pd(dppf)Cl₂ (712 mg. 0.97 mmol) and K₂CO₃ (8.00 g, 58.34 mmol). The mixture was stirred at 50° C. for 1 h. LCMS showed the starting material was consumed completely. The mixture was filtered and the filtrate was concentrate in vacuo. The residue was purified by silica gel column chromatography (PE:EA=1:1) to give title product (4.0 g, Y: 58%) as a brown solid. ESI-MS (M+H+): 363.0. ¹H NMR (400 MHz, DMSO-d₆) δ 8.36 (s, 1H), 7.85-7.69 (d, J=13.9 Hz, 1H), 7.70-7.59 (s, 1H), 7.51-7.45 (s, 1H), 7.33 (s, 1H), 7.28-7.19 (m, 1H), 5.38 (s, 2H), 4.18 (s, 3H), 3.43 (s, 3H), 2.55 (s, 3H).

### Step 2: Preparation of 5-(3-(methoxymethoxy)-4-(4,4,5,5-tetramethyl-1, 3,2-dioxaborolan-2-yl)phenyl)-2, 7-dimethyl-2H-indazole

To a mixture of 5-(4-bromo-3-(methoxymethoxy)phenyl)-2.7-dimethyl-2H-indazole (4.00 g, 11.11 mmol) and B₂pin₂ (28.00 g, 111.11 mmol) in dioxane (200 mL) were added Pd(dppf)Cl₂ (406 mg, 0.55 mmol) and KOAc (8.70 g, 88.88 mmol). The mixture was stirred at 110° C. for 16 h. LCMS showed the starting material was consumed completely. The mixture was filtered and the filtrate was concentrate in vacuo. The residue was purified by Flash chromatography (PE:EA=1:1) to give title product (1.6 g, Y: 35.5%) as a brown solid. ESI-MS (M+H+): 409.0. ¹H NMR (400 MHz, CDCl₃) δ 7.91 (s, 1H), 7.76 (d, J=7.5 Hz, 1H), 7.68 (s, 1H), 7.31 (d, J=1.5 Hz, 1H), 7.29 (d, 1H), 7.26 (s, 1H), 5.28 (s, 2H). 4.25 (s, 3H), 3.56 (s. 3H), 2.68 (s, 3H), 1.37 (s, 12H).

### Step 3: Preparation of tert-butyl 3-(6-(4-(2, 7-dimethyl-2H-indazol-5-yl)-2-(methoxymethoxy)phenyl)pyridazin-3-yl)azetidine-1-carboxylate

To a mixture of 5-(3-(methoxymethoxy)-4-(4,4,5,5-tetramethyl-1,3,2-dioxaborolan-2-yl)phenyl)-2,7-dimethyl-2H-indazole (1.60 g, 3.92 mmol) and tert-butyl 3-(6-chloropyridazin-3-yl) azetidine-1-carboxylate (1.06 g, 3.92 mmol) in dioxane:H₂O (63 mL: 9 mL) were added Pd(dppf)Cl₂ (196 mg, 0.20 mmol) and K₂CO₃ (1.62 g, 11.76 mmol). The mixture was stirred at 80° C. for 2 h. LCMS showed the starting material was consumed completely. The mixture was filtered and the filtrate was concentrate in vacuo. The residue was purified by silica gel column chromatography (DCM:MeOH=40:1) to give title product (1.40 g, Y: 70%) as a yellow solid. ESI-MS (M+H+): 516.2. ¹H NMR (400 MHz, CDCl₃) δ 8.10-8.05 (m, 2H), 7.95 (d, J=4.1 Hz, 1H), 7.75-7.72 (m, 1H), 7.51 (d, J=8.8 Hz, 2H), 7.39-7.34 (m, 2H), 5.31 (s, 2H), 4.48-4.37 (m, 3H), 4.27 (s, 3H), 4.21-4.09 (m, 2H), 3.49 (s, 3H). 2.70 (s, 3H). 1.48 (s. 9H).

### Step 4: 2-(6-(azetidin-3-yl)pyridazin-3-yl)-5-(2-methyl-2H-pyrazolo[3,4-c]pyridin-5-yl)phenol hydrochloride

A mixture of tert-butyl 3-(6-(4-(2,7-dimethyl-2H-indazol-5-yl)-2-(methoxymethoxy)phenyl)pyridazin-3-yl)azetidine-1-carboxylate (1.30 g, 2.52 mmol) in TFA (15 mL) was stirred at rt for 16 h. LCMS showed the starting material was consumed completely. The mixture was concentrated in vacuo and the crude was purified by pre-HPLC to give title product (500 mg, Y: 54%) as a yellow solid. ESI-MS (M+H+): 372.1. ¹H NMR (400 MHz, MeOD-d₄) δ 9.02-8.88 (m, 2H), 8.51 (d, J=8.9 Hz, 1H), 8.17 (s, 1H), 8.00 (d, J=8.2 Hz, 1H), 7.91 (s, 1H), 7.53 (d, J=8.1 Hz, 1H), 7.47 (s, 1H), 4.75-4.67 (m, 1H), 4.63-4.54 (m, 4H), 4.47 (s, 3H), 2.72 (s, 3H).

### Step 5: Preparation of 5-(2, 7-dimethyl-2H-indazol-5-yl)-2-(6-(I-ethylazetidin-3-yl)pyridazin-3-yl)phenol hydrochloride

To a mixture of 2-(6-(azetidin-3-yl) pyridazin-3-yl)-5-(2, 8-dimethylimidazo [1,2-b]pyridazin-6-yl) phenol (500 mg, 1.35 mmol) and CH₃CHO (296 mg, 6.75 mmol) in MeOH (20 mL) and HOAc (500 mg, 8.34 mmol) were added NaBH₃CN (258 mg, 4.04 mmol). The mixture was stirred at RT for 5 h. LCMS showed the starting material was consumed completely. The mixture was concentrated in vacuo. The residue was purified by prep-HPLC (0.05% NH₃·H₂O in/CH₃CN) to give the solid. Dissolved in 0.05% HCl in water (20 mL) and lyophilized to give title product (185 mg, Y: 34.5%) as a yellow solid. ESI-MS (M+H+): 400.2. ¹H NMR (400 MHz, MeOD-d₄) δ 8.93-8.87 (m, 2H), 8.43 (t, J=8.1 Hz, 1H), 8.15 (s, 1H), 8.04-7.97 (m, 1H), 7.88 (s, 1H), 7.52 (d, J=8.3 Hz, 1H), 7.46 (s, 1H), 4.79-4.68 (m, 2H), 4.67-4.46 (m, 3H), 4.45 (s, 3H), 3.50-3.39 (m, 2H), 2.71 (s, 3H), 1.34-1.27 (m, 3H).

Sequence Table

| | SEQ | |
|---|---|---|
| Construct | ID | |
| Number | NO | Sequence |
| RS1-10 | 21 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGGAATTGTGTGAAA |
| | | TTTCAAATATTGGATTAGGAAATACAAAGTTACTGAAAGTGAGGTACTAATGTTTATAAA |
| | | ATAAAAACTTTTTCTTGCCATTTGCAGATTTAACATTTTTGGAGTCAATCCACACCATGG |
| | | AGGAGGTTCATGATTGTGTAGAGTAAGACATAATTTTGTTGAGGTTTAACTCTGAATACT |
| | | TAATGTGGTACTGAATACTTggTGTGGTACTGAGAGGCAGCCTAACTGACCACACAGCAT |
| | | TCACGCCTGGCTAATTTTTGTATTTTTAGTAGAGACGGGGTTTCACCATGGTGGCCAGGC |
| | | TGGGTTCTGGTTGTTTATGATCTTTATTTTTTGGTGATCTAGGAACCAACAACAAGAAAT |
| | | TGTTGTTTCCCGTGGG |
| RS1-11 | 22 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGGAATTGTGTGAAA |
| | | TTTCAAATATTGGATTAGGAAATACAAAGTTACTGAAAGTGAGGTACTAATGTTTATAAA |
| | | ATAAAAACTTTTTCTTGCCATTTGCAGATTTAACATTTTTGGAGTCAATCCAAGTAATGC |
| | | AGGAGGTTCATGATTGTGTAGAGTAAGACATAATTTTGTTGAGGTTTAACTCTGAATACT |
| | | TAATGTGGTACTGAATACTTAATGTGGTACTGAGAGGCAGCCTAACTGACCACACAGCAT |
| | | TCACGCCTGGCTAATTTTTGTATTTTTAGTAGAGACGGGGTTTCACCATGGTGGCCAGGC |
| | | TGGGTTCTGGTTGTTTATGATCTTTATTTTTTGGTGATCTAGGAACCAACAACAAGAAAT |
| | | TGTTGTTTCCCGTGGG |
| RS1-12 | 23 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGGAATTGTGTGAAA |
| | | TTTCAAATATTGGATTAGGAAATACAAAGTTACTGAAAGTGAGGTACTAATGTTTATAAA |
| | | ATAAAAACTTTTTCTTGCCATTTGCAGATTTAACATTTTTGGAGTCAATCCAAGTAACGC |
| | | AGGACACCATGGATTGTGTAGAGTAAGACATAATTTTGTTGAGGTTTAACTCTGAATACT |
| | | TAATGTGGTACTGAATACTTAATGTGGTACTGAGAGGCAGCCTAACTGACCACACAGCAT |
| | | TCACGCCTGGCTAATTTTTGTATTTTTAGTAGAGACGGGGTTTCACCATGGTGGCCAGGC |
| | | TGGGTTCTGGTTGTTTATGATCTTTATTTTTTGGTGATCTAGGAACCAACAACAAGAAAT |
| | | TGTTGTTTCCCGTGGG |
| RS1-13 | 24 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGGAATTGTGTGAAA |
| | | TTTCAAATATTGGATTAGGAAATACAAAGTTACTGAAAGTGAGGTACTAATGTTTATAAA |
| | | ATAAAAACTTTTTCTTGCCATTTGCAGATTTAACATTTTTGGAGTCAATCCAAGTAACGC |
| | | AGGAGGTTCACACCATGGTAGAGTAAGACATAATTTTGTTGAGGTTTAACTCTGAATACT |
| | | TAATGTGGTACTGAATACTTAATGTGGTACTGAGAGGCAGCCTAACTGACCACACAGCAT |
| | | TCACGCCTGGCTAATTTTTGTATTTTTAGTAGAGACGGGGTTTCACCATGGTGGCCAGGC |
| | | TGGGTTCTGGTTGTTTATGATCTTTATTTTTTGGTGATCTAGGAACCAACAACAAGAAAT |
| | | TGTTGTTTCCCGTGGG |
| RS1-14 | 25 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGGAATTGTGTGAAA |
| | | TTTCAAATATTGGATTAGGAAATACAAAGTTACTGAAAGTGAGGTACTAATGTTTATAAA |
| | | ATAAAAACTTTTTCTTGCCATTTGCAGATTTAACATTTTTGGAGTCAATCCACACCATGG |
| | | AGGAGGTTCATGAAAGGGATGAGTAAGCAGTCATTTTGTTGAGGTTTAACTCTGAATACT |
| | | TAATGTGGTACTGAATACTTGGTGTGGTACTGAGAGGCAGCCTAACTGACCACACAGCAT |
| | | TCACGCCTGGCTAATTTTTGTATTTTTAGTAGAGACGGGGTTTCACCATGGTGGCCAGGC |
| | | TGGGTTCTGGTTGTTTATGATCTTTATTTTTTGGTGATCTAGGAACCAACAACAAGAAAT |
| | | TGTTGTTTCCCGTGGG |
| RS1-1 | 26 | atgTTTCTGTACAACTTAACCTTGCAGAGAGCCACTGGCATCAGCTTTGCCATTCTTGGA |
| | | AACTTTTCTGGTAAGTTCTCTCGTTACCATCTTTTGAAATTTTAAGTGAATTAATACATA |
| | | TCTTGCTTAGTCTCTTGTGCAGGAAATGTTTTCCATTTATGACAAAACAGTTGTTGGTAA |
| | | ATGTAAACAATTCAAATTATGAGATGAGGTGTTAAGTTCTGGTGCATTATCTGTTACCTA |
| | | TTTCAGATGCATTTCCTAGTTCACAAATTGTGTAATGATTCTTGTCAGGGCACACTTTTC |
| | | TTGGCTGCTTACCTAGTGCCAAGTCGTGTGCCTATTGCTGTAAGACATACTGAGATGTGT |
| | | AAGACCTGATCTTTGATCTTGAATTTGACATCAGGTTGGGTAAAAAGAATGTCTGAAGGT |
| | | GTTTGTCTGGTTATCAGAGAGTAATTTAAATAAAGGGAAGGTTGGACCTTGGTTTGGTGC |
| | | TTGAAGAGATAGAACTTTTGTATGAGAGAAAAGGGAAAATGCAGTTTAGGTAGGGAGTGC |
| | | TGTCTGCATAGAAACAGATAATTTGCTTACGTTTACCATGTGGGGAATATTTTTGTAAAG |
| | | ATGGATTAAGGCTAGGTTTGAATTGTGTGAAATTTCAAATATTGGATTAGGAAATACAAA |
| | | GTTACTGAAAGTGAGGTACTAATGTTTATAAAATAAAAACTTTTTCTTGCCATTTGCAGA |
| | | TTTAACATTTTTGGAGTCAATCCAAGTAATGCAGGAGGTTCATGATTGTGTAGAGTAAGA |
| | | CATAATTTTGTTGAGGTTTAACTCTGAATACTTAATGTGGTACTGAATACTTAATGTGGT |
| | | ACTGAGAGGCAGCCTAACTGACCACACAGCATTCACGCCTGGCTAATTTTTGTATTTTTA |
| | | GTAGAGACGGGGTTTCACCATGGTGGCCAGGCTGGGTTCTGGTTGTTTATGATCTTTATT |
| | | TTTTGGTGATCTAGGAACCAACAACAAGAAATTGTTGTTTCCCGTGGG |
| RS1-2 | 27 | atgTTTCTGTACAACTTAACCTTGCAGAGAGCCACTGGCATCAGCTTTGCCATTCTTGGA |
| | | AACTTTTCTGGTAAGTTCTCTCGTTACCATCTTTTGAAATTTTAAGTGAATTAATACATA |
| | | TCTTGCTTAGTCTCTTGTGCAGGAAATGTTTTCCATTTATGACAAAACAGTTGTTGGTAA |
| | | ATGTAAACAATTCAAATTATGAGATGAGGTGTTAAGTTCTGGTGCATTATCTGTTACCTA |
| | | TTTCAGATGCATTTCCTAGTTCACAAATTGTGTAATGATTCTTGTCAGGGCACACTTTTC |
| | | TTGGCTGCTTACCTAGTGCCAAGTCGTGTGCCTATTGCTGTAAGACATACTGAGATGTGT |
| | | AAGACCTGATCTTTGATCTTGAATTTGACATCAGGTTGGGTAAAAAGAATGTCTGAAGGT |
| | | GTTTGTCTGGTTATCAGAGAGTAATTTAAATAAAGGGAAGGTTGGACCTTGGTTTGGTGC |
| | | TTGAAGAGATAGAACTTTTGTATGAGAGAAAAGGGAAAATGCAGTTTAGGTAGGGAGTGC |
| | | TGTCTGCATAGAAACAGATAATTTGCTTACGTTTACCATGTGGGGAATATTTTTGTAAAG |
| | | ATGGATTAAGGCTAGGTTTGAATTGTGTGAAATTTCAAATATTGGATTAGGAAATACAAA |
| | | GTTACTGAAAGTGAGGTACTAATGTTTATAAAATAAAAACTTTTTCTTGCCATTTGCAGA |
| | | TTTAACATTTTTGGAGTCAATCCAAGTAATGCAGGAGGTTCATGATTGTGTAGAGTAAGA |
| | | CATAATTTTGTTGAGGTTTAACTCTGAATACTTAATGTGGTACTGAATACTTAATGTGGT |
| | | ACTGAGAGGCAGCCTAACTGACCACACAGCATTCAGTGGCACAGTCTTGGCTCACAGCAA |
| | | CCTCTACCTCCTGGGTTCAAGCGATTCTCCTGCCTTAGCCTCCCGAGTAGCTGGGATTAC |
| | | AGGCATGTGCCACCACGCCTGGCTAATTTTTGTATTTTTAGTAGAGACGGGGTTTCACCA |
| | | TGGTGGCCAGGCTGGGTTCTGGTTGTTTATGATCTTTATTTTTTGGTGATCTAGGAACCA |
| | | ACAACAAGAAATTGTTGTTTCCCGTGGG |
| RS1-3 | 28 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGTTGTTGGTAAATG |
| | | TAAACAATTCAAATTATGAGATGAGGTGTTAAGTTCTGGTGCATTATCTGTTACCTATTT |
| | | CAGATGCATTTCCTAGTTCACAAATTGTGTAATGATTCTTGTCAGGGCACACTTTTCTTG |
| | | GCTGCTTACCTAGTGCCAAGTCGTGTGCCTATTGCTGTAAGACATACTGAGATGTGTAAG |
| | | ACCTGATCTTTGATCTTGAATTTGACATCAGGTTGGGTAAAAAGAATGTCTGAAGGTGTT |
| | | TGTCTGGTTATCAGAGAGTAATTTAAATAAAGGGAAGGTTGGACCTTGGTTTGGTGCTTG |
| | | AAGAGATAGAACTTTTGTATGAGAGAAAAGGGAAAATGCAGTTTAGGTAGGGAGTGCTGT |
| | | CTGCATAGAAACAGATAATTTGCTTACGTTTACCATGTGGGGAATATTTTTGTAAAGATG |
| | | GATTAAGGCTAGGTTTGAATTGTGTGAAATTTCAAATATTGGATTAGGAAATACAAAGTT |
| | | ACTGAAAGTGAGGTACTAATGTTTATAAAATAAAAACTTTTTCTTGCCATTTGCAGATTT |
| | | AACATTTTTGGAGTCAATCCAAGTAATGCAGGAGGTTCATGATTGTGTAGAGTAAGACAT |
| | | AATTTTGTTGAGGTTTAACTCTGAATACTTAATGTGGTACTGAATACTTAATGTGGTACT |
| | | GAGAGGCAGCCTAACTGACCACACAGCATTCACGCCTGGCTAATTTTTGTATTTTTAGTA |
| | | GAGACGGGGTTTCACCATGGTGGCCAGGCTGGGTTCTGGTTGTTTATGATCTTTATTTTT |
| | | TGGTGATCTAGGAACCAACAACAAGAAATTGTTGTTTCCCGTGGG |
| RS1-4 | 29 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGTTGTTGGTAAATG |
| | | TAAACAATTCAAATTATGAGATGAGGTGTTAAGTTCTGGTGCATTATCTGTTACCTATTT |
| | | CAGATGCATTTCCTAGTTCACAAATTGTGTAATGATTCTTGTCAGGGCACACTTTTCTTG |
| | | GCTGCTTACCTAGTGCCAAGTCGTGTGCCTATTGCTGTAAGACATACTGAGATGTGTAAG |
| | | ACCTGATCTTTGATCTTGAATTTGACATCAGGTTGGGTAAAAAGAATGTCTGAAGGTGTT |
| | | TGTCTGGTTATCAGAGAGTAATTTAAATAAAGGGAAGGTTGGACCTTGGTTTGGTGCTTG |
| | | AAGAGATAGAACTTTTGTATGAGAGAAAAGGGAAAATGCAGTTTAGGTAGGGAGTGCTGT |
| | | CTGCATAGAAACAGATAATTTGCTTACGTTTACCATGTGGGGAATATTTTTGTAAAGATG |
| | | GATTAAGGCTAGGTTTGAATTGTGTGAAATTTCAAATATTGGATTAGGAAATACAAAGTT |
| | | ACTGAAAGTGAGGTACTAATGTTTATAAAATAAAAACTTTTTCTTGCCATTTGCAGATTT |
| | | AACATTTTTGGAGTCAATCCACACCATGGAGGAGGTTCATGATTGTGTAGAGTAAGACAT |
| | | AATTTTGTTGAGGTTTAACTCTGAATACTTAATGTGGTACTGAATACTTAATGTGGTACT |
| | | GAGAGGCAGCCTAACTGACCACACAGCATTCACGCCTGGCTAATTTTTGTATTTTTAGTA |
| | | GAGACGGGGTTTCACCATGGTGGCCAGGCTGGGTTCTGGTTGTTTATGATCTTTATTTTT |
| | | TGGTGATCTAGGAACCAACAACAAGAAATTGTTGTTTCCCGTGGG |
| RS1-5 | 30 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGTTGTTGGTAAATG |
| | | TAAACAATTCAAATTATGAGATGAGGTGTTAAGTTCTGGTGCATTATCTGTTACCTATTT |
| | | CAGATGCATTTCCTAGTTCACAAATTGTGTAATGATTCTTGTCAGGGCACACTTTTCTTG |
| | | GCTGCTTACCTAGTGCCAAGTCGTGTGCCTATTGCTGTAAGACATACTGAGATGTGTAAG |
| | | ACCTGATCTTTGATCTTGAATTTGACATCAGGTTGGGTAAAAAGAATGTCTGAAGGTGTT |
| | | TGTCTGGTTATCAGAGAGTAATTTAAATAAAGGGAAGGTTGGACCTTGGTTTGGTGCTTG |
| | | AAGAGATAGAACTTTTGTATGAGAGAAAAGGGAAAATGCAGTTTAGGTAGGGAGTGCTGT |
| | | CTGCATAGAAACAGATAATTTGCTTACGTTTACCATGTGGGGAATATTTTTGTAAAGATG |
| | | GATTAAGGCTAGGTTTGAATTGTGTGAAATTTCAAATATTGGATTAGGAAATACAAAGTT |
| | | ACTGAAAGTGAGGTACTAATGTTTATAAAATAAAAACTTTTTCTTGCCATTTGCAGATTT |
| | | AACATTTTTGGAGTCAATCCACACCATGGAGGAGGTTCATGATTGTGTAGAGTAAGACAT |
| | | AATTTTGTTGAGGTTTAACTCTGAATACTTAATGTGGTACTGGCCAGGCTGGGTTCTGGT |
| | | TGTTTATGATCTTTATTTTTTGGTGATCTAGGAACCAACAACAAGAAATTGTTGTTTCCC |
| | | GTGGG |
| RS1-6 | 31 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGTTGTTGGTAAATG |
| | | TAAACAATTCAAATTATGAGATGAGGTGTTAAGTTCTGGTGCATTATCTGTTACCTATTT |
| | | CAGATGCATTTCCTAGTTCACAAATTGTGTAATGATTCTTGTCAGGGCACACTTTTCTTG |
| | | GCTGCTTACCTAGTGCCAAGTCGTGTGCCTATTGCTGTAAGACATACTGAGATGTGTAAG |
| | | ACCTGATCTTTGATCTTGAATTTGACATCAGGTTGGGTAAAAAGAATGTCTGAAGGTGTT |
| | | TGTCTGGTTATCAGAGAGTAATTTAAATAAAGGGAAGGTTGGACCTTGGTTTGGTGCTTG |
| | | AAGAGATAGAACTTTTGTATGAGAGAAAAGGGAAAATGCAGTTTAGGTAGGGAGTGCTGT |
| | | CTGCATAGAAACAGATAATTTGCTTACGTTTACCATGTGGGGAATATTTTTGTAAAGATG |
| | | GATTAAGGCTAGGTTTGAATTGTGTGAAATTTCAAATATTGGATTAGGAAATACAAAGTT |
| | | ACTGAAAGTGAGGTACTAATGTTTATAAAATAAAAACTTTTTCTTGCCATTTGCAGATTT |
| | | AACATTTTTGGAGTCAATCCACACCATGGAGGAGGTTCATGATTGTGTAGAGTAAGACAT |
| | | AATTTTGTTGAGGTTTAACTCTGAATACTTAATGTGGTACTTAGAGACGGGGTTTCACCA |
| | | TGGTGGCCAGGCTGGGTTCTGGTTGTTTATGATCTTTATTTTTTGGTGATCTAGGAACCA |
| | | ACAACAAGAAATTGTTGTTTCCCGTGGG |
| RS1-7 | 32 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATAGAA |
| | | TTGTGTGAAATTTCAAATATTGGATTAGGAAATACAAAGTTACTGAAAGTGAGGTACTAA |
| | | TGTTTATAAAATAAAAACTTTTTCTTGCCATTTGCAGATTTAACATTTTTGGAGTCAATC |
| | | CACACCATGGAGGAGGTTCATGATTGTGTAGAGTAAGACATAATTTTGTTGAGGTTTAAC |
| | | TCTGAATACTTAATGTGGTACTGAATACTTAATGTGGTACTGAGAGGCAGCCTAACTGAC |
| | | CACACAGCATTCACGCCTGGCTAATTTTTGTATTTTTAGTAGAGACGGGGTTTCACCATG |
| | | GTGGCCAGGCTGGGTTCTGGTTGTTTATGATCTTTATTTTTTGGTGATCTAGGAACCAAC |
| | | AACAAGAAATTGTTGTTTCCCGTGGG |
| RS1-8 | 33 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGTTGTTGGTAAATG |
| | | TAAACAATTCAAATTATGAGATGAGGTGTTAAGTTCTGGTGCATTATCTGTTACCTATTT |
| | | CAGATGCATTTCCTAGTTCACAAATTGTGTAATGATTCTTGTCAGGGCACACTTTTCTTG |
| | | GCTGCTTACCTAGTGCCAAGTCGTGTGCCTATTGCTGTAAGACATACTGAGATGTGTAAG |
| | | ACCTGATCTTTGATCTTGAATTTGACATCAGGTTGGGTAAAAAGAATGTCTGAAGGTGTT |
| | | TGTCTGGTTATCAGAGAGTAATTTAAATAAAGGGAAGGTTGGACCTTGGTTTGGTGCTTG |
| | | AAGAGATAGAACTTTTGTATGAGAGAAAAGGGAAAATGCAGTTTAGGTAGGGAGTGCTGT |
| | | CTGCATAGAAACAGATAATTTGCTTACGTTTACCATGTGGGGAATATTTTTGTAAAGATG |
| | | GATTAAGGCTAGGTTTGAATTGTGTGAAATTTCAAATATTGGATTAGGAAATACAAAGTT |
| | | ACTGAAAGTGAGGTACTAATGTTTATAAAATAAAAACTTTTTCTTGCCATTTGCAGATTT |
| | | AACACACACCATGGAGGTGTAGAGTAAGACATAATTTTGTTGAGGTTTAACTCTGAATAC |
| | | TTAATGTGGTACTGAATACTTAATGTGGTACTGAGAGGCAGCCTAACTGACCACACAGCA |
| | | TTCACGCCTGGCTAATTTTTGTATTTTTAGTAGAGACGGGGTTTCACCATGGTGGCCAGG |
| | | CTGGGTTCTGGTTGTTTATGATCTTTATTTTTTGGTGATCTAGGAACCAACAACAAGAAA |
| | | TTGTTGTTTCCCGTGGG |
| RS1-9 | 34 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGTTGTTGGTAAATG |
| | | TAAACAATTCAAATTATGAGATGAGGTGTTAAGTTCTGGTGCATTATCTGTTACCTATTT |
| | | CAGATGCATTTCCTAGTTCACAAATTGTGTAATGATTCTTGTCAGGGCACACTTTTCTTG |
| | | GCTGCTTACCTAGTGCCAAGTCGTGTGCCTATTGCTGTAAGACATACTGAGATGTGTAAG |
| | | ACCTGATCTTTGATCTTGAATTTGACATCAGGTTGGGTAAAAAGAATGTCTGAAGGTGTT |
| | | TGTCTGGTTATCAGAGAGTAATTTAAATAAAGGGAAGGTTGGACCTTGGTTTGGTGCTTG |
| | | AAGAGATAGAACTTTTGTATGAGAGAAAAGGGAAAATGCAGTTTAGGTAGGGAGTGCTGT |
| | | CTGCATAGAAACAGATAATTTGCTTACGTTTACCATGTGGGGAATATTTTTGTAAAGATG |
| | | GATTAAGGCTAGGTTTATAAAATGCTTAAATTATGGATAACATTTTCAATGAAATTAACT |
| | | ATATTTTCAAATGTTCTGCCTTTTTTCTCTGTTTCTTTTGCCCTTCCTGATCGAAGTCAT |
| | | GGGAGAGTAAGTCAATACTTTTTATGAGAATATATTGTCCTACTTGTATGTCTAAAGACC |
| | | ATGTTCTCGTTCTGCAGTTCAACGTTGTTGATTTTATTTCTTACATACGCCTGGCTAATT |
| | | TTTGTATTTTTAGTAGAGACGGGGTTTCACCATGGTGGCCAGGCTGGGTTCTGGTTGTTT |
| | | ATGATCTTTATTTTTTGGTGATCTAGGAACCAACAACAAGAAATTGTTGTTTCCCGTGGG |
| RS1-X1 | 35 | GGCTAGCGCTTTTCTGTACAACTTAACCTTGCAGAGAGCCACTGGCATCAGCTTTGCCAT |
| | | TCTTGGAAACTTTTCTGGTAAGTTCTCTCGTTACCATCTTTTGAAATTTTAAGTGAATTA |
| | | ATACATATCTTGCTTAGTCTCTTGTGCAGGAAATGTTTTCCATTTATGACAAAACAGGAA |
| | | TTGTGTGAAATTTCAAATATTGGATTAGGAAATACAAAGTTACTGAAAGTGAGGTACTAA |
| | | TGTTTATAAAATAAAAACTTTTTCTTGCCATTTGCAGATTTAACATTTTTGGAGTCAATC |
| | | CACACCATGGAGGAGGTTCATGATTGTGTAGAGTAAGACATAATTTTGTTGAGGTTTAAC |
| | | TCTGAATACTTAATGTGGTACTGAATACTTGGTGTGGTACTGAGAGGCAGCCTAACTGAC |
| | | CACACAGCATTAGTAGAGACGGGGTTTCACCATGGTGGCCAGGCTGGGTTCTGGTTGTTT |
| | | ATGATCTTTATTTTTTGGTGATCTAGGAACCAACAACAAGAAATTGTTGTTTCCCGTGGG |
| RS2-1 | 36 | ATGGTCTTCTTAAACACTTCCAGTAACCATGAAAACTCAGACTTGGAAATGCCTTCTTTA |
| | | ACTTCCACCCCCCTCATTGGTCACAAATTGACTGTTACAACACCATTTCATAGAGACCAG |
| | | ACTGTGAAAACTCAAAAGGAAAATACTGTGTAAGTCTTTGGTTCAGGAATAAAATGATTT |
| | | ATCTTATTTACTTATATTTAATTAATGGAATATAGTTCCCAGATGTCTGATCCACTTGTA |
| | | TGTGCATAATTTTACTGACCATTTATATATAATCATGTCTATTCCCACATTGAATATATC |
| | | TGTCCTGGGCATATTACAACTACCTACAGGTAGAAGATGGGAAGATAAACTAGATTCCTT |
| | | TTGTAACTTACTCCAAAATTTCCTATATATGCACAAGTAGTTTTCATAAAACTTTTCCAT |
| | | TCGGTTTGTTTGTTCCCTCCATAAGGCCTTCTTATGAAAGCCAGATTTTCAACCTCCTTT |
| | | CCTCTCTAATTGTAAGCTTCAGAATAAGAACGAGTATGGTTTCTTACCTAGTTTGTATTT |
| | | TTATTTAATTTTATTTGTTTATTATTTATTTATTTTTAGTTTGTATTTTTTAATCTTATT |
| | | TTTTCTTTTTGAATTTATGTGTAGATCGGACCTCTTCTCACATCCCCAGGAATATTATAT |
| | | GATTAGAAGCCAAGGGAtgagtaagcagTCATTTTTATTCAATAAAGCCTTAATCAATTC |
| | | AGAGAAATGGTAGAGAAAGTGTGAATTATCAGAAACCTTGAAAAGAAATGCACTGTTATT |
| | | TCGAAGTACAAAAAGTAAATTTTAAAAGTTTAACATGTTTTGTAGTATGGGCTCTGTTTT |
| | | CATGAATAGGGAAACCTGATTTGTAAATGACATCAGGTCTTCTTGATGCTCATAGTCTTA |
| | | TACAGATCATAGGCACTACATGTGTCTTCAGAGCCATTAAGGAAAGATGTTAAATCATTT |
| | | AATGCTTTTAGTCTTCGAGGTATAATATTTTGAAATATTTGAAAATATGTGATATGCATA |
| | | TTCTCTTTTTCTTAACGAATAACGTGATTAATCAGCACACCCCAATTCCATATCCGGAAC |
| | | AGAGTTTATTGTATTCAGTGGAAAAATGTTAAATGTAACAGGTAAAGCCATGTAGGGACA |
| | | TTTATTCTTTTGTATTGAGCCACTGCTTGTTTTCTTTTTAAAGTTTTAGAACCCCAGGG |
| RS2-2 | 37 | ATGGTCCCATTTCATAGAGACCAGACTGTGAAAACTCAAAAGGAAAATACTGTGTAAGTC |
| | | TTTGGTTCAGGAATAAAATGATTTATCTTATTTACTTATATTTAATTAATGGAATATAGT |
| | | TCCCAGATGTCTGATCCACTTGTATGTGCATAATACCTAGTTTGTATTTTTATTTAATTT |
| | | TATTTGTTTATTATTTATTTATTTTTAGTTTGTATTTTTTAATCTTATTTTTTCTTTTTG |
| | | AATTTATGTGTAGATCGGACCTCTTCTCACATCCCCAGGAATATTATATGATTAGAAGCC |
| | | AAGGGAtgagtaagcagTCATTTTTATTCAATAAAGCCTTAATCAATTCAGAGAAATGGT |
| | | AGAGAAAGTGTGAATTATCAGAAACCTTGAAAAGAAATGCACTGTTATTGTTTATTGTAT |
| | | TCAGTGGAAAAATGTTAAATGTAACAGGTAAAGCCATGTAGGGACATTTATTCTTTTGTA |
| | | TTGAGCCACTGCTTGTTTTCTTTTTAAAGTTTTAGAACCCCAG |
| RS2-3 | 38 | ATGGTCCCATTTCATAGAGACCAGACTGTGAAAACTCAAAAGGAAAATACTGTGTAAGTC |
| | | TTTGGTTCAGGAATAAAATGATTTATCTTATTTACTTATATTTAATTAATGGAATATAGT |
| | | TCCCAGATGTCTGATCCACTTGTATGTGCATAATCCTTTCCTCTCTAATTGTAAGCTTCA |
| | | GAATAAGAACGAGTATGGTTTCTTACCTAGTTTGTATTTTTATTTAATTTTATTTGTTTA |
| | | TTATTTATTTATTTTTAGTTTGTATTTTTTAATCTTATTTTTTCTTTTTGAATTTATGTG |
| | | TAGATCGGACCTCTTCTCACATCCCCAGGAATATTATATGATTAGAAGCCAAGGGAtgag |
| | | taagcagTCATTTTTATTCAATAAAGCCTTAATCAATTCAGAGAAATGGTAGAGAAAGTG |
| | | TGAATTATCAGAAACCTTGAAAAGAAATGCACTGTTATTAACGAATAACGTGATTAATCA |
| | | GCACACCCCAATTCCATATCCGGAACAGAGTTTATTGTATTCAGTGGAAAAATGTTAAAT |
| | | GTAACAGGTAAAGCCATGTAGGGACATTTATTCTTTTGTATTGAGCCACTGCTTGTTTTC |
| | | TTTTTAAAGTTTTAGAACCCCAG |
| RS2-4 | 39 | ATGGTCCCATTTCATAGAGACCAGACTGTGAAAACTCAAAAGGAAAATACTGTGTAAGTC |
| | | TTTGGTTCAGGAATAAAATGATTTATCTTATTTACTTATATTTAATTAATGGAATATAGT |
| | | TCCCAGATGTCTGATCCACTTGTATGTGCATAATCCTTTCCTCTCTAATTGTAAGCTTCA |
| | | GAATAAGAACGAGTATGGTTTCTTACCTAGTTTGTATTTTTATTTAATTTTATTTGTTTA |
| | | TTATTTATTTATTTTTAGTTTGTATTTTTTAATCTTATTTTTTCTTTTTGAATTTATGTG |
| | | TAGATCGGACCTCTTCTCACATCCCCAGGAATATTATATGATTAGAAGCCAAGGGAtgag |
| | | taagcagTCATTTTTATTCAATAAAGCCTTAATCAATTCAGAGAAATGGTAGAGAAAGTG |
| | | TGAATTATCAGAAACCTTGAAAAGAAATGCACTGTTATTAACGAATAACGTGATTAATCA |
| | | GCACACCCCAATTCCATATCCGGAACAGAGTTTATTGTATTCAGTGGAAAAATGTTAAAT |
| | | GTAACAGGTAAAGCCATGTAGGGACATTTATTCTTTTGTATTGAGCCACTGCTTGTTTTC |
| | | TTTTTAAAGTTTTAGAACCCCAGGGCTCGAGCGagggcaggggaagtcttctaacatgcg |
| | | gggacgtggaggaaaatcccggccca |
| RS2-5 | 40 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAagggAtgagtaagca |
| | | gTCATTTTTATTCAATAAAGCCTTAATCAATTCAGAGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTTTAGAACCCCAGGG |
| RS2-6 | 41 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAcaccAtgagtaagca |
| | | gTCATTTTTATTCAATAAAGCCTTAATCAATTCAGAGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTTTAGAACCCCAG |
| RS2-7 | 42 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAagggAtgagtaagca |
| | | gTCATTTTTATTCAATAAAGCCTTAATCAATTCAGAGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTGGCGGCTCGAGCGGCgTG |
| RS2-8 | 43 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAagggAtgagtaagca |
| | | gTCATTTTTATTCAATAAAGCCTTAATCAATTCAGAGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTTTAGGGGCTCGAGCGGCgTG |
| RS2-9 | 44 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAagggAtgagtaagca |
| | | gTCATTTTTATTCAATAAAGCCTTAATCAATTCAGAGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTTTAGAAGGGGCTCGAGCGGCgTG |
| RS2-10 | 45 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAagggAtgagtaagca |
| | | gTCATTTTTATTCAATAAAGCCTTAATCAATTCAGAGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTTTAGAACCCGGGGCTCGAGCGGCgTG |
| RS2-11 | 46 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAagggAtgagtaagca |
| | | gTCATTTTTATTCAATAAAGCCTTAATCAATTCAGAGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTTTAGAACCCCAGgTG |
| RS2-12 | 47 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAagggAtgagtaagca |
| | | gTCATTTTTATTCAATAAAGCCTTAATCAATTCAGAGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTgTGGGCTCGAGCGGCgTG |
| RS2-13 | 48 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAagggAtgagtaagca |
| | | gTCATTTTTATTCAATAAAGCCTTAATCAATTCAGAGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTTTAgTG |
| RS2-14 | 49 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAagggAtgagtaagca |
| | | gTCATTTTTATTCAATAAAGCCTTAATCAATTCAGAGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTTTAGAAgTG |
| RS2-15 | 50 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAagggAtgagtaagca |
| | | gTCATTTTTATTCAATAAAGCCTTAATCAATTCAGAGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTTTAGAACCCgTG |
| RS2-X1 | 51 | CCATTTCATAGAGACCAGACTGTGAAAACTCAAAAGGAAAATACTGTGTAAGTCTTTGGT |
| | | TCAGGAATAAAATGATTTATCTTATTTACTTATATTTAATTAATGGAATATAGTTCCCAG |
| | | ATGTCTGATCCACTTGTATGTGCATAATCCTTTCCTCTCTAATTGTAAGCTTCAGAATAA |
| | | GAACGAGTATGGTTTCTTACCTAGTTTGTATTTTTATTTAATTTTATTTGTTTATTATTT |
| | | ATTTATTTTTAGTTTGTATTTTTTAATCTTATTTTTTCTTTTTGAATTTATGTGTAGATC |
| | | GGACCTCTTCTCACATCCCCAGGAATATTATATGATTAGAAGCCAAGGGATGAGTAAGCA |
| | | GTCATTTTTATTCAATAAAGCCTTAATCAATTCCATGAAATGGTAGAGAAAGTGTGAATT |
| | | ATCAGAAACCTTGAAAAGAAATGCACTGTTATTAACGAATAACGTGATTAATCAGCACAC |
| | | CCCAATTCCATATCCGGAACAGAGTTTATTGTATTCAGTGGAAAAATGTTAAATGTAACA |
| | | GGTAAAGCCATGTAGGGACATTTATTCTTTTGTATTGAGCCACTGCTTGTTTTCTTTTTA |
| | | AAGTTTTAGAACCCCAGG |
| RS3-1 | 52 | ATGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTTTGTGA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTTGTCTTA |
| | | GTCTCAGGATGCAGGGG |
| RS3-2 | 53 | ACGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTTTGcGA |
| | | CTTAATTATTTTGGGGGCTGCTATGTTAAAGTGTGTcAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTTGTCTTA |
| | | GTCTCAGGATGCAGGGG |
| RS3-3 | 54 | ATGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTTTGTGA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTaTGtTCT |
| | | TAGTCTCAGGATGCAGGGG |
| RS3-4 | 55 | ATGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTTTGTGA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAtgcTTTCCCCCCTTGTCTTA |
| | | GTCTCAGGATGCAGGGG |
| RS3-5 | 56 | ATGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTTTGTGA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATGAGCTTTTCCCCCCTTGTCTTA |
| | | GTCTCAGGATGCAGGGG |
| RS3-6 | 57 | ATGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTTTGTGA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTATGG |
| | | GTTTTGTTTCTGTTGTTTTTGGTTTGTTTTGTTGTACTATCAGCTTTTCCCCCCTTGTCT |
| | | TAGTCTCAGGATGCAGGGG |
| RS3-7 | 58 | ATGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTTTGTGA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGATGGCTCTTGTAGG |
| | | TTTTGTTTCTGTTGTTTTTGGTTTGTTTTGTTGTACTATCAGCTTTTCCCCCCTTGTCTT |
| | | AGTCTCAGGATGCAGGGG |
| RS3-8 | 59 | ATGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTaaGTGA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTTGTCTTA |
| | | GTCTCAGGATGCAGGGG |
| RS3-9 | 60 | ATGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTaTGTGA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTTGTCTTA |
| | | GTCTCAGGATGCAGGGG |
| RS3-10 | 61 | ATGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTCAGAGTaAGTGA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTTGTCTTA |
| | | GTCTCAGGATGCAGGGG |
| RS3-11 | 62 | GGTTTACCATTGAGGTAAGAAAATGTTAGTCATCAACTTAAGTATAGAAATTTATAGGAG |
| | | CGATCCTAATACTTCTGGCTGGAATTAGTATTAAAGGGTAATGTATCAATATGCTTTATT |
| | | ATTTGAAATTAACAGTTGCTGAAATGTCAAACCATGCACTAATGGGAATTTTCTGTTTTT |
| | | CCCTTTAACAGTATTGGATATGACAGGGAAGATGAATTCACTATGAGTaTGTGACTTAAT |
| | | TATTTTGGGGGCTGCTATGTTAAAGTGTGTTAGTTTAAGAATACTAGTTCTGTCTGTAGC |
| | | TCTAATCCTCTGAGAGCTTGGGTTTCTAAATTATTCTGTGTTGCTCTTGTAGGTTTTGTT |
| | | TCTGTTGTTTTTGGTTTGTTTTGTTGTACTATCAGCTTTTCCCCCCTTGTCTTAGTCTCA |
| | | GGATGCAGGGG |
| RS3-12 | 63 | ATGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTaTGTCA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTTGTCTTA |
| | | GTCTCAGGATGCAGGGG |
| RS3-13 | 64 | ATGGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTaTGacA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTTGTCTTA |
| | | GTCTCAGGATGCAGGGG |
| RS3-14 | 65 | AgtGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACTATGAGTaTGacA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTTGTCTTA |
| | | GTCTCAGGATGCAGGGG |
| RS3-15 | 66 | AgtGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAATTCACCATGAGTaTGacA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTTGTCTTA |
| | | GTCTCAGGATGCAGGGG |
| RS3-16 | 67 | AgtGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGATGAAaTgACTATGAGTaTGacA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTTGTCTTA |
| | | GTCTCAGGATGCAGGGG |
| RS3-17 | 68 | AgtGTCGGTTTACCATTGAGGTAAGAAAATGTTAGTCATCAACTTAAGTATAGAAATTTA |
| | | TAGGAGCGATCCTAATACTTCTGGCTGGAATTAGTATTAAAGGGTAATGTATCAATATGC |
| | | TTTATTATTTGAAATTAACAGTTGCTGAAATGTCAAACCATGCACTAATGGGAATTTTCT |
| | | GTTTTTCCCTTTAACAGTATTGGATATGACAGGGAAGAcaccaTggCTATGAGTaTGacA |
| | | CTTAATTATTTTGGGGGCTGCTATGTTAAAGTGTGTTAGTTTAAGAATACTAGTTCTGTC |
| | | TGTAGCTCTAATCCTCTGAGAGCTTGGGTTTCTAAATTATTCTGTGTTGCTCTTGTAGGT |
| | | TTTGTTTCTGTTGTTTTTGGTTTGTTTTGTTGTACTATCAGCTTTTCCCCCCTTGTCTTA |
| | | GTCTCAGGATGCAGGGG |
| RS4-1 | 69 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGGAATTGTGTGAAA |
| | | TTTCAAATATTGGATTAGGAAATACAAAGTTACTGAAAGTGAGGTACTAATGTTTATAAA |
| | | ATAAAAACTTTTTCTTGCCATTTGCAGATTTAACATTTTTGGAGTCAATCCAAGTAAcGC |
| | | AGGAGGTTCATGATTGTGTAGAGTAAGACATAATTTTGTTGAGGTTTAACTCTGAATACT |
| | | TAATGTGGTACTGAATACTTGGTGTGGTACTGAGAGGCAGCCTAACTGACCACACAGCAT |
| | | TCACGCCTGGCTAATTTTTGTATTTTTAGTAGAGACGGGGTTTCACCATGGTGGCCAGGC |
| | | TGGGTTCTGGTTGTTTATGATCTTTATTTTTTGGTGATCTAGtggCCAACAACAAGAAAT |
| | | TGTTGTTTCCCGTG |
| RS4-2 | 70 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGGAATTGTGTGAAA |
| | | TTTCAAATATTGGATTAGGAAATACAAAGTTACTGAAAGTGAGGTACTAATGTTTATAAA |
| | | ATAAAAACTTTTTCTTGCCATTTGCAGATTTAACATTTTTGGAGTCAATCCAAGTAAcGC |
| | | AGGAGGTTCtTGATTGTGTAGAGTAAGACATAATTTTGTTGAGGTTTAACTCTGAATACT |
| | | TAATGTGGTACTGAATACTTGGTGTGGTACTGAGAGGCAGCCTAACTGACCACACAGCAT |
| | | TCACGCCTGGCTAATTTTTGTATTTTTAGTAGAGACGGGGTTTCACCATGGTGGCCAGGC |
| | | TGGGTTCTGGTTGTTTATGATCTTTATTTTTTGGTGATCTAGtggCCAACAACAAGAAAT |
| | | TGTTGTTTCCCGTG |
| RS4-3 | 71 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGGAATTGTGTGAAA |
| | | TTTCAAATATTGGATTAGGAAATACAAAGTTACTGAAAGTGAGGTACTAATGTTTATAAA |
| | | ATAAAAACTTTTTCTTGCCATTTGCAGATTTAACATTTTTGGAGTCAATCCAAGTAACGC |
| | | AGGAGGTTCTTGATTGTCAAGAGTAAGACATAATTTTGTTGAGGTTTAACTCTGAATACT |
| | | TAATGTGGTACTGAATACTTGGTGTGGTACTGAGAGGCAGCCTAACTGACCACACAGCAT |
| | | TCACGCCTGGCTAATTTTTGTATTTTTAGTAGAGACGGGGTTTCACCATGGTGGCCAGGC |
| | | TGGGTTCTGGTTGTTTATGATCTTTATTTTTTGGTGATCTAGtggCCAACAACAAGAAAT |
| | | TGTTGTTTCCCGTG |
| RS4-4 | 72 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGGAATTGTGTGAAA |
| | | TTTCAAATATTGGATTAGGAAATACAAAGTTACTGAAAGTGAGGTACTAATGTTTATAAA |
| | | ATAAAAACTTTTTCTTGCCATTTGCAGATTTAACATTTTTGGAGTCAATCCAAGTAAcGC |
| | | AGGAGGTTCtTGgccgccaAGAGTAAGACATAATTTTGTTGAGGTTTAACTCTGAATACT |
| | | TAATGTGGTACTGAATACTTggTGTGGTACTGAGAGGCAGCCTAACTGACCACACAGCAT |
| | | TCACGCCTGGCTAATTTTTGTATTTTTAGTAGAGACGGGGTTTCACCATGGTGGCCAGGC |
| | | TGGGTTCTGGTTGTTTATGATCTTTATTTTTTGGTGATCTAGtggCCAACAACAAGAAAT |
| | | TGTTGTTTCCCGTG |
| RS4-5 | 73 | TTTCTGTACAACTTAACCTTGCAGAGAGCCACTGGCATCAGCTTTGCCATTCTTGGAAAC |
| | | TTTTCTGGTAAGTTCTCTCGTTACCATCTTTTGAAATTTTAAGTGAATTAATACATATCT |
| | | TGCTTAGTCTCTTGTGCAGGAAATGTTTTCCATTTATGACAAAACAGGAATTGTGTGAAA |
| | | TTTCAAATATTGGATTAGGAAATACAAAGTTACTGAAAGTGAGGTACTAATGTTTATAAA |
| | | ATAAAAACTTTTTCTTGCCATTTGCAGATTTAACATTTTTGGAGTCAATCCAAGTAAcGC |
| | | AGGAGGTTCtTGgccgccaAGAGTAAGACATAATTTTGTTGAGGTTTAACTCTGAATACT |
| | | TAATGTGGTACTGAATACTTGGTGTGGTACTGAGAGGCAGCCTAACTGACCACACAGCAT |
| | | TCACGCCTGGCTAATTTTTGTATTTTTAGTAGAGACGGGGTTTCACCATGGTGGCCAGGC |
| | | TGGGTTCTGGTTGTTTATGATCTTTATTTTTTGGTGATCTAGtgg |
| Luciferase | 74 | GAAGATGCCAAAAACATTAAGAAGGGCCCAGCGCCATTCTACCCACTCGAAGACGGGACC |
| (DNA) | | GCCGGCGAGCAGCTGCACAAAGCCATGAAGCGCTACGCCCTGGTGCCCGGCACCATCGCCT |
| | | TTACCGACGCACATATCGAGGTGGACATTACCTACGCCGAGTACTTCGAGATGAGCGTTC |
| | | GGCTGGCAGAAGCTATGAAGCGCTATGGGCTGAATACAAACCATCGGATCGTGGTGTGCA |
| | | GCGAGAATAGCTTGCAGTTCTTCATGCCCGTGTTGGGTGCCCTGTTCATCGGTGTGGCTG |
| | | TGGCCCCAGCTAACGACATCTACAACGAGCGCGAGCTGCTGAACAGCATGGGCATCAGCC |
| | | AGCCCACCGTCGTATTCGTGAGCAAGAAAGGGCTGCAAAAGATCCTCAACGTGCAAAAGA |
| | | AGCTACCGATCATACAAAAGATCATCATCATGGATAGCAAGACCGACTACCAGGGCTTCC |
| | | AAAGCATGTACACCTTCGTGACTTCCCATTTGCCACCCGGCTTCAACGAGTACGACTTCG |
| | | TGCCCGAGAGCTTCGACCGGGACAAAACCATCGCCCTGATCATGAACAGTAGTGGCAGTA |
| | | CCGGATTGCCCAAGGGCGTAGCCCTACCGCACCGCACCGCTTGTGTCCGATTCAGTCATG |
| | | CCCGCGACCCCATCTTCGGCAACCAGATCATCCCCGACACCGCTATCCTCAGCGTGGTGC |
| | | CATTTCACCACGGCTTCGGCATGTTCACCACGCTGGGCTACTTGATCTGCGGCTTTCGGG |
| | | TCGTGCTCATGTACCGCTTCGAGGAGGAGCTATTCTTGCGCAGCTTGCAAGACTATAAGA |
| | | TTCAATCTGCCCTGCTGGTGCCCACACTATTTAGCTTCTTCGCTAAGAGCACTCTCATCG |
| | | ACAAGTACGACCTAAGCAACTTGCACGAGATCGCCAGCGGCGGGGCGCCGCTCAGCAAGG |
| | | AGGTAGGTGAGGCCGTGGCCAAACGCTTCCACCTACCAGGCATCCGCCAGGGCTACGGCC |
| | | TGACAGAAACAACCAGCGCCATTCTGATCACCCCCGAAGGGGACGACAAGCCTGGCGCAG |
| | | TAGGCAAGGTGGTGCCCTTCTTCGAGGCTAAGGTGGTGGACTTGGACACCGGTAAGACAC |
| | | TGGGTGTGAACCAGCGCGGCGAGCTGTGCGTCCGTGGCCCCATGATCATGAGCGGCTACG |
| | | TTAACAACCCCGAGGCTACAAACGCTCTCATCGACAAGGACGGCTGGCTGCACAGCGGCG |
| | | ACATCGCCTACTGGGACGAGGACGAGCACTTCTTCATCGTGGACCGGCTGAAGAGCCTGA |
| | | TCAAATACAAGGGCTACCAGGTAGCCCCAGCCGAACTGGAGAGCATCCTGCTGCAACACC |
| | | CCAACATCTTCGACGCCGGGGTCGCCGGCCTGCCCGACGACGATGCCGGCGAGCTGCCCG |
| | | CCGCAGTCGTCGTGCTGGAACACGGTAAAACCATGACCGAGAAGGAGATCGTGGACTATG |
| | | TGGCCAGCCAGGTTACAACCGCCAAGAAGCTGCGCGGTGGTGTTGTGTTCGTGGACGAGG |
| | | TGCCTAAAGGACTGACCGGCAAGTTGGACGCCCGCAAGATCCGCGAGATTCTCATTAAGG |
| | | CCAAGAAGGGCGGCAAGATCGCCGTGTAA |
| Luciferase | 75 | EDAKNIKKGPAPFYPLEDGTAGEQLHKAMKRYALVPGTIAFTDAHIEVDITYAEYFEMS |
| (Amino | | VRLAEAMKRYGLNTNHRIVVCSENSLQFFMPVLGALFIGVAVAPANDIYNERELLNSMGI |
| Acid) | | SQPTVVFVSKKGLQKILNVQKKLPIIQKIIIMDSKTDYQGFQSMYTFVTSHLPPGFNEYD |
| | | FVPESFDRDKTIALIMNSSGSTGLPKGVALPHRTACVRFSHARDPIFGNQIIPDTAILSV |
| | | VPFHHGFGMFTTLGYLICGFRVVLMYRFEEELFLRSLQDYKIQSALLVPTLFSFFAKSTL |
| | | IDKYDLSNLHEIASGGAPLSKEVGEAVAKRFHLPGIRQGYGLTETTSAILITPEGDDKPG |
| | | AVGKVVPFFEAKVVDLDTGKTLGVNQRGELCVRGPMIMSGYVNNPEATNALIDKDGWLHS |
| | | GDIAYWDEDEHFFIVDRLKSLIKYKGYQVAPAELESILLQHPNIFDAGVAGLPDDDAGEL |
| | | PAAVVVLEHGKTMTEKEIVDYVASQVTTAKKLRGGVVFVDEVPKGLTGKLDARKIREILI |
| | | KAKKGGKIAV |

What is claimed is:

1. A nucleic acid molecule comprising a minigene positioned immediately 5′ to a transgene, the minigene comprising:
(a) a first exon immediately 5′ to a first intron;
(b) a start codon; and
(c) a splice modulator binding site, the splice modulator binding site comprises the nucleic acid sequence of DGAGTDDGHV (SEQ ID NO: 82) or DGAGTDDNHV (SEQ ID NO: 83),
wherein D is A, G, or T; wherein R is A, or G; wherein N is A, C, G, or T; wherein H is A, C, or T; and wherein V is A, G, or C.

2. The nucleic acid molecule of claim 1, wherein the splice modulator binding site comprises the nucleic acid sequence of DGAGTRRGHV (SEQ ID NO: 1) or DGAGTRRNHV (SEQ ID NO: 2), wherein D is A, G, or T; wherein R is A or G; wherein N is A, C, G, or T; wherein H is A, C, or T; and wherein V is A, G, or C.

3. The nucleic acid molecule of claim 1, wherein the splice modulator binding site comprises the nucleic acid sequence of DGAGTTTGHV(SEQ ID NO: 84), wherein D is A, G, or T; wherein H is A, C, or T; and wherein V is A, G, or C.

4. The nucleic acid molecule of any one of claims 1-4, wherein the nucleic acid comprises the splice modulator binding site comprising any one of SEQ ID NOs: 75-81.

5. The nucleic acid molecule of any one of claims 1-4, wherein the nucleic acid comprises in 5′ to 3′ order the first exon comprising the start codon, the first intron, and the transgene.

6. The nucleic acid molecule of any one of claims 1-5, further comprising a stop codon.

7. The nucleic acid molecule of any one of claims 1-5, further comprising a second exon.

8. The nucleic acid molecule of claim 7, wherein the nucleic acid comprises in 5′ to 3′ order the first exon comprising the start codon, the second exon, and the transgene.

9. The nucleic acid molecule of claim 7, wherein the nucleic acid comprises in 5′ to 3′ order the first exon comprising the start codon, the first intron, the second, and the transgene.

10. The nucleic acid molecule of claim 7, wherein the nucleic acid comprises in 5′ to 3′ order the first exon, the second exon comprising the start codon, and the transgene.

11. The nucleic acid molecule of claim 7, wherein the nucleic acid comprises in 5′ to 3′ order the first exon, the second exon comprising the start codon, the first intron, and the transgene.

12. The nucleic acid molecule of claim 7, wherein the nucleic acid comprises in 5′ to 3′ order the first exon, the first intron, the second exon comprising the start codon, a second intron, and the transgene.

13. The nucleic acid molecule of claim 8, wherein the nucleic acid comprises at least about 80% sequence identity to any one of SEQ ID NOs: 21-73.

14. The nucleic acid molecule of claim 8, wherein the nucleic acid comprises at least about 90% sequence identity to any one of SEQ ID NOs: 21-73.

15. The nucleic acid molecule of claim 8, wherein the nucleic acid comprises any one of SEQ ID NOs: 21-73.

16. The nucleic acid molecule of any one of claims 6-15, further comprising a third exon.

17. The nucleic acid molecule of any one of claims 6-16, further comprising a third intron.

18. The nucleic acid molecule of claim 16, wherein the nucleic acid comprises in 5′ to 3′ order the first exon, the second exon comprising the stop codon, the third exon comprising the start codon, and the transgene.

19. The nucleic acid molecule of claim 17, wherein the nucleic acid comprises in 5′ to 3′ order the first exon, the first intron, the second exon comprising the stop codon, the second intron, the third exon comprising the start codon, the third intron, and the transgene.

20. The nucleic acid molecule of claim 16, wherein the nucleic acid comprises in 5′ to 3′ order the first exon, the first intron, the second exon comprising the stop codon, the third exon comprising the start codon, the second intron, and the transgene.

21. The nucleic acid molecule of claim 16, wherein the nucleic acid comprises in 5′ to 3′ order the first exon, the second exon comprising the start codon, the third exon comprising the stop codon, and the transgene.

22. The nucleic acid molecule of claim 16, wherein the nucleic acid comprises in 5′ to 3′ order the first exon, the first intron, the second exon comprising the start codon, the second intron, the third exon comprising the stop codon, the third intron, and the transgene.

23. The nucleic acid molecule of claim 16, wherein the nucleic acid comprises in 5′ to 3′ order the first exon, the first intron, the second exon comprising the start codon, the third exon comprising the stop codon, the second intron, and the transgene.

24. A nucleic acid molecule comprising a minigene positioned immediately 5′ to a transgene, the minigene comprising:
(a) a first exon 5′ to a first intron;
(b) a start codon comprising a first portion and a second portion, wherein the first portion and second portion of the start codon are not in the same exon, and
(c) a splice modulator binding site, In some embodiments, the splice modulator binding site comprises the nucleic acid sequence of DGAGTDDGHV (SEQ ID NO: 82) or DGAGTDDNHV (SEQ ID NO: 83), wherein D is A, G, or T; wherein R is A, or G; wherein N is A, C, G, or T; wherein H is A, C, or T; and wherein V is A, G, or C.

25. The nucleic acid molecule of claim 24, wherein the splice modulator binding site comprises the nucleic acid sequence of DGAGTRRGHV (SEQ ID NO: 1) or DGAGTRRNHV (SEQ ID NO: 2), wherein D is A, G, or T; wherein R is A or G; wherein N is A, C, G, or T;
wherein H is A, C, or T; and wherein V is A, G, or C.

26. The nucleic acid molecule of claim 24, wherein the splice modulator binding site comprises the nucleic acid sequence of DGAGTTTGHV (SEQ ID NO: 84), wherein D is A, G, or T; wherein H is A, C, or T; and wherein V is A, G, or C.

27. The nucleic acid molecule of any one of claims 24-26, wherein the first portion of the start codon comprises one or two nucleotides of the start codon, and the second portion of the start codon has one or two nucleotides of the start codon.

28. The nucleic acid molecule of any one of claims 24-27, wherein the first portion of the start codon is located in the first exon, and wherein the second portion of the start codon is located in the transgene.

29. The nucleic acid molecule of claim 28, wherein the nucleic acid comprises in 5′ to 3′ order the first exon comprising the first portion of the start codon, the first intron, and the transgene comprising the second portion of the start codon.

30. The nucleic acid molecule of any one of claims 24-29, further comprising a second exon.

31. The nucleic acid molecule of any one of claims 24-30, further comprising a second intron.

32. The nucleic acid molecule of claim 31, wherein the nucleic acid comprises in 5′ to 3′ order the first exon comprising the first portion of the start codon, the second exon comprising the second portion of the start codon, and the transgene.

33. The nucleic acid molecule of claim 31, wherein the nucleic acid comprises in 5′ to 3′ order the first exon comprising the first portion of the start codon, the first intron, the second exon comprising the second portion of the start codon, the second intron, and the transgene.

34. The nucleic acid molecule of claim 31, wherein the nucleic acid comprises in 5′ to 3′ order the first exon, the second exon comprising the first portion of the start codon, and the transgene comprising the second portion of the start codon.

35. The nucleic acid molecule of claim 31, wherein the nucleic acid comprises in 5′ to 3′ order the first exon, the second exon comprising the first portion of the start codon, the first intron, and the transgene comprising the second portion of the start codon.

36. The nucleic acid molecule of claim 31, wherein the nucleic acid comprises in 5′ to 3′ order the first exon comprising the first portion of the start codon, the second exon, and the transgene comprising the second portion of the start codon.

37. The nucleic acid molecule of claim 31, wherein the nucleic acid comprises in 5′ to 3′ order the first exon comprising the first portion of the start codon, the second exon, the first intron, and the transgene comprising the second portion of the start codon.

38. The nucleic acid molecule of claim 31, wherein the nucleic acid comprises in 5′ to 3′ order the first exon comprising the first portion of the start codon, the second exon comprising the second portion of the start codon, and the transgene.

39. The nucleic acid molecule of claim 31, wherein the nucleic acid comprises in 5′ to 3′ order the first exon comprising the first portion of the start codon, the first intron, the second exon comprising the second portion of the start codon, and the transgene.

40. The nucleic acid molecule of any one of claims 24-39, further comprising a stop codon.

41. The nucleic acid molecule of claim 40, wherein the nucleic acid comprises in 5′ to 3′ order the first exon comprising the first portion of the start codon, the second exon comprising the stop codon, and the transgene comprising the second portion of the start codon.

42. The nucleic acid molecule of claim 40, wherein the nucleic acid comprises in 5′ to 3′ order the first exon comprising the first portion of the start codon, the first intron, the second exon comprising the stop codon, and the transgene comprising the second portion of the start codon.

43. The nucleic acid molecule of any one of claims 30-42, wherein the second exon comprises the splice modulator binding site.

44. The nucleic acid molecule of any one of claims 30-43, further comprising a third exon.

45. The nucleic acid molecule of any one of claims 30-44, further comprising a third intron.

46. The nucleic acid molecule of claim 45, wherein the nucleic acid comprises in 5′ to 3′ order the first exon comprising the first portion of the start codon, the second exon comprising the stop codon, the third exon comprising the second portion of the start codon, and the transgene.

47. The nucleic acid molecule of claim 45, wherein the nucleic acid comprises in 5′ to 3′ order the first exon comprising the first portion of the start codon, the first intron, the second exon comprising the stop codon, the third exon comprising the second portion of the start codon, the second intron, and the transgene.

48. The nucleic acid molecule of claim 45, wherein the nucleic acid comprises in 5′ to 3′ order the first exon, the second exon comprising first portion of the start codon, the third exon comprising the stop codon, and the transgene comprising the second portion of the start codon.

49. The nucleic acid molecule of claim 45, wherein the nucleic acid comprises in 5′ to 3′ order the first exon, the first intron, the second exon comprising first portion of the start codon, the third exon comprising the stop codon, the second intron, and the transgene comprising the second portion of the start codon.

50. The nucleic acid molecule of claim 45, wherein the nucleic acid comprises in 5′ to 3′ order the first exon, the second exon comprising the stop codon, the third exon comprising first portion of the start codon, and the transgene comprising the second portion of the start codon.

51. The nucleic acid molecule of claim 45, wherein the nucleic acid comprises in 5′ to 3′ order the first exon, the first intron, the second exon comprising the stop codon, the third exon comprising first portion of the start codon, the second intron, and the transgene comprising the second portion of the start codon.

52. The nucleic acid molecule of claim 45, wherein the nucleic acid comprises in 5′ to 3′ order the first exon, the second exon, the third exon comprising the first portion of the start codon, and the transgene comprising the second portion of the start codon.

53. The nucleic acid molecule of claim 45, wherein the nucleic acid comprises in 5′ to 3′ order the first exon, the first intron, the second exon, the third exon comprising the first portion of the start codon, the second intron, and the transgene comprising the second portion of the start codon.

54. The nucleic acid molecule of claim 45, wherein the nucleic acid comprises in 5′ to 3′ order the first exon, the second exon comprising the stop codon, the third exon comprising the first portion of the start codon, and the transgene comprising the second portion of the start codon.

55. The nucleic acid molecule of claim 45, wherein the nucleic acid comprises in 5′ to 3′ order the first exon, the first intron, the second exon comprising the stop codon, the second intron, the third exon comprising the first portion of the start codon, the third intron, and the transgene comprising the second portion of the start codon.

56. The nucleic acid molecule of claim 45, wherein the nucleic acid comprises in 5′ to 3′ order the first exon, the second exon comprising the first portion of the start codon, the third exon comprising the stop codon, and the transgene comprising the second portion of the start codon.

57. The nucleic acid molecule of claim 45, wherein the nucleic acid comprises in 5′ to 3′ order the first exon, the first intron, the second exon comprising the first portion of the start codon, the second intron, the third exon comprising the stop codon, the third intron, and the transgene comprising the second portion of the start codon.

58. The nucleic acid molecule of any one of claims 24-58, wherein the first intron comprises the splice modulator binding site.

59. The nucleic acid molecule of any one of claims 24-58, wherein the splice modulator binding site is located at the junction between the second exon and the second intron.

60. The nucleic acid molecule of any one of claims 24-58, wherein the splice modulator binding site is located within the second intron.

61. A nucleic acid molecule comprising in 5′ to 3′:
(a) a first exon comprising a first portion of a start codon at the 3′ end of the first exon;
(b) a second exon comprising a second portion of a start codon at the 5′ end of the second exon; and
(c) a third exon.

62. The nucleic acid molecule of claim 61, wherein the third exon is a transgene.

63. A nucleic acid molecule comprising in 5′ to 3′ order a first exon, a second exon comprising a stop codon, a third exon comprising a first portion of a start codon, and a transgene comprising a second portion of the start codon.

64. A nucleic acid molecule comprising in 5′ to 3′ order a first exon, a second exon comprising a first portion of a start codon, a third exon comprising a stop codon, and a transgene comprising a second portion of the start codon.

65. The nucleic acid molecule of any one of claims 63-64, wherein the nucleic acid molecule further comprises a first intron, a second intron, and a third intron interposed between the first exon, the second exon, the third exon, and the transgene, respectively.

66. The nucleic acid molecule of any one of claims 1-65, wherein the splice modulator binding site does not comprise the nucleic acid sequence AAGAGT (SEQ ID NO: 3), ATGAGT (SEQ ID NO: 4), TAGAGT (SEQ ID NO: 5), TTGAGT (SEQ ID NO: 6), GAGAGT (SEQ ID NO: 7), GTGAGT (SEQ ID NO: 8), AAGAGT (SEQ ID NO: 9), ATGAGT (SEQ ID NO: 10), ACGAGT (SEQ ID NO: 11), AGGAGT (SEQ ID NO: 12), AGAGGTAGAG (SEQ ID NO: 13), TGAGGTTGAG (SEQ ID NO: 14), GGAGGTGGAG (SEQ ID NO: 15), TAG (SEQ ID NO: 16), CAG (SEQ ID NO: 17), TAG (SEQ ID NO: 18), or NAGAGTNNNN (SEQ ID NO: 19), wherein N is A, C, G, or T.

67. The nucleic acid molecule of any one of claims 1-66, wherein the splice modulator binding site comprises the splice modulator binding site comprising any one of SEQ ID NOs: 75-81.

68. A composition comprising the nucleic acid molecule of any one of claims 1-67 and a splice modulator, wherein the splice modulator comprises a compound of Formula (I): 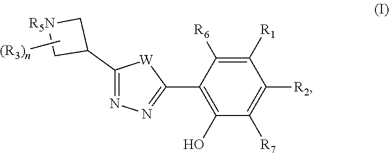
or a pharmaceutically acceptable salt, solvate, or prodrug thereof, wherein:
W is —S— or —HC═CH—;
R₁ is H, halogen, hydroxyl, cyano, C₁-C₆ alkyl, C₂-C₆ alkenyl, C₂-C₆ alkynyl, C₁-C₆ haloalkyl, C₁-C₆ alkoxyl, C₁-C₆ haloalkoxyl, —(CH₂)₀₋₂—C₃-C₈ cycloalkyl, NH₂, NH(C₁-C₆ alkyl), N(C₁-C₆ alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈ cycloalkyl, aryl, or 4- to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆ alkyl, C₂-C₆ alkenyl, C₂-C₆ alkynyl, C₁-C₆ alkoxyl, C₃-C₈ cycloalkyl, NH₂, NH(C₁-C₆ alkyl), or N(C₁-C₆ alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆ alkyl, C₂-C₆ alkenyl, C₂-C₆ alkynyl, C₁-C₆ haloalkyl, C₁-C₆ alkoxyl, C₁-C₆ haloalkoxyl, C₃-C₈ cycloalkyl, NH₂, NH(C₁-C₆ alkyl), N(C₁-C₆ alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆ alkyl), or N(C₁-C₆ alkyl)₂;
R₅ is H, C₁-C₆ alkyl, C₂-C₆ alkenyl, C₂-C₆ alkynyl, C₁-C₆ alkoxyl, C₃-C₈ cycloalkyl, —CH₂C₃-C₈ cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆ alkyl, C₁-C₆ haloalkyl, C₁-C₆ heteroalkyl, C₁-C₆ alkoxyl, C₃-C₈ cycloalkyl, spiro C₃-C₈ cycloalkyl, spiro 4-7 membered heterocyclyl, 5-6 membered heteroaryl, oxo, cyano, or hydroxyl;
R₆ is H, halogen, C₁-C₆ alkyl or C₁-C₆ haloalkyl:
R₇ is H, halogen, C₁-C₆ alkyl or C₁-C₆haloalkyl; and
nis 0, 1, 2, 3, 4, or 5.

69. A composition comprising:
(i) a nucleic acid molecule comprising a minigene positioned immediately 5′ to a transgene, the minigene comprising:
a) an exon positioned 5′ to an intron; and
b) a start codon having a first portion and a second portion, wherein the first portion and second portion of the start codon are not in the same exon;
c) a splice modulator binding site; and
(ii) a splice modulator that binds the splice modulator binding site and comprises a structure according to Formula (I): 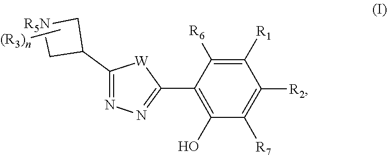
or a pharmaceutically acceptable salt, solvate, or prodrug thereof, wherein:
W is —S— or —HC═CH—;
R₁ is H, halogen, hydroxyl, cyano, C₁-C₆ alkyl, C₂-C₆ alkenyl, C₂-C₆ alkynyl, C₁-C₆ haloalkyl, C₁-C₆ alkoxyl, C₁-C₆ haloalkoxyl, —(CH₂)₀₋₂—C₃-C₈ cycloalkyl, NH₂, NH(C₁-C₆ alkyl), N(C₁-C₆ alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈ cycloalkyl, aryl, or 4- to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆ alkyl, C₂-C₆ alkenyl, C₂-C₆ alkynyl, C₁-C₆ alkoxyl, C₃-C₈ cycloalkyl, NH₂, NH(C₁-C₆ alkyl), or N(C₁-C₆ alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, C₁-C₆ alkyl, C₂-C₆ alkenyl, C₂-C₆ alkynyl, C₁-C₆haloalkyl, C₁-C₆ alkoxyl, C₁-C₆ haloalkoxyl, C₃-C₈ cycloalkyl, NH₂, NH(C₁-C₆ alkyl), N(C₁-C₆ alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆ alkyl), or N(C₁-C₆ alkyl)₂;
R₅ is H, C₁-C₆ alkyl, C₂-C₆ alkenyl, C₂-C₆ alkynyl, C₁-C₆ alkoxyl, C₃-C₈ cycloalkyl, —CH₂C₃-C₈ cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆ alkyl, C₁-C₆haloalkyl, C₁-C₆ heteroalkyl, C₁-C₆ alkoxyl, C₃-C₈ cycloalkyl, spiro C₃-C₈ cycloalkyl, spiro 4-7 membered heterocyclyl, 5-6 membered heteroaryl, oxo, cyano, or hydroxyl;
R₆ is H, halogen, C₁-C₆ alkyl or C₁-C₆ haloalkyl;
R₇ is H, halogen, C₁-C₆ alkyl or C₁-C₆ haloalkyl; and
n is 0, 1, 2, 3, 4, or 5.

70. A composition comprising:
(i) a nucleic acid molecule comprising a minigene positioned immediately 5′ to a transgene, the minigene comprising:
(a) a first exon immediately 5′ to a first intron:
(b) a start codon; and
(c) a splice modulator binding site;
(ii) a splice modulator that binds the splice modulator binding site and comprises a structure according to Formula (I): 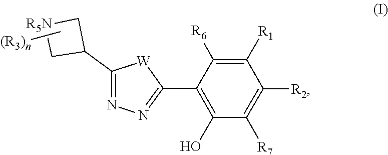
or a pharmaceutically acceptable salt, solvate, or prodrug thereof, wherein:
W is —S— or —HC═CH—;
R₁ is H, halogen, hydroxyl, cyano, C₁-C₆ alkyl, C₂-C₆ alkenyl, C₂-C₆ alkynyl, C₁-C₆ haloalkyl, C₁-C₆ alkoxyl, C₁-C₆ haloalkoxyl, —(CH₂)₀₋₂—C₃-C₈ cycloalkyl, NH₂, NH(C₁-C₆ alkyl), N(C₁-C₆ alkyl)₂, or —(CH₂)₀₋₂-heterocyclyl, wherein heterocyclyl is a 4- to 7-membered ring and comprises 1, 2, or 3 heteroatoms independently selected from N, O, and S, wherein the alkyl, alkenyl, alkynyl, alkoxyl, cycloalkyl, and heterocyclyl are optionally substituted with one or more C₃-C₈ cycloalkyl, aryl, or 4- to 7-membered heterocyclyl comprising 1, 2, or 3 heteroatoms independently selected from N, O, and S;
R₂ is aryl, 5- to 7-membered cycloalkyl, 5-, 6-, or 9-membered heterocyclyl comprising 1, 2, or 3 heteroatoms independently selected from N, O, and S, or 5-, 6-, or 9-membered heteroaryl comprising 2, 3, or 4 heteroatoms independently selected from N, O, and S, wherein the aryl, cycloalkyl, heterocyclyl, or heteroaryl is optionally substituted with one or more R₄;
each R₃ is independently halogen, C₁-C₆ alkyl, C₂-C₆ alkenyl, C₂-C₆ alkynyl, C₁-C₆ alkoxyl, C₃-C₈ cycloalkyl, NH₂, NH(C₁-C₆ alkyl), or N(C₁-C₆ alkyl)₂, wherein the alkyl, alkenyl, alkynyl, alkoxyl, and cycloalkyl are optionally substituted with one or more hydroxyl or NH₂;
each R₄ is independently halogen, hydroxyl, cyano, nitro, CL-C₆ alkyl, C₂-C₆ alkenyl, C₂-C₆ alknyl, C₁-C₆haloalkyl, CL-C₆ alkoxyl, C₁-C₆ haloalkoxyl, C₃-C₈ cycloalkyl, NH₂, NH(C₁-C₆ alkyl), N(C₁-C₆ alkyl)₂, or C(O)NH₂, wherein the alkyl, alkenyl, alkynyl, alkoxyl, and cycloalkyl are optionally substituted with one or more hydroxyl, 4- to 7-membered heterocyclyl comprising 1, 2, or 3 heteroatoms independently selected from N, O, and S, NH₂, NH(C₁-C₆ alkyl), or N(C₁-C₆ alkyl)₂;
R₅ is H, C₁-C₆ alkyl, C₂-C₆ alkenyl, C₂-C₆ alkynyl, C₁-C₆ alkoxyl, C₃-C₈ cycloalkyl, —CH₂C₃-C₈ cycloalkyl, heterocyclyl, —CH₂heterocycyl, —CH₂CH₂heterocycyl, —CH₂-(5-6 membered heteroaryl) wherein heterocyclyl is a 4- to 7-membered ring and comprises 1, 2, or 3 heteroatoms independently selected from N, O, and S, wherein R₅ is optionally substituted with one or more halogen, C₁-C₆ alkyl, C₁-C₆ haloalkyl, C₁-C₆ heteroalkyl, C₁-C₆ alkoxyl, C₃-C₈ cycloalkyl, spiro C₃-C₈ cycloalkyl, spiro 4-7 membered heterocyclyl, 5-6 membered heteroaryl, oxo, cyano, or hydroxyl:
R₆ is H, halogen, C₁-C₆ alkyl or CL-C₆haloalkyl;
R₇ is H, halogen, C₁-C₆ alkyl or C₁-C₆ haloalkyl; and
n is 0, 1, 2, 3,4, or 5.

71. A composition comprising:
(i) a nucleic acid molecule comprising a minigene positioned immediately 5′ to a transgene, the minigene comprising:
(a) a first exon immediately 5′ to a first intron;
(b) a start codon; and
(c) a splice modulator binding site:
(ii) a splice modulator that binds the splice modulator binding site and comprises a structure according to Formula (II): 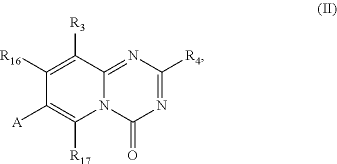
or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently selected from the group consisting of halogen, hydroxy, cyano, —COOH, —C(O)—C₁-C₆alkyl, —C(O)O—C₁-C₆alkyl, C₁-C₇alkyl, C₁-C₈heteroalkyl, C₁₋₇alkoxy-heterocycloalkyl, C₂-C₆alkenyl, C₂-C₆alkynyl, C₁-C₆alkoxy, —(CH₂)₀₋₂—C₃-C₈cycloalkyl, 4-7-membered monocyclic heterocycloalkyl, NH₂, NH(C₁-C₆alkyl), N(C₁-C₆alkyl)₂, —NHC(O)—C₁-C₆alkyl, —N(C₁-C₆alkyl)-C(O)—C₁-C₆alkyl, —C(O)—NH₂, —C(O)—NH(C₁-C₆alkyl), and —C(O)—N(C₁-C₆alkyl)₂, wherein the alkyl, alkenyl, alkynyl, and alkoxy are optionally substituted with one or more halogen, hydroxyl or NH₂, and wherein the cycloalkyl and heterocycloalkyl are optionally substituted with one or more halogen, hydroxyl, C₁-C₆alkyl, C₁-C₆heteroalkyl, C₁-C₆alkoxy, or NH₂; or
two R⁶ on the same carbon can be taken together as keto (═O); or
two R₆ together form C₁₋₇alkylene;
each R₇ is independently halo, cyano, C₁₋₇alkyl, C₁₋₂haloalkyl, C₁₋₇alkoxy, C₁₋₇haloalkoxy, or C₃₋₈cycloalkyl, wherein the C₁₋₇alkyl is optionally substituted with OH;
R₁₆ is H, halo, C₁₋₇alkyl, OR₅, N(R₅)₂, C₃₋₈cycloalkyl, or heterocycloalkyl; and
R₁₇ is H, halo, C₁₋₇alkyl, OR₅, N(R₅)₂, C₃₋₈cycloalkyl, or heterocycloalkyl.

72. A composition comprising:
(i) a nucleic acid molecule comprising a minigene positioned immediately 5′ to a transgene, the minigene comprising:
(a) a first exon immediately 5′ to a first intron:
(b) a start codon; and
(c) a splice modulator binding site;
(ii) a splice modulator that binds the splice modulator binding site and comprises a structure according to Formula (III): 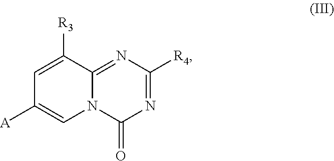
or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-CI-7alkyl, C₃₋₈cycloalkyl, heterocycloalkyl, or C₁₋₇alkoxy-heterocycloalkyl, or two R₆ together form C₁₋₇alkylene; and
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH.

73. The composition of any one of claims 69-72, wherein the splice modulator that binds the splice modulator binding site is selected from the group consisting of compounds TA-192 A and 100B-135B.

74. The composition of any one of claims 69-73, wherein the splice modulator that binds the splice modulator binding site is selected from the group comprising 3A, 6A, 8A, 10A, 15A, 24A, 86A, 100B, 11B, 117B, 121B, 135B, and 192 A.

75. The composition of any one of claims 69-73, wherein the splice modulator that binds the splice modulator binding site is selected from the group comprising 116B, 100B, 1A, 22A, 24A, 2A, and 34 A.

76. The composition of any one of claims 69-73, wherein the splice modulator binding site comprises the nucleic acid sequence of DGAGTDDGHV (SEQ ID NO: 82) or DGAGTDDNHV (SEQ ID NO: 83), wherein D is A, G, or T; wherein R is A, or G; wherein N is A, C, G, or T; wherein H is A, C, or T; and wherein V is A, G, or C.

77. The composition of claim 76, wherein the splice modulator binds to the splice modulator binding site comprising the nucleic acid sequence of DGAGTRRGHV (SEQ ID NO: 1) or DGAGTRRNHV (SEQ ID NO: 2), wherein D is A, G, or T; and wherein R is A, or G; and wherein N is A, C, G, or T; wherein H is A, C, or T; wherein V is A, G, or C.

78. The composition of claim 76, wherein the splice modulator binding site comprises the nucleic acid sequence of DGAGTTTGHV (SEQ ID NO: 84), wherein D is A, G, or T; wherein H is A, C, or T; and wherein V is A, G, or C.

79. The composition of any one of claim 76, wherein the splice modulator binding site does not comprise the nucleic acid sequence AAGAGT (SEQ ID NO: 3), ATGAGT (SEQ ID NO: 4), TAGAGT (SEQ ID NO: 5), TTGAGT (SEQ ID NO: 6), GAGAGT (SEQ ID NO: 7), GTGAGT (SEQ ID NO: 8), AAGAGT (SEQ ID NO: 9), ATGAGT (SEQ ID NO: 10), ACGAGT (SEQ ID NO: 11), AGGAGT (SEQ ID NO: 12), AGAGGTAGAG (SEQ ID NO: 13), TGAGGTTGAG (SEQ ID NO: 14), GGAGGTGGAG (SEQ ID NO: 15), TAG (SEQ ID NO: 16), CAG (SEQ ID NO: 17), TAG (SEQ ID NO: 18), or NAGAGTNNNN (SEQ ID NO: 19), wherein N is A, C, G, or T.

80. The composition of claim 79, wherein the splice modulator binds to the splice modulator binding site comprising a nucleic acid sequence of any one of SEQ ID NOs: 75-81.

81. The nucleic acid molecule of any one of claims 1-67 or the composition of any one of claims 68-80, wherein the transgene encodes a protein of interest.

82. The nucleic acid molecule of any one of claims 1-67 or the composition of any one of claims 68-80, wherein the transgene encodes a miRNA of interest.

83. The nucleic acid molecule of any one of claims 1-67 or the composition of any one of claims 68-80, wherein the transgene encodes a shRNA of interest.

84. The nucleic acid molecule of any one of claims 1-67 or the composition of any one of claims 68-80, wherein the transgene encodes a functional or regulatory RNA of interest.

85. The nucleic acid molecule of any one of claims 1-67 or the composition of any one of claims 68-80, wherein the nucleic acid molecule further comprises a promoter.

86. The nucleic acid molecule of any one of claims 1-67 or the composition of any one of claims 68-85, wherein the promoter is a GFAP promoter, Nestin promoter, S100B promoter, Nefh promoter, dystrophin promoter, H1 promoter, 7SK promoter, apolipoprotein E-human-alpha 1-antitrypsin promoter, CK8 promoter, mU1a, EF-1α promoter, TBG promoter, PKG promoter, CAG, the SV40 early promoter, murine mammary tumor virus LTR promoter, Ad MLP; HSV promoter, a CMV promoter such as CMV-IE, RSV promoter, U6 promoter or variants thereof, hSyn promoter, hexaribonucleotide binding protein-3 (NeuN) promoter, CaMKII promoter, Tα-1 promoter, neuron-specific enolase (NSE) promoter, PDGFβ promoter, VGLUT promoter, SST promoter, NPY promoter, VIP promoter, PV promoter, GAD65 or GAD67 promoter, promoter of DRD1 and DRD2, MAP1B, Clql2 promoter, POMC promoter, PROX1 promoter, or any suitable promoter.

87. The nucleic acid molecule of any one of claims 1-67 or the composition of any one of claims 68-80, wherein the molecule comprises a polyA, optionally wherein the polyA is an SV40 polyA, a HGH polyA, a BGH polyA, a beta-globin polyA, an alpha-globin polyA, an ovalbumin polyA, a kappa-light chain polyA, a synthetic polyA, or any suitable polyA.

88. A vector comprising the nucleic acid molecule of any one of claims 1-67 or the composition of any one of claims 68-80, optionally wherein the vector is a plasmid, a DNA vector, an RNA vector, a virion, or a viral vector.

89. The vector of claim 88, wherein the vector is a viral vector.

90. The vector of claim 89, wherein the viral vector is an adeno-associated virus (AAV), lentivirus, adenovirus, simian virus 40, vaccinia virus, measles virus, herpes virus, or poxvirus.

91. The vector of claim 90, wherein the viral vector is an AAV.

92. The vector of claim 91, wherein the AAV comprises capsid proteins from an AAV serotype selected from the group consisting of AAV1, AAV2, AAV3, AAV4, AAV5, AAV6, AAV7, AAV8, AAV9, AAVrh10, and AAVrh74.

93. The vector of any one of claims 91-92, wherein the AAV is a pseudotyped AAV.

94. A pharmaceutical composition comprising the nucleic acid molecule of claims 1-67 or the composition of any one of claims 68-80, or the vector of any one of claims 88-93 and a pharmaceutically acceptable carrier, diluent, or excipient.

95. A method of modulating the expression of a protein, RNA, or other biomolecule in a subject in need thereof, the method comprising administering to the subject a therapeutically effective amount of the nucleic acid molecule of any one of claims 1-67 or the composition of any one of claims 68-80, or the vector of any one of claims 88-93 or the pharmaceutical composition of claim 94.

96. The method of claim 95, wherein the method further comprises administering to the subject a therapeutically effective amount of a splice modulator.

97. The method of claim 95, wherein the protein is expressed in the presence of the splice modulator.

98. The method of any one of claims 96-97, wherein administering to the subject a therapeutically effective amount of the splice modulator causes an inclusion of one of the two or more exons, one or more exons, the first exon, or the second exon.

99. The method of claim 98, wherein one of the two or more exons or one of the one or more exons is the second exon.

100. The method of any one of claims 96-99, wherein the splice modulator binds to an RNA binding protein and/or a segment of the splice modulator binding site of the nucleic acid of any one of claims 1-67.
